(19) United States
(12) Patent Application Publication
(10) Pub. No.: US 2025/0270304 A1
(43) Pub. Date: Aug. 28, 2025

(54) ANTI-ACTIVIN ANTIBODIES AND METHODS OF USING THE SAME

(71) Applicant: PHENOMIC AI, Toronto (CA)

(72) Inventors: Sam COOPER, Toronto (CA); Christopher HARVEY, Toronto (CA); Arif JETHA, Toronto (CA); Max LONDON, Toronto (CA); Allison NIXON, Toronto (CA); Elizabeth KOCH, Toronto (CA); Mike BRISKIN, Boston, MA (US)

(21) Appl. No.: 18/269,539

(22) PCT Filed: Dec. 23, 2021
(86) PCT No.: PCT/US21/65079

§ 371 Date: Jun. 23, 2023

Related U.S. Application Data
(60) Provisional application No. 63/129,947, filed on Dec. 23, 2020.

(51) Int. Cl. C07K 16/22; A61K 35/17; A61K 39/00; A61K 40/11; A61K 40/15; A61K 40/17; A61K 40/31; A61K 40/42; A61K 47/68; A61P 35/00

(52) U.S. Cl. CPC C07K 16/22; A61K 35/17; A61K 40/11; A61K 40/15; A61K 40/17; A61K 40/31; A61K 40/4225; A61K 47/6845; A61P 35/00; A61K 2039/505; A61K 2239/13; C07K 2317/33; C07K 2317/565; C07K 2317/569; C07K 2317/622

(57) ABSTRACT

The present invention is directed to anti-activin antibodies, compositions comprising the same, and methods of using such antibodies and compositions for the prevention, diagnosis and treatment of a disease or disorder, such as, e.g., cancer, bone disease, cachexia, fibrotic disease, immune disorders, muscle atrophy, neurological diseases, renal diseases, reproductive diseases, and sarcopenia.

### SEQUENCE LISTING

The instant application contains a Sequence Listing which has been submitted electronically in ASCII format and is hereby incorporated by reference in its entirety. Said ASCII copy, created on Feb. 10, 2022, is named PHN-002-PCT_SL.txt and is 259,255 bytes in size.

### FIELD OF THE INVENTION

The present invention relates to antibodies and antibody domains that specifically bind to TGF-β family proteins, to compositions thereof, and methods of using the same.

### BACKGROUND OF THE INVENTION

Activins are members of the TGF-β family that functions in cell proliferation, differentiation, apoptosis, immune responses, and wound healing, including playing an important role in regulating the menstrual cycle by stimulating secretion of Follicle Stimulating Hormone (FSH). Inhibins are closely related TGF-β family proteins which share beta-subunits with Activins; however, Inhibins can function directly opposite to Activins, such as by inhibiting FSH synthesis and release.

Activins are homo- or heterodimers of Inhibin subunits, e.g., InhibinβA, InhibinβB, InhibinβC and InhibinβE in different combinations (Bloise, E. et al., Physiol Rev 99:739-780 (2019)). Inhibins are heterodimers composed of an alpha subunit and a β subunit e.g., InhibinβA, InhibinβB, InhibinβC or InhibinβE. Thus, Activins and Inhibins have nearly identical structures despite having different and often opposing functions. The structural similarity but antagnostic actions of Activin and Inhibin, e.g., toward the hypothalamo-pituitary-gonadal axis, render specificity in selectively targeting Activin but not Inhibin paramount for the most parsimonious therapeutic interventions that minimize cross-reactivity and off-target effects.

Activin A is a homodimer of INHBA (InhibinβA), which is initially generated with a prodomain region immediately followed by a mature domain. Similar to TGF-beta, Activin A is secreted as a precursor complex (“proprotein” form) and is processed extracellularly to remove the prodomain and release the mature and active form of Activin A (Wang et al., Nat Commun 7:12052 (2016)). INHBA can also form heterodimers with either INHA (Inhibin-alpha) to generate Inhibin A or INHBB (InhibinβB) to generate Activin A-B. Activin B is a homodimer of INHBB (InhibinβB), and INHBB can also form heterodimers with INHA to generate Inhibin B. Activin C is a homodimer of INHBC (InhibinβC) which has antagonistic activity to that of Activin A, and Activin E is a homodimer of INHBE (InhibinβE).

Activin A can be categorized as hormone, a growth factor, and a cytokine. Canonical Activin A signaling involves binding to one of its cognate type II receptors (ACVR2A/B), which then recruit the type I activin receptors (ALK4/7) to initiate downstream signaling that is mediated in a stepwise manner by i) the phosphorylation of SMAD2/3 proteins, ii) assembly with SMAD4 to generate the SMAD2/3/4 complex, and iii) nuclear translocation of the complex and subsequent transcriptional regulation (Namwanje et al., Cold Spring Harb Perspect Biol 8: a021881 (2016)). This is in contrast to Inhibin A which binds to the same activin type II receptors but uses beta-glycan as its co-receptor as opposed to ALK4/7. As a result, Inhibin antagonizes activin signaling by sequestering the type II activin receptors. Therefore, a proper balance in the expression of activins and inhibins is thought to play an important role in regulating signaling.

There is a need to therapeutically target Activin in various diseases, such as atrophy, bone diseases, cachexia, cancer, fibrotic disease, immune disorders, neurological diseases, renal diseases, reproductive diseases, and sarcopenia. Antibodies which bind and inhibit Activin A have been described (WO2014121221; WO2015017576) but any cross-reactivity and functional effects of such antibodies to Inhibins and/or other Activin(s) are not clear. It is unclear which anti-Activin antibodies might have Activin specificity (e.g., from one or more of Activin-A, A-B, and/or C) and which might inhibit both Activin(s) and Inhibin(s) (e.g., Inhibin-A or -B). Moreover, the potential therapeutic consequences of these varying specificities is also unknown and entirely uncertain.

### SUMMARY OF THE INVENTION

The present invention addresses the foregoing shortcomings in the prior art via the provision and characterization of a range of antibodies to Activins, as well as more informed and therefore efficacious methods of using the same, for example in the prevention, diagnosis, and treatment of cancer. As demonstrated herein for the first time, a more thorough understanding of specificity and functional effects of various anti-Activin antibodies is important in the context of cancer, and particularly in cancers where both Activin A and B are upregulated. In the preferred embodiments disclosed herein, anti-Activin antibodies capable of targeting both Activin A and Activin B are provided. Without being bound by theory, such antibodies may reduce the risk of tumor escape mechanisms and improve cancer treatment, and particularly in cancers where both Activin A and Activin B are upregulated.

In one aspect, the invention provides anti-activin antibodies that bind both Activin A and Activin B. In embodiments, the anti-activin antibodies bind latent Activin A and/or active Activin A, and Activin B.

In one embodiment, the anti-activin antibodies that bind both Activin A and Activin B comprise a heavy chain variable region comprising an amino acid sequence selected from the group consisting of: SEQ ID NOs: 39, 41, 43, 45, 47, 49, 63, 65, and 69.

In one embodiment, the anti-activin antibodies that bind both Activin A and Activin B comprise a light chain variable region comprising an amino acid sequence selected from the group consisting of SEQ ID NOs: 40, 42, 44, 46, 48, 50, 64, 66, and 70.

In one embodiment, the anti-activin antibodies that bind both Activin A and Activin B comprise a heavy chain variable region comprising a CDR1 sequence selected from the group consisting of SEQ ID NOs: 229-234, 241-242, and 244; a CDR2 sequence selected from the group consisting of SEQ ID NOs: 264-269, 276-277, and 279; and a CDR3 sequence selected from the group consisting of SEQ ID NOs: 299-304, 311-312, and 314.

In one embodiment, the anti-activin antibodies that bind both Activin A and Activin B comprise a light chain variable region comprising a CDR1 sequence selected from the group consisting of SEQ ID NOs: 334-339, 346-347, and 349; a CDR2 sequence selected from the group consisting of SEQ ID NOs: 369-374, 381-382, and 384; and a CDR3 sequence selected from the group consisting of SEQ ID NOs: 404-409, 416-417, and 419.

In one embodiment, the anti-activin antibodies that bind both Activin A and Activin B comprise a heavy chain variable region comprising a CDR1 sequence selected from the group consisting of SEQ ID NOs: 439-444, 452, and 454; a CDR2 sequence selected from the group consisting of SEQ ID NOs: 474-479, 487, and 489; and a CDR3 sequence selected from the group consisting of SEQ ID NOs: 509-514, 522, and 524.

In one embodiment, the anti-activin antibodies that bind both Activin A and Activin B comprise a light chain variable region comprising a CDR1 sequence selected from the group consisting of SEQ ID NOs: 544-549, 556-557, and 559; a CDR2 sequence selected from the group consisting of SEQ ID NOs: 80-85, 92-93, and 95; and a CDR3 sequence selected from the group consisting of SEQ ID NOs: 185-190, 197-198, and 209.

In one embodiment, the anti-activin antibodies that bind both Activin A and Activin B comprise a heavy chain variable region comprising SEQ ID NO: 39 and a light chain variable region comprising SEQ ID NO: 40.

In one embodiment, the anti-activin antibodies that bind both Activin A and Activin B comprise a heavy chain variable region comprising SEQ ID NO: 41 and a light chain variable region comprising SEQ ID NO: 42.

In one embodiment, the anti-activin antibodies that bind both Activin A and Activin B comprise a heavy chain variable region comprising SEQ ID NO: 43 and a light chain variable region comprising SEQ ID NO: 44.

In one embodiment, the anti-activin antibodies that bind both Activin A and Activin B comprise a heavy chain variable region comprising SEQ ID NO: 45 and a light chain variable region comprising SEQ ID NO: 46.

In one embodiment, the anti-activin antibodies that bind both Activin A and Activin B comprise a heavy chain variable region comprising SEQ ID NO: 47 and a light chain variable region comprising SEQ ID NO: 48.

In one embodiment, the anti-activin antibodies that bind both Activin A and Activin B comprise a heavy chain variable region comprising SEQ ID NO: 49 and a light chain variable region comprising SEQ ID NO: 50.

In one embodiment, the anti-activin antibodies that bind both Activin A and Activin B comprise a heavy chain variable region comprising SEQ ID NO: 63 and a light chain variable region comprising SEQ ID NO: 64.

In one embodiment, the anti-activin antibodies that bind both Activin A and Activin B comprise a heavy chain variable region comprising SEQ ID NO: 65 and a light chain variable region comprising SEQ ID NO: 66.

In one embodiment, the anti-activin antibodies that bind both Activin A and Activin B comprise a heavy chain variable region comprising SEQ ID NO:69 and a light chain variable region comprising SEQ ID NO:70.

In one embodiment, the invention provides an anti-activin antibody that competes with an antibody comprising a heavy chain variable region comprising SEQ ID NO: 39, 41, 43, 45, 47, 49, 63, 65, or 69, and a light chain variable region comprising SEQ ID NO: 40, 42, 44, 46, 48, 50, 64, 66, or 70 for binding to an activin and/or inhibin epitope.

In embodiments, anti-activin antibodies of the invention bind to human activin. In preferred embodiments the anti-activin antibody binds to both human Activin A and human

Activin B. In further embodiments, the anti-activin antibody binds latent human Activin A and active human Activin A.

In one embodiment, the anti-activin antibodies of the invention bind Inhibin A. In further embodiments, the anti-activin antibodies bind human Inhibin A.

Anti-activin antibodies of the invention include, for example, monoclonal antibodies, antibody fragments, including Fab, Fab′, F(ab′)2, and Fv fragments, single-chain antibodies, diabodies, single domain antibodies, chimeric antibodies, humanized antibodies and antibodies that competitively inhibit the binding of an antibody comprising a heavy chain variable region comprising SEQ ID NO: 71 or 73 and a light chain variable region comprising SEQ ID NO: 72 or 74 to an activin tumor epitope, and/or a heavy chain variable region comprising SEQ ID NO: 39, 41, 43, 45, 47, 49, 63, 65, or 69, and a light chain variable region comprising SEQ ID NO: 40, 42, 44, 46, 48, 50, 64, 66, or 70 for binding to an activin tumor epitope. In preferred embodiments, the activin tumor epitope is an epitope shared between Activin A and Activin B.

In one embodiment, the activin tumor epitope comprises a post-translational modification of an activin polypeptide. In one embodiment, the post-translational modification of the activin polypeptide comprises a sialylated O-glycosyl moiety. In one embodiment, the post-translational modification of the activin polypeptide comprises an O-linked glycan moiety that is linked to activin. In one embodiment, the post-translational modification of the activin polypeptide comprises a glycan moiety comprising beta-N-acetyl-galactosamine. In one embodiment, the beta-N-acetyl-galactosamine is a terminal beta-N-acetyl-galactosamine.

Accordingly, in one embodiment, an anti-activin antibody of the invention binds to a moiety that is a post-translational modification of activin. In one embodiment, an anti-activin antibody of the invention binds to a sialylated O-glycosyl moiety attached to activin. In one embodiment, an anti-activin antibody of the invention binds to an O-linked glycan moiety that is linked to activin. In one embodiment, an anti-activin antibody of the invention binds to a glycan moiety that is linked to activin, wherein the glycan moiety has at its terminus beta-N-acetyl-galactosamine.

In one embodiment, the invention provides an anti-activin antibody that competes with follistatin for binding to an activin and/or inhibin epitope.

In one embodiment, an anti-activin antibody comprises a heavy chain variable region comprising an amino acid sequence selected from the group consisting of Table I or Table III (below).

In one embodiment, an anti-activin antibody comprises a light chain variable region comprising the amino acid sequence selected from the group consisting of Table II or Table IV (below).

In one embodiment, an anti-activin antibody comprises a heavy chain variable region comprising a CDR1 selected from Table I; a CDR2 selected from Table I; and a CDR3 selected from Table I.

In one embodiment, an anti-activin antibody comprises a heavy chain variable region comprising a CDR1 selected from Table III; a CDR2 selected from Table III; and a CDR3 selected from Table III.

In one embodiment, an anti-activin antibody comprises a light chain variable region comprising a CDR1 selected from Table II; a CDR2 selected from Table II; and a CDR3 selected from Table II.

In one embodiment, an anti-activin antibody comprises a light chain variable region comprising a CDR1 selected from Table IV; a CDR2 selected from Table IV; and a CDR3 selected from Table IV.

In one embodiment, an anti-activin antibody comprises a heavy chain variable region comprising a CDR1 selected from Table I; a CDR2 selected from Table I; and a CDR3 selected from Table I; and further comprises a light chain variable region comprising a CDR1 selected from Table II; a CDR2 selected from Table II; and a CDR3 selected from Table II.

In one embodiment, an anti-activin antibody comprises a heavy chain variable region comprising a CDR1 selected from Table III; a CDR2 selected from Table III; and a CDR3 selected from Table III; and further comprises a light chain variable region comprising a CDR1 selected from Table IV; a CDR2 selected from Table IV; and a CDR3 selected from Table IV.

In one embodiment, an anti-activin antibody comprises a heavy chain variable region comprising any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, and 69.

In one embodiment, an anti-activin antibody comprises a light chain variable region comprising any one of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, and 70.

In one embodiment, an anti-activin antibody comprises a heavy chain variable region comprising SEQ ID NO: 1 and a light chain variable region comprising SEQ ID NO: 2.

In one embodiment, an anti-activin antibody comprises a heavy chain variable region comprising SEQ ID NO: 3 and a light chain variable region comprising SEQ ID NO: 4.

In one embodiment, an anti-activin antibody comprises a heavy chain variable region comprising SEQ ID NO: 5 and a light chain variable region comprising SEQ ID NO: 6.

In one embodiment, an anti-activin antibody comprises a heavy chain variable region comprising SEQ ID NO: 7 and a light chain variable region comprising SEQ ID NO: 8. In one embodiment, an anti-activin antibody comprises a heavy chain variable region comprising SEQ ID NO: 9 and a light chain variable region comprising SEQ ID NO: 10. In one embodiment, an anti-activin antibody comprises a heavy chain variable region comprising SEQ ID NO: 11 and a light chain variable region comprising SEQ ID NO: 12.

In one embodiment, an anti-activin antibody comprises a heavy chain variable region comprising SEQ ID NO: 13 and a light chain variable region comprising SEQ ID NO: 14. In one embodiment, an anti-activin antibody comprises a heavy chain variable region comprising SEQ ID NO: 15 and a light chain variable region comprising SEQ ID NO: 16. In one embodiment, an anti-activin antibody comprises a heavy chain variable region comprising SEQ ID NO: 17 and a light chain variable region comprising SEQ ID NO: 18.

In one embodiment, an anti-activin antibody comprises a heavy chain variable region comprising SEQ ID NO: 19 and a light chain variable region comprising SEQ ID NO: 20. In one embodiment, an anti-activin antibody comprises a heavy chain variable region comprising SEQ ID NO: 21 and a light chain variable region comprising SEQ ID NO: 22. In one embodiment, an anti-activin antibody comprises a heavy chain variable region comprising SEQ ID NO: 23 and a light chain variable region comprising SEQ ID NO: 24.

In one embodiment, an anti-activin antibody comprises a heavy chain variable region comprising SEQ ID NO: 25 and a light chain variable region comprising SEQ ID NO: 26. In one embodiment, an anti-activin antibody comprises a heavy chain variable region comprising SEQ ID NO: 27 and a light chain variable region comprising SEQ ID NO: 28.

In one embodiment, an anti-activin antibody comprises a heavy chain variable region comprising SEQ ID NO: 29 and a light chain variable region comprising SEQ ID NO: 30.

In one embodiment, an anti-activin antibody comprises a heavy chain variable region comprising SEQ ID NO: 31 and a light chain variable region comprising SEQ ID NO: 32. In one embodiment, an anti-activin antibody comprises a heavy chain variable region comprising SEQ ID NO: 33 and a light chain variable region comprising SEQ ID NO: 34. In one embodiment, an anti-activin antibody comprises a heavy chain variable region comprising SEQ ID NO: 35 and a light chain variable region comprising SEQ ID NO: 36.

In one embodiment, an anti-activin antibody comprises a heavy chain variable region comprising SEQ ID NO: 37 and a light chain variable region comprising SEQ ID NO: 38.

In one embodiment, an anti-activin antibody comprises a heavy chain variable region comprising SEQ ID NO: 39 and a light chain variable region comprising SEQ ID NO: 40.

In one embodiment, an anti-activin antibody comprises a heavy chain variable region comprising SEQ ID NO: 41 and a light chain variable region comprising SEQ ID NO: 42. In one embodiment, an anti-activin antibody comprises a heavy chain variable region comprising SEQ ID NO: 43 and a light chain variable region comprising SEQ ID NO: 44. In one embodiment, an anti-activin antibody comprises a heavy chain variable region comprising SEQ ID NO: 45 and a light chain variable region comprising SEQ ID NO: 46.

In one embodiment, an anti-activin antibody comprises a heavy chain variable region comprising SEQ ID NO: 47 and a light chain variable region comprising SEQ ID NO: 48. In one embodiment, an anti-activin antibody comprises a heavy chain variable region comprising SEQ ID NO: 49 and a light chain variable region comprising SEQ ID NO: 50. In one embodiment, an anti-activin antibody comprises a heavy chain variable region comprising SEQ ID NO: 51 and a light chain variable region comprising SEQ ID NO: 52.

In one embodiment, an anti-activin antibody comprises a heavy chain variable region comprising SEQ ID NO: 53 and a light chain variable region comprising SEQ ID NO: 54. In one embodiment, an anti-activin antibody comprises a heavy chain variable region comprising SEQ ID NO: 55 and a light chain variable region comprising SEQ ID NO: 56. In one embodiment, an anti-activin antibody comprises a heavy chain variable region comprising SEQ ID NO: 57 and a light chain variable region comprising SEQ ID NO: 58.

In one embodiment, an anti-activin antibody comprises a heavy chain variable region comprising SEQ ID NO: 59 and a light chain variable region comprising SEQ ID NO: 60. In one embodiment, an anti-activin antibody comprises a heavy chain variable region comprising SEQ ID NO: 61 and a light chain variable region comprising SEQ ID NO: 62. In one embodiment, an anti-activin antibody comprises a heavy chain variable region comprising SEQ ID NO: 63 and a light chain variable region comprising SEQ ID NO: 64.

In one embodiment, an anti-activin antibody comprises a heavy chain variable region comprising SEQ ID NO: 65 and a light chain variable region comprising SEQ ID NO: 66. In one embodiment, an anti-activin antibody comprises a heavy chain variable region comprising SEQ ID NO: 67 and a light chain variable region comprising SEQ ID NO: 68.

In one embodiment, an anti-activin antibody comprises a heavy chain variable region comprising SEQ ID NO: 69 and a light chain variable region comprising SEQ ID NO: 70.

In one embodiment, an anti-activin antibody comprises SEQ ID NO: 75 or 77 and SEQ ID NO: 76.

In one embodiment, an anti-activin antibody inhibits or neutralizes one or more human Activin A functions, optionally further inhibiting or neutralizing one or more human Activin B functions. In further embodiments, the anti-activin antibody is capable of inducing SMAD nuclear localization and/or fibroblast activation.

In one embodiment, an anti-activin antibody is a chimeric, humanized, or human antibody.

In one embodiment, an anti-activin antibody is a monoclonal antibody.

In one embodiment, an anti-activin antibody is an antibody fragment.

In one embodiment, the anti-activin antibody is an antibody fragment.

In one embodiment, the anti-activin antibody is a single-chain variable fragment.

In one embodiment, the anti-activin antibody is an antibody-drug conjugate (ADC).

In one aspect, the invention provides a CAR modified immune cell, such as a CAR-T or CAR-NK cell, or a CAR-macrophage, comprising a chimeric antigen receptor capable of binding to the activin tumor epitope.

In one aspect, the invention provides a CAR modified immune cell, such as a CAR-T or CAR-NK cell, or a CAR-macrophage, comprising a chimeric antigen receptor, wherein the chimeric antigen receptor comprises a light chain variable region of an anti-activin antibody and a heavy chain variable region of an anti-activin antibody.

In one aspect, the invention provides a CAR modified immune cell, such as a CAR-T or CAR-NK cell, or a CAR-macrophage, comprising an anti-activin antibody. In one embodiment, the anti-activin antibody is an antibody fragment. In one embodiment, the anti-activin antibody is an scFv.

In one aspect, the invention provides a method of inhibiting the growth of a cell that displays the activin tumor epitope, comprising contacting the cell with an anti-activin antibody or CAR modified immune cell, such as a CAR-T or CAR-NK cell, or a CAR-macrophage, of the invention.

In one aspect, the invention provides a method of inhibiting the proliferation of a cell that displays the activin tumor epitope, comprising contacting the cell with an anti-activin antibody or CAR modified immune cell, such as a CAR-T or CAR-NK cell, or a CAR-macrophage, of the invention. In one embodiment, the anti-activin antibody is used in the form of an ADC.

In one aspect, the invention provides a method of inducing death of a cell that displays the activin tumor epitope, comprising contacting the cell with an anti-activin antibody or CAR modified immune cell, such as a CAR-T or CAR-NK cell, or a CAR-macrophage, of the invention. In one embodiment, the anti-activin antibody is used in the form of an ADC.

In one aspect, the invention provides a method of inhibiting delamination of a cell that displays the activin tumor epitope, comprising contacting the cell with an anti-activin antibody or CAR modified immune cell, such as a CAR-T or CAR-NK cell, or a CAR-macrophage, of the invention. In one embodiment, the anti-activin antibody is used in the form of an ADC.

In one aspect, the invention provides a method of inhibiting vascularization of a tumor comprising a cell that displays the activin tumor epitope, comprising contacting the cell with an anti-activin antibody or CAR modified immune cell, such as a CAR-T or CAR-NK cell, or a CAR-macrophage, of the invention. In one embodiment, the anti-activin antibody is used in the form of an ADC.

In certain embodiments of the methods of the invention, the cell displaying the activin tumor epitope is a cancer cell.

In one aspect, the invention provides a method for treating a subject having cancer, comprising administering to the subject an effective amount of an anti-activin antibody or CAR modified immune cell, such as a CAR-T or CAR-NK cell, or a CAR-macrophage, of the invention. In one embodiment, the anti-activin antibody is used in the form of an ADC.

In one aspect, the invention provides a method of inhibiting tumor metastasis in a subject having cancer, comprising administering to the subject an effective amount of an anti-activin antibody or CAR modified immune cell, such as a CAR-T or CAR-NK cell, or a CAR-macrophage, of the invention. In one embodiment, the anti-activin antibody is used in the form of an ADC.

In one aspect, the invention provides a method of decreasing tumor size in a subject having cancer, comprising administering to the subject an effective amount of an anti-activin antibody or CAR modified immune cell, such as a CAR-T or CAR-NK cell, or a CAR-macrophage, of the invention. In one embodiment, the anti-activin antibody is used in the form of an ADC.

In one embodiment, the subject is a human subject. In one embodiment, the cancer is selected from the group consisting of cholangiocarcinoma, colon adenocarcinoma, B-cell lymphoma, esophageal carcinoma, glioblastoma, head and neck cancer, kidney clear cell cancer, low grade glioma, pancreatic adenocarcinoma, paraganglioma, prostate adenocarcinoma, rectal adenocarcinoma, sarcoma, stomach adenocarcinoma, and thyroid carcinoma. In additional or alternative embodiments, the cancer is selected from the group consisting of adrenocortical cancer, cholangiocarcinoma, colon adenocarcinoma, B-cell lymphoma, esophageal carcinoma, glioblastoma, head and neck cancer, kidney chromophobe, kidney clear cell cancer, kidney papillary cell cancer, low grade glioma, liver hepatocellular cancer, lung adenocarcinoma, ovarian cancer, pancreatic adenocarcinoma, paraganglioma, rectal adenocarcinoma, sarcoma, stomach adenocarcinoma, thyroid carcinoma, and uterine corpus cancer.

In one aspect, the invention provides a pharmaceutical composition, comprising an anti-activin antibody and a pharmaceutically acceptable carrier. In one aspect, the invention provides a pharmaceutical composition, comprising a CAR modified immune cell, such as a CAR-T or CAR-NK cell, of the invention and a pharmaceutically acceptable carrier. In one embodiment, the anti-activin antibody is used in the form of an ADC.

In one aspect, the invention provides methods for making an anti-activin antibody. In one aspect, the invention provides methods for making a CAR modified immune cell disclosed herein. In one embodiment, the invention provides methods for making an ADC comprising an anti-activin antibody.

In one aspect, the invention provides a method for the preparation of a medicament for the treatment of cancer.

In one aspect, the invention provides a method of determining the presence of activin A, B, and/or inhibin tumor epitope in a subject or in a biological sample from a subject. In one embodiment, the method comprises contacting a sample with an anti-activin antibody and determining binding of the anti-activin antibody to the sample, wherein binding of the anti-activin antibody to the sample is indicative of the presence of the activin tumor epitope in the sample.

In one aspect, the invention provides a method for diagnosing cancer in a subject, comprising detecting the presence of the activin and/or inhibin tumor epitope in the subject or in a biological sample from the subject.

In one aspect, the invention provides a method for determining the prognosis for a subject diagnosed with cancer, comprising detecting the presence of an activin and/or inhibin tumor epitope in the subject or in a biological sample from the subject. In one embodiment, the method involves detecting the presence of the activin and/or inhibin tumor epitope in the subject or in a biological sample from the subject after the subject has received a therapeutic agent for the treatment of cancer.

Also provided herein are kits and methods of using the same.

### BRIEF DESCRIPTION OF THE DRAWINGS

FIG. 1 shows a schematic drawing of how at least five different Activin and Inhibin complexes are assembled from three different pro-proteins after processing into mature monomers which are associated to form various homo or hetero-dimers with identical subunits.

FIG. 2 shows the percent inhibition of both latent (left) and mature (right) Activin A in a screen by SMAD nuclear localization assay at 1 hour and fibroblast activation at 48 hours for different antibodies from the six antibody discovery campaign. Antibodies were tested at a single concentration based on expression in 293 HEK cell cultures. The label POS refers to positive controls Control 1 and Control 2, each used in duplicate. The label NEG refers to negative control Control 3 used in quadruplicate.

FIG. 3 shows INHBA is highly expressed in multiple solid tumor indications compared to normal tissue (breast, colon, pancreatic, and stomach).

FIG. 4 shows expression of INHBA is prognostic in multiple solid tumor indications (breast, colon, and pancreatic cancers). FIG. 4. For three solid tumor types that express high levels of INHBA, hazard ratios were calculated between the patients expressing greater or less than the median level of INHBA; resulting in a 50:50 split. Survival curves were plotted highlighting differences between the two groups (solid line), alongside confidence intervals at P=0.05, indicating where significant divergence is seen between the two groups. For all three tumor types, statistically significant variation between survival times in the two groups were observed.

FIG. 5 shows INHBA expression in colorectal cancer (CRC) correlates with the stage of disease.

FIG. 6 shows INHBA is expressed in co-cultures of fibroblast/tumor cells but not significantly expressed in individual fibroblast or tumor cell monocultures, unlike TGF-β, which is expressed in individual fibroblast and tumor cell monocultures as well as co-cultures of fibroblast/tumor cells.

FIG. 7 shows Activin A treatment stimulates SMAD2/3 and SMAD4 nuclear localization (1 hr) and markers of fibroblast activation (CD248 and αSMA, 48 hours) comparable to levels stimulated by TGF-β ligand treatment.

FIG. 8 shows binding potency and specificity of anti-Activin antibodies to Activin A and/or B that are demonstrates certain clones are highly potent and cross-reactive.

FIG. 9 shows ability of anti-Activin antibody clone 843 to inhibit SMAD phosphorylation in fibroblasts, endothelial cells, and macrophages in colorectal tumor explants at levels comparable to or greater than comparator anti-Activin antibody.

FIG. 10 illustrates data from The Cancer Genome Atlas (TCGA) showing expression levels of Activin A, Activin B, and inhibin in various oncology indications.

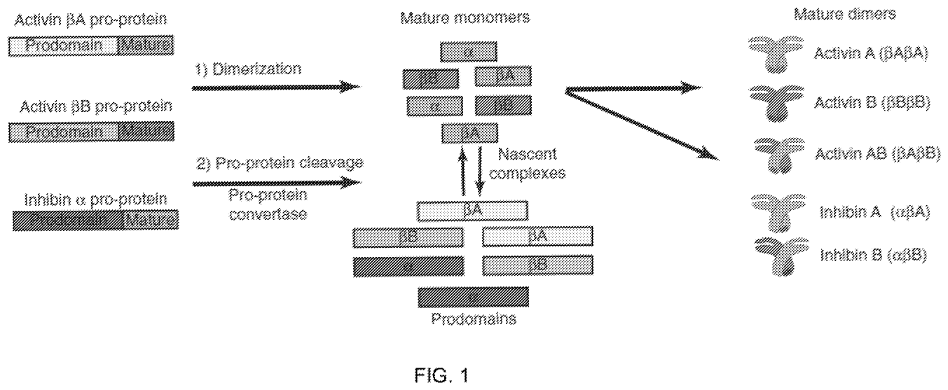

### DETAILED DESCRIPTION OF THE INVENTION

### I. General Techniques

The practice of the present invention will employ, unless otherwise indicated, conventional techniques of molecular biology (including recombinant techniques), microbiology, cell biology, biochemistry, and immunology, which are within the skill of the art. Such techniques are explained fully in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Current Protocols in Molecular Biology” (F. M. Ausubel et al., eds., 1987, and periodic updates); “PCR: The Polymerase Chain Reaction”, (Mullis et al., ed., 1994); “A Practical Guide to Molecular Cloning” (Perbal Bernard V., 1988); “Phage Display: A Laboratory Manual” (Barbas et al., 2001).

One skilled in the art will recognize many methods and materials similar or equivalent to those described herein, which could be used in the practice of the present invention. Indeed, the present invention is in no way limited to the methods and materials described. For purposes of the present invention, the following terms are defined below.

### II. Definitions

For purposes of interpreting this specification, the following definitions will apply and whenever appropriate, terms used in the singular will also include the plural and vice versa. In the event that any definition set forth conflicts with any document incorporated herein by reference, the definition set forth below shall control.

The term “activin”, as used herein, refers to any native activin from any vertebrate source, including mammals such as primates (e.g., humans, primates, and rodents (e.g., mice and rats), unless otherwise indicated. The activin molecule is also referred to as inhibin beta-1, Follicle-Stimulating Hormone-Releasing Protein (FRP), FSH-Releasing Protein, FSH-Releasing Factor, Erythroid Differentiation Factor (EDF) (see, e.g., SEQ ID NOs: 205-206). Human activin is encoded by the nucleotide sequence corresponding to activin INHBA isoform (GenBank Accession No. EAW94141.1).

The term “activin” encompasses “full-length,” unprocessed activin as well as any form of activin that results from processing in the cell. The term “activin” encompasses both latent and mature activin unless a specificity is indicated. The term also encompasses naturally occurring variants of activin, e.g., splice variants, allelic variants and isoforms. The activin polypeptides described herein may be isolated from a variety of sources, such as from human tissue types or from another source, or prepared by recombinant or synthetic methods. A “native sequence activin polypeptide” comprises a polypeptide having the same amino acid sequence as the corresponding activin polypeptide derived from nature. Such native sequence activin polypeptides can be isolated from nature or can be produced by recombinant or synthetic means. The term “native sequence activin polypeptide” specifically encompasses naturally-occurring truncated or secreted forms of the specific activin polypeptide (e.g., an extracellular domain sequence), naturally-occurring variant forms (e.g., alternatively spliced forms) and naturally-occurring allelic variants of the polypeptide. In certain embodiments of the invention, the native sequence activin polypeptides disclosed herein are mature or full-length native sequence polypeptides comprising the full-length amino acid sequences shown in the accompanying figures.

A “modification” of an amino acid residue/position, as used herein, refers to a change of a primary amino acid sequence as compared to a starting amino acid sequence, wherein the change results from a sequence alteration involving said amino acid residue/positions. For example, typical modifications include substitution of the residue (or at said position) with another amino acid (e.g., a conservative or non-conservative substitution), insertion of one or more (generally fewer than 5 or 3) amino acids adjacent to said residue/position, and deletion of said residue/position. An “amino acid substitution”, or variation thereof, refers to the replacement of an existing amino acid residue in a predetermined (starting) amino acid sequence with a different amino acid residue. Generally, the modification results in alteration in at least one physicobiochemical activity of the variant polypeptide compared to a polypeptide comprising the starting (or “wild type”) amino acid sequence. For example, in the case of an antibody, a physicobiochemical activity that is altered can be binding affinity, binding capability and/or binding effect upon a target molecule.

The term “antibody” is used in the broadest sense and specifically covers, for example, single anti-activin monoclonal antibodies (including agonist, antagonist, neutralizing antibodies, full length or intact monoclonal antibodies), anti-activin antibody compositions with polyepitopic specificity, polyclonal antibodies, multivalent antibodies, multispecific antibodies (e.g., bispecific antibodies so long as they exhibit the desired biological activity), formed from at least two intact antibodies, single chain anti-activin antibodies, and fragments of anti-activin antibodies (see below), including Fab, Fab′, F(ab′)2 and Fv fragments, diabodies, single domain antibodies (sdAbs), as long as they exhibit the desired biological or immunological activity. Also included among anti-activin antibodies, and among fragments in particular, are portions of anti-activin antibodies (and combinations of portions of anti-activin antibodies, for example, scFv) that may be used as targeting arms, directed to activin tumor epitope, in chimeric antigenic receptors of CAR-T cells, CAR-NK cells, or CAR-macrophages. Such fragments are not necessarily proteolytic fragments but rather portions of polypeptide sequences that can confer affinity for target. The term “immunoglobulin” (Ig) is used interchangeably with antibody herein. An antibody can be, for example, human, humanized and/or affinity matured.

The terms “anti-activin antibody”, “activin antibody”, and “an antibody that binds to activin” are used interchangeably. Anti-activin antibodies are preferably capable of binding with sufficient affinity such that the antibody is useful as a diagnostic and/or therapeutic agent, whether in isolation or as part of fusion protein, cell, or cell composition.

The terms “anti-inhibin antibody”, “inhibin antibody”, and “an antibody that binds to inhibin” are used interchangeably. Anti-inhibin antibodies are preferably capable of binding with sufficient affinity such that the antibody is useful as a diagnostic and/or therapeutic agent, whether in isolation or as part of fusion protein, cell, or cell composition.

In one embodiment, activin antibody is used herein to specifically refer to an anti-activin monoclonal antibody that (i) comprises the heavy chain variable domain of any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, and 69; and/or the light chain variable domain of any one of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, and 70 or (ii) comprises one, two, three, four, five, or six of the CDRs shown in Tables I and II, or Tables III and IV.

An “isolated antibody” is one which has been identified and separated and/or recovered from a component of its natural environment. Contaminant components of its natural environment are materials which would interfere with therapeutic uses for the antibody, and may include enzymes, hormones, and other proteinaceous or nonproteinaceous solutes.

The basic 4-chain antibody unit is a heterotetrameric glycoprotein composed of two identical light (L) chains and two identical heavy (H) chains. In the case of IgGs, the 4-chain unit is generally about 150,000 daltons. Each L chain is linked to a H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (VH) followed by three constant domains (CH) for each of the α and γ chains and four CH domains for μ and ε isotypes. Each L chain has at the N-terminus, a variable domain (VL) followed by a constant domain (CL) at its other end. The VL is aligned with the VH and the CL is aligned with the first constant domain of the heavy chain (CH1). Particular amino acid residues are believed to form an interface between the light chain and heavy chain variable domains. The pairing of a VH and VL together forms a single antigen-binding site. For the structure and properties of the different classes of antibodies, see, e.g., Basic and Clinical Immunology, 8th edition, Daniel P. Stites, Abba I. Terr and Tristram G. Parslow (eds.), Appleton & Lange, Norwalk, CT, 1994, at page 71 and Chapter 6.

The L chain from any vertebrate species can be assigned to one of two clearly distinct types, called kappa and lambda, based on the amino acid sequences of their constant domains. Depending on the amino acid sequence of the constant domain of their heavy chains (CH), immunoglobulins can be assigned to different classes or isotypes. There are five classes of immunoglobulins: IgA, IgD, IgE, IgG, and IgM, having heavy chains designated α, δ, ε, γ, and μ, respectively. The γ and α classes are further divided into subclasses on the basis of relatively minor differences in CH sequence and function, e.g., humans express the following subclasses: IgG1, IgG2, IgG3, IgG4, IgA1, and IgA2.

The “variable region” or “variable domain” of an antibody refers to the amino-terminal domains of the heavy or light chain of the antibody. The variable domain of the heavy chain may be referred to as “VH” or “V_{H}” The variable domain of the light chain may be referred to as “VL” or “V_{L}”. These domains are generally the most variable parts of an antibody and contain the antigen-binding sites.

The term “variable” refers to the fact that certain segments of the variable domains differ extensively in sequence among antibodies. The V domain mediates antigen binding and defines specificity of a particular antibody for its particular antigen. However, the variability is not evenly distributed across the 110-amino acid span of the variable domains. Instead, the V regions consist of relatively invariant stretches called framework regions (FRs) of 15-30 amino acids separated by shorter regions of extreme variability called “hypervariable regions” that are each 9-12 amino acids long. The variable domains of native heavy and light chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, MD. (1991)).

An “intact” antibody is one which comprises an antigen-binding site as well as a CL and at least heavy chain constant domains, CH1, CH2 and CH3. The constant domains may be native sequence constant domains (e.g., human native sequence constant domains) or amino acid sequence variant thereof. Preferably, the intact antibody has one or more effector functions.

“Antibody fragments” comprise a portion of an intact antibody, preferably the antigen binding or one or more variable regions of the intact antibody. Examples of antibody fragments include Fab, Fab′, F(ab′)2, and Fv fragments; diabodies; linear antibodies (see U.S. Pat. No. 5,641,870, Example 2; Zapata et al., Protein Eng. 8 (10): 1057-62 (1995)); single-chain antibody molecules; and multispecific antibodies formed from antibody fragments. In one embodiment, an antibody fragment comprises an antigen binding site of the intact antibody and thus retains the ability to bind antigen. Also included among anti-activin antibody fragments are portions of anti-activin antibodies (and combinations of portions of anti-activin antibodies, for example, scFv) that may be used as targeting arms, directed to activin tumor epitope, in chimeric antigenic receptors of CAR-T cells or CAR-NK cells. Such fragments are not necessarily proeteolytic fragments but rather portions of polypeptide sequences that can confer affinity for target.

Papain digestion of antibodies produces two identical antigen-binding fragments, called “Fab” fragments, and a residual “Fc” fragment, a designation reflecting the ability to crystallize readily. The Fab fragment consists of an entire L chain along with the variable region domain of the H chain (VH), and the first constant domain of one heavy chain (CH1). Each Fab fragment is monovalent with respect to antigen binding, i.e., it has a single antigen-binding site. Pepsin treatment of an antibody yields a single large F(ab′)2 fragment which roughly corresponds to two disulfide linked Fab fragments having divalent antigen-binding activity and is still capable of cross-linking antigen. Fab′ fragments differ from Fab fragments by having additional few residues at the carboxy terminus of the CH1 domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are also known.

The Fc fragment comprises the carboxy-terminal portions of both H chains held together by disulfides. The effector functions of antibodies are determined by sequences in the Fc region, which region is also the part recognized by Fc receptors (FcR) found on certain types of cells.

“Fv” is the minimum antibody fragment which contains a complete antigen-recognition and -binding site. This fragment consists of a dimer of one heavy- and one light-chain variable region domain in tight, non-covalent association. In a single-chain Fv (scFv) species, one heavy- and one light-chain variable domain can be covalently linked by a flexible peptide linker such that the light and heavy chains can associate in a “dimeric” structure analogous to that in a two-chain Fv species. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three CDRs specific for an antigen) has the ability to recognize and bind antigen, although at a lower affinity than the entire binding site.

“Single-chain Fv” also abbreviated as “sFv” or “scFv” are antibody fragments that comprise the VH and VL antibody domains connected into a single polypeptide chain. In some embodiments, the sFv polypeptide further comprises a polypeptide linker between the VH and VL domains which enables the sFv to form a desired structure for antigen binding. For a review of sFv, see, e.g., Pluckthun in The Pharmacology of Monoclonal Antibodies, vol. 113, Rosenburg and Moore eds., Springer-Verlag, New York, pp. 269-315 (1994); Borrebaeck 1995, infra. In one embodiment, an anti-activin antibody derived scFv is used as the targeting arm of a CAR-T cell or a CAR-NK cell disclosed herein.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” is not to be construed as requiring production of the antibody by any particular method. For example, the monoclonal antibodies useful in the present invention may be prepared by the hybridoma methodology first described by Kohler et al., Nature, 256:495 (1975), or may be made using recombinant DNA methods in bacterial, eukaryotic animal or plant cells (e.g., U.S. Pat. No. 4,816,567). The “monoclonal antibodies” may also be isolated from phage antibody libraries using the techniques described in Clackson et al., Nature, 352:624-8 (1991) and Marks et al., J. Mol. Biol., 222:581-97 (1991), for example.

The term “hypervariable region”, “HVR”, or “HV”, when used herein refers to the regions of an antibody variable domain which are hypervariable in sequence and/or form structurally defined loops. Generally, antibodies comprise six hypervariable regions; three in the VH (H1, H2, H3), and three in the VL (L1, L2, L3). A number of hypervariable region delineations are in use and are encompassed herein. The Kabat Complementarity Determining Regions (CDRs) are based on sequence variability and are the most commonly used (Kabat et al., Sequences of Proteins of Immunological Interest, 5th Ed. Public Health Service, National Institutes of Health, Bethesda, MD. (1991)). Chothia refers instead to the location of the structural loops (Chothia and Lesk J. Mol. Biol. 196:901-917 (1987)). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software. The “contact” hypervariable regions are based on an analysis of the available complex crystal structures. The residues from each of these hypervariable regions are noted below.

| Loop | Kabat | AbM | Chothia | Contact |
|---|---|---|---|---|
| L1 | L24-L34 | L24-L34 | L24-L34 | L30-L36 |
| L2 | L50-L56 | L50-L56 | L50-L56 | L46-L55 |
| L3 | L89-L97 | L89-L97 | L89-L97 | L89-L96 |
| H1 | H31-H35B | H26-H35B | H26-H32 . . . 34 | H30-H35B |

(Kabat Numbering)

| | | | | |
|---|---|---|---|---|
| H1 | H31-H35 | H26-H35 | H26-H32 | H30-H35 |

(Chothia Numbering)

| | | | | |
|---|---|---|---|---|
| H2 | H50-H65 | H50-H58 | H52-H56 | H47-H58 |
| H3 | H95-H102 | H95-H102 | H95-H102 | H93-H101 |

Hypervariable regions may comprise “extended hypervariable regions” as follows: 24-36 or 24-34 (L1), 46-56 or 50-56 (L2) and 89-97 (L3) in the VL and 26-35B (H1), 50-65, 47-65 or 49-65 (H2) and 93-102, 94-102 or 95-102 (H3) in the VH. The variable domain residues are numbered according to Kabat et al., supra, for each of these definitions.

“Framework” or “FR” residues are those variable domain residues other than the hypervariable region residues herein defined.

The term “variable domain residue numbering as in Kabat” or “amino acid position numbering as in Kabat”, and variations thereof, refers to the numbering system used for heavy chain variable domains or light chain variable domains of the compilation of antibodies in Kabat et al., supra. Using this numbering system, the actual linear amino acid sequence may contain fewer or additional amino acids corresponding to a shortening of, or insertion into, a FR or CDR of the variable domain. For example, a heavy chain variable domain may include a single amino acid insert (residue 52a according to Kabat) after residue 52 of H2 and inserted residues (e.g., residues 82a, 82b, and 82c, etc according to Kabat) after heavy chain FR residue 82. The Kabat numbering of residues may be determined for a given antibody by alignment at regions of homology of the sequence of the antibody with a “standard” Kabat numbered sequence.

The Kabat numbering system is generally used when referring to a residue in the variable domain (approximately residues 1-107 of the light chain and residues 1-113 of the heavy chain) (e.g, Kabat et al., supra). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG1 EU antibody. Unless stated otherwise herein, references to residue numbers in the variable domain of antibodies means residue numbering by the Kabat numbering system.

A “blocking” antibody or an “antagonist” antibody is one which inhibits or reduces biological activity of the antigen it binds. Preferred blocking antibodies or antagonist antibodies substantially or completely inhibit the biological activity of the antigen. In one embodiment, an anti-activin antibody is provided, which is an antagonist antibody.

An antibody that “binds” an antigen or epitope of interest is one that binds the antigen or epitope with sufficient affinity that is measurably different from a non-specific interaction. Specific binding can be measured, for example, by determining binding of a molecule compared to binding of a control molecule, which generally is a molecule of similar structure that does not have binding activity.

An antibody that inhibits the growth of tumor cells is one that results in measurable growth inhibition of cancer cells. In one embodiment, an anti-activin antibody is capable of inhibiting the growth of cancer cells displaying the activin tumor epitope. Preferred growth inhibitory anti-activin antibodies inhibit growth of activin-expressing tumor cells by greater than 20%, preferably from about 20% to about 50%, and even more preferably, by greater than 50% (e.g., from about 50% to about 100%) as compared to the appropriate control, the control typically being tumor cells not treated with the antibody being tested.

Anti-activin antibodies may (i) inhibit the growth or proliferation of a cell to which they bind; (ii) induce the death of a cell to which they bind; (iii) inhibit the delamination of a cell to which they bind; (iv) inhibit the metastasis of a cell to which they bind; or (v) inhibit the vascularization of a tumor comprising a cell to which they bind.

The term “antagonist” is used in the broadest sense, and includes any molecule that partially or fully blocks, inhibits, or neutralizes a biological activity of antigen. Suitable antagonist molecules specifically include antagonist antibodies or antibody fragments, fragments or amino acid sequence variants of native activin polypeptides, peptides, antisense oligonucleotides, small organic molecules, etc. Methods for identifying antagonists of an activin polypeptide, may comprise contacting an activin polypeptide, with a candidate antagonist molecule and measuring a detectable change in one or more biological activities normally associated with the activin polypeptide.

The terms “cancer” and “cancerous” refer to or describe the physiological condition in mammals that is typically characterized by unregulated cell growth. A “tumor” comprises one or more cancerous cells. Examples of cancer include, but are not limited to, carcinoma, lymphoma, blastoma, sarcoma, and leukemia or lymphoid malignancies. More particular examples of such cancers include squamous cell cancer (e.g., epithelial squamous cell cancer), skin cancer, melanoma, lung cancer including small-cell lung cancer, non-small cell lung cancer (“NSCLC”), adenocarcinoma of the lung and squamous carcinoma of the lung, cancer of the peritoneum, hepatocellular cancer, gastric or stomach cancer including gastrointestinal cancer, pancreatic cancer (e.g., pancreatic ductal adenocarcinoma), glioblastoma, cervical cancer, ovarian cancer (e.g., high grade serous ovarian carcinoma), liver cancer (e.g., hepatocellular carcinoma (HCC)), bladder cancer (e.g., urothelial bladder cancer), testicular (germ cell tumor) cancer, hepatoma, breast cancer, brain cancer (e.g., astrocytoma), colon cancer, rectal cancer, colorectal cancer, endometrial or uterine carcinoma, salivary gland carcinoma, kidney or renal cancer (e.g., renal cell carcinoma, nephroblastoma or Wilms' tumor), prostate cancer, vulval cancer, thyroid cancer, hepatic carcinoma, anal carcinoma, penile carcinoma, as well as head and neck cancer. Additional examples of cancer include, without limitation, retinoblastoma, thecomas, arrhenoblastomas, hepatoma, hematologic malignancies including non-Hodgkins lymphoma (NHL), multiple myeloma and acute hematologic malignancies, endometrial or uterine carcinoma, endometriosis, fibrosarcomas, choriocarcinoma, salivary gland carcinoma, vulval cancer, thyroid cancer, esophageal carcinomas, hepatic carcinoma, anal carcinoma, penile carcinoma, nasopharyngeal carcinoma, laryngeal carcinomas, Kaposi's sarcoma, melanoma, skin carcinomas, Schwannoma, oligodendroglioma, neuroblastomas, rhabdomyosarcoma, osteogenic sarcoma, leiomyosarcomas, and urinary tract carcinomas. Still further examples of cancer include, without limitation, adrenocortical cancer, cholangiocarcinoma, colon adenocarcinoma, B-cell lymphoma, esophageal carcinoma, glioblastoma, head and neck cancer, kidney chromophobe, kidney clear cell cancer, kidney papillary cell cancer, low grade glioma, pancreatic adenocarcinoma, liver hepatocellular cancer, lung adenocarcinoma, ovarian cancer, paraganglioma, pancreatic adenocarcinoma, prostate adenocarcinoma, rectal adenocarcinoma, sarcoma, stomach adenocarcinoma, uterine corpus cancer and thyroid carcinoma.

The term “metastatic cancer” means the state of cancer where the cancer cells of a tissue of origin are transmitted from the original site to one or more sites elsewhere in the body, by the blood vessels or lymphatics, to form one or more secondary tumors in one or more organs besides the tissue of origin. A prominent example is metastatic breast cancer.

As used herein, an “activin-associated cancer” is a cancer that is associated with overexpression of an activin gene or gene product and/or is associated with display of the activin tumor epitope. Suitable control cells can be, for example, cells from an individual who is not affected with cancer or non-cancerous cells from the subject who has cancer.

The present methods include methods of treating a subject having cancer. Particularly cancer that is associated with expression of an activin tumor epitope, and preferably a shared epitope between Activin A and Activin B. The present methods also include methods for modulating certain cell behaviours, particularly cancer cell behaviours, particularly cancer cells displaying the activin tumor epitope.

In one embodiment, the activin tumor epitope comprises a post-translational modification of activin. In one embodiment, the activin tumor epitope comprises a sialylated O-glycosyl moiety attached to activin. In one embodiment, the activin tumor epitope comprises an O-linked glycan moiety that is linked to activin. In one embodiment, the activin tumor epitope comprises a glycan moiety that is linked to activin, wherein the glycan moiety has at its terminus beta-N-acetyl-galactosamine.

The terms “cell proliferative disorder” and “proliferative disorder” refer to disorders that are associated with some degree of abnormal cell proliferation. In one embodiment, the cell proliferative disorder is cancer.

“Tumor”, as used herein, refers to all neoplastic cell growth and proliferation, whether malignant or benign, and all pre-cancerous and cancerous cells and tissues.

The terms “predictive” and “prognostic” as used herein are also interchangeable. In one sense, the methods for prediction or prognostication are to allow the person practicing a predictive/prognostic method of the invention to select patients that are deemed (usually in advance of treatment, but not necessarily) more likely to respond to treatment with an anti-cancer agent, preferably an anti-activin antibody or a CAR engineered cell of the invention.

### III. Compositions and Methods of the Invention

In one aspect, the invention provides anti-activin antibodies, including fragments thereof, compositions comprising the same, and methods of using the same for various purposes, including the treatment of cancer.

In one aspect, the invention provides an antibody that binds to the activin tumor epitope. In one aspect, an antibody competes for binding to, or binds substantially to, the activin tumor epitope. Optionally, the antibody is a monoclonal antibody, antibody fragment, including Fab, Fab′, F(ab′)2, and Fv fragment, diabody, single domain antibody, chimeric antibody, humanized antibody, single-chain antibody or antibody that competitively inhibits the binding of an anti-activin epitope antibody to its respective antigenic epitope. The antibodies of the present invention may optionally be produced in CHO cells or bacterial cells or by other means. In one embodiment, an anti-activin antibody induces death of a cell to which it binds. For detection purposes, the anti-activin antibodies of the present invention may be detectably labeled, attached to a solid support, or the like.

In one aspect, the invention provides anti-activin antibodies that inhibit or neutralize one or more human Activin A functions. In further embodiments, the anti-activin antibody is capable of inducing SMAD nuclear localization and/or fibroblast activation using an assay known to the skilled worker, such as using a BJ fibroblast assay described herein.

In one aspect, a functional anti-activin antibody is provided, wherein the antibody has one or more of the following activities: (i) inhibits delamination; (ii) inhibits tumor metastasis in vivo; (iii) inhibits tumor growth in vivo; (iv) decreases tumor size in vivo; (v) inhibits tumor vascularization in vivo; (vi) exhibits cytotoxic activity on tumor cell expressing activin in vivo; or (vii) exhibits cytostatic activity on a tumor cell expressing activin in vivo.

TABLE I
Complementarity Determining Regions, Heavy Variable Region (IMGT)

| | Heavy Chain Variable Region CDRs | | |
|---|---|---|---|
| | CDR1 | CDR2 | CDR3 |
| AB448 | GLSLSNAKMG | VSTTGTT | ARAGYPDV |
| | (SEQ ID NO: 210) | (SEQ ID NO: 245) | (SEQ ID NO: 280) |
| AB465 | GASVSNARTG | INPSGRI | ARAGYPDV |
| | (SEQ ID NO: 211) | (SEQ ID NO: 246) | (SEQ ID NO: 281) |
| AB340 | GFTASRNY | ISGSGDYT | ARSPRGPRLLGYYYGMDV |
| | (SEQ ID NO: 212) | (SEQ ID NO: 247) | (SEQ ID NO: 282) |
| AB342 | GFDFGEHW | IKGDASDT | VKDLSKQWLGIDY |
| | (SEQ ID NO: 213) | (SEQ ID NO: 248) | (SEQ ID NO: 283) |
| AB344 | GYTFSNYG | ISKSGSSI | ARLPRLAWNYYYGMDV |
| | (SEQ ID NO: 214) | (SEQ ID NO: 249) | (SEQ ID NO: 284) |
| AB353 | GLSLSNAKMG | VSTTGTT | ARAGYPDV |
| | (SEQ ID NO: 215) | (SEQ ID NO: 250) | (SEQ ID NO: 285) |
| AB438 | GVSLSNARLG | GSVTGST | TTSYLVDTAMVTR |
| | (SEQ ID NO: 216) | (SEQ ID NO: 251) | (SEQ ID NO: 286) |
| AB366 | GYSLTDARIG | INPSGRT | ARAGYPDV |
| | (SEQ ID NO: 217) | (SEQ ID NO: 252) | (SEQ ID NO: 287) |
| AB341 | GYTFSNYG | ISKSGSSI | ARLPRLAWNYYYGMDV |
| | (SEQ ID NO: 218) | (SEQ ID NO: 253) | (SEQ ID NO: 288) |
| AB430 | GFTLSNYP | TTSGSSTI | ARGPDYYAPLRSYMDV |
| | (SEQ ID NO: 219) | (SEQ ID NO: 254) | (SEQ ID NO: 289) |
| AB349 | GVSLSNARLG | GSVTGST | TTSYLVDTAMVTR |
| | (SEQ ID NO: 220) | (SEQ ID NO: 255) | (SEQ ID NO: 290) |
| AB449 | GLSLSNAKMG | VSTTGTT | ARAGYPDV |
| | (SEQ ID NO: 221) | (SEQ ID NO: 256) | (SEQ ID NO: 291) |
| AB338 | GFTASRNY | ISGSGDYT | ARSPRGPRLLGYYYGMDV |
| | (SEQ ID NO: 222) | (SEQ ID NO: 257) | (SEQ ID NO: 292) |
| AB435 | GYSLTDARIG | INPSGRT | ARAGYPDV |
| | (SEQ ID NO: 223) | (SEQ ID NO: 258) | (SEQ ID NO: 293) |
| AB372 | GFSLSTSGAT | ITSSGR | TTTKIVGATLRDY |
| | (SEQ ID NO: 224) | (SEQ ID NO: 259) | (SEQ ID NO: 294) |
| AB439 | GYTYTGYR | IDPKSGRT | AREVKQWLVPGRGVYWFDP |
| | (SEQ ID NO: 225) | (SEQ ID NO: 260) | (SEQ ID NO: 295) |
| AB350 | GVSLSDARMG | VSTTGTT | ARAGYPDV |
| | (SEQ ID NO: 226) | (SEQ ID NO: 261) | (SEQ ID NO: 296) |
| AB363 | GFSLSTSGAT | ITSSGR | TTTKIVGATLRDY |
| | (SEQ ID NO: 227) | (SEQ ID NO: 262) | (SEQ ID NO: 297) |
| AB367 | GFTLSNPRMG | ISTSGRT | ARAGYPDV |
| | (SEQ ID NO: 228) | (SEQ ID NO: 263) | (SEQ ID NO: 298) |
| AB352 | GFPLSTSRMG | INPSGAT | ARAGYPDV |
| | (SEQ ID NO: 229) | (SEQ ID NO: 264 | (SEQ ID NO: 299) |
| AB357 | GYTFPSHY | MNPITGHT | ARDMRYDILPGRSYYYGMDV |
| | (SEQ ID NO: 230) | (SEQ ID NO: 265) | (SEQ ID NO: 300) |
| AB347 | GFPLSTSRMG | INPSGAT | ARAGYPDV |
| | (SEQ ID NO: 231) | (SEQ ID NO: 266) | (SEQ ID NO: 301) |
| AB346 | GFPLSTSRMG | INPSGAT | ARAGYPDV |
| | (SEQ ID NO: 232) | (SEQ ID NO: 267) | (SEQ ID NO: 302) |
| AB433 | GFPLSTSRMG | INPSGAT | ARAGYPDV |
| | (SEQ ID NO: 233) | (SEQ ID NO: 268) | (SEQ ID NO: 303) |
| AB432 | GVSLSNVRMG | VNPSGST | ARAGYPDV |
| | (SEQ ID NO: 234) | (SEQ ID NO: 269) | (SEQ ID NO: 304) |
| AB326 | GYTFTSSA | IYPGSGHT | AREGDIFVMDV |
| | (SEQ ID NO: 235) | (SEQ ID NO: 270) | (SEQ ID NO: 305) |
| AB428 | GFTSSNHW | IGPKGLGT | ASIRDYGDKKGGSWFDP |
| | (SEQ ID NO: 236) | (SEQ ID NO: 271) | (SEQ ID NO: 306) |
| AB426 | GFTFSDHH | ISPSGDFT | ARDVSPVYY |
| | (SEQ ID NO: 237) | (SEQ ID NO: 272) | (SEQ ID NO: 307) |
| AB345 | GFTSSGYW | IGTAGVT | ARGPHGPQQRLRFDP |
| | (SEQ ID NO: 238) | (SEQ ID NO: 273) | (SEQ ID NO: 308) |
| AB431 | GFTSSNHW | IGPKGLGT | ARDVRGWRALYYYYGMDV |
| | (SEQ ID NO: 239) | (SEQ ID NO: 274) | (SEQ ID NO: 309) |
| AB330 | GYPSPAT | MNPKSGYT | ATLHGSGLAFDI |
| | (SEQ ID NO: 240) | (SEQ ID NO: 275) | (SEQ ID NO: 310) |
| AB731 | GYTFTSYW | INPSNGGT | SITTVVAYWYFDV |
| | (SEQ ID NO: 241) | (SEQ ID NO: 276) | (SEQ ID NO: 311) |
| AB843 | GDTSSHSH | INPRSGAT | ARDRGGRTNGVGAFDI |
| | (SEQ ID NO: 242) | (SEQ ID NO: 277) | (SEQ ID NO: 312) |
| AB852 | GFSLSDTRWG | INDRGTT | AHTLHTREFVGMDV |
| | (SEQ ID NO: 243) | (SEQ ID NO: 278) | (SEQ ID NO: 313) |
| AB854 | GYTFPSHY | MNPITGHT | ARDMRYDILPGRSYYYGMDV |
| | (SEQ ID NO: 244) | (SEQ ID NO: 279) | (SEQ ID NO: 314) |

TABLE II
Complementarity Determining Regions, Light Variable Region (IMGT)

| | Light Chain Variable Region CDRs | | |
|---|---|---|---|
| Name | CDR1 | CDR2 | CDR3 |
| AB448 | QNIGAA | GAS | QQYTAYPLT |
| | (SEQ ID NO: 315) | (SEQ ID NO: 350) | (SEQ ID NO: 385) |
| AB465 | QTVSSH | DVS | QQYFTLPT |
| | (SEQ ID NO: 316) | (SEQ ID NO: 351) | (SEQ ID NO: 386) |
| AB340 | RSLVHGSGHNY | GAS | MQGTQAPRT |
| | (SEQ ID NO: 317) | (SEQ ID NO: 352) | (SEQ ID NO: 387) |
| AB342 | QSLLHSNEKNY | DAS | VQDLHTPFT |
| | (SEQ ID NO: 318) | (SEQ ID NO: 353) | (SEQ ID NO: 388) |
| AB344 | RSLVHGSGDNY | MAS | VQTLQTPST |
| | (SEQ ID NO: 319) | (SEQ ID NO: 354) | (SEQ ID NO: 389) |
| AB353 | QNVYTN | GAS | LQYGSSPPL |
| | (SEQ ID NO: 320) | (SEQ ID NO: 355) | (SEQ ID NO: 390) |
| AB438 | QSLGTN | GAS | QQYDTHPLS |
| | (SEQ ID NO: 321) | (SEQ ID NO: 356) | (SEQ ID NO: 391) |
| AB366 | QTISRS | DAS | QQYSSYPSL |
| | (SEQ ID NO: 322) | (SEQ ID NO: 357) | (SEQ ID NO: 392) |
| AB341 | QSLLHSNEKNY | DAS | VQDLHTPFT |
| | (SEQ ID NO: 323) | (SEQ ID NO: 358) | (SEQ ID NO: 393) |
| AB430 | QSLQQSEGRTY | DAS | MQVLRTPIT |
| | (SEQ ID NO: 324) | (SEQ ID NO: 359) | (SEQ ID NO: 394) |
| AB349 | QRIGSD | SAS | STMAIP |
| | (SEQ ID NO: 325) | (SEQ ID NO: 360) | (SEQ ID NO: 395) |
| AB449 | QGISRD | AAS | QQYVSAPPYT |
| | (SEQ ID NO: 326) | (SEQ ID NO: 361) | (SEQ ID NO: 396) |
| AB338 | QSLLHSNEKNY | DAS | VQDLHTPFT |
| | (SEQ ID NO: 327) | (SEQ ID NO: 362) | (SEQ ID NO: 397) |
| AB435 | QSIGTK | DAS | QQYGSSPPVYS |
| | (SEQ ID NO: 328) | (SEQ ID NO: 363) | (SEQ ID NO: 398) |
| AB372 | QSVLDTSTNRNY | GAS | QQSYSHPPT |
| | (SEQ ID NO: 329) | (SEQ ID NO: 364) | (SEQ ID NO: 399) |
| AB439 | NIGLRS | DTN | SSYTSSSTDV |
| | (SEQ ID NO: 330) | (SEQ ID NO: 365) | (SEQ ID NO: 400) |
| AB350 | ESVRSN | GAS | QQYHTWPPWA |
| | (SEQ ID NO: 331) | (SEQ ID NO: 366) | (SEQ ID NO: 401) |
| AB363 | QGISND | DVS | QQYDNPPS |
| | (SEQ ID NO: 332) | (SEQ ID NO: 367) | (SEQ ID NO: 402) |
| AB367 | QGIGID | DAS | RQYATFPQT |
| | (SEQ ID NO: 333) | (SEQ ID NO: 368) | (SEQ ID NO: 403) |
| AB352 | QSVSTH | DAT | LQYTNYPWT |
| | (SEQ ID NO: 334) | (SEQ ID NO: 369) | (SEQ ID NO: 404) |
| AB357 | DIRGMS | EVN | AAWDASVAGYV |
| | (SEQ ID NO: 335) | (SEQ ID NO: 370) | (SEQ ID NO: 405) |
| AB347 | QNIGHS | GAS | QQYITWPST |
| | (SEQ ID NO: 336) | (SEQ ID NO: 371) | (SEQ ID NO: 406) |
| AB346 | RSVSSD | DVS | QQYGSSLPPT |
| | (SEQ ID NO: 337) | (SEQ ID NO: 372) | (SEQ ID NO: 407) |
| AB433 | QSVRTS | DTS | QQYGNWH |
| | (SEQ ID NO: 338) | (SEQ ID NO: 373) | (SEQ ID NO: 408) |
| AB432 | QSISSSQ | GAS | QQYNDRPRT |
| | (SEQ ID NO: 339) | (SEQ ID NO: 374) | (SEQ ID NO: 409) |
| AB326 | MDIRND | DAS | QQAKSFPPT |
| | (SEQ ID NO: 340) | (SEQ ID NO: 375) | (SEQ ID NO: 410) |
| AB428 | QSLLHSSGYNF | MAT | VQDLHTLPQ |
| | (SEQ ID NO: 341) | (SEQ ID NO: 376) | (SEQ ID NO: 411) |
| AB426 | QSL | MGS | QSGVT |
| | (SEQ ID NO: 342) | (SEQ ID NO: 377) | (SEQ ID NO: 412) |
| AB345 | QSLLSSYGYHN | MGS | MQGTQAPRT |
| | (SEQ ID NO: 343) | (SEQ ID NO: 378) | (SEQ ID NO: 413) |
| AB431 | QSLLHVTGYNY | GAS | MQSTQWPHT |
| | (SEQ ID NO: 344) | (SEQ ID NO: 379) | (SEQ ID NO: 414) |
| AB330 | QDIKSD | RAS | QQYRSFPWS |
| | (SEQ ID NO: 345) | (SEQ ID NO: 380) | (SEQ ID NO: 415) |
| AB731 | SSVSY | RTY | QQRSSYPWT |
| | (SEQ ID NO: 346) | (SEQ ID NO: 381) | (SEQ ID NO: 416) |
| AB843 | SGDIGRYNY | EDI | QVWDATLDLV |
| | (SEQ ID NO: 347) | (SEQ ID NO: 382) | (SEQ ID NO: 417) |
| AB852 | QSVLDTSTNRNY | GAS | QQYETWPWT |
| | (SEQ ID NO: 348) | (SEQ ID NO: 383) | (SEQ ID NO: 418) |
| AB854 | TGSIASNY | DTT | QSTERGRSDVI |
| | (SEQ ID NO: 349) | (SEQ ID NO: 384) | (SEQ ID NO: 419) |

TABLE III
Complementarity Determining Regions, Heavy Variable Region (Kabat)

| | Heavy Chain Variable Region CDRs | | |
|---|---|---|---|
| Name | CDR1 | CDR2 | CDR3 |
| AB448 | NAKMGVN | YVSTTGTTDYNPSLKS | AGYPDV |
| | (SEQ ID NO: | (SEQ ID NO: 455) | (SEQ ID NO: 490) |
| | 420) | | |
| AB465 | NARTGVN | YINPSGRIDYNPSLKS | AGYPDV |
| | (SEQ ID NO: | (SEQ ID NO: 456) | (SEQ ID NO: 491) |
| | 421) | | |
| AB340 | RNYMS | SISGSGDYTYYPDSVKG | SPRGPRLLGYYYGMDV |
| | (SEQ ID NO: | (SEQ ID NO: 457) | (SEQ ID NO: 492) |
| | 422) | | |
| AB342 | EHWMS | SIKGDASDTYYPDSVKG | DLSKQWLGIDY |
| | (SEQ ID NO: | (SEQ ID NO: 458) | (SEQ ID NO: 493) |
| | 423) | | |
| AB344 | NYGMS | SISKSGSSIYYPDSVKG | LPRLAWNYYYGMDV |
| | (SEQ ID NO: | (SEQ ID NO: 459) | (SEQ ID NO: 494) |
| | 424) | | |
| AB353 | NAKMGVN | YVSTTGTTDYNPSLKS | AGYPDV |
| | (SEQ ID NO: | (SEQ ID NO: 460) | (SEQ ID NO: 495) |
| | 425) | | |
| AB438 | NARLGVN | YGSVTGSTDYNPSLKS | SYLVDTAMVTR |
| | (SEQ ID NO: | (SEQ ID NO: 461) | (SEQ ID NO: 496) |
| | 426) | | |
| AB366 | DARIGVN | YINPSGRTDYNPSLKS | AGYPDV |
| | (SEQ ID NO: | (SEQ ID NO: 462) | (SEQ ID NO: 497) |
| | 427) | | |
| AB341 | NYGMS | SISKSGSSIYYPDSVKG | LPRLAWNYYYGMDV |
| | (SEQ ID NO: | (SEQ ID NO: 463) | (SEQ ID NO: 498) |
| | 428) | | |
| AB430 | NYPMS | STTSGSSTIYYPDSVKG | GPDYYAPLRSYMDV |
| | (SEQ ID NO: | (SEQ ID NO: 464) | (SEQ ID NO: 499) |
| | 429) | | |
| AB349 | NARLGVN | YGSVTGSTDYNPSLKS | SYLVDTAMVTR |
| | (SEQ ID NO: | (SEQ ID NO: 465) | (SEQ ID NO: 500) |
| | 430) | | |
| AB449 | NAKMGVN | YVSTTGTTDYNPSLKS | AGYPDV |
| | (SEQ ID NO: | (SEQ ID NO: 466) | (SEQ ID NO: 501) |
| | 431) | | |
| AB338 | RNYMS | SISGSGDYTYYPDSVKG | SPRGPRLLGYYYGMDV |
| | (SEQ ID NO: | (SEQ ID NO: 467) | (SEQ ID NO: 502) |
| | 432) | | |
| AB435 | DARIGVN | YINPSGRTDYNPSLKS | AGYPDV |
| | (SEQ ID NO: | (SEQ ID NO: 468) | (SEQ ID NO: 503) |
| | 433) | | |
| AB372 | TSGATVN | YITSSGRTVYNSALKS | TKIVGATLRDY |
| | (SEQ ID NO: | (SEQ ID NO: 469) | (SEQ ID NO: 504) |
| | 434) | | |
| AB439 | GYRIS | WIDPKSGRTNYAQKLQG | EVKQWLVPGRGVYWFDP |
| | (SEQ ID NO: | (SEQ ID NO: 470) | (SEQ ID NO: 505) |
| | 435) | | |
| AB350 | DARMGVN | YVSTTGTTDYNPSLKS | AGYPDV |
| | (SEQ ID NO: | (SEQ ID NO: 471) | (SEQ ID NO: 506) |
| | 436) | | |
| AB363 | TSGATVN | YITSSGRTVYNSALKS | TKIVGATLRDY |
| | (SEQ ID NO: | (SEQ ID NO: 472) | (SEQ ID NO: 507) |
| | 437) | | |
| AB367 | NPRMGVN | YISTSGRTDYNPSLKS | AGYPDV |
| | (SEQ ID NO: | (SEQ ID NO: 473) | (SEQ ID NO: 508) |
| | 438) | | |
| AB352 | TSRMGVN | YINPSGATDYNPSLKS | AGYPDV |
| | (SEQ ID NO: | (SEQ ID NO: 474) | (SEQ ID NO: 509) |
| | 439) | | |
| AB357 | SHYIS | GMNPITGHTIYAQKLQG | DMRYDILPGRSYYYGMDV |
| | (SEQ ID NO: | (SEQ ID NO: 475) | (SEQ ID NO: 510) |
| | 440) | | |
| AB347 | TSRMGVN | YINPSGATDYNPSLKS | AGYPDV |
| | (SEQ ID NO: | (SEQ ID NO: 476) | (SEQ ID NO: 511) |
| | 441) | | |
| AB346 | TSRMGVN | YINPSGATDYNPSLKS | AGYPDV |
| | (SEQ ID NO: | (SEQ ID NO: 477) | (SEQ ID NO: 512) |
| | 442) | | |
| AB433 | TSRMGVN | YINPSGATDYNPSLKS | AGYPDV |
| | (SEQ ID NO: | (SEQ ID NO: 478) | (SEQ ID NO: 513) |
| | 443) | | |
| AB432 | NVRMGVN | YVNPSGSTDYNPSLKS | AGYPDV |
| | (SEQ ID NO: | (SEQ ID NO: 479) | (SEQ ID NO: 514) |
| | 444) | | |
| AB326 | SSAIS | GIYPGSGHTIYAQKFQG | EGDIFVMDV |
| | (SEQ ID NO: | (SEQ ID NO: 480) | (SEQ ID NO: 515) |
| | 445) | | |
| AB428 | NHWMS | SIGPKGLGTYYPDSVKG | IRDYGDKKGGSWFDP |
| | (SEQ ID NO: | (SEQ ID NO: 481) | (SEQ ID NO: 516) |
| | 446) | | |
| AB426 | DHHMS | SISPSGDFTYYPDSVKG | DVSPVYY |
| | (SEQ ID NO: | (SEQ ID NO: 482) | (SEQ ID NO: 517) |
| | 447) | | |
| AB345 | GYWMS | SIGTAGVTYYPDSVKG | GPHGPQQRLRFDP |
| | (SEQ ID NO: | (SEQ ID NO: 483) | (SEQ ID NO: 518) |
| | 448) | | |
| AB431 | NHWMS | SIGPKGLGTYYPDSVKG | DVRGWRALYYYYGMDV |
| | (SEQ ID NO: | (SEQ ID NO: 484) | (SEQ ID NO: 519) |
| | 449) | | |
| AB330 | ATIS | WMNPKSGYTNYAQKFQG | LHGSGLAFDI |
| | (SEQ ID NO: | (SEQ ID NO: 485) | (SEQ ID NO: 520) |
| | 450) | | |
| AB731 | SYWMH | NINPSNGGTNYNEKFKS | TTVVAYWYFDV |
| | (SEQ ID NO: | (SEQ ID NO: 486) | (SEQ ID NO: 521) |
| | 451) | | |
| AB843 | HSHIH | WINPRSGATNYAQKLQG | DRGGRTNGVGAFDI |
| | (SEQ ID NO: | (SEQ ID NO: 487) | (SEQ ID NO: 522) |
| | 452) | | |
| AB852 | DTRWGVN | YINDRGTTDYNSALKS | TLHTREFVGMDV |
| | (SEQ ID NO: | (SEQ ID NO: 488) | (SEQ ID NO: 523) |
| | 453) | | |
| AB854 | SHYIS | GMNPITGHTIYAQKLQG | DMRYDILPGRSYYYGMDV |
| | (SEQ ID NO: | (SEQ ID NO: 489) | (SEQ ID NO: 524) |
| | 454) | | |

TABLE IV
Complementarity Determining Regions, Light Variable Region (Kabat)
CDRs

| | Light Chain Variable Region CDRs | | |
|---|---|---|---|
| Name | CDR1 | CDR2 | CDR3 |
| AB448 | RASQNIGAALA | GASTREY | QQYTAYPLT |
| | (SEQ ID NO: 525) | (SEQ ID NO: 560) | (SEQ ID NO: 96) |
| AB465 | RASQTVSSHLA | DVSHRAS | QQYFTLPT |
| | (SEQ ID NO: 526) | (SEQ ID NO: 561) | (SEQ ID NO: 97) |
| AB340 | RSSRSLVHGSGHNYLH | GASTRVA | MQGTQAPRT |
| | (SEQ ID NO: 527) | (SEQ ID NO: 562) | (SEQ ID NO: 98) |
| AB342 | RSSQSLLHSNEKNYLH | DASKRAS | VQDLHTPFT |
| | (SEQ ID NO: 528) | (SEQ ID NO: 563) | (SEQ ID NO: 99) |
| AB344 | RSSRSLVHGSGDNYLH | MASNRAP | VQTLQTPST |
| | (SEQ ID NO: 529) | (SEQ ID NO: 564) | (SEQ ID NO: 170) |
| AB353 | RASQNVYTNLA | GASARAS | LQYGSSPPL |
| | (SEQ ID NO: 530) | (SEQ ID NO: 565) | (SEQ ID NO: 171) |
| AB438 | RASQSLGTNLA | GASSRPS | QQYDTHPLS |
| | (SEQ ID NO: 531) | (SEQ ID NO: 566) | (SEQ ID NO: 172) |
| AB366 | RASQTISRSLA | DASSLAG | QQYSSYPSL |
| | (SEQ ID NO: 532) | (SEQ ID NO: 567) | (SEQ ID NO: 173) |
| AB341 | RSSQSLLHSNEKNYLH | DASKRAS | VQDLHTPFT |
| | (SEQ ID NO: 533) | (SEQ ID NO: 568) | (SEQ ID NO: 174) |
| AB430 | RSSQSLQQSEGRTYLH | DASKRAS | MQVLRTPIT |
| | (SEQ ID NO: 534) | (SEQ ID NO: 569) | (SEQ ID NO: 175) |
| AB349 | RASQRIGSDLA | SASSRAP | STMAIP |
| | (SEQ ID NO: 535) | (SEQ ID NO: 570) | (SEQ ID NO: 176) |
| AB449 | RASQGISRDLA | AASTLVS | QQYVSAPPYT |
| | (SEQ ID NO: 536) | (SEQ ID NO: 571) | (SEQ ID NO: 177) |
| AB338 | RSSQSLLHSNEKNYLH | DASKRAS | VQDLHTPFT |
| | (SEQ ID NO: 537) | (SEQ ID NO: 572) | (SEQ ID NO: 178) |
| AB435 | RASQSIGTKLA | DASSRVP | QQYGSSPPVYS |
| | (SEQ ID NO: 538) | (SEQ ID NO: 573) | (SEQ ID NO: 179) |
| AB372 | KSSQSVLDTSTNRNYLA | GASIRAG | QQSYSHPPT |
| | (SEQ ID NO: 539) | (SEQ ID NO: 574) | (SEQ ID NO: 180) |
| AB439 | TGTNIGLRSVS | DTNIRSS | SSYTSSSTDV |
| | (SEQ ID NO: 540) | (SEQ ID NO: 575) | (SEQ ID NO: 181) |
| AB350 | RASESVRSNLA | GASNRAS | QQYHTWPPWA |
| | (SEQ ID NO: 541) | (SEQ ID NO: 576) | (SEQ ID NO: 182) |
| AB363 | RASQGISNDLA | DVSNRAS | QQYDNPPS |
| | (SEQ ID NO: 542) | (SEQ ID NO: 78) | (SEQ ID NO: 183) |
| AB367 | RASQGIGIDLA | DASKLQT | RQYATFPQT |
| | (SEQ ID NO: 543) | (SEQ ID NO: 79) | (SEQ ID NO: 184) |
| AB352 | RASQSVSTHLA | DATNRAT | LQYTNYPWT |
| | (SEQ ID NO: 544) | (SEQ ID NO: 80) | (SEQ ID NO: 185) |
| AB357 | TGTDIRGMSVS | EVNQRPS | AAWDASVAGYV |
| | (SEQ ID NO: 545) | (SEQ ID NO: 81) | (SEQ ID NO: 186) |
| AB347 | RASQNIGHSLA | GASIRAP | QQYITWPST |
| | (SEQ ID NO: 546) | (SEQ ID NO: 82) | (SEQ ID NO: 187) |
| AB346 | RASRSVSSDLA | DVSTRAT | QQYGSSLPPT |
| | (SEQ ID NO: 547) | (SEQ ID NO: 83) | (SEQ ID NO: 188) |
| AB433 | RASQSVRTSLA | DTSIRAA | QQYGNWH |
| | (SEQ ID NO: 548) | (SEQ ID NO: 84) | (SEQ ID NO: 189) |
| AB432 | RASQSISSSQLA | GASTRAT | QQYNDRPRT |
| | (SEQ ID NO: 549) | (SEQ ID NO: 85) | (SEQ ID NO: 190) |
| AB326 | RASMDIRNDLA | DASKLET | QQAKSFPPT |
| | (SEQ ID NO: 550) | (SEQ ID NO: 86) | (SEQ ID NO: 191) |
| AB428 | RSSQSLLHSSGYNFLH | MATNRAS | VQDLHTLPQ |
| | (SEQ ID NO: 551) | (SEQ ID NO: 87) | (SEQ ID NO: 192) |
| AB426 | RSSQSLLH | MGSQRAA | QSGVT |
| | (SEQ ID NO: 552) | (SEQ ID NO: 88) | (SEQ ID NO: 193) |
| AB345 | RSSQSLLSSYGYHNLH | MGSIRAP | MQGTQAPRT |
| | (SEQ ID NO: 553) | (SEQ ID NO: 89) | (SEQ ID NO: 194) |
| AB431 | RSSQSLLHVTGYNYLH | GASSRAA | MQSTQWPHT |
| | (SEQ ID NO: 554) | (SEQ ID NO: 90) | (SEQ ID NO: 195) |
| AB330 | RASQDIKSDLA | RASARES | QQYRSFPWS |
| | (SEQ ID NO: 555) | (SEQ ID NO: 91) | (SEQ ID NO: 196) |
| AB731 | SASSSVSYMY | RTYNLAS | QQRSSYPWT |
| | (SEQ ID NO: 556) | (SEQ ID NO: 92) | (SEQ ID NO: 197) |
| AB843 | TGTSGDIGRYNYVS | EDIERPS | QVWDATLDLV |
| | (SEQ ID NO: 557) | (SEQ ID NO: 93) | (SEQ ID NO: 198) |
| AB852 | KSSQSVLDTSTNRNYLA | GASIRAG | QQYETWPWT |
| | (SEQ ID NO: 558) | (SEQ ID NO: 94) | (SEQ ID NO: 199) |
| AB854 | TGTTGSIASNYVS | DTTNRPP | QSTERGRSDVI |
| | (SEQ ID NO: 559) | (SEQ ID NO: 95) | (SEQ ID NO: 209) |

In one aspect, an antibody that binds to Activin A and/or Activin B is provided, wherein the antibody comprises a heavy chain variable region comprising SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, or 69.

In one aspect, an antibody that binds to Activin A and/or Activin B is provided, wherein the antibody comprises a light chain variable region comprising SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, or 70.

In one embodiment, an antibody of the invention comprising these sequences (in combination as described herein) is a humanized or human antibody.

In one aspect, the invention includes an anti-activin antibody comprising (i) a heavy chain variable domain comprising SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, or 69; and/or (ii) a light chain variable domain comprising SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, or 70.

In some embodiments, these antibodies further comprise a human subgroup III heavy chain framework consensus sequence. In one embodiments of these antibodies, these antibodies further comprise a human κI light chain framework consensus sequence.

In one aspect, an anti-activin antibody competes for binding to a tumor displayed activin (for example, as displayed on fibroblast cells) with an anti-activin antibody comprising a heavy chain variable region comprising SEQ ID NO: 71 or 73 and a light chain variable region comprising SEQ ID NO: 72 or 74, and/or a heavy chain variable region comprising SEQ ID NO: 39, 41, 43, 45, 47, 49, 63, 65, or 69, and a light chain variable region comprising SEQ ID NO: 40, 42, 44, 46, 48, 50, 64, 66, or 70 for binding to an activin epitope.

In another aspect, an anti-activin antibody competes for binding to a tumor displayed activin (for example, as displayed on fibroblast cells) with follistatin.

In some embodiments, the therapeutic agent for use in a host subject elicits little to no immunogenic response against the agent in said subject. In one embodiment, the invention provides such an agent. For example, in one embodiment, the invention provides a humanized antibody that elicits and/or is expected to elicit a human anti-mouse antibody response (HAMA) at a substantially reduced level compared to an antibody comprising the sequence of SEQ ID NO: 71, 72, 73, and/or 74 in a host subject. In another example, the invention provides a humanized antibody that elicits and/or is expected to elicit minimal or no human anti-mouse antibody response (HAMA). In one example, an antibody of the invention elicits anti-mouse antibody response that is at or less than a clinically-acceptable level.

A humanized antibody of the invention may comprise one or more human and/or human consensus non-hypervariable region (e.g., framework) sequences in its heavy and/or light chain variable domain. In some embodiments, one or more additional modifications are present within the human and/or human consensus non-hypervariable region sequences. In one embodiment, the heavy chain variable domain of an antibody of the invention comprises a human consensus framework sequence, which in one embodiment is the subgroup III consensus framework sequence. In one embodiment, an antibody of the invention comprises a variant subgroup III consensus framework sequence modified at least one amino acid position.

As is known in the art, and as described in greater detail herein, the amino acid position/boundary delineating a hypervariable region of an antibody can vary, depending on the context and the various definitions known in the art (as described below). Some positions within a variable domain may be viewed as hybrid hypervariable positions in that these positions can be deemed to be within a hypervariable region under one set of criteria while being deemed to be outside a hypervariable region under a different set of criteria. One or more of these positions can also be found in extended hypervariable regions (as further defined below). The invention provides antibodies comprising modifications in these hybrid hypervariable positions. In one embodiment, these hypervariable positions include one or more positions 26-30, 33-35B, 47-49, 57-65, 93, 94 and 101-102 in a heavy chain variable domain. In one embodiment, these hybrid hypervariable positions include one or more of positions 24-29, 35-36, 46-49, 56 and 97 in a light chain variable domain. In one embodiment, an antibody of the invention comprises a human variant human subgroup consensus framework sequence modified at one or more hybrid hypervariable positions.

An antibody of the invention can comprise any suitable human or human consensus light chain framework sequences, provided the antibody exhibits the desired biological characteristics (e.g., a desired binding affinity). In one embodiment, an antibody of the invention comprises at least a portion (or all) of the framework sequence of human κ light chain. In one embodiment, an antibody of the invention comprises at least a portion (or all) of human κ subgroup I framework consensus sequence.

In some aspects, the invention provides vectors comprising DNA encoding any of the herein described anti-activin antibodies or portions thereof. Host cells comprising any such vector are also provided. By way of example, the host cells may be CHO cells, E. coli cells, or yeast cells. A process for producing any of the herein described polypeptides is further provided and comprises culturing host cells under conditions suitable for expression of the desired polypeptide and recovering the desired polypeptide from the cell culture.

The antibody of the present invention may be employed in any known assay method, such as ELISA, competitive binding assays, direct and indirect sandwich assays, and immunoprecipitation assays (Zola, (1987) Monoclonal Antibodies: A Manual of Techniques, pp. 147-158, CRC Press, Inc.).

A detection label may be useful for localizing, visualizing, and quantitating a binding or recognition event. The labelled antibodies of the invention can detect cell-surface receptors. Another use for detectably labelled antibodies is a method of bead-based immunocapture comprising conjugating a bead with a fluorescent labelled antibody and detecting a fluorescence signal upon binding of a ligand. Similar binding detection methodologies utilize the surface plasmon resonance (SPR) effect to measure and detect antibody-antigen interactions.

Detection labels such as fluorescent dyes and chemiluminescent dyes (Briggs et al (1997) J. Chem. Soc., Perkin-Trans. 1:1051-8) provide a detectable signal and are generally applicable for labelling antibodies, preferably with the following properties: (i) the labelled antibody should produce a very high signal with low background so that small quantities of antibodies can be sensitively detected in both cell-free and cell-based assays; and (ii) the labelled antibody should be photostable so that the fluorescent signal may be observed, monitored and recorded without significant photo bleaching. For applications involving cell surface binding of labelled antibody to membranes or cell surfaces, especially live cells, the labels preferably (iii) have good water-solubility to achieve effective conjugate concentration and detection sensitivity and (iv) are non-toxic to living cells so as not to disrupt the normal metabolic processes of the cells or cause premature cell death.

Direct quantification of cellular fluorescence intensity and enumeration of fluorescently labelled events, e.g., cell surface binding of peptide-dye conjugates may be conducted on an system (FMAT® 8100 HTS System, Applied Biosystems, Foster City, Calif.) that automates mix-and-read, non-radioactive assays with live cells or beads (Miraglia, “Homogeneous cell- and bead-based assays for high throughput screening using fluorometric microvolume assay technology”, (1999) J. of Biomolecular Screening 4:193-204). Uses of labelled antibodies also include cell surface receptor binding assays, inmmunocapture assays, fluorescence linked immunosorbent assays (FLISA), caspase-cleavage (Zheng, “Caspase-3 controls both cytoplasmic and nuclear events associated with Fas-mediated apoptosis in vivo”, (1998) Proc. Natl. Acad. Sci. USA 95:618-23; U.S. Pat. No. 6,372,907), apoptosis (Vermes, “A novel assay for apoptosis. Flow cytometric detection of phosphatidylserine expression on early apoptotic cells using fluorescein labelled Annexin V” (1995) J. Immunol. Methods 184:39-51) and cytotoxicity assays. Fluorometric microvolume assay technology can be used to identify the up or down regulation by a molecule that is targeted to the cell surface (Swartzman, “A homogeneous and multiplexed immunoassay for high-throughput screening using fluorometric microvolume assay technology”, (1999) Anal. Biochem. 271:143-51).

Labelled antibodies of the invention are useful as imaging biomarkers and probes by the various methods and techniques of biomedical and molecular imaging such as: (i) MRI (magnetic resonance imaging); (ii) MicroCT (computerized tomography); (iii) SPECT (single photon emission computed tomography); (iv) PET (positron emission tomography) Chen et al Bioconjugate Chem. 15:41-9 (2004); (v) bioluminescence; (vi) fluorescence; and (vii) ultrasound. Immunoscintigraphy is an imaging procedure in which antibodies labeled with radioactive substances are administered to an animal or human patient and a picture is taken of sites in the body where the antibody localizes (U.S. Pat. No. 6,528,624). Imaging biomarkers may be objectively measured and evaluated as an indicator of normal biological processes, pathogenic processes, or pharmacological responses to a therapeutic intervention.

Peptide labelling methods are well known (e.g., Haugland, 2003, Molecular Probes Handbook of Fluorescent Probes and Research Chemicals, Molecular Probes, Inc.; Brinkley, 1992, Bioconjugate Chem. 3:2; Garman, (1997) Non-Radioactive Labelling: A Practical Approach, Academic Press, London; Means (1990) Bioconjugate Chem. 1:2; Glazer et al. (1975) Chemical Modification of Proteins. Laboratory Techniques in Biochemistry and Molecular Biology (T. S. Work and E. Work, Eds.) American Elsevier Publishing Co., New York; Lundblad, R. L. and Noyes, C. M. (1984) Chemical Reagents for Protein Modification, Vols. I and II, CRC Press, New York; Pfleiderer, G. (1985) “Chemical Modification of Proteins”, Modern Methods in Protein Chemistry, H. Tschesche, Ed., Walter DeGryter, Berlin and New York; and Wong (1991) Chemistry of Protein Conjugation and Cross-linking, CRC Press, Boca Raton, Fla.); De Leon-Rodriguez et al. (2004) Chem. Eur. J. 10:1149-1155; Lewis et al. (2001) Bioconjugate Chem. 12:320-324; Li et al. (2002) Bioconjugate Chem. 13:110-115; Mier et al. (2005) Bioconjugate Chem. 16:240-237).

Peptides and proteins labelled with two moieties, a fluorescent reporter and quencher in sufficient proximity undergo fluorescence resonance energy transfer (FRET). Reporter groups are typically fluorescent dyes that are excited by light at a certain wavelength and transfer energy to an acceptor, or quencher, group, with the appropriate Stokes shift for emission at maximal brightness. Fluorescent dyes include molecules with extended aromaticity, such as fluorescein and rhodamine, and their derivatives. The fluorescent reporter may be partially or significantly quenched by the quencher moiety in an intact peptide. Upon cleavage of the peptide by a peptidase or protease, a detectable increase in fluorescence may be measured (Knight, C. (1995) “Fluorimetric Assays of Proteolytic Enzymes”, Methods in Enzymology, Academic Press, 248:18-34).

The labelled antibodies of the invention may also be used as an affinity purification agent. In this process, the labelled antibody is immobilized on a solid phase such a Sephadex resin or filter paper, using methods well known in the art. The immobilized antibody is contacted with a sample containing the antigen to be purified, and thereafter the support is washed with a suitable solvent that will remove substantially all the material in the sample except the antigen to be purified, which is bound to the immobilized polypeptide variant. Finally, the support is washed with another suitable solvent, such as glycine buffer, pH 5.0, that will release the antigen from the polypeptide variant.

In one aspect, an anti-activin antibody of the invention binds to the same epitope on activin bound by another activin antibody. In another embodiment, an activin antibody of the invention binds to the same epitope on activin bound by a fragment (e.g., a Fab fragment) of a monoclonal antibody comprising the variable domains of any one of SEQ ID NOs: 1-70 or a chimeric antibody comprising the variable domain of the monoclonal antibody comprising the sequences of Table I and Table II, or Table III and Table IV, and constant domains from a human IgG1 or IgG4.

In one aspect, the invention provides compositions comprising one or more antibodies of the invention and a carrier. In one embodiment, the carrier is pharmaceutically acceptable.

In one aspect, the invention provides nucleic acids encoding an activin antibody (or portion(s) thereof) of the invention (see Table 7). In some embodiments, the nucleic acids comprise any one of SEQ ID NOs: 100-169.

In one aspect, the invention provides vectors comprising a nucleic acid of the invention. In one embodiment, the vectors comprise any one of SEQ ID NOs: 100-169.

In one aspect, the invention provides host cells comprising a nucleic acid or a vector of the invention. A vector can be of any type, for example a recombinant vector such as an expression vector. Any of a variety of host cells can be used. In one embodiment, a host cell is a prokaryotic cell, for example, E. coli. In one embodiment, a host cell is a eukaryotic cell, for example a mammalian cell such as Chinese Hamster Ovary (CHO) cell.

In one aspect, the invention provides methods for making an antibody of the invention. For example, the invention provides a method of making an activin antibody (which, as defined herein includes full length and fragments thereof), said method comprising expressing in a suitable host cell a recombinant vector of the invention encoding said antibody (or fragment thereof), and recovering said antibody.

In one aspect, the invention provides an article of manufacture comprising a container; and a composition contained within the container, wherein the composition comprises one or more activin antibodies or CAR modified immune cell, preferably a CAR-T or CAR-NK cell, or CAR-macrophage, of the invention. In one embodiment, the composition comprises a nucleic acid of the invention. In one embodiment, a composition comprising an antibody or CAR modified immune cell, such as CAR-T or CAR-NK cell, or CAR-macrophage, further comprises a carrier, which in some embodiments is pharmaceutically acceptable. In one embodiment, an article of manufacture of the invention further comprises instructions for administering the composition (e.g., the antibody) to a subject.

In one aspect, the invention provides a kit comprising a first container comprising a composition comprising one or more activin antibodies or CAR modified immune cells, such as CAR-T or CAR-NK cells, or CAR macrophages, of the invention; and a second container comprising a buffer. In one embodiment, the buffer is pharmaceutically acceptable. In one embodiment, a kit further comprises instructions for administering the composition (e.g., the antibody) to a subject.

In one aspect, the invention provides use of an activin antibody or CAR modified immune cells, preferably a CAR-T or CAR-NK cells, or CAR macrophages, of the invention in the preparation of a medicament for the therapeutic and/or prophylactic treatment of a disease or disorder, such as a cancer, a tumor and/or a cell proliferative disorder.

In one aspect, the invention provides use of a nucleic acid of the invention in the preparation of a medicament for the therapeutic and/or prophylactic treatment of a disease or disorder, such as a cancer, a tumor and/or a cell proliferative disorder.

In one aspect, the invention provides use of an expression vector of the invention in the preparation of a medicament for the therapeutic and/or prophylactic treatment of a disease or disorder, such as a cancer, a tumor and/or a cell proliferative disorder.

In one aspect, the invention provides use of a host cell of the invention in the preparation of a medicament for the therapeutic and/or prophylactic treatment of a disease or disorder, such as a cancer, a tumor and/or a cell proliferative disorder.

In one aspect, the invention provides use of an article of manufacture of the invention in the preparation of a medicament for the therapeutic and/or prophylactic treatment of a disease or disorder, such as a cancer, a tumor and/or a cell proliferative disorder.

In one aspect, the invention provides use of a kit of the invention in the preparation of a medicament for the therapeutic and/or prophylactic treatment of a disease or disorder, such as a cancer, a tumor and/or a cell proliferative disorder.

In one aspect, the invention provides a method of inhibiting the growth of a cell that expresses activin, said method comprising contacting said cell with an antibody or CAR modified immune cells, such as CAR-T or CAR-NK cells, or CAR macrophages, of the invention thereby causing an inhibition of growth of said cell.

In one aspect, the invention provides a method of therapeutically treating a mammal having a cancerous tumor comprising a cell that expresses activin, said method comprising administering to said mammal a therapeutically effective amount of an antibody or CAR modified immune cells, such as CAR-T or CAR-NK cells, or CAR macrophages, of the invention, thereby effectively treating said mammal.

In one aspect, the invention provides use of an activin antibody of the invention in the preparation of a medicament for (i) inhibiting the vascularization of a tumor comprising cells expressing activin; (ii) inhibiting the delamination of cells expressing activin; (iii) inhibiting tumor metastasis in a patient having cancer; (iv) decreasing tumor size in a patient having cancer.

In one aspect, the invention provides a method for treating or preventing a cell proliferative disorder associated with increased expression of activin, said method comprising administering to a subject in need of such treatment an effective amount of an antibody or CAR modified immune cells, such as CAR-T or CAR-NK cells, or CAR macrophages, of the invention, thereby effectively treating or preventing said cell proliferative disorder. In one embodiment, said cell proliferative disorder is cancer.

In one aspect, the invention provides a method of determining the presence of activin in a sample suspected of containing activin, said method comprising exposing said sample to an antibody of the invention, and determining binding of said antibody to activin in said sample wherein binding of said antibody to activin in said sample is indicative of the presence of said protein in said sample. In one embodiment, the sample is a biological sample. In a further embodiment, the biological sample comprises breast cancer cells. In one embodiment, the biological sample is from a mammal experiencing or suspected of experiencing a breast cancer disorder and/or a breast cancer cell proliferative disorder. In a further embodiment, the biological sample comprises ovarian cancer cells. In one embodiment, the biological sample is from a mammal experiencing or suspected of experiencing an ovarian cancer disorder and/or an ovarian cancer cell proliferative disorder. In a further embodiment, the biological sample comprises melanoma cells. In one embodiment, the biological sample is from a mammal experiencing or suspected of experiencing a melanoma disorder and/or a melanoma cell proliferative disorder. In a further embodiment, the biological sample comprises glioblastoma cells. In one embodiment, the biological sample is from a mammal experiencing or suspected of experiencing a glioblastoma disorder and/or a glioblastoma cell proliferative disorder.

In one aspect, a method of diagnosing a cell proliferative disorder associated with (i) an increase in cells, such as, e.g., breast cancer cells, ovarian cancer cells, melanoma cells, or glioblastoma cells, expressing activin, or (ii) an increase in activin expression within a tumor, is provided. In one embodiment, the method comprises contacting a test cell in a biological sample with any of the above antibodies; determining the level of antibody bound to test cells in the sample by detecting binding of the antibody to activin; and comparing the level of antibody bound to cells in a control sample, wherein the level of antibody bound is normalized to the number of activin-expressing cells in the test and control samples, and wherein a higher level of antibody bound in the test sample as compared to the control sample indicates the presence of a cell proliferative disorder associated with cells expressing activin.

In one aspect, the invention provides a method of inhibiting the vascularization of a tumor comprising cells expressing activin, comprising administering to a patient an effective amount of an antibody or CAR modified immune cells, such as CAR-T or CAR-NK cells, or CAR macrophages, described herein, thereby effectively inhibiting vascularization of the tumor.

In one aspect, the invention provides a method of inhibiting the delamination of cells expressing activin, comprising administering to a patient an effective amount of an antibody or CAR modified immune cells, such as CAR-T or CAR-NK cells, or CAR macrophages, described herein, thereby effectively inhibiting delamination of the cells.

In one aspect, the invention provides a method of inhibiting tumor metastasis in a patient having cancer, comprising administering to a patient an effective amount of an antibody or CAR modified immune cells, such as CAR-T or CAR-NK cells, or CAR macrophages, described herein, thereby effectively inhibiting tumor metastasis.

In one aspect, the invention provides a method of decreasing tumor size, comprising administering to a patient an effective amount of an antibody or CAR modified immune cells, such as CAR-T or CAR-NK cells, or CAR macrophages, described herein, thereby effectively decreasing tumor size.

In one aspect, the invention provides a method for treating or preventing a cell proliferative disorder associated with increased expression of activin, said method comprising administering to a subject in need of such treatment an effective amount of an antibody or CAR modified immune cells, such as CAR-T or CAR-NK cells, or CAR macrophages, of the invention, thereby effectively treating or preventing said cell proliferative disorder. In one embodiment, said proliferative disorder is cancer.

In one aspect, the invention provides a method of binding an antibody of the invention to a cell that expresses activin, said method comprising contacting said cell with an antibody of the invention.

In other aspects of the present invention, the invention provides vectors comprising DNA encoding any of the herein described antibodies (or portion(s) thereof). Host cell comprising any such vector are also provided. By way of example, the host cells may be CHO cells, E. coli cells, or yeast cells. A process for producing any of the herein described antibodies is further provided and comprises culturing host cells under conditions suitable for expression of the desired antibody and recovering the desired antibody from the cell culture.

In a still further aspect, the invention concerns a composition of matter comprising an anti-activin antibody as described herein, in combination with a carrier. Optionally, the carrier is a pharmaceutically acceptable carrier. In a still further aspect, the invention concerns a composition of matter comprising a CAR modified immune cells, such as CAR-T or CAR-NK cells, or CAR macrophages, as described herein, in combination with a carrier. Optionally, the carrier is a pharmaceutically acceptable carrier.

Another aspect of the present invention is directed to the use of an anti-activin epitope antibody as described herein, for the preparation of a medicament useful in the treatment of a condition which is responsive to the anti-activin epitope antibody.

In another aspect, the invention provides immunoconjugates, or antibody-drug conjugates (ADC), comprising an anti-activin antibody conjugated to a cytotoxic agent such as a chemotherapeutic agent, a drug, a growth inhibitory agent, a toxin (e.g., an enzymatically active toxin of bacterial, fungal, plant, or animal origin, or fragments thereof), or a radioactive isotope (i.e., a radioconjugate). In another aspect, the invention further provides methods of using the immunoconjugates. In one aspect, an immunoconjugate comprises any of the above anti-activin antibodies covalently attached to a cytotoxic agent or a detectable agent.

### A. Anti-Activin Antibodies

In one embodiment, the present invention provides anti-activin antibodies which may find use herein as therapeutic agents. Exemplary antibodies include polyclonal, monoclonal, chimeric, humanized, and human antibodies.

### 1. Polyclonal Antibodies

Polyclonal antibodies may be raised in animals by multiple subcutaneous (sc) or intraperitoneal (ip) injections of the relevant antigen and an adjuvant. It may be useful to conjugate the relevant antigen (especially when synthetic peptides are used) to a protein that is immunogenic in the species to be immunized. For example, the antigen can be conjugated to keyhole limpet hemocyanin (KLH), serum albumin, bovine thyroglobulin, or soybean trypsin inhibitor, using a bifunctional or derivatizing agent, e.g., maleimidobenzoyl sulfosuccinimide ester (conjugation through cysteine residues), N-hydroxysuccinimide (through lysine residues), glutaraldehyde, succinic anhydride, SOCl2, or R′N═C═NR, where R and R1 are different alkyl groups.

Animals are immunized against the antigen, immunogenic conjugates, or derivatives by combining, e.g., 100 μg or 5 μg of the protein or conjugate (for rabbits or mice, respectively) with 3 volumes of Freund's complete adjuvant and injecting the solution intradermally at multiple sites. One month later, the animals are boosted with ⅕ to 1/10 the original amount of peptide or conjugate in Freund's complete adjuvant by subcutaneous injection at multiple sites. Seven to 14 days later, the animals are bled and the serum is assayed for antibody titer. Animals are boosted until the titer plateaus. Conjugates also can be made in recombinant cell culture as protein fusions. Also, aggregating agents such as alum are suitably used to enhance the immune response.

### 2. Monoclonal Antibodies

A monoclonal antibody (mAb) to an antigen-of-interest can be prepared by using any technique known in the art. These include, but are not limited to, the hybridoma technique originally described by Kohler and Milstein (1975, Nature 256, 495-497), the human B cell hybridoma technique (Kozbor et al., 1983, Immunology Today 4:72), and the EBV-hybridoma technique (Cole et al., 1985, Monoclonal Antibodies and Cancer Therapy, Alan R. Liss, Inc., pp. 77-96). The Selected Lymphocyte Antibody Method (SLAM) (Babcook, J. S., et al., A novel strategy for generating monoclonal antibodies from single, isolated lymphocytes producing antibodies of defined specificities. Proc Natl Acad Sci USA, 1996. 93 (15): p. 7843-8.) and (McLean G et al., 2005, J Immunol. 174 (8): 4768-78. Such antibodies may be of any immunoglobulin class including IgG, IgM, IgE, IgA, and IgD and any subclass thereof. The hybridoma producing the mAbs of use in this invention may be cultivated in vitro or in vivo.

Monoclonal antibodies may be made using the hybridoma method first described by Kohler et al., Nature, 256:495 (1975), or may be made by recombinant DNA methods (U.S. Pat. No. 4,816,567).

In the hybridoma method, a mouse or other appropriate host animal, such as a hamster, is immunized as described above to elicit lymphocytes that produce or are capable of producing antibodies that will specifically bind to the protein used for immunization. Alternatively, lymphocytes may be immunized in vitro. After immunization, lymphocytes are isolated and then fused with a myeloma cell line using a suitable fusing agent, such as polyethylene glycol, to form a hybridoma cell (Goding, Monoclonal Antibodies: Principles and Practice, pp. 59-103 (Academic Press, 1986)).

The hybridoma cells thus prepared are seeded and grown in a suitable culture medium which may contain one or more substances that inhibit the growth or survival of the unfused, parental myeloma cells (also referred to as fusion partner). For example, if the parental myeloma cells lack the enzyme hypoxanthine guanine phosphoribosyl transferase (HGPRT or HPRT), the selective culture medium for the hybridomas typically will include hypoxanthine, aminopterin, and thymidine (HAT medium), which substances prevent the growth of HGPRT-deficient cells.

Preferred fusion partner myeloma cells are those that fuse efficiently, support stable high-level production of antibody by the selected antibody-producing cells, and are sensitive to a selective medium that selects against the unfused parental cells. Preferred myeloma cell lines are murine myeloma lines, such as those derived from MOPC-21 and MPC-11 mouse tumors available from the Salk Institute Cell Distribution Center, San Diego, Calif. USA, and SP-2 and derivatives e.g., X63-Ag8-653 cells available from the American Type Culture Collection, Manassas, Va., USA. Human myeloma and mouse-human heteromyeloma cell lines also have been described for the production of human monoclonal antibodies (Kozbor, J. Immunol., 133:3001 (1984); and Brodeur et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunosorbent assay (ELISA).

The binding affinity of the monoclonal antibody can, for example, be determined by the Scatchard analysis described in Munson et al., Anal. Biochem. 107:220 (1980).

Once hybridoma cells that produce antibodies of the desired specificity, affinity, and/or activity are identified, the clones may be subcloned by limiting dilution procedures and grown by standard methods (Goding, Monoclonal Antibodies: Principles and Practice, pp. 59-103 (Academic Press, 1986)). Suitable culture media for this purpose include, for example, D-MEM or RPMI-1640 medium. In addition, the hybridoma cells may be grown in vivo as ascites tumors in an animal, e.g., by intraperitoneal injection of the cells into mice.

The monoclonal antibodies secreted by the subclones are suitably separated from the culture medium, ascites fluid, or serum by conventional antibody purification procedures such as, for example, affinity chromatography (e.g., using protein A or protein G-Sepharose) or ion-exchange chromatography, hydroxylapatite chromatography, gel electrophoresis, dialysis, etc.

DNA encoding the monoclonal antibodies is readily isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA may be placed into expression vectors, which are then transfected into host cells such as E. coli cells, simian COS cells, Chinese Hamster Ovary (CHO) cells, or myeloma cells that do not otherwise produce antibody protein, to obtain the synthesis of monoclonal antibodies in the recombinant host cells. Review articles on recombinant expression in bacteria of DNA encoding the antibody include Skerra et al., Curr. Opinion in Immunol. 5:256-62 (1993) and Plückthun, Immunol. Rev. 130:151-88 (1992).

In a further embodiment, monoclonal antibodies or antibody fragments can be isolated from antibody phage libraries generated using the techniques described in McCafferty et al., Nature, 348:552-54 (1990). Clackson et al., Nature, 352:624-28 (1991) and Marks et al., J. Mol. Biol., 222:581-97 (1991) describe the isolation of murine and human antibodies, respectively, using phage libraries. Subsequent publications describe the production of high affinity (nM range) human antibodies by chain shuffling (Marks et al., Bio/Technology, 10: 779-83 (1992)), as well as combinatorial infection and in vivo recombination as a strategy for constructing very large phage libraries (Waterhouse et al., Nuc. Acids. Res. 21:2265-6 (1993)). Thus, these techniques are viable alternatives to traditional monoclonal antibody hybridoma techniques for isolation of monoclonal antibodies.

The DNA that encodes the antibody may be modified to produce chimeric or fusion antibody polypeptides, for example, by substituting human heavy chain and light chain constant domain (CH and CO sequences for the homologous murine sequences (U.S. Pat. No. 4,816,567; and Morrison, et al., Proc. Natl. Acad. Sci. USA, 81:6851 (1984)), or by fusing the immunoglobulin coding sequence with all or part of the coding sequence for a non-immunoglobulin polypeptide (heterologous polypeptide). The non-immunoglobulin polypeptide sequences can substitute for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

### 3. Chimeric, Humanized, and Human Antibodies

In some embodiments, the anti-activin antibody is a chimeric antibody. Certain chimeric antibodies are described, e.g., in U.S. Pat. No. 4,816,567; and Morrison et al., Proc. Natl. Acad. Sci. USA, 81:6851-5 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In some embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the CDR residues are derived), e.g., to restore or improve antibody specificity or affinity.

The anti-activin antibodies of the invention may further comprise humanized antibodies or human antibodies. Humanized forms of non-human (e.g., murine or rabbit) antibodies are chimeric immunoglobulins, immunoglobulin chains or fragments thereof (such as Fv, Fab, Fab′, F(ab′)2 or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin (Jones et al., Nature, 321:522-5 (1986); Riechmann et al., Nature, 332:323-9 (1988); and Presta, Curr. Op. Struct. Biol., 2:593-6 (1992)).

Methods for humanizing non-human antibodies are well known in the art. Generally, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

The choice of human variable domains, both light and heavy, to be used in making the humanized antibodies is very important to reduce antigenicity and HAMA response (human anti-mouse antibody) when the antibody is intended for human therapeutic use. Reduction or elimination of a HAMA response is a significant aspect of clinical development of suitable therapeutic agents (see, e.g., Khaxzaeli et al., J. Natl. Cancer Inst. (1988), 80:937; Jaffers et al., Transplantation (1986), 41:572; Shawler et al., J. Immunol. (1985), 135:1530; Sears et al., J. Biol. Response Mod. (1984), 3:138; Miller et al., Blood (1983), 62:988; Hakimi et al., J. Immunol. (1991), 147:1352; Reichmann et al., Nature (1988), 332:323; Junghans et al., Cancer Res. (1990), 50:1495). As described herein, the invention provides antibodies that are humanized such that HAMA response is reduced or eliminated. Variants of these antibodies can further be obtained using routine methods known in the art, some of which are further described below. According to the so-called “best-fit” method, the sequence of the variable domain of a rodent antibody is screened against the entire library of known human variable domain sequences. The human V domain sequence which is closest to that of the rodent is identified and the human framework region (FR) within it accepted for the humanized antibody (Sims et al., J. Immunol. 151:2296 (1993); Chothia et al., J. Mol. Biol., 196:901 (1987)). Another method uses a particular framework region derived from the consensus sequence of all human antibodies of a particular subgroup of light or heavy chains. The same framework may be used for several different humanized antibodies (Carter et al., Proc. Natl. Acad. Sci. USA, 89:4285 (1992); Presta et al., J. Immunol. 151:2623 (1993)).

For example, an amino acid sequence from an antibody as described herein can serve as a starting (parent) sequence for diversification of the framework and/or hypervariable sequence(s). A selected framework sequence to which a starting hypervariable sequence is linked is referred to herein as an acceptor human framework. While the acceptor human frameworks may be from, or derived from, a human immunoglobulin (the VL and/or VH regions thereof), preferably the acceptor human frameworks are from, or derived from, a human consensus framework sequence as such frameworks that have been demonstrated to have minimal, or no, immunogenicity in human patients.

Where the acceptor is derived from a human immunoglobulin, one may optionally select a human framework sequence that is selected based on its homology to the donor framework sequence by aligning the donor framework sequence with various human framework sequences in a collection of human framework sequences, and select the most homologous framework sequence as the acceptor.

In one embodiment, human consensus frameworks herein are from, or derived from, VH subgroup III and/or VL kappa subgroup I consensus framework sequences.

While the acceptor may be identical in sequence to the human framework sequence selected, whether that be from a human immunoglobulin or a human consensus framework, the present invention contemplates that the acceptor sequence may comprise pre-existing amino acid substitutions relative to the human immunoglobulin sequence or human consensus framework sequence. These pre-existing substitutions are preferably minimal; usually four, three, two or one amino acid differences only relative to the human immunoglobulin sequence or consensus framework sequence.

Hypervariable region residues of the non-human antibody are incorporated into the VL and/or VH acceptor human frameworks. For example, one may incorporate residues corresponding to the Kabat CDR residues, the Chothia hypervariable loop residues, the Abm residues, and/or contact residues. Optionally, the extended hypervariable region residues as follows are incorporated: 24-34 (L1), 50-56 (L2) and 89-97 (L3), 26-35B (H1), 50-65, 47-65 or 49-65 (H2) and 93-102, 94-102, or 95-102 (H3).

While “incorporation” of hypervariable region residues is discussed herein, it will be appreciated that this can be achieved in various ways, for example, nucleic acid encoding the desired amino acid sequence can be generated by mutating nucleic acid encoding the mouse variable domain sequence so that the framework residues thereof are changed to acceptor human framework residues, or by mutating nucleic acid encoding the human variable domain sequence so that the hypervariable domain residues are changed to non-human residues, or by synthesizing nucleic acid encoding the desired sequence, etc.

As described herein, hypervariable region-grafted variants may be generated by Kunkel mutagenesis of nucleic acid encoding the human acceptor sequences, using a separate oligonucleotide for each hypervariable region. Kunkel et al., Methods Enzymol. 154:367-382 (1987). Appropriate changes can be introduced within the framework and/or hypervariable region, using routine techniques, to correct and re-establish proper hypervariable region-antigen interactions.

Phage (mid) display (also referred to herein as phage display in some contexts) can be used as a convenient and fast method for generating and screening many different potential variant antibodies in a library generated by sequence randomization. However, other methods for making and screening altered antibodies are available to the skilled person.

Phage (mid) display technology has provided a powerful tool for generating and selecting novel proteins which bind to a ligand, such as an antigen. Using the techniques of phage (mid) display allows the generation of large libraries of protein variants which can be rapidly sorted for those sequences that bind to a target molecule with high affinity. Nucleic acids encoding variant polypeptides are generally fused to a nucleic acid sequence encoding a viral coat protein, such as the gene III protein or the gene VIII protein. Monovalent phagemid display systems where the nucleic acid sequence encoding the protein or polypeptide is fused to a nucleic acid sequence encoding a portion of the gene III protein have been developed. (Bass, S., Proteins, 8:309 (1990); Lowman and Wells, Methods: A Companion to Methods in Enzymology, 3:205 (1991)). In a monovalent phagemid display system, the gene fusion is expressed at low levels and wild type gene III proteins are also expressed so that infectivity of the particles is retained. Methods of generating peptide libraries and screening those libraries have been disclosed in many patents (e.g., U.S. Pat. Nos. 5,723,286, 5,432,018, 5,580,717, 5,427,908 and 5,498,530).

Libraries of antibodies or antigen binding polypeptides have been prepared in a number of ways including by altering a single gene by inserting random DNA sequences or by cloning a family of related genes. Methods for displaying antibodies or antigen binding fragments using phage (mid) display have been described in U.S. Pat. Nos. 5,750,373, 5,733,743, 5,837,242, 5,969,108, 6,172,197, 5,580,717, and 5,658,727. The library is then screened for expression of antibodies or antigen binding proteins with the desired characteristics.

Methods of substituting an amino acid of choice into a template nucleic acid are well established in the art, some of which are described herein. For example, hypervariable region residues can be substituted using the Kunkel method (e.g., Kunkel et al., Methods Enzymol. 154:367-382 (1987)).

The sequence of oligonucleotides includes one or more of the designed codon sets for the hypervariable region residues to be altered. A codon set is a set of different nucleotide triplet sequences used to encode desired variant amino acids. Codon sets can be represented using symbols to designate particular nucleotides or equimolar mixtures of nucleotides as shown in below according to the IUB code.
IUB Codes
G Guanine
A Adenine
T Thymine
C Cytosine
R (A or G)
Y (C or T)
M (A or C)
K (G or T)
S (C or G)
W (A or T)
H (A or C or T)
B (C or G or T)
V (A or C or G)
D (A or G or T) H
N (A or C or G or T)

For example, in the codon set DVK, D can be nucleotides A or G or T; V can be A or G or C; and K can be G or T. This codon set can present 18 different codons and can encode amino acids Ala, Trp, Tyr, Lys, Thr, Asn, Lys, Ser, Arg, Asp, Glu, Gly, and Cys.

Oligonucleotide or primer sets can be synthesized using standard methods. A set of oligonucleotides can be synthesized, for example, by solid phase synthesis, containing sequences that represent all possible combinations of nucleotide triplets provided by the codon set and that will encode the desired group of amino acids. Synthesis of oligonucleotides with selected nucleotide “degeneracy” at certain positions is well known in that art. Such sets of nucleotides having certain codon sets can be synthesized using commercial nucleic acid synthesizers (available from, for example, Applied Biosystems, Foster City, Calif.), or can be obtained commercially (for example, from Life Technologies, Rockville, Md.). Therefore, a set of oligonucleotides synthesized having a particular codon set will typically include a plurality of oligonucleotides with different sequences, the differences established by the codon set within the overall sequence. Oligonucleotides, as used according to the invention, have sequences that allow for hybridization to a variable domain nucleic acid template and also can include restriction enzyme sites for cloning purposes.

In one method, nucleic acid sequences encoding variant amino acids can be created by oligonucleotide-mediated mutagenesis. This technique is well known in the art as described by Zoller et al. Nucleic Acids Res. 10:6487-6504 (1987). Briefly, nucleic acid sequences encoding variant amino acids are created by hybridizing an oligonucleotide set encoding the desired codon sets to a DNA template, where the template is the single-stranded form of the plasmid containing a variable region nucleic acid template sequence. After hybridization, DNA polymerase is used to synthesize an entire second complementary strand of the template that will thus incorporate the oligonucleotide primer and will contain the codon sets as provided by the oligonucleotide set.

Generally, oligonucleotides of at least 25 nucleotides in length are used. An optimal oligonucleotide will have 12 to 15 nucleotides that are completely complementary to the template on either side of the nucleotide(s) coding for the mutation(s). This ensures that the oligonucleotide will hybridize properly to the single-stranded DNA template molecule. The oligonucleotides are readily synthesized using techniques known in the art such as that described by Crea et al., Proc. Nat'l. Acad. Sci. USA, 75:5765 (1978).

The DNA template is generated by those vectors that are either derived from bacteriophage M13 vectors (the commercially available M13 mp 18 and M13 mp 19 vectors are suitable), or those vectors that contain a single-stranded phage origin of replication as described by Viera et al., Meth. Enzymol., 153:3 (1987). Thus, the DNA that is to be mutated can be inserted into one of these vectors in order to generate single-stranded template. Production of the single-stranded template is described in sections 4.21-4.41 of Sambrook et al., above.

To alter the native DNA sequence, the oligonucleotide is hybridized to the single stranded template under suitable hybridization conditions. A DNA polymerizing enzyme, usually T7 DNA polymerase or the Klenow fragment of DNA polymerase I, is then added to synthesize the complementary strand of the template using the oligonucleotide as a primer for synthesis. A heteroduplex molecule is thus formed such that one strand of DNA encodes the mutated form of gene 1, and the other strand (the original template) encodes the native, unaltered sequence of gene 1. This heteroduplex molecule is then transformed into a suitable host cell, usually a prokaryote such as E. coli JM101. After growing the cells, they are plated onto agarose plates and screened using the oligonucleotide primer radiolabelled with a 32-Phosphate to identify the bacterial colonies that contain the mutated DNA.

The method described immediately above may be modified such that a homoduplex molecule is created wherein both strands of the plasmid contain the mutation(s). The modifications are as follows: The single stranded oligonucleotide is annealed to the single-stranded template as described above. A mixture of three deoxyribonucleotides, deoxyriboadenosine (dATP), deoxyriboguanosine (dGTP), and deoxyribothymidine (dTT), is combined with a modified thiodeoxyribocytosine called dCTP-(aS) (which can be obtained from Amersham). This mixture is added to the template-oligonucleotide complex. Upon addition of DNA polymerase to this mixture, a strand of DNA identical to the template except for the mutated bases is generated. In addition, this new strand of DNA will contain dCTP-(aS) instead of dCTP, which serves to protect it from restriction endonuclease digestion. After the template strand of the double-stranded heteroduplex is nicked with an appropriate restriction enzyme, the template strand can be digested with ExoIII nuclease or another appropriate nuclease past the region that contains the site(s) to be mutagenized. The reaction is then stopped to leave a molecule that is only partially single-stranded. A complete double-stranded DNA homoduplex is then formed using DNA polymerase in the presence of all four deoxyribonucleotide triphosphates, ATP, and DNA ligase. This homoduplex molecule can then be transformed into a suitable host cell.

As indicated previously the sequence of the oligonucleotide set is of sufficient length to hybridize to the template nucleic acid and may also, but does not necessarily, contain restriction sites. The DNA template can be generated by those vectors that are either derived from bacteriophage M13 vectors or vectors that contain a single-stranded phage origin of replication as described by Viera et al. Meth. Enzymol., 153:3 (1987). Thus, the DNA that is to be mutated must be inserted into one of these vectors in order to generate single-stranded template. Production of the single-stranded template is described in sections 4.21-4.41 of Sambrook et al., supra.

According to another method, antigen binding may be restored during humanization of antibodies through the selection of repaired hypervariable regions (see, e.g., U.S. application Ser. No. 11/061,841, filed Feb. 18, 2005). The method includes incorporating non-human hypervariable regions onto an acceptor framework and further introducing one or more amino acid substitutions in one or more hypervariable regions without modifying the acceptor framework sequence. Alternatively, the introduction of one or more amino acid substitutions may be accompanied by modifications in the acceptor framework sequence.

According to another method, a library can be generated by providing upstream and downstream oligonucleotide sets, each set having a plurality of oligonucleotides with different sequences, the different sequences established by the codon sets provided within the sequence of the oligonucleotides. The upstream and downstream oligonucleotide sets, along with a variable domain template nucleic acid sequence, can be used in a polymerase chain reaction to generate a “library” of PCR products. The PCR products can be referred to as “nucleic acid cassettes”, as they can be fused with other related or unrelated nucleic acid sequences, for example, viral coat proteins and dimerization domains, using established molecular biology techniques.

The sequence of the PCR primers includes one or more of the designed codon sets for the solvent accessible and highly diverse positions in a hypervariable region. As described above, a codon set is a set of different nucleotide triplet sequences used to encode desired variant amino acids.

Antibody selectants that meet the desired criteria, as selected through appropriate screening/selection steps can be isolated and cloned using standard recombinant techniques.

It is further important that antibodies be humanized with retention of high binding affinity for the antigen and other favorable biological properties. To achieve this goal, according to a preferred method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

Various forms of a humanized anti-activin antibody are contemplated. For example, the humanized antibody may be an antibody fragment, such as a Fab. Alternatively, the humanized antibody may be an intact antibody, such as an intact IgG1 antibody.

As an alternative to humanization, human antibodies can be generated. For example, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array into such germ-line mutant mice will result in the production of human antibodies upon antigen challenge (see, e.g., Jakobovits et al., Proc. Natl. Acad. Sci. USA, 90:2551 (1993); Jakobovits et al., Nature, 362:255-8 (1993); Bruggemann et al., Year in Immuno. 7:33 (1993); U.S. Pat. Nos. 5,545,806, 5,569,825, 5,591,669; 5,545,807; and WO 97/17852).

Alternatively, phage display technology (McCafferty et al., Nature 348:552-53 (1990)) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B-cell. Phage display can be performed in a variety of formats, reviewed in, e.g., Johnson, Kevin S, and Chiswell, David J., Current Opinion in Structural Biology 3:564-571 (1993). Several sources of V-gene segments can be used for phage display. Clackson et al., Nature, 352:624-628 (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Marks et al., J. Mol. Biol. 222:581-97 (1991), or Griffith et al., EMBO J. 12:725-34 (1993) (see also, U.S. Pat. Nos. 5,565,332 and 5,573,905).

As discussed above, human antibodies may also be generated by in vitro activated B cells (see, e.g., U.S. Pat. Nos. 5,567,610 and 5,229,275).

In another embodiment, the antibodies of this disclosure are human monoclonal antibodies. Such human monoclonal antibodies directed against activin can be generated using transgenic or transchromosomic mice carrying parts of the human immune system rather than the mouse system. These transgenic and transchromosomic mice include mice referred to herein as the HuMAb Mouse™ and KM Mouse™, respectively, and are collectively referred to herein as “human Ig mice.”

The HuMAb Mouse™ (Medarex, Inc.) contains human immunoglobulin gene miniloci that encode unrearranged human heavy (μ and γ) and κ light chain immunoglobulin sequences, together with targeted mutations that inactivate the endogenous μ and κ chain loci (see e.g., Lonberg, et al. (1994) Nature 368 (6474): 856-9). Accordingly, the mice exhibit reduced expression of mouse IgM or κ, and in response to immunization, the introduced human heavy and light chain transgenes undergo class switching and somatic mutation to generate high affinity human IgGκ monoclonal antibodies (Lonberg, N. et al. (1994), supra; reviewed in Lonberg, N. (1994) Handbook of Experimental Pharmacology 113:49-101; Lonberg, N. and Huszar, D. (1995) Intern. Rev. Immunol. 13:65-93, and Harding, F. and Lonberg, N. (1995) Ann. N.Y. Acad. Sci. 764:536-46). Preparation and use of the HuMAb Mouse™, and the genomic modifications carried by such mice, is further described in Taylor, L. et al. (1992) Nucleic Acids Research 20:6287-6295; Chen, J. et al. (1993) International Immunology 5:647-656; Tuaillon et al. (1993) Proc. Natl. Acad. Sci. USA 90:3720-4; Choi et al. (1993) Nature Genetics 4:117-23; Chen, J. et al. (1993) EMBO J. 12:21-830; Tuaillon et al., (1994) J. Immunol. 152:2912-20; Taylor, L. et al. (1994) International Immunology 6:579-91; and Fishwild, D. et al. (1996) Nature Biotechnology 14:845-51, the contents of all of which are hereby specifically incorporated by reference in their entirety. See further, U.S. Pat. Nos. 5,545,806; 5,569,825; 5,625,126; 5,633,425; 5,789,650; 5,877,397; 5,661,016; 5,814,318; 5,874,299; and 5,770,429; 5,545,807; PCT Publication Nos. WO 92/03918, WO 93/12227, WO 94/25585, WO 97/13852, WO 98/24884 and WO 99/45962; and PCT Publication No. WO 01/14424.

In another embodiment, human antibodies of this disclosure can be raised using a mouse that carries human immunoglobulin sequences on transgenes and transchomosomes, such as a mouse that carries a human heavy chain transgene and a human light chain transchromosome. This mouse is referred to herein as a “KM Mouse™” and is described in detail in PCT Publication WO 02/43478.

Still further, alternative transgenic animal systems expressing human immunoglobulin genes are available in the art and can be used to raise anti-activin antibodies of this disclosure. For example, an alternative transgenic system referred to as the Xenomouse (Abgenix, Inc.) can be used; such mice are described in, for example, U.S. Pat. Nos. 5,939,598; 6,075,181; 6,114,598; 6,150,584 and 6,162,963.

Moreover, alternative transchromosomic animal systems expressing human immunoglobulin genes are available in the art and can be used to raise anti-activin antibodies of this disclosure. For example, mice carrying both a human heavy chain transchromosome and a human light chain tranchromosome, referred to as “TC mice” can be used; such mice are described in Tomizuka et al. (2000) Proc. Natl. Acad. Sci. USA 97:722-7. Furthermore, cows carrying human heavy and light chain transchromosomes have been described in the art (e.g., Kuroiwa et al. (2002) Nature Biotechnology 20:889-94 and PCT application No. WO 2002/092812) and can be used to raise anti-activin antibodies of this disclosure.

### 4. Antibody Fragments

In certain circumstances there are advantages of using antibody fragments, rather than whole antibodies. The smaller size of the fragments allows for rapid clearance, and may lead to improved access to solid tumors.

Various techniques have been developed for the production of antibody fragments. Traditionally, these fragments were derived via proteolytic digestion of intact antibodies (see, e.g., Morimoto et al., Journal of Biochemical and Biophysical Methods 24:107-7 (1992); and Brennan et al., Science, 229:81 (1985)). However, these fragments can now be produced directly by recombinant host cells. Fab, Fv and scFv antibody fragments can all be expressed in and secreted from E. coli, thus allowing the facile production of large amounts of these fragments. Antibody fragments can be isolated from the antibody phage libraries discussed above. Alternatively, Fab′-SH fragments can be directly recovered from E. coli and chemically coupled to form F(ab′)2 fragments (Carter et al., Bio/Technology 10:163-7 (1992)). According to another approach, F(ab′)2 fragments can be isolated directly from recombinant host cell culture. Fab and F(ab′)2 fragment with increased in vivo half-life comprising a salvage receptor binding epitope residues are described in U.S. Pat. No. 5,869,046. Other techniques for the production of antibody fragments will be apparent to the skilled practitioner. In other embodiments, the antibody of choice is a single chain Fv fragment (scFv) (see WO 93/16185; U.S. Pat. Nos. 5,571,894; and 5,587,458). Fv and sFv are the only species with intact combining sites that are devoid of constant regions; thus, they are suitable for reduced nonspecific binding during in vivo use. sFv fusion proteins may be constructed to yield fusion of an effector protein at either the amino or the carboxy terminus of an sFv (see Antibody Engineering, ed. Borrebaeck, supra. The antibody fragment may also be a “linear antibody”, e.g., as described in U.S. Pat. No. 5,641,870 for example.

In one embodiment, an anti-activin antibody derived scFv is used in a CAR modified immune cell, such as a CAR-T or CAR-NK cell, or CAR macrophage. Included among anti-activin antibody fragments are portions of anti-activin antibodies (and combinations of portions of anti-activin antibodies, for example, scFv) that may be used as targeting arms, directed to activin tumor epitope, in chimeric antigenic receptors of CAR-T or CAR-NK cells, or CAR macrophages. Such fragments are not necessarily proeteolytic fragments but rather portions of polypeptide sequences that can confer affinity for target.

### 5. Bispecific Antibodies

Bispecific antibodies are antibodies that have binding specificities for at least two different epitopes. Exemplary bispecific antibodies may bind to two different epitopes of an activin protein as described herein. Other such antibodies may combine an activin binding site with a binding site for another protein. Alternatively, an anti-activin arm may be combined with an arm which binds to a triggering molecule on a leukocyte such as a T-cell receptor molecule (e.g., CD3), or Fc receptors for IgG (FcγR), such as FcγRI (CD64), FcγRII (CD32) and FcγRIII (CD16), so as to focus and localize cellular defense mechanisms to the activin-expressing cell. Bispecific antibodies may also be used to localize cytotoxic agents to cells which express activin. These antibodies possess an activin-binding arm and an arm which binds the cytotoxic agent (e.g., saporin, anti-interferon-α, vinca alkaloid, ricin A chain, methotrexate or radioactive isotope hapten). Bispecific antibodies can be prepared as full length antibodies or antibody fragments (e.g., F(ab′)2 bispecific antibodies).

WO 96/16673 describes a bispecific anti-ErbB2/anti-FcγRIII antibody and U.S. Pat. No. 5,837,234 discloses a bispecific anti-ErbB2/anti-FcγRI antibody. A bispecific anti-ErbB2/Fcα antibody is shown in WO98/02463. U.S. Pat. No. 5,821,337 teaches a bispecific anti-ErbB2/anti-CD3 antibody.

Methods for making bispecific antibodies are known in the art. Traditional production of full length bispecific antibodies is based on the co-expression of two immunoglobulin heavy chain-light chain pairs, where the two chains have different specificities (Millstein et al., Nature 305:537-9 (1983)). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low. Similar procedures are disclosed in WO 93/08829, and in Traunecker et al., EMBO J. 10:3655-3659 (1991).

### 6 Effector Function Engineering

It may be desirable to modify the antibody of the invention with respect to effector function, e.g., so as to enhance antigen-dependent cell-mediated cyotoxicity (ADCC) and/or complement dependent cytotoxicity (CDC) of the antibody. This may be achieved by introducing one or more amino acid substitutions in an Fc region of the antibody. Alternatively or additionally, cysteine residue(s) may be introduced in the Fc region, thereby allowing interchain disulfide bond formation in this region. The homodimeric antibody thus generated may have improved internalization capability and/or increased complement-mediated cell killing and antibody-dependent cellular cytotoxicity (ADCC) (see Caron et al., J. Exp Med. 176:1191-5 (1992); Shopes, B. J. Immunol. 148:2918-22 (1992). Homodimeric antibodies with enhanced anti-tumor activity may also be prepared using heterobifunctional cross-linkers as described in Wolff et al., Cancer Research 53:2560-5 (1993). Alternatively, an antibody can be engineered which has dual Fc regions and may thereby have enhanced complement lysis and

ADCC capabilities. See Stevenson et al., Anti-Cancer Drug Design 3:219-30 (1989). To increase the serum half life of the antibody, one may incorporate a salvage receptor binding epitope into the antibody (especially an antibody fragment) as described in U.S. Pat. No. 5,739,277, for example. As used herein, the term “salvage receptor binding epitope” refers to an epitope of the Fc region of an IgG molecule (e.g., IgG1, IgG2, IgG3, or IgG4) that is responsible for increasing the in vivo serum half-life of the IgG molecule.

### B. Certain Methods of Making Antibodies

1. Screening for Anti-Activin Antibodies with the Desired Properties

Techniques for generating antibodies that bind to activin polypeptides have been described above. One may further select antibodies with certain biological characteristics, as desired.

The growth inhibitory effects of an anti-activin antibody of the invention may be assessed by methods known in the art, e.g., using cells which express an activin polypeptide either endogenously or following transfection with the activin gene. For example, appropriate tumor cell lines and activin-transfected cells may be treated with an anti-activin monoclonal antibody of the invention at various concentrations for a few days (e.g., 2-7) days and stained with crystal violet or MTT or analyzed by some other colorimetric assay. Another method of measuring proliferation would be by comparing 3H-thymidine uptake by the cells treated in the presence or absence an anti-activin antibody of the invention. After treatment, the cells are harvested and the amount of radioactivity incorporated into the DNA quantitated in a scintillation counter. Appropriate positive controls include treatment of a selected cell line with a growth inhibitory antibody known to inhibit growth of that cell line. Growth inhibition of tumor cells in vivo can be determined in various ways known in the art. The tumor cell may be one that overexpresses an activin polypeptide. The anti-activin antibody will inhibit cell proliferation of an activin-expressing tumor cell in vitro or in vivo by about 25-100% compared to the untreated tumor cell, more preferably, by about 30-100%, and even more preferably by about 50-100% or 70-100%, in one embodiment, at an antibody concentration of about 0.5 to 30 μg/mL. Growth inhibition can be measured at an antibody concentration of about 0.5 to 30 μg/mL or about 0.5 nM to 200 nM in cell culture, where the growth inhibition is determined 1-10 days after exposure of the tumor cells to the antibody. The antibody is growth inhibitory in vivo if administration of the anti-activin antibody at about 1 μg/kg to about 100 mg/kg body weight results in reduction in tumor size or reduction of tumor cell proliferation within about 5 days to 3 months from the first administration of the antibody, preferably within about 5 to 30 days.

To select for an anti-activin antibody which induces cell death, loss of membrane integrity as indicated by, e.g., propidium iodide (PI), trypan blue or 7AAD uptake may be assessed relative to control. A PI uptake assay can be performed in the absence of complement and immune effector cells. Activin polypeptide-expressing tumor cells are incubated with medium alone or medium containing the appropriate anti-activin antibody (e.g, at about 10 μg/mL). The cells are incubated for a 3 day time period. Following each treatment, cells are washed and aliquoted into 35 mm strainer-capped 12×75 tubes (1 mL per tube, 3 tubes per treatment group) for removal of cell clumps. Tubes then receive PI (10 μg/mL). Samples may be analyzed using a FACSCAN® flow cytometer and FACSCONVERT® CellQuest software (Becton Dickinson). Those anti-activin antibodies that induce statistically significant levels of cell death as determined by PI uptake may be selected as cell death-inducing anti-activin antibodies.

To screen for antibodies which bind to an epitope on an activin polypeptide bound by an antibody of interest, a routine cross-blocking assay such as that described in Antibodies, A Laboratory Manual, Cold Spring Harbor Laboratory, Ed Harlow and David Lane (1988), can be performed. This assay can be used to determine if a test antibody binds the same site or epitope as a known anti-Activin antibody. Alternatively, or additionally, epitope mapping can be performed by methods known in the art. For example, the antibody sequence can be mutagenized such as by alanine scanning, to identify contact residues. The mutant antibody is initially tested for binding with polyclonal antibody to ensure proper folding. In a different method, peptides corresponding to different regions of an activin polypeptide can be used in competition assays with the test antibodies or with a test antibody and an antibody with a characterized or known epitope.

In addition, candidate antibodies may also be screened for function using one or more of the following: in vivo screening for inhibition of metastasis, inhibition of chemotaxis by an in vitro method (e.g., U.S. 2010/0061978, incorporated herein by reference in its entirety), inhibition of vascularization, inhibition of tumor growth, and decrease in tumor size.

### 2. Certain Library Screening Methods

Anti-activin antibodies of the invention can be made by using combinatorial libraries to screen for antibodies with the desired activity or activities. For example, a variety of methods are known in the art for generating phage display libraries and screening such libraries for antibodies possessing the desired binding characteristics. Such methods are described generally in Hoogenboom et al. (2001) in Methods in Molecular Biology 178:1-37 (O'Brien et al., ed., Human Press, Totowa, NJ), and in certain embodiments, in Lee et al. (2004) J. Mol. Biol. 340:1073-93.

In principle, synthetic antibody clones are selected by screening phage libraries containing phage that display various fragments of antibody variable region (Fv) fused to phage coat protein. Such phage libraries are panned by affinity chromatography against the desired antigen. Clones expressing Fv fragments capable of binding to the desired antigen are adsorbed to the antigen and thus separated from the non-binding clones in the library. The binding clones are then eluted from the antigen, and can be further enriched by additional cycles of antigen adsorption/elution. Any of the anti-activin antibodies of the invention can be obtained by designing a suitable antigen screening procedure to select for the phage clone of interest followed by construction of a full length anti-activin antibody clone using the Fv sequences from the phage clone of interest and suitable constant region (Fc) sequences described in Kabat et al., Sequences of Proteins of Immunological Interest, Fifth Edition, NIH Publication 91-3242, Bethesda MD (1991), vols. 1-3.

In certain embodiments, the antigen-binding domain of an antibody is formed from two variable (V) regions of about 110 amino acids, one each from the light (VL) and heavy (VH) chains, that both present three hypervariable loops (HVRs) or complementarity-determining regions (CDRs). Variable domains can be displayed functionally on phage, either as single-chain Fv (scFv) fragments, in which VH and VL are covalently linked through a short, flexible peptide, or as Fab fragments, in which they are each fused to a constant domain and interact non-covalently, as described in Winter et al., Ann. Rev. Immunol., 12:433-55 (1994). As used herein, scFv encoding phage clones and Fab encoding phage clones are collectively referred to as “Fv phage clones” or “Fv clones.”

Repertoires of VH and VL genes can be separately cloned by polymerase chain reaction (PCR) and recombined randomly in phage libraries, which can then be searched for antigen-binding clones as described in Winter et al., Ann. Rev. Immunol., 12:433-55 (1994). Libraries from immunized sources provide high-affinity antibodies to the immunogen without the requirement of constructing hybridomas. Alternatively, the naive repertoire can be cloned to provide a single source of human antibodies to a wide range of non-self and also self antigens without any immunization as described by Griffiths et al., EMBO J, 12:725-34 (1993). Finally, naive libraries can also be made synthetically by cloning the unrearranged V-gene segments from stem cells and using PCR primers containing random sequence to encode the highly variable CDR3 regions and to accomplish rearrangement in vitro as described by Hoogenboom and Winter, J. Mol. Biol., 227:381-8 (1992).

In certain embodiments, filamentous phage is used to display antibody fragments by fusion to the minor coat protein pIII. The antibody fragments can be displayed as single chain Fv fragments, in which VH and VL domains are connected on the same polypeptide chain by a flexible polypeptide spacer, e.g., as described by Marks et al., J. Mol. Biol., 222:581-97 (1991), or as Fab fragments, in which one chain is fused to pIII and the other is secreted into the bacterial host cell periplasm where assembly of a Fab-coat protein structure which becomes displayed on the phage surface by displacing some of the wild type coat proteins, e.g., as described in Hoogenboom et al., Nucl. Acids Res., 19:4133-7 (1991).

In general, nucleic acids encoding antibody gene fragments are obtained from immune cells harvested from humans or animals. If a library biased in favor of anti-activin clones is desired, the subject is immunized with activin to generate an antibody response, and spleen cells and/or circulating B cells other peripheral blood lymphocytes (PBLs) are recovered for library construction. In some embodiments, a human antibody gene fragment library biased in favor of anti-activin clones is obtained by generating an anti-activin antibody response in transgenic mice carrying a functional human immunoglobulin gene array (and lacking a functional endogenous antibody production system) such that activin immunization gives rise to B cells producing human antibodies against Activin. The generation of human antibody-producing transgenic mice is described below.

Additional enrichment for anti-activin reactive cell populations can be obtained by using a suitable screening procedure to isolate B cells expressing activin-specific membrane bound antibody, e.g., by cell separation using activin affinity chromatography or adsorption of cells to fluorochrome-labeled activin followed by flow-activated cell sorting (FACS).

Alternatively, the use of spleen cells and/or B cells or other PBLs from an unimmunized donor provides a better representation of the possible antibody repertoire, and also permits the construction of an antibody library using any animal (human or non-human) species in which activin is not antigenic. For libraries incorporating in vitro antibody gene construction, stem cells are harvested from the subject to provide nucleic acids encoding unrearranged antibody gene segments. The immune cells of interest can be obtained from a variety of animal species, such as human, mouse, rat, lagomorpha, luprine, canine, feline, porcine, bovine, equine, and avian species, etc.

Nucleic acid encoding antibody variable gene segments (including VH and VL segments) are recovered from the cells of interest and amplified. In the case of rearranged VH and VL gene libraries, the desired DNA can be obtained by isolating genomic DNA or mRNA from lymphocytes followed by polymerase chain reaction (PCR) with primers matching the 5′ and 3′ ends of rearranged VH and VL genes as described in Orlandi et al., Proc. Natl. Acad. Sci. (USA), 86:3833-7 (1989), thereby making diverse V gene repertoires for expression.

The V genes can be amplified from cDNA and genomic DNA, with back primers at the 5′ end of the exon encoding the mature V-domain and forward primers based within the J-segment as described in Orlandi et al. (1989) and in Ward et al., Nature, 341:544-6 (1989). However, for amplifying from cDNA, back primers can also be based in the leader exon as described in Jones et al., Biotechnol., 9:88-9 (1991), and forward primers within the constant region as described in Sastry et al., Proc. Natl. Acad. Sci. (USA), 86:5728-32 (1989). To maximize complementarity, degeneracy can be incorporated in the primers as described in Orlandi et al. (1989) or Sastry et al. (1989). In certain embodiments, library diversity is maximized by using PCR primers targeted to each V-gene family in order to amplify all available VH and VL arrangements present in the immune cell nucleic acid sample, e.g., as described in the method of Marks et al., J. Mol. Biol., 222:581-97 (1991) or as described in the method of Orum et al., Nucleic Acids Res., 21:4491-98 (1993). For cloning of the amplified DNA into expression vectors, rare restriction sites can be introduced within the PCR primer as a tag at one end as described in Orlandi et al. (1989), or by further PCR amplification with a tagged primer as described in Clackson et al., Nature, 352:624-628 (1991).

Repertoires of synthetically rearranged V genes can be derived in vitro from V gene segments. Most of the human VH-gene segments have been cloned and sequenced (reported in Tomlinson et al., J. Mol. Biol., 227:776-98 (1992)), and mapped (reported in Matsuda et al., Nature Genet., 3:88-94 (1993); these cloned segments (including all the major conformations of the H1 and H2 loop) can be used to generate diverse VH gene repertoires with PCR primers encoding H3 loops of diverse sequence and length as described in Hoogenboom and Winter, J. Mol. Biol., 227:381-388 (1992). VH repertoires can also be made with all the sequence diversity focused in a long H3 loop of a single length as described in Barbas et al., Proc. Natl. Acad. Sci. USA, 89:4457-61 (1992). Human Vκ and Vλ segments have been cloned and sequenced (reported in Williams and Winter, Eur. J. Immunol., 23:1456-61 (1993)) and can be used to make synthetic light chain repertoires. Synthetic V gene repertoires, based on a range of VH and VL folds, and L3 and H3 lengths, will encode antibodies of considerable structural diversity. Following amplification of V-gene encoding DNAs, germline V-gene segments can be rearranged in vitro according to the methods of Hoogenboom and Winter, J. Mol. Biol., 227:381-8 (1992).

Repertoires of antibody fragments can be constructed by combining VH and VL gene repertoires together in several ways. Each repertoire can be created in different vectors, and the vectors recombined in vitro, e.g., as described in Hogrefe et al., Gene, 128:119-26 (1993), or in vivo by combinatorial infection, e.g., the loxP system described in Waterhouse et al., Nucl. Acids Res., 21:2265-66 (1993). The in vivo recombination approach exploits the two-chain nature of Fab fragments to overcome the limit on library size imposed by E. coli transformation efficiency. Naive VH and VL repertoires are cloned separately, one into a phagemid and the other into a phage vector. The two libraries are then combined by phage infection of phagemid-containing bacteria so that each cell contains a different combination and the library size is limited only by the number of cells present (about 1012 clones). Both vectors contain in vivo recombination signals so that the VH and VL genes are recombined onto a single replicon and are co-packaged into phage virions. These huge libraries provide large numbers of diverse antibodies of good affinity (Kd-1 of about 10-8 M).

Alternatively, the repertoires may be cloned sequentially into the same vector, e.g., as described in Barbas et al., Proc. Natl. Acad. Sci. USA, 88:7978-7982 (1991), or assembled together by PCR and then cloned, e.g., as described in Clackson et al., Nature, 352:624-628 (1991). PCR assembly can also be used to join VH and VL DNAs with DNA encoding a flexible peptide spacer to form single chain Fv (scFv) repertoires. In yet another technique, “in cell PCR assembly” is used to combine VH and VL genes within lymphocytes by PCR and then clone repertoires of linked genes as described in Embleton et al., Nucl. Acids Res., 20:3831-3837 (1992).

The antibodies produced by naive libraries (either natural or synthetic) can be of moderate affinity (Kd-1 of about 10⁶ to 10⁷ M-1), but affinity maturation can also be mimicked in vitro by constructing and reselecting from secondary libraries as described in Winter et al. (1994), supra. For example, mutation can be introduced at random in vitro by using error-prone polymerase (reported in Leung et al., Technique, 1:11-5 (1989)) in the method of Hawkins et al., J. Mol. Biol., 226:889-96 (1992) or in the method of Gram et al., Proc. Natl. Acad. Sci USA, 89:3576-80 (1992). Additionally, affinity maturation can be performed by randomly mutating one or more CDRs, e.g., using PCR with primers carrying random sequence spanning the CDR of interest, in selected individual Fv clones and screening for higher affinity clones. WO 9607754 described a method for inducing mutagenesis in a complementarity determining region of an immunoglobulin light chain to create a library of light chain genes. Another effective approach is to recombine the VH or VL domains selected by phage display with repertoires of naturally occurring V domain variants obtained from unimmunized donors and screen for higher affinity in several rounds of chain reshuffling as described in Marks et al., Biotechnol., 10:779-83 (1992). This technique allows the production of antibodies and antibody fragments with affinities of about 10-9 M or less.

Screening of the libraries can be accomplished by various techniques known in the art. For example, Activin can be used to coat the wells of adsorption plates, expressed on host cells affixed to adsorption plates or used in cell sorting, or conjugated to biotin for capture with streptavidin-coated beads, or used in any other method for panning phage display libraries.

The phage library samples are contacted with immobilized activin under conditions suitable for binding at least a portion of the phage particles with the adsorbent. Normally, the conditions, including pH, ionic strength, temperature and the like are selected to mimic physiological conditions. The phages bound to the solid phase are washed and then eluted by acid, e.g., as described in Barbas et al., Proc. Natl. Acad. Sci USA, 88:7978-82 (1991), or by alkali, e.g., as described in Marks et al., J. Mol. Biol., 222:581-97 (1991), or by Activin antigen competition, e.g., in a procedure similar to the antigen competition method of Clackson et al., Nature, 352:624-8 (1991). Phages can be enriched 20 to 1,000-fold in a single round of selection. Moreover, the enriched phages can be grown in bacterial culture and subjected to further rounds of selection.

The efficiency of selection depends on many factors, including the kinetics of dissociation during washing, and whether multiple antibody fragments on a single phage can simultaneously engage with antigen. Antibodies with fast dissociation kinetics (and weak binding affinities) can be retained by use of short washes, multivalent phage display and high coating density of antigen in solid phase. The high density not only stabilizes the phage through multivalent interactions but favors rebinding of phage that has dissociated. The selection of antibodies with slow dissociation kinetics (and good binding affinities) can be promoted by use of long washes and monovalent phage display as described in Bass et al., Proteins, 8:309-314 (1990) and in WO 92/09690, and a low coating density of antigen as described in Marks et al., Biotechnol., 10:779-783 (1992).

It is possible to select between phage antibodies of different affinities, even with affinities that differ slightly, for activin. However, random mutation of a selected antibody (e.g., as performed in some affinity maturation techniques) is likely to give rise to many mutants, most binding to antigen, and a few with higher affinity. With limiting activin, rare high affinity phage could be competed out. To retain all higher affinity mutants, phages can be incubated with excess biotinylated activin, but with the biotinylated activin at a concentration of lower molarity than the target molar affinity constant for activin. The high affinity-binding phages can then be captured by streptavidin-coated paramagnetic beads. Such “equilibrium capture” allows the antibodies to be selected according to their affinities of binding, with sensitivity that permits isolation of mutant clones with as little as two-fold higher affinity from a great excess of phages with lower affinity. Conditions used in washing phages bound to a solid phase can also be manipulated to discriminate on the basis of dissociation kinetics.

Anti-activin clones may be selected based on activity. In certain embodiments, the invention provides anti-activin antibodies that bind to living cells that naturally express activin. In one embodiment, the invention provides anti-activin antibodies that block the binding between an activin ligand and activin, but do not block the binding between an activin ligand and a second protein. Fv clones corresponding to such anti-activin antibodies can be selected by (1) isolating anti-activin clones from a phage library as described above, and optionally amplifying the isolated population of phage clones by growing up the population in a suitable bacterial host; (2) selecting activin and a second protein against which blocking and non-blocking activity, respectively, is desired; (3) adsorbing the anti-activin phage clones to immobilized activin; (4) using an excess of the second protein to elute any undesired clones that recognize activin-binding determinants which overlap or are shared with the binding determinants of the second protein; and (5) eluting the clones which remain adsorbed following step (4). Optionally, clones with the desired blocking/non-blocking properties can be further enriched by repeating the selection procedures described herein one or more times.

DNA encoding hybridoma-derived monoclonal antibodies or phage display Fv clones of the invention is readily isolated and sequenced using conventional procedures (e.g., by using oligonucleotide primers designed to specifically amplify the heavy and light chain coding regions of interest from hybridoma or phage DNA template). Once isolated, the DNA can be placed into expression vectors, which are then transfected into host cells such as E. coli cells, simian COS cells, Chinese hamster ovary (CHO) cells, or myeloma cells that do not otherwise produce immunoglobulin protein, to obtain the synthesis of the desired monoclonal antibodies in the recombinant host cells. Review articles on recombinant expression in bacteria of antibody-encoding DNA include Skerra et al., Curr. Opinion in Immunol. 5:256 (1993) and Pluckthun, Immunol. Rev. 130:151 (1992).

DNA encoding the Fv clones of the invention can be combined with known DNA sequences encoding heavy chain and/or light chain constant regions (e.g., the appropriate DNA sequences can be obtained from Kabat et al., supra) to form clones encoding full or partial length heavy and/or light chains. It will be appreciated that constant regions of any isotype can be used for this purpose, including IgG, IgM, IgA, IgD, and IgE constant regions, and that such constant regions can be obtained from any human or animal species. An Fv clone derived from the variable domain DNA of one animal (such as human) species and then fused to constant region DNA of another animal species to form coding sequence(s) for “hybrid,” full length heavy chain and/or light chain is included in the definition of “chimeric” and “hybrid” antibody as used herein. In certain embodiments, an Fv clone derived from human variable DNA is fused to human constant region DNA to form coding sequence(s) for full- or partial-length human heavy and/or light chains.

DNA encoding anti-activin antibody derived from a hybridoma can also be modified, for example, by substituting the coding sequence for human heavy- and light-chain constant domains in place of homologous murine sequences derived from the hybridoma clone (e.g., as in the method of Morrison et al., Proc. Natl. Acad. Sci. USA, 81:6851-5 (1984)). DNA encoding a hybridoma- or Fv clone-derived antibody or fragment can be further modified by covalently joining to the immunoglobulin coding sequence all or part of the coding sequence for a non-immunoglobulin polypeptide. In this manner, “chimeric” or “hybrid” antibodies are prepared that have the binding specificity of the Fv clone or hybridoma clone-derived antibodies of the invention.

### 3. Generation of Antibodies Using CAR T-Cells

Anti-activin antibodies of the invention can be made by using CAR T-cell platforms to screen for antibodies with the desired activity or activities. Chimeric antigen receptors (CARs) are composed of an extracellular antigen recognition domain (usually a single-chain variable fragment (scFv) antibody) attached to transmembrane and cytoplasmic signaling domains. Alvarez-Vallina, L, Curr Gene Ther 1:385-97 (2001). CAR-mediated recognition converts tumor-associated antigens (TAA) expressed on the cell surface into recruitment points of effector functions, addressing the goal of major histocompatibility complex-independent activation of effector cells. First-generation CARs were constructed through the fusion of a scFv-based TAA-binding domain to a cytoplasmic signaling domain typically derived either from the ζ chain of the T cell receptor (TCR)/CD3 complex or from the γ chain associated with some Fc receptors (Gross, G. et al., Proc Natl Acad Sci USA 86:10024-8 (1989)). Second-generation CARs (CARv2) comprising the signaling region of the TCR ζ in series with the signaling domain derived from the T-cell co-stimulatory receptors CD28, 4-1BB (CD137) or OX40 (CD134) have also been developed (Sanz, L. et al., Trends Immunol 25:85-91 (2004)).

Upon encountering antigen, the interaction of a genetically transferred CAR triggers effector functions and can mediate cytolysis of tumor cells. The utility and effectiveness of the CAR approach have been demonstrated in a variety of animal models, and ongoing clinical trials using CAR-based genetically engineered T lymphocytes for the treatment of cancer patients. Lipowska-Bhalla, G. et al., Cancer Immunol Immunother 61:953-62 (2012). CARs enable targeting of effector cells toward any native extracellular antigen for which a suitable antibody exists. Engineered cells can be targeted not only to proteins but also to structures such as carbohydrate and glycolipid tumor antigens (Mezzanzanica, D. et al., Cancer Gene Ther 5:401-7 (1998); Kershaw, M H. et al., Nat Rev Immunol 5:928-40 (2005)).

Current methods for the generation of recombinant antibodies are mainly based on the use of purified proteins. Hoogenboom, H. R. et al., Nat Biotechnol 23:1105-1116 (2005). However, a mammalian cell-based antibody display platform has recently been described, which takes advantage of the functional capabilities of T lymphocytes. Alonso-Camino et al, Molecular Therapy Nucleic Acids (2013) 2, e93. The display of antibodies on the surface of T lymphocytes, as a part of a CAR-mediating signaling, may ideally link the antigen-antibody interaction to a demonstrable change in cell phenotype, due to the surface expression of activation markers. Alonso-Camino, V. et al., PLOS ONE 4: e7174 (2009). By using a scFv-based CAR that recognizes a TAA, it has been demonstrated that combining CAR-mediated activation with fluorescence-activated cell sorting (FACS) of CD69+ T cells makes it possible to isolate binders to surface TAA, with an enrichment factor of at least 103-fold after two rounds, resulting in a homogeneous population of T cells expressing TAA-specific CAR. Alonso-Camino, V, et al., PLOS ONE 4: e7174 (2009).

### D. Anti-Activin Antibody Variants and Modifications

### 1. Variants

In addition to the anti-activin antibodies described herein, it is contemplated that anti-activin antibody variants can be prepared. Anti-activin antibody variants can be prepared by introducing appropriate nucleotide changes into the encoding DNA, and/or by synthesis of the desired antibody or polypeptide. Those skilled in the art will appreciate that amino acid changes may alter post-translational processes of the anti-activin antibody, such as changing the number or position of glycosylation sites or altering the membrane anchoring characteristics.

Variations in the anti-activin antibodies described herein, can be made, for example, using any of the techniques and guidelines for conservative and non-conservative mutations set forth, for instance, in U.S. Pat. No. 5,364,934. Variations may be a substitution, deletion or insertion of one or more codons encoding the antibody or polypeptide that results in a change in the amino acid sequence as compared with the native sequence antibody or polypeptide. Optionally the variation is by substitution of at least one amino acid with any other amino acid in one or more of the domains of the anti-activin antibody. Guidance in determining which amino acid residue may be inserted, substituted or deleted without adversely affecting the desired activity may be found by comparing the sequence of the anti-activin antibody with that of homologous known protein molecules and minimizing the number of amino acid sequence changes made in regions of high homology. Amino acid substitutions can be the result of replacing one amino acid with another amino acid having similar structural and/or chemical properties, such as the replacement of a leucine with a serine, i.e., conservative amino acid replacements. Insertions or deletions may optionally be in the range of about 1 to 5 amino acids. The variation allowed may be determined by systematically making insertions, deletions or substitutions of amino acids in the sequence and testing the resulting variants for activity exhibited by the full-length or mature native sequence.

Anti-activin antibody fragments are provided herein. Such fragments may be truncated at the N-terminus or C-terminus, or may lack internal residues, for example, when compared with a full-length native antibody or protein. Certain fragments lack amino acid residues that are not essential for a desired biological activity of the anti-activin antibody.

Anti-activin antibody fragments may be prepared by any of a number of conventional techniques. Desired peptide fragments may be chemically synthesized. An alternative approach involves generating antibody or polypeptide fragments by enzymatic digestion, e.g., by treating the protein with an enzyme known to cleave proteins at sites defined by particular amino acid residues, or by digesting the DNA with suitable restriction enzymes and isolating the desired fragment. Yet another suitable technique involves isolating and amplifying a DNA fragment encoding a desired antibody or polypeptide fragment, by polymerase chain reaction (PCR). Oligonucleotides that define the desired termini of the DNA fragment are employed at the 5′ and 3′ primers in the PCR. Preferably, anti-activin antibody fragments share at least one biological and/or immunological activity with the native anti-activin antibody disclosed herein.

In particular embodiments, conservative substitutions of interest are shown in Table 1 under the heading of preferred substitutions. If such substitutions result in a change in biological activity, then more substantial changes, denominated exemplary substitutions in Table 1, or as further described below in reference to amino acid classes, are introduced and the products screened.

TABLE 1

| Original | Exemplary | Preferred |
|---|---|---|
| Residue | Substitutions | Substitutions |
| Ala (A) | val; leu; ile | val |
| Arg (R) | lys; gln; asn | lys |
| Asn (N) | gln; his; lys; arg | gln |
| Asp (D) | glu | glu |
| Cys (C) | ser | ser |
| Gln (Q) | asn | asn |
| Glu (E) | asp | asp |
| Gly (G) | pro; ala | ala |
| His (H) | asn; gln; lys; arg | arg |
| Ile (I) | leu; val; met; ala; phe; | leu |
| | norleucine | |
| Leu (L) | norleucine; ile; val; | ile |
| | met; ala; phe | |
| Lys (K) | arg; gln; asn | arg |
| Met (M) | leu; phe; ile | leu |
| Phe (F) | leu; val; ile; ala; tyr | leu |
| Pro (P) | ala | ala |
| Ser (S) | thr | thr |
| Thr (T) | ser | ser |
| Trp (W) | tyr; phe | tyr |
| Tyr (Y) | trp; phe; thr; ser | phe |
| Val (V) | ile; leu; met; phe; | leu |
| | ala; norleucine | |

Substantial modifications in function or immunological identity of the anti-activin antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:
(1) hydrophobic: norleucine, met, ala, val, leu, ile;
(2) neutral hydrophilic: cys, ser, thr;
(3) acidic: asp, glu;
(4) basic: asn, gln, his, lys, arg;
(5) residues that influence chain orientation: gly, pro; and
(6) aromatic: trp, tyr, phe.

Non-conservative substitutions will entail exchanging a member of one of these classes for another class. Such substituted residues also may be introduced into the conservative substitution sites or, more preferably, into the remaining (non-conserved) sites.

The variations can be made using methods known in the art such as oligonucleotide-mediated (site-directed) mutagenesis, alanine scanning, and PCR mutagenesis. Site-directed mutagenesis (Carter et al., Nucl. Acids Res., 13:4331 (1986); Zoller et al., Nucl. Acids Res., 10:6487 (1987)), cassette mutagenesis (Wells et al., Gene, 34:315 (1985)), restriction selection mutagenesis (Wells et al., Philos. Trans. R. Soc. London SerA, 317:415 (1986)) or other known techniques can be performed on the cloned DNA to produce the anti-activin antibody variant DNA.

Scanning amino acid analysis can also be employed to identify one or more amino acids along a contiguous sequence. Among the preferred scanning amino acids are relatively small, neutral amino acids. Such amino acids include alanine, glycine, serine, and cysteine. Alanine is typically a preferred scanning amino acid among this group because it eliminates the side-chain beyond the beta-carbon and is less likely to alter the main-chain conformation of the variant (Cunningham and Wells, Science, 244:1081-5 (1989)). Alanine is also typically preferred because it is the most common amino acid. Further, it is frequently found in both buried and exposed positions (Creighton, The Proteins, (W.H. Freeman & Co., N.Y.); Chothia, J. Mol. Biol., 150:1 (1976)). If alanine substitution does not yield adequate amounts of variant, an isoteric amino acid can be used.

Any cysteine residue not involved in maintaining the proper conformation of the anti-activin antibody also may be substituted, generally with serine, to improve the oxidative stability of the molecule and prevent aberrant crosslinking. Conversely, cysteine bond(s) may be added to the anti-activin antibody to improve its stability (particularly where the antibody is an antibody fragment such as an Fv fragment).

A particularly preferred type of substitutional variant involves substituting one or more hypervariable region residues of a parent antibody (e.g., a humanized or human antibody). Generally, the resulting variant(s) selected for further development will have improved biological properties relative to the parent antibody from which they are generated. A convenient way for generating such substitutional variants involves affinity maturation using phage display. Briefly, several hypervariable region sites (e.g., 6-7 sites) are mutated to generate all possible amino substitutions at each site. The antibody variants thus generated are displayed in a monovalent fashion from filamentous phage particles as fusions to the gene III product of M13 packaged within each particle. The phage-displayed variants are then screened for their biological activity (e.g., binding affinity) as herein disclosed. In order to identify candidate hypervariable region sites for modification, alanine scanning mutagenesis can be performed to identify hypervariable region residues contributing significantly to antigen binding. Alternatively, or additionally, it may be beneficial to analyze a crystal structure of the antigen-antibody complex to identify contact points between the antibody and activin polypeptide. Such contact residues and neighboring residues are candidates for substitution according to the techniques elaborated herein. Once such variants are generated, the panel of variants is subjected to screening as described herein and antibodies with superior properties in one or more relevant assays may be selected for further development.

Nucleic acid molecules encoding amino acid sequence variants of the anti-activin antibody are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of the anti-activin antibody.

### 2. Modifications

Covalent modifications of anti-activin antibodies are included within the scope of this invention. One type of covalent modification includes reacting targeted amino acid residues of an anti-activin antibody with an organic derivatizing agent that is capable of reacting with selected side chains or the N- or C-terminal residues of the anti-activin antibody. Derivatization with bifunctional agents is useful, for instance, for crosslinking anti-activin antibody to a water-insoluble support matrix or surface for use in the method for purifying anti-activin antibodies, and vice-versa. Commonly used crosslinking agents include, e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), bifunctional maleimides such as bis-N-maleimido-1,8-octane and agents such as methyl-3-[(p-azidophenyl)dithio]propioimidate.

Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the α-amino groups of lysine, arginine, and histidine side chains (T. E. Creighton, Proteins: Structure and Molecular Properties, W.H. Freeman & Co., San Francisco, pp. 79-86 (1983)), acetylation of the N-terminal amine, and amidation of any C-terminal carboxyl group.

Another type of covalent modification of the anti-activin antibody included within the scope of this invention comprises altering the native glycosylation pattern of the antibody or polypeptide. “Altering the native glycosylation pattern” is intended for purposes herein to mean deleting one or more carbohydrate moieties found in native sequence anti-activin antibody (either by removing the underlying glycosylation site or by deleting the glycosylation by chemical and/or enzymatic means), and/or adding one or more glycosylation sites that are not present in the native sequence anti-activin antibody. In addition, the phrase includes qualitative changes in the glycosylation of the native proteins, involving a change in the nature and proportions of the various carbohydrate moieties present.

Glycosylation of antibodies and other polypeptides is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used.

Addition of glycosylation sites to the anti-activin antibody is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original anti-activin antibody (for O-linked glycosylation sites). The anti-activin antibody amino acid sequence may optionally be altered through changes at the DNA level, particularly by mutating the DNA encoding the anti-activin antibody at preselected bases such that codons are generated that will translate into the desired amino acids.

Another means of increasing the number of carbohydrate moieties on the anti-activin antibody is by chemical or enzymatic coupling of glycosides to the polypeptide. Such methods are described in the art, e.g., in WO 87/05330 published 11 Sep. 1987, and in Aplin and Wriston, CRC Crit. Rev. Biochem., pp. 259-306 (1981).

Removal of carbohydrate moieties present on the anti-activin antibody may be accomplished chemically or enzymatically or by mutational substitution of codons encoding for amino acid residues that serve as targets for glycosylation. Chemical deglycosylation techniques are known in the art and described, for instance, by Hakimuddin, et al., Arch. Biochem. Biophys., 259:52 (1987) and by Edge et al., Anal. Biochem., 118:131 (1981). Enzymatic cleavage of carbohydrate moieties on polypeptides can be achieved by the use of a variety of endo- and exo-glycosidases as described by Thotakura et al., Meth. Enzymol., 138:350 (1987).

### E. Preparation of Anti-Activin Antibodies

The description below relates primarily to production of anti-activin antibodies by culturing cells transformed or transfected with a vector containing anti-activin antibody-encoding nucleic acid. It is, of course, contemplated that alternative methods, which are well known in the art, may be employed to prepare anti-activin antibodies. For instance, the appropriate amino acid sequence, or portions thereof, may be produced by direct peptide synthesis using solid-phase techniques (e.g., Stewart et al., Solid-Phase Peptide Synthesis, W.H. Freeman Co., San Francisco, CA (1969); Merrifield, J. Am. Chem. Soc., 85:2149-2154 (1963)). In vitro protein synthesis may be performed using manual techniques or by automation. Automated synthesis may be accomplished, for instance, using an Applied Biosystems Peptide Synthesizer (Foster City, CA) using manufacturer's instructions. Various portions of the anti-activin antibody may be chemically synthesized separately and combined using chemical or enzymatic methods to produce the desired anti-activin antibody.

### 1. Isolation of DNA Encoding Anti-Activin Antibody

DNA encoding anti-activin antibody may be obtained from a cDNA library prepared from tissue believed to possess the anti-activin antibody mRNA and to express it at a detectable level. Accordingly, human anti-activin antibody DNA can be conveniently obtained from a cDNA library prepared from human tissue. The anti-activin antibody-encoding gene may also be obtained from a genomic library or by known synthetic procedures (e.g., automated nucleic acid synthesis).

Libraries can be screened with probes (such as oligonucleotides of at least about 20-80 bases) designed to identify the gene of interest or the protein encoded by it. Screening the cDNA or genomic library with the selected probe may be conducted using standard procedures, such as described in Sambrook et al., Molecular Cloning: A Laboratory Manual (New York: Cold Spring Harbor Laboratory Press, 1989). An alternative means to isolate the gene encoding anti-Activin antibody is to use PCR methodology (Sambrook et al., supra; Dieffenbach et al., PCR Primer: A Laboratory Manual (Cold Spring Harbor Laboratory Press, 1995)).

Techniques for screening a cDNA library are well known in the art. The oligonucleotide sequences selected as probes should be of sufficient length and sufficiently unambiguous that false positives are minimized. The oligonucleotide is preferably labeled such that it can be detected upon hybridization to DNA in the library being screened. Methods of labeling are well known in the art, and include the use of radiolabels like 32P-labeled ATP, biotinylation or enzyme labeling. Hybridization conditions, including moderate stringency and high stringency, are provided in Sambrook et al., supra.

Sequences identified in such library screening methods can be compared and aligned to other known sequences deposited and available in public databases such as GenBank or other private sequence databases. Sequence identity (at either the amino acid or nucleotide level) within defined regions of the molecule or across the full-length sequence can be determined using methods known in the art and as described herein.

Nucleic acid having protein coding sequence may be obtained by screening selected cDNA or genomic libraries using the deduced amino acid sequence disclosed herein for the first time, and, if necessary, using conventional primer extension procedures as described in Sambrook et al., supra, to detect precursors and processing intermediates of mRNA that may not have been reverse-transcribed into cDNA.

### 2 Selection and Transformation of Host Cells

Host cells are transfected or transformed with expression or cloning vectors described herein for anti-activin antibody production and cultured in conventional nutrient media modified as appropriate for inducing promoters, selecting transformants, or amplifying the genes encoding the desired sequences. The culture conditions, such as media, temperature, pH and the like, can be selected by the skilled artisan without undue experimentation. In general, principles, protocols, and practical techniques for maximizing the productivity of cell cultures can be found in Mammalian Cell Biotechnology: a Practical Approach, M. Butler, ed. (IRL Press, 1991) and Sambrook et al., supra.

Methods of eukaryotic cell transfection and prokaryotic cell transformation, which means introduction of DNA into the host so that the DNA is replicable, either as an extrachromosomal or by chromosomal integrant, are known to the ordinarily skilled artisan, for example, CaCl2, CaPO4, liposome-mediated, polyethylene-gycol/DMSO and electroporation. Depending on the host cell used, transformation is performed using standard techniques appropriate to such cells. The calcium treatment employing calcium chloride, as described in Sambrook et al., supra, or electroporation is generally used for prokaryotes. Infection with Agrobacterium tumefaciens is used for transformation of certain plant cells, as described by Shaw et al., Gene, 23:315 (1983) and WO 89/05859 published 29 Jun. 1989. For mammalian cells without such cell walls, the calcium phosphate precipitation method of Graham and van der Eb, Virology, 52:456-457 (1978) can be employed. General aspects of mammalian cell host system transfections have been described in U.S. Pat. No. 4,399,216. Transformations into yeast are typically carried out according to the method of Van Solingen et al., J. Bact., 130:946 (1977) and Hsiao et al., Proc. Natl. Acad. Sci. (USA), 76:3829 (1979). However, other methods for introducing DNA into cells, such as by nuclear microinjection, electroporation, bacterial protoplast fusion with intact cells, or polycations, e.g., polybrene, polyornithine, may also be used. For various techniques for transforming mammalian cells, see Keown et al., Methods in Enzymology, 185:527-537 (1990) and Mansour et al., Nature, 336:348-352 (1988).

Suitable host cells for cloning or expressing the DNA in the vectors herein include prokaryote, yeast, or higher eukaryote cells.

a. Prokaryotic Host Cells

Suitable prokaryotes include but are not limited to archaebacteria and eubacteria, such as Gram-negative or Gram-positive organisms, for example, Enterobacteriaceae such as E. coli. Various E. coli strains are publicly available, such as K12 strain MM294 (ATCC 31,446); X1776 (ATCC 31,537); W3110 (ATCC 27,325) and K5 772 (ATCC 53,635). Other suitable prokaryotic host cells include Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformis 41P disclosed in DD 266,710 published 12 Apr. 1989), Pseudomonas such as P. aeruginosa, Rhizobia, Vitreoscilla, Paracoccus and Streptomyces. These examples are illustrative rather than limiting. E. coli strain W3110 is one particularly preferred host or parent host because it is a common host strain for recombinant DNA product fermentations. Preferably, the host cell secretes minimal amounts of proteolytic enzymes. For example, strain W3110 (Bachmann, Cellular and Molecular Biology, vol. 2 (Washington, D.C.: American Society for Microbiology, 1987), pp. 1190-1219; ATCC Deposit No. 27,325) may be modified to effect a genetic mutation in the genes encoding proteins endogenous to the host, with examples of such hosts including E. coli W3110 strain 1A2, which has the complete genotype tonA; E. coli W3110 strain 9E4, which has the complete genotype tonA ptr3; E. coli W3110 strain 27C7 (ATCC 55,244), which has the complete genotype tonA ptr3 phoA E15 (argF-lac) 169 degP ompT kanr; E. coli W3110 strain 37D6, which has the complete genotype tonA ptr3 phoA E15 (argF-lac) 169 degP ompT rbs7 ilvG kanr; E. coli W3110 strain 40B4, which is strain 37D6 with a non-kanamycin resistant degP deletion mutation; E. coli W3110 strain 33D3 having genotype W3110 ΔfhuA (ΔtonA) ptr3 lac Iq lacL8 ΔompTΔ (nmpc-fepE) degP41 kanR (U.S. Pat. No. 5,639,635) and an E. coli strain having mutant periplasmic protease disclosed in U.S. Pat. No. 4,946,783 issued 7 Aug. 1990. Other strains and derivatives thereof, such as E. coli 294 (ATCC 31,446), E. coli B, E. coli λ 1776 (ATCC 31,537) and E. coli RV308 (ATCC 31,608) are also suitable. These examples are illustrative rather than limiting. Methods for constructing derivatives of any of the above-mentioned bacteria having defined genotypes are known in the art and described in, for example, Bass et al., Proteins, 8:309-314 (1990). It is generally necessary to select the appropriate bacteria taking into consideration replicability of the replicon in the cells of a bacterium. For example, E. coli, Serratia, or Salmonella species can be suitably used as the host when well known plasmids such as pBR322, pBR325, pACYC177, or pKN410 are used to supply the replicon. Typically the host cell should secrete minimal amounts of proteolytic enzymes, and additional protease inhibitors may desirably be incorporated in the cell culture. Alternatively, in vitro methods of cloning, e.g., PCR or other nucleic acid polymerase reactions, are suitable.

Full length antibody, antibody fragments, and antibody fusion proteins can be produced in bacteria, in particular when glycosylation and Fc effector function are not needed. Full length antibodies have greater half life in circulation. Production in E. coli is faster and more cost efficient. For expression of antibody fragments and polypeptides in bacteria, see, e.g., U.S. Pat. Nos. 5,648,237; 5,789,199 and 5,840,523, which describe translation initiation regio (TIR) and signal sequences for optimizing expression and secretion, these patents incorporated herein by reference. After expression, the antibody is isolated from the E. coli cell paste in a soluble fraction and can be purified through, e.g., a protein A or G column depending on the isotype. Final purification can be carried out similar to the process for purifying antibody expressed e.g., in CHO cells.

b. Eukaryotic Host Cells

In addition to prokaryotes, eukaryotic microbes such as filamentous fungi or yeast are suitable cloning or expression hosts for anti-activin antibody-encoding vectors. Saccharomyces cerevisiae is a commonly used lower eukaryotic host microorganism. Others include Schizosaccharomyces pombe (Beach and Nurse, Nature, 290:140 (1981); EP 139,383 published 2 May 1985); Kluyveromyces hosts (U.S. Pat. No. 4,943,529; Fleer et al., Bio/Technology, 9:968-75 (1991)) such as, e.g., K. lactis (MW98-8C, CBS683, CBS4574; Louvencourt et al., J. Bacteriol., 154 (2): 737-742 (1983)), K. fragilis (ATCC 12,424), K. bulgaricus (ATCC 16,045), K. wickeramii (ATCC 24,178), K. waltii (ATCC 56,500), K. drosophilarum (ATCC 36,906; Van den Berg et al., Bio/Technology, 8:135 (1990)), K. thermotolerans, and K. marxianus; yarrowia (EP 402,226); Pichia pastoris (EP 183,070; Sreekrishna et al., J. Basic Microbiol., 28:265-278 (1988)); Candida; Trichoderma reesia (EP 244,234); Neurospora crassa (Case et al., Proc. Natl. Acad. Sci. USA, 76:5259-5263 (1979)); Schwanniomyces such as Schwanniomyces occidentalis (EP 394,538 published 31 Oct. 1990); and filamentous fungi such as, e.g., Neurospora, Penicillium, Tolypocladium (WO 91/00357 published 10 Jan. 1991), and Aspergillus hosts such as A. nidulans (Ballance et al., Biochem. Biophys. Res. Commun., 112:284-289 (1983); Tilburn et al., Gene, 26:205-221 (1983); Yelton et al., Proc. Natl. Acad. Sci. USA, 81:1470-1474 (1984)) and A. niger (Kelly and Hynes, EMBO J., 4:475-479 (1985)). Methylotropic yeasts are suitable herein and include, but are not limited to, yeast capable of growth on methanol selected from the genera consisting of Hansenula, Candida, Kloeckera, Pichia, Saccharomyces, Torulopsis, and Rhodotorula. A list of specific species that are exemplary of this class of yeasts may be found in C. Anthony, The Biochemistry of Methylotrophs, 269 (1982).

Suitable host cells for the expression of glycosylated anti-activin antibody are derived from multicellular organisms. Examples of invertebrate cells include insect cells such as Drosophila S2 and Spodoptera Sf9, as well as plant cells, such as cell cultures of cotton, corn, potato, soybean, petunia, tomato, and tobacco. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection are publicly available, e.g., the L-1 variant of Autographa californica NPV and the Bm-5 strain of Bombyx mori NPV, and such viruses may be used as the virus herein according to the present invention, particularly for transfection of Spodoptera frugiperda cells.

However, interest has been greatest in vertebrate cells, and propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR (CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2).

Host cells are transformed with the above-described expression or cloning vectors for anti-activin antibody production and cultured in conventional nutrient media modified as appropriate for inducing promoters, selecting transformants, or amplifying the genes encoding the desired sequences.

### 3. Selection and Use of a Replicable Vector

For recombinant production of an antibody of the invention, the nucleic acid (e.g., cDNA or genomic DNA) encoding it is isolated and inserted into a replicable vector for further cloning (amplification of the DNA) or for expression. DNA encoding the antibody is readily isolated and sequenced using conventional procedures (e.g., by using oligonucleotide probes that are capable of binding specifically to genes encoding the heavy and light chains of the antibody). Many vectors are available. The choice of vector depends in part on the host cell to be used. Generally, preferred host cells are of either prokaryotic or eukaryotic (generally mammalian) origin.

The vector may, for example, be in the form of a plasmid, cosmid, viral particle, or phage. The appropriate nucleic acid sequence may be inserted into the vector by a variety of procedures. In general, DNA is inserted into an appropriate restriction endonuclease site(s) using techniques known in the art. Vector components generally include, but are not limited to, one or more of a signal sequence, an origin of replication, one or more marker genes, an enhancer element, a promoter, and a transcription termination sequence. Construction of suitable vectors containing one or more of these components employs standard ligation techniques which are known to the skilled artisan.

The activin may be produced recombinantly not only directly, but also as a fusion polypeptide with a heterologous polypeptide, which may be a signal sequence or other polypeptide having a specific cleavage site at the N-terminus of the mature protein or polypeptide. In general, the signal sequence may be a component of the vector, or it may be a part of the anti-activin antibody-encoding DNA that is inserted into the vector. The signal sequence may be a prokaryotic signal sequence selected, for example, from the group of the alkaline phosphatase, penicillinase, lpp, or heat-stable enterotoxin II leaders. For yeast secretion the signal sequence may be, e.g., the yeast invertase leader, alpha factor leader (including Saccharomyces and Kluyveromyces α-factor leaders, the latter described in U.S. Pat. No. 5,010,182), or acid phosphatase leader, the C. albicans glucoamylase leader (EP 362,179 published 4 Apr. 1990), or the signal described in WO 90/13646 published 15 Nov. 1990. In mammalian cell expression, mammalian signal sequences may be used to direct secretion of the protein, such as signal sequences from secreted polypeptides of the same or related species, as well as viral secretory leaders.

a. Prokaryotic Host Cells

Polynucleotide sequences encoding polypeptide components of the antibody of the invention can be obtained using standard recombinant techniques. Desired polynucleotide sequences may be isolated and sequenced from antibody producing cells such as hybridoma cells. Alternatively, polynucleotides can be synthesized using nucleotide synthesizer or PCR techniques. Once obtained, sequences encoding the polypeptides are inserted into a recombinant vector capable of replicating and expressing heterologous polynucleotides in prokaryotic hosts. Many vectors that are available and known in the art can be used for the purpose of the present invention. Selection of an appropriate vector will depend mainly on the size of the nucleic acids to be inserted into the vector and the particular host cell to be transformed with the vector. Each vector contains various components, depending on its function (amplification or expression of heterologous polynucleotide, or both) and its compatibility with the particular host cell in which it resides.

In general, plasmid vectors containing replicon and control sequences which are derived from species compatible with the host cell are used in connection with these hosts. Both expression and cloning vectors contain a nucleic acid sequence that enables the vector to replicate in one or more selected host cells, as well as marking sequences which are capable of providing phenotypic selection in transformed cells. Such sequences are well known for a variety of bacteria, yeast, and viruses. The origin of replication from the plasmid pBR322, which contains genes encoding ampicillin (Amp) and tetracycline (Tet) resistance and thus provides easy means for identifying transformed cells, is suitable for most Gram-negative bacteria, the 2μ plasmid origin is suitable for yeast, and various viral origins (SV40, polyoma, adenovirus, VSV or BPV) are useful for cloning vectors in mammalian cells. pBR322, its derivatives, or other microbial plasmids or bacteriophage may also contain, or be modified to contain, promoters which can be used by the microbial organism for expression of endogenous proteins. Examples of pBR322 derivatives used for expression of particular antibodies are described in detail in Carter et al., U.S. Pat. No. 5,648,237.

In addition, phage vectors containing replicon and control sequences that are compatible with the host microorganism can be used as transforming vectors in connection with these hosts. For example, bacteriophage such as λGEM™-11 may be utilized in making a recombinant vector which can be used to transform susceptible host cells such as E. coli LE392.

The expression vector of the invention may comprise two or more promoter-cistron pairs, encoding each of the polypeptide components. A promoter is an untranslated regulatory sequence located upstream (5′) to a cistron that modulates its expression. Prokaryotic promoters typically fall into two classes, inducible and constitutive. Inducible promoter is a promoter that initiates increased levels of transcription of the cistron under its control in response to changes in the culture condition, e.g., the presence or absence of a nutrient or a change in temperature.

A large number of promoters recognized by a variety of potential host cells are well known. The selected promoter can be operably linked to cistron DNA encoding the light or heavy chain by removing the promoter from the source DNA via restriction enzyme digestion and inserting the isolated promoter sequence into the vector of the invention. Both the native promoter sequence and many heterologous promoters may be used to direct amplification and/or expression of the target genes. In some embodiments, heterologous promoters are utilized, as they generally permit greater transcription and higher yields of expressed target gene as compared to the native target polypeptide promoter.

Promoters recognized by a variety of potential host cells are well known. Promoters suitable for use with prokaryotic hosts include the PhoA promoter, the β-galactamase and lactose promoter systems (Chang et al., Nature, 275:615 (1978); Goeddel et al., Nature, 281:544 (1979)), alkaline phosphatase, a tryptophan (trp) promoter system (Goeddel, Nucleic Acids Res., 8:4057 (1980); EP 36,776) and hybrid promoters such as the tac (deBoer et al., Proc. Natl. Acad. Sci. USA, 80:21-25 (1983)) or the trc promoter. Promoters for use in bacterial systems also will contain a Shine-Dalgarno (S.D.) sequence operably linked to the DNA encoding anti-activin antibody. However, other promoters that are functional in bacteria (such as other known bacterial or phage promoters) are suitable as well. Their nucleotide sequences have been published, thereby enabling a skilled worker operably to ligate them to cistrons encoding the target light and heavy chains (Siebenlist et al. (1980) Cell 20:269) using linkers or adaptors to supply any required restriction sites.

In one aspect of the invention, each cistron within the recombinant vector comprises a secretion signal sequence component that directs translocation of the expressed polypeptides across a membrane. In general, the signal sequence may be a component of the vector, or it may be a part of the target polypeptide DNA that is inserted into the vector. The signal sequence selected for the purpose of this invention should be one that is recognized and processed (i.e. cleaved by a signal peptidase) by the host cell. For prokaryotic host cells that do not recognize and process the signal sequences native to the heterologous polypeptides, the signal sequence is substituted by a prokaryotic signal sequence selected, for example, from the group consisting of the alkaline phosphatase, penicillinase, Ipp, or heat-stable enterotoxin II (STII) leaders, LamB, PhoE, PelB, OmpA and MBP. In one embodiment of the invention, the signal sequences used in both cistrons of the expression system are STII signal sequences or variants thereof.

In another aspect, the production of the immunoglobulins according to the invention can occur in the cytoplasm of the host cell, and therefore does not require the presence of secretion signal sequences within each cistron. In that regard, immunoglobulin light and heavy chains are expressed, folded and assembled to form functional immunoglobulins within the cytoplasm. Certain host strains (e.g., the E. coli trxB-strains) provide cytoplasm conditions that are favorable for disulfide bond formation, thereby permitting proper folding and assembly of expressed protein subunits. Proba and Pluckthun Gene, 159:203 (1995).

The present invention provides an expression system in which the quantitative ratio of expressed polypeptide components can be modulated in order to maximize the yield of secreted and properly assembled antibodies of the invention. Such modulation is accomplished at least in part by simultaneously modulating translational strengths for the polypeptide components.

One technique for modulating translational strength is disclosed in Simmons et al., U.S. Pat. No. 5,840,523. It utilizes variants of the translational initiation region (TIR) within a cistron. For a given TIR, a series of amino acid or nucleic acid sequence variants can be created with a range of translational strengths, thereby providing a convenient means by which to adjust this factor for the desired expression level of the specific chain. TIR variants can be generated by conventional mutagenesis techniques that result in codon changes which can alter the amino acid sequence, although silent changes in the nucleotide sequence are preferred. Alterations in the TIR can include, for example, alterations in the number or spacing of Shine-Dalgarno sequences, along with alterations in the signal sequence. One method for generating mutant signal sequences is the generation of a “codon bank” at the beginning of a coding sequence that does not change the amino acid sequence of the signal sequence (i.e., the changes are silent). This can be accomplished by changing the third nucleotide position of each codon; additionally, some amino acids, such as leucine, serine, and arginine, have multiple first and second positions that can add complexity in making the bank. This method of mutagenesis is described in detail in Yansura et al. (1992) METHODS: A Companion to Methods in Enzymol. 4:151-158.

Preferably, a set of vectors is generated with a range of TIR strengths for each cistron therein. This limited set provides a comparison of expression levels of each chain as well as the yield of the desired antibody products under various TIR strength combinations. TIR strengths can be determined by quantifying the expression level of a reporter gene as described in detail in Simmons et al. U.S. Pat. No. 5,840,523. Based on the translational strength comparison, the desired individual TIRs are selected to be combined in the expression vector constructs of the invention.

b. Eukaryotic Host Cells

The vector components generally include, but are not limited to, one or more of the following: a signal sequence, an origin of replication, one or more marker genes, an enhancer element, a promoter, and a transcription termination sequence.

### (1) Signal Sequence Component

A vector for use in a eukaryotic host cell may also contain a signal sequence or other polypeptide having a specific cleavage site at the N-terminus of the mature protein or polypeptide of interest. The heterologous signal sequence selected preferably is one that is recognized and processed (i.e., cleaved by a signal peptidase) by the host cell. In mammalian cell expression, mammalian signal sequences as well as viral secretory leaders, for example, the herpes simplex gD signal, are available.

The DNA for such precursor region is ligated in reading frame to DNA encoding the antibody.

### (2) Origin of Replication

Generally, an origin of replication component is not needed for mammalian expression vectors. For example, the SV40 origin may typically be used only because it contains the early promoter.

### (3) Selection Gene Component

Expression and cloning vectors will typically contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli.

One example of a selection scheme utilizes a drug to arrest growth of a host cell. Those cells that are successfully transformed with a heterologous gene produce a protein conferring drug resistance and thus survive the selection regimen. Examples of such dominant selection use the drugs neomycin, mycophenolic acid and hygromycin.

An example of suitable selectable markers for mammalian cells are those that enable the identification of cells competent to take up the anti-Activin antibody-encoding nucleic acid, such as DHFR or thymidine kinase, metallothionein-I and -II, preferably primate metallothionein genes, adenosine deaminase, ornithine decarboxylase, etc. An appropriate host cell when wild-type DHFR is employed is the CHO cell line deficient in DHFR activity (e.g., ATCC CRL-9096), prepared and propagated as described by Urlaub et al., Proc. Natl. Acad. Sci. USA, 77:4216 (1980). For example, cells transformed with the DHFR selection gene are first identified by culturing all of the transformants in a culture medium that contains methotrexate (Mtx), a competitive antagonist of DHFR. Alternatively, host cells (particularly wild-type hosts that contain endogenous DHFR) transformed or co-transformed with DNA sequences encoding an antibody, wild-type DHFR protein, and another selectable marker such as aminoglycoside 3′-phosphotransferase (APH) can be selected by cell growth in medium containing a selection agent for the selectable marker such as an aminoglycosidic antibiotic, e.g., kanamycin, neomycin, or G418. See U.S. Pat. No. 4,965,199.

A suitable selection gene for use in yeast is the trp1 gene present in the yeast plasmid YRp7 (Stinchcomb et al., Nature, 282:39 (1979); Kingsman et al., Gene, 7:141 (1979); Tschemper et al., Gene, 10:157 (1980)). The trp1 gene provides a selection marker for a mutant strain of yeast lacking the ability to grow in tryptophan, for example, ATCC No. 44076 or PEP4-1 (Jones, Genetics, 85:12 (1977)).

### (4) Promoter Component

Expression and cloning vectors usually contain a promoter operably linked to the anti-Activin antibody-encoding nucleic acid sequence to direct mRNA synthesis. Promoters recognized by a variety of potential host cells are well known.

Virtually alleukaryotic genes have an AT-rich region located approximately 25 to 30 bases upstream from the site where transcription is initiated. Another sequence found 70 to 80 bases upstream from the start of transcription of many genes is a CNCAAT region where N may be any nucleotide. At the 3′ end of most eukaryotic genes is an AATAAA sequence that may be the signal for addition of the poly A tail to the 3′ end of the coding sequence. All of these sequences are suitably inserted into eukaryotic expression vectors.

Examples of suitable promoting sequences for use with yeast hosts include the promoters for 3-phosphoglycerate kinase (Hitzeman et al., J. Biol. Chem., 255:2073 (1980)) or other glycolytic enzymes (Hess et al., J. Adv. Enzyme Reg., 7:149 (1968); Holland, Biochemistry, 17:4900 (1978)), such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase.

Other yeast promoters, which are inducible promoters having the additional advantage of transcription controlled by growth conditions, are the promoter regions for alcohol dehydrogenase 2, isocytochrome C, acid phosphatase, degradative enzymes associated with nitrogen metabolism, metallothionein, glyceraldehyde-3-phosphate dehydrogenase, and enzymes responsible for maltose and galactose utilization. Suitable vectors and promoters for use in yeast expression are further described in EP 73,657.

Anti-activin antibody transcription from vectors in mammalian host cells is controlled, for example, by promoters obtained from the genomes of viruses such as polyoma virus, fowlpox virus (UK 2,211,504 published 5 Jul. 1989), adenovirus (such as Adenovirus 2), bovine papilloma virus, avian sarcoma virus, cytomegalovirus, a retrovirus, hepatitis-B virus and Simian Virus 40 (SV40), from heterologous mammalian promoters, e.g., the actin promoter or an immunoglobulin promoter, and from heat-shock promoters, provided such promoters are compatible with the host cell systems.

The early and late promoters of the SV40 virus are conveniently obtained as an SV40 restriction fragment that also contains the SV40 viral origin of replication. The immediate early promoter of the human cytomegalovirus is conveniently obtained as a HindIII E restriction fragment. A system for expressing DNA in mammalian hosts using the bovine papilloma virus as a vector is disclosed in U.S. Pat. No. 4,419,446. A modification of this system is described in U.S. Pat. No. 4,601,978. See also Reyes et al., Nature 297:598-601 (1982) on expression of human β-interferon cDNA in mouse cells under the control of a thymidine kinase promoter from herpes simplex virus. Alternatively, the Rous Sarcoma Virus long terminal repeat can be used as the promoter.

### (5) Enhancer Element Component

Transcription of a DNA encoding the anti-activin antibody by higher eukaryotes may be increased by inserting an enhancer sequence into the vector. Enhancers are cis-acting elements of DNA, usually about from 10 to 300 bp, that act on a promoter to increase its transcription. Many enhancer sequences are now known from mammalian genes (globin, elastase, albumin, α-fetoprotein, and insulin). Typically, however, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers. See also Yaniv, Nature 297:17-18 (1982) on enhancing elements for activation of eukaryotic promoters. The enhancer may be spliced into the vector at a position 5′ or 3′ to the anti-activin antibody coding sequence, but is preferably located at a site 5′ from the promoter.

### (6) Transcription Termination Component

Expression vectors used in eukaryotic host cells (yeast, fungi, insect, plant, animal, human, or nucleated cells from other multicellular organisms) will also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding anti-Activin antibody. One useful transcription termination component is the bovine growth hormone polyadenylation region. See WO94/11026 and the expression vector disclosed therein.

Still other methods, vectors, and host cells suitable for adaptation to the synthesis of anti-activin antibody in recombinant vertebrate cell culture are described in Gething et al., Nature, 293:620-625 (1981); Mantei et al., Nature, 281:40-46 (1979); EP 117,060; and EP 117,058.

### 4. Culturing the Host Cells

The host cells used to produce the anti-Activin antibody of this invention may be cultured in a variety of media.

a. Prokaryotic Host Cells

Prokaryotic cells used to produce the polypeptides of the invention are grown in media known in the art and suitable for culture of the selected host cells. Examples of suitable media include luria broth (LB) plus necessary nutrient supplements. In some embodiments, the media also contains a selection agent, chosen based on the construction of the expression vector, to selectively permit growth of prokaryotic cells containing the expression vector. For example, ampicillin is added to media for growth of cells expressing ampicillin resistant gene.

Any necessary supplements besides carbon, nitrogen, and inorganic phosphate sources may also be included at appropriate concentrations introduced alone or as a mixture with another supplement or medium such as a complex nitrogen source. Optionally the culture medium may contain one or more reducing agents selected from the group consisting of glutathione, cysteine, cystamine, thioglycollate, dithioerythritol and dithiothreitol.

The prokaryotic host cells are cultured at suitable temperatures. For E. coli growth, for example, the preferred temperature ranges from about 20° C. to about 39° C., more preferably from about 25° C. to about 37° C., even more preferably at about 30° C. The pH of the medium may be any pH ranging from about 5 to about 9, depending mainly on the host organism. For E. coli, the pH is preferably from about 6.8 to about 7.4, and more preferably about 7.0.

If an inducible promoter is used in the expression vector of the invention, protein expression is induced under conditions suitable for the activation of the promoter. In one aspect of the invention, PhoA promoters are used for controlling transcription of the polypeptides. Accordingly, the transformed host cells are cultured in a phosphate-limiting medium for induction. In some embodiments, the phosphate-limiting medium is the C.R.A.P medium (see, e.g., Simmons et al., J. Immunol. Methods (2002), 263:133-47). A variety of other inducers may be used, according to the vector construct employed, as is known in the art.

In one embodiment, the expressed polypeptides of the present invention are secreted into and recovered from the periplasm of the host cells. Protein recovery typically involves disrupting the microorganism, generally by such means as osmotic shock, sonication or lysis. Once cells are disrupted, cell debris or whole cells may be removed by centrifugation or filtration. The proteins may be further purified, for example, by affinity resin chromatography. Alternatively, proteins can be transported into the culture media and isolated therein. Cells may be removed from the culture and the culture supernatant being filtered and concentrated for further purification of the proteins produced. The expressed polypeptides can be further isolated and identified using commonly known methods such as polyacrylamide gel electrophoresis (PAGE) and Western blot assay.

In one aspect of the invention, antibody production is conducted in large quantity by a fermentation process. Various large-scale fed-batch fermentation procedures are available for production of recombinant proteins. Large-scale fermentations have at least 1000 liters of capacity, preferably about 1,000 to 100,000 liters of capacity. These fermentors use agitator impellers to distribute oxygen and nutrients, especially glucose (the preferred carbon/energy source). Small scale fermentation refers generally to fermentation in a fermentor that is no more than approximately 100 liters in volumetric capacity, and can range from about 1 liter to about 100 liters.

In a fermentation process, induction of protein expression is typically initiated after the cells have been grown under suitable conditions to a desired density, e.g., an OD550 of about 180-220, at which stage the cells are in the early stationary phase. A variety of inducers may be used, according to the vector construct employed, as is known in the art and described above. Cells may be grown for shorter periods prior to induction. Cells are usually induced for about 12-50 hours, although longer or shorter induction time may be used.

To improve the production yield and quality of the polypeptides of the invention, various fermentation conditions can be modified. For example, to improve the proper assembly and folding of the secreted antibody polypeptides, additional vectors overexpressing chaperone proteins, such as Dsb proteins (DsbA, DsbB, DsbC, DsbD and or DsbG) or FkpA (a peptidylprolyl cis, trans-isomerase with chaperone activity) can be used to co-transform the host prokaryotic cells. The chaperone proteins have been demonstrated to facilitate the proper folding and solubility of heterologous proteins produced in bacterial host cells. Chen et al. (1999) J Bio Chem 274:19601-5; U.S. Pat. Nos. 6,083,715; 6,027,888; Bothmann and Pluckthun (2000) J. Biol. Chem. 275:17100-5; Ramm and Pluckthun (2000) J. Biol. Chem. 275:17106-13; Arie et al. (2001) Mol. Microbiol. 39:199-210.

To minimize proteolysis of expressed heterologous proteins (especially those that are proteolytically sensitive), certain host strains deficient for proteolytic enzymes can be used for the present invention. For example, host cell strains may be modified to effect genetic mutation(s) in the genes encoding known bacterial proteases such as Protease III, OmpT, DegP, Tsp, Protease I, Protease Mi, Protease V, Protease VI and combinations thereof. Some E. coli protease-deficient strains are available and described in, for example, Joly et al. (1998), supra; U.S. Pat. Nos. 5,264,365; 5,508,192; Hara et al., Microbial Drug Resistance, 2:63-72 (1996).

In one embodiment, E. coli strains deficient for proteolytic enzymes and transformed with plasmids overexpressing one or more chaperone proteins are used as host cells in the expression system of the invention.

b. Eukaryotic Host Cells

Commercially available media such as Ham's F10 (Sigma), Minimal Essential Medium ((MEM), (Sigma), RPMI-1640 (Sigma), and Dulbecco's Modified Eagle Medium ((DMEM), Sigma) are suitable for culturing the host cells. In addition, any of the media described in Ham et al., Meth. Enz. 58:44 (1979), Barnes et al., Anal. Biochem. 102:255 (1980), U.S. Pat. Nos. 4,767,704; 4,657,866; 4,927,762; 4,560,655; or 5,122,469; WO 90/03430; WO 87/00195; or U.S. Pat. No. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

### 5. Detecting Gene Amplification/Expression

Gene amplification and/or expression may be measured in a sample directly, for example, by conventional Southern blotting, Northern blotting to quantitate the transcription of mRNA (Thomas, Proc. Natl. Acad. Sci. USA, 77:5201-5 (1980)), dot blotting (DNA analysis), or in situ hybridization, using an appropriately labeled probe, based on the sequences provided herein. Alternatively, antibodies may be employed that can recognize specific duplexes, including DNA duplexes, RNA duplexes, and DNA-RNA hybrid duplexes or DNA-protein duplexes. The antibodies in turn may be labeled and the assay may be carried out where the duplex is bound to a surface, so that upon the formation of duplex on the surface, the presence of antibody bound to the duplex can be detected.

Gene expression, alternatively, may be measured by immunological methods, such as immunohistochemical staining of cells or tissue sections and assay of cell culture or body fluids, to quantitate directly the expression of gene product. Antibodies useful for immunohistochemical staining and/or assay of sample fluids may be either monoclonal or polyclonal, and may be prepared in any mammal. Conveniently, the antibodies may be prepared against a native sequence activin polypeptide or against a synthetic peptide based on the DNA sequences provided herein or against exogenous sequence fused to Activin DNA and encoding a specific antibody epitope.

### 6. Purification of Anti-Activin Antibody

Forms of anti-activin antibody may be recovered from culture medium or from host cell lysates. If membrane-bound, it can be released from the membrane using a suitable detergent solution (e.g., Triton-X 100) or by enzymatic cleavage. Cells employed in expression of anti-Activin antibody can be disrupted by various physical or chemical means, such as freeze-thaw cycling, sonication, mechanical disruption, or cell lysing agents.

It may be desired to purify anti-activin antibody from recombinant cell proteins or polypeptides. The following procedures are exemplary of suitable purification procedures: by fractionation on an ion-exchange column; ethanol precipitation; reverse phase HPLC; chromatography on silica or on a cation-exchange resin such as DEAE; chromatofocusing; SDS-PAGE; ammonium sulfate precipitation; gel filtration using, for example, Sephadex G-75; protein A Sepharose columns to remove contaminants such as IgG; and metal chelating columns to bind epitope-tagged forms of the anti-activin antibody. Various methods of protein purification may be employed and such methods are known in the art and described for example in Deutscher, Methods in Enzymology, 182 (1990); Scopes, Protein Purification: Principles and Practice, Springer-Verlag, New York (1982). The purification step(s) selected will depend, for example, on the nature of the production process used and the particular anti-activin antibody produced.

When using recombinant techniques, the antibody can be produced intracellularly, in the periplasmic space, or directly secreted into the medium. If the antibody is produced intracellularly, as a first step, the particulate debris, either host cells or lysed fragments, are removed, for example, by centrifugation or ultrafiltration. Carter et al., Bio/Technology 10:163-7 (1992) describe a procedure for isolating antibodies which are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation. Where the antibody is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The antibody composition prepared from the cells can be purified using, for example, hydroxylapatite chromatography, gel electrophoresis, dialysis, and affinity chromatography, with affinity chromatography being the preferred purification technique. The suitability of protein A as an affinity ligand depends on the species and isotype of any immunoglobulin Fc domain that is present in the antibody. Protein A can be used to purify antibodies that are based on human γ1, γ2 or γ4 heavy chains (Lindmark et al., J. Immunol. Meth. 62:1-13 (1983)). Protein G is recommended for all mouse isotypes and for human γ3 (Guss et al., EMBO J. 5:15671575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a CH3 domain, the Bakerbond ABX™ resin (J. T. Baker, Phillipsburg, NJ) is useful for purification. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the antibody to be recovered.

Following any preliminary purification step(s), the mixture comprising the antibody of interest and contaminants may be subjected to low pH hydrophobic interaction chromatography using an elution buffer at a pH between about 2.5-4.5, and generally at low salt concentrations (e.g., from about 0-0.25M salt).

### F. Pharmaceutical Formulations

The antibodies of the invention may be administered by any route appropriate to the condition to be treated. The antibody will typically be administered parenterally, i.e. infusion, subcutaneous, intramuscular, intravenous, intradermal, intrathecal and epidural.

For treating these cancers, in one embodiment, the antibody is administered via intravenous infusion. The dosage administered via infusion is in the range of about 1 μg/m2 to about 10,000 μg/m² per dose, generally one dose per week for a total of one, two, three or four doses. Alternatively, the dosage range is of about 1 μg/m² to about 1000 μg/m², about 1 μg/m² to about 800 μg/m², about 1 μg/m² to about 600 μg/m², about 1 μg/m² to about 400 μg/m², about 10 μg/m² to about 500 μg/m², about 10 μg/m² to about 300 μg/m², about 10 μg/m² to about 200 μg/m², and about 1 μg/m² to about 200 μg/m². The dose may be administered once per day, once per week, multiple times per week, but less than once per day, multiple times per month but less than once per day, multiple times per month but less than once per week, once per month or intermittently to relieve or alleviate symptoms of the disease. Administration may continue at any of the disclosed intervals until remission of the tumor or symptoms of the cancer being treated. Administration may continue after remission or relief of symptoms is achieved where such remission or relief is prolonged by such continued administration.

The invention also provides a method of treating breast cancer comprising administering to a patient suffering from breast cancer, a therapeutically effective amount of a humanized activin antibody of any one of the preceding embodiments. The antibody will typically be administered in a dosage range of about 1 μg/m² to about 1000 mg/m².

In one aspect, the invention further provides pharmaceutical formulations comprising at least one anti-activin antibody of the invention. In some embodiments, a pharmaceutical formulation comprises (1) an antibody of the invention, and (2) a pharmaceutically acceptable carrier.

Therapeutic formulations comprising an anti-activin antibody used in accordance with the present invention are prepared for storage by mixing the antibody having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as acetate, Tris, phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; tonicifiers such as trehalose and sodium chloride; sugars such as sucrose, mannitol, trehalose or sorbitol; surfactant such as polysorbate; salt-forming counter-ions such as sodium; metal complexes (e.g., Zn-protein complexes); and/or non-ionic surfactants such as TWEEN®, PLURONICS® or polyethylene glycol (PEG). Pharmaceutical formulations to be used for in vivo administration are generally sterile. This is readily accomplished by filtration through sterile filtration membranes.

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences, 16th edition, Osol, A. Ed. (1980).

Sustained-release preparations may be prepared. Suitable examples of sustained-release preparations include semi-permeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT® (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated immunoglobulins remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

An antibody may be formulated in any suitable form for delivery to a target cell/tissue. For example, antibodies may be formulated as immunoliposomes. A “liposome” is a small vesicle composed of various types of lipids, phospholipids and/or surfactant which is useful for delivery of a drug to a mammal. The components of the liposome are commonly arranged in a bilayer formation, similar to the lipid arrangement of biological membranes. Liposomes containing the antibody are prepared by methods known in the art, such as described in Epstein et al., Proc. Natl. Acad. Sci. USA 82:3688 (1985); Hwang et al., Proc. Natl Acad. Sci. USA 77:4030 (1980); U.S. Pat. Nos. 4,485,045 and 4,544,545; and WO97/38731 published Oct. 23, 1997. Liposomes with enhanced circulation time are disclosed in U.S. Pat. No. 5,013,556.

Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter. Fab′ fragments of the antibody of the present invention can be conjugated to the liposomes as described in Martin et al., J. Biol. Chem. 257:286-8 (1982) via a disulfide interchange reaction. A chemotherapeutic agent is optionally contained within the liposome (See Gabizon et al., J. National Cancer Inst. 81 (19): 1484 (1989)).

The formulations to be used for in vivo administration must be sterile. This is readily accomplished by filtration through sterile filtration membranes.

G. Treatment with Anti-Activin Antibodies

To determine activin expression in a cancer, various detection assays are available. In one embodiment, activin polypeptide overexpression may be analyzed by immunohistochemistry (IHC). Parrafin embedded tissue sections from a tumor biopsy may be subjected to the IHC assay and accorded an activin protein staining intensity criteria. In a preferred embodiment, determining whether a cancer is amenable to treatment by methods disclosed herein involves detecting the presence of the activin tumor epitope in a subject or in a sample from a subject.

Alternatively, or additionally, FISH assays such as the INFORM® (sold by Ventana, Arizona) or PATHVISION® (Vysis, Illinois) may be carried out on formalin-fixed, paraffin-embedded tumor tissue to determine the extent (if any) of activin overexpression in the tumor.

Activin overexpression or amplification may be evaluated using an in vivo detection assay, e.g., by administering a molecule (such as an antibody) which binds the molecule to be detected and is tagged with a detectable label (e.g., a radioactive isotope or a fluorescent label) and externally scanning the patient for localization of the label.

As described above, the anti-activin antibodies of the invention have various non-therapeutic applications. The anti-activin antibodies of the present invention can be useful for staging of activin epitope expressing cancers (e.g., in radioimaging). The antibodies are also useful for purification or immunoprecipitation of activin epitope from cells, for detection and quantitation of activin epitope in vitro, e.g., in an ELISA or a Western blot, to kill and eliminate activin-expressing cells from a population of mixed cells as a step in the purification of other cells.

Currently, depending on the stage of the cancer, cancer treatment involves one or a combination of the following therapies: surgery to remove the cancerous tissue, radiation therapy, and chemotherapy. Anti-activin antibody therapy may be especially desirable in elderly patients who do not tolerate the toxicity and side effects of chemotherapy well and in metastatic disease where radiation therapy has limited usefulness. The tumor targeting anti-activin antibodies of the invention are useful to alleviate activin-expressing cancers upon initial diagnosis of the disease or during relapse.

The anti-activin antibodies are administered to a human patient, in accordance with known methods, such as intravenous administration, e.g., as a bolus or by continuous infusion over a period of time, by intramuscular, intraperitoneal, intracerobrospinal, subcutaneous, intra-articular, intrasynovial, intrathecal, oral, topical, or inhalation routes. In some embodiments, intravenous or subcutaneous administration of the antibody is preferred.

The antibody composition of the invention will be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

For the prevention or treatment of disease, the dosage and mode of administration will be chosen by the physician according to known criteria. The appropriate dosage of antibody will depend on the type of disease to be treated, as defined above, the severity and course of the disease, whether the antibody is administered for preventive or therapeutic purposes, previous therapy, the patient's clinical history and response to the antibody, and the discretion of the attending physician. The antibody is suitably administered to the patient at one time or over a series of treatments. Preferably, the antibody is administered by intravenous infusion or by subcutaneous injections. Depending on the type and severity of the disease, about 1 μg/kg to about 50 mg/kg body weight (e.g., about 0.1-15 mg/kg/dose) of antibody can be an initial candidate dosage for administration to the patient, whether, for example, by one or more separate administrations, or by continuous infusion. A dosing regimen can comprise administering an initial loading dose of about 4 mg/kg, followed by a weekly maintenance dose of about 2 mg/kg of the anti-activin antibody. However, other dosage regimens may be useful. A typical daily dosage might range from about 1 μg/kg to 100 mg/kg or more, depending on the factors mentioned above. For repeated administrations over several days or longer, depending on the condition, the treatment is sustained until a desired suppression of disease symptoms occurs. The progress of this therapy can be readily monitored by conventional methods and assays and based on criteria known to the physician or other persons of skill in the art.

The anti-activin antibodies of the invention can be in the different forms encompassed by the definition of “antibody” herein. Thus, the antibodies include full length or intact antibody, antibody fragments, native sequence antibody or amino acid variants, humanized, chimeric or fusion antibodies, and functional fragments thereof. In fusion antibodies an antibody sequence is fused to a heterologous polypeptide sequence. The antibodies can be modified in the Fc region to provide desired effector functions. As discussed in more detail in the sections herein, with the appropriate Fc regions, the naked antibody bound on the cell surface can induce cytotoxicity, e.g., via antibody-dependent cellular cytotoxicity (ADCC) or by recruiting complement in complement dependent cytotoxicity, or some other mechanism. Alternatively, where it is desirable to eliminate or reduce effector function, so as to minimize side effects or therapeutic complications, certain other Fc regions may be used.

In one embodiment, the antibody (i) competes for binding to the same epitope, and/or (ii) binds substantially to the same epitope, as the antibodies of the invention. Antibodies having the biological characteristics of the present anti-activin antibodies of the invention are also contemplated, specifically including the in vivo tumor targeting and any cell proliferation inhibition or cytotoxic characteristics.

Methods of producing the above antibodies are described in detail herein.

The present anti-activin antibodies are useful for treating an activin-expressing cancer or alleviating one or more symptoms of the cancer in a mammal. The cancers encompass metastatic cancers of any of the cancers described herein. The antibody is able to bind to at least a portion of the cancer cells that express activin epitope in the mammal. In a preferred embodiment, the antibody is effective to destroy or kill activin-expressing tumor cells or inhibit the growth of such tumor cells, in vitro or in vivo, upon binding to activin epitope on the cell. In other preferred embodiments, the antibodies are effective to (i) inhibit the growth or proliferation of a cell to which they bind; (ii) induce the death of a cell to which they bind; (iii) inhibit the delamination of a cell to which they bind; (iv) inhibit the metastasis of a cell to which they bind; or (v) inhibit the vascularization of a tumor comprising a cell to which they bind.

The invention provides a composition comprising an anti-activin antibody of the invention, and a carrier. The invention also provides formulations comprising an anti-activin antibody of the invention, and a carrier. In one embodiment, the formulation is a therapeutic formulation comprising a pharmaceutically acceptable carrier.

Another aspect of the invention is isolated nucleic acids encoding the anti-activin antibodies. Nucleic acids encoding both the H and L chains and especially the hypervariable region residues, chains which encode the native sequence antibody as well as variants, modifications and humanized versions of the antibody, are encompassed.

The invention also provides methods useful for treating an activin polypeptide-expressing cancer or alleviating one or more symptoms of the cancer in a mammal, comprising administering a therapeutically effective amount of an anti-activin antibody to the mammal. The antibody therapeutic compositions can be administered short term (acute) or chronic, or intermittent as directed by physician. Also provided are methods of inhibiting the growth of, and killing an activin polypeptide-expressing cell.

The invention also provides kits and articles of manufacture comprising at least one anti-activin antibody. Kits containing anti-activin antibodies find use, e.g., for activin cell killing assays, for purification or immunoprecipitation of activin polypeptide from cells. For example, for isolation and purification of activin, the kit can contain an anti-activin antibody coupled to beads (e.g., sepharose beads). Kits can be provided which contain the antibodies for detection and quantitation of activin in vitro, e.g., in an ELISA or a Western blot. Such antibody useful for detection may be provided with a label such as a fluorescent or radiolabel.

### Effector Function Engineering

It may be desirable to modify the antibody of the invention with respect to effector function, e.g., so as to enhance antigen-dependent cell-mediated cyotoxicity (ADCC) and/or complement dependent cytotoxicity (CDC) of the antibody. This may be achieved by introducing one or more amino acid substitutions in an Fc region of the antibody. Alternatively or additionally, cysteine residue(s) may be introduced in the Fc region, thereby allowing interchain disulfide bond formation in this region. The homodimeric antibody thus generated may have improved internalization capability and/or increased complement-mediated cell killing and antibody-dependent cellular cytotoxicity (ADCC) (see Caron et al., J. Exp Med. 176:1191-5 (1992) and Shopes, B. J. Immunol. 148:2918-22 (1992)). Homodimeric antibodies with enhanced anti-tumor activity may also be prepared using heterobifunctional cross-linkers as described in Wolff et al., Cancer Research 53:2560-5 (1993). Alternatively, an antibody can be engineered which has dual Fc regions and may thereby have enhanced complement lysis and ADCC capabilities (see Stevenson et al., Anti-Cancer Drug Design 3:219-30 (1989)). To increase the serum half life of the antibody, one may incorporate a salvage receptor binding epitope into the antibody (especially an antibody fragment), e.g., as described in U.S. Pat. No. 5,739,277. As used herein, the term “salvage receptor binding epitope” refers to an epitope of the Fc region of an IgG molecule (e.g., IgG₁, IgG₂, IgG₃, or IgG₄) that is responsible for increasing the in vivo serum half-life of the IgG molecule.

### Immunoconjugates

The invention also pertains to immunoconjugates (interchangeably referred to as “antibody-drug conjugates,” or “ADCs”) comprising an antibody conjugated to a cytotoxic agent such as a chemotherapeutic agent, a growth inhibitory agent, a toxin (e.g., an enzymatically active toxin of bacterial, fungal, plant, or animal origin, or fragments thereof), or a radioactive isotope (i.e., a radioconjugate).

In certain embodiments, an immunoconjugate comprises an antibody and a chemotherapeutic agent or other toxin. Chemotherapeutic agents useful in the generation of such immunoconjugates have been described above. Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ¹³¹In, ⁹⁰Y, and ¹⁸⁶Re. Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science, 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026.

Conjugates of an antibody and one or more small molecule toxins, such as a calicheamicin, auristatin peptides, such as monomethylauristatin (MMAE) (synthetic analog of dolastatin), maytansinoids, such as DM1, a trichothene, and CC1065, and the derivatives of these toxins that have toxin activity, are also contemplated herein. Additional non-limiting examples of toxins include those described in WO 2014144871, the disclosure of which is herein incorporated by reference in its entirety.

### Exemplary Immunoconjugates-Antibody-Drug Conjugates

An immunoconjugate (or “antibody-drug conjugate” (“ADC”)) of the invention may be of Formula I, below, wherein an antibody is conjugated (i.e., covalently attached) to one or more drug moieties (D) through an optional linker (L). ADCs may include thioMAb drug conjugates (“TDC”).

Ab-(L-D)ₚ I

Accordingly, the antibody may be conjugated to the drug either directly or via a linker. In Formula I, p is the average number of drug moieties per antibody, which can range, e.g., from about 1 to about 20 drug moieties per antibody, and in certain embodiments, from 1 to about 8 drug moieties per antibody. The invention includes a composition comprising a mixture of antibody-drug compounds of Formula I where the average drug loading per antibody is about 2 to about 5, or about 3 to about 4.

a. Exemplary Linkers

A linker may comprise one or more linker components. Exemplary linker components include 6-maleimidocaproyl (“MC”), maleimidopropanoyl (“MP”), valine-citrulline (“val-cit” or “vc”), alanine-phenylalanine (“ala-phe”), p-aminobenzyloxycarbonyl (a “PAB”), and those resulting from conjugation with linker reagents: N-Succinimidyl 4-(2-pyridylthio) pentanoate forming linker moiety 4-mercaptopentanoic acid (“SPP”), N-succinimidyl 4-(N-maleimidomethyl)cyclohexane-1 carboxylate forming linker moiety 4-((2,5-dioxopyrrolidin-1-yl)methyl)cyclohexanecarboxylic acid (“SMCC”, also referred to herein as “MCC”), 2,5-dioxopyrrolidin-1-yl 4-(pyridin-2-yldisulfanyl) butanoate forming linker moiety 4-mercaptobutanoic acid (“SPDB”), N-Succinimidyl (4-iodo-acetyl) aminobenzoate (“SIAB”), ethyleneoxy —CH₂CH₂O— as one or more repeating units (“EO” or “PEO”). Additional linker components are known in the art and some are described herein. Various linker components are known in the art, some of which are described below.

A linker may be a “cleavable linker,” facilitating release of a drug in the cell. For example, an acid-labile linker (e.g., hydrazone), protease-sensitive (e.g., peptidase-sensitive) linker, photolabile linker, dimethyl linker or disulfide-containing linker (Chari et al., Cancer Research 52:127-31 (1992); U.S. Pat. No. 5,208,020) may be used.

In certain embodiments, a linker is as shown in the following Formula II:

-Aₐ-W_{w}—Y_{y}— II

wherein A is a stretcher unit, and a is an integer from 0 to 1; W is an amino acid unit, and w is an integer from 0 to 12; Y is a spacer unit, and y is 0, 1, or 2; and Ab, D, and p are defined as above for Formula I. Exemplary embodiments of such linkers are described in US 2005-0238649 A1, which is expressly incorporated herein by reference.

In some embodiments, a linker component may comprise a “stretcher unit” that links an antibody to another linker component or to a drug moiety. Exemplary stretcher units are shown below (wherein the wavy line indicates sites of covalent attachment to an antibody):

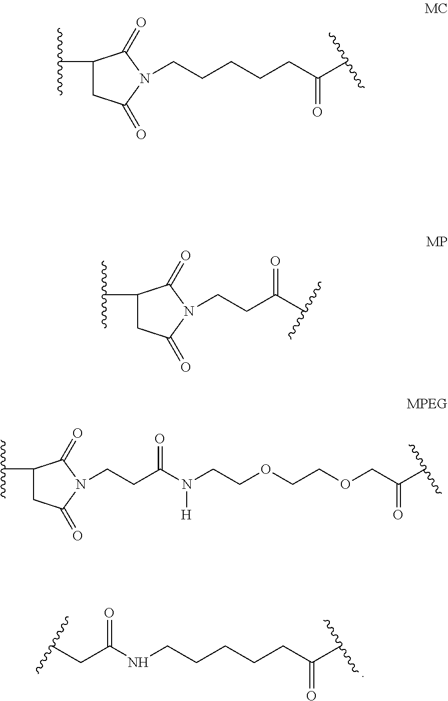 [SMILES: *C(=O)CCCCCN1C(=O)CC(*)C1=O ; *C(=O)CCN1C(=O)CC(*)C1=O ; *C(=O)COCCOCCNC(=O)CCN1C(=O)CC(*)C1=O ; *CC(=O)NCCCCCC(*)=O]

In some embodiments, a linker component may comprise an amino acid unit. In one such embodiment, the amino acid unit allows for cleavage of the linker by a protease, thereby facilitating release of the drug from the immunoconjugate upon exposure to intracellular proteases, such as lysosomal enzymes (see, e.g., Doronina et al. (2003) Nat. Biotechnol. 21:778-4. Exemplary amino acid units include, but are not limited to, a dipeptide, a tripeptide, a tetrapeptide, and a pentapeptide. Exemplary dipeptides include: valine-citrulline (vc or val-cit), alanine-phenylalanine (af or ala-phe); phenylalanine-lysine (fk or phe-lys); or N-methyl-valine-citrulline (Me-val-cit). Exemplary tripeptides include: glycine-valine-citrulline (gly-val-cit) and glycine-glycine-glycine (gly-gly-gly). An amino acid unit may comprise amino acid residues that occur naturally, as well as minor amino acids and non-naturally occurring amino acid analogs, such as citrulline. Amino acid units can be designed and optimized in their selectivity for enzymatic cleavage by a particular enzyme, for example, a tumor-associated protease, cathepsin B, C and D, or a plasmin protease.

In some embodiments, a linker component may comprise a “spacer” unit that links the antibody to a drug moiety, either directly or by way of a stretcher unit and/or an amino acid unit. A spacer unit may be “self-immolative” or a “non-self-immolative.” A “non-self-immolative” spacer unit is one in which part or all of the spacer unit remains bound to the drug moiety upon enzymatic (e.g., proteolytic) cleavage of the ADC. Examples of non-self-immolative spacer units include, but are not limited to, a glycine spacer unit and a glycine-glycine spacer unit. Other combinations of peptidic spacers susceptible to sequence-specific enzymatic cleavage are also contemplated. For example, enzymatic cleavage of an ADC containing a glycine-glycine spacer unit by a tumor-cell associated protease would result in release of a glycine-glycine-drug moiety from the remainder of the ADC. In one such embodiment, the glycine-glycine-drug moiety is then subjected to a separate hydrolysis step in the tumor cell, thus cleaving the glycine-glycine spacer unit from the drug moiety.

A “self-immolative” spacer unit allows for release of the drug moiety without a separate hydrolysis step. In certain embodiments, a spacer unit of a linker comprises a p-aminobenzyl unit. In one such embodiment, a p-aminobenzyl alcohol is attached to an amino acid unit via an amide bond, and a carbamate, methylcarbamate, or carbonate is made between the benzyl alcohol and a cytotoxic agent (see, e.g., Hamann et al. (2005) Expert Opin. Ther. Patents (2005) 15:1087-103. In one embodiment, the spacer unit is p-aminobenzyloxycarbonyl (PAB). In certain embodiments, the phenylene portion of a p-amino benzyl unit is substituted with Qm, wherein Q is —C₁-C₈ alkyl, —O—(C₁-C₈ alkyl), -halogen, -nitro or -cyano; and m is an integer ranging from 0-4. Examples of self-immolative spacer units further include, but are not limited to, aromatic compounds that are electronically similar to p-aminobenzyl alcohol (see, e.g., US 2005/0256030 A1), such as 2-aminoimidazol-5-methanol derivatives (Hay et al. (1999) Bioorg. Med. Chem. Lett. 9:2237) and ortho- or para-aminobenzylacetals. Spacers can be used that undergo cyclization upon amide bond hydrolysis, such as substituted and unsubstituted 4-aminobutyric acid amides (Rodrigues et al., Chemistry Biology, 1995, 2, 223); appropriately substituted bicyclo[2.2.1] and bicyclo[2.2.2] ring systems (Storm, et al., J. Amer. Chem. Soc., 1972, 94:5815); and 2-aminophenylpropionic acid amides (Amsberry, et al., J. Org. Chem., 1990, 55:5867). Elimination of amine-containing drugs that are substituted at the α-position of glycine (Kingsbury, et al., J. Med. Chem., 1984, 27:1447) are also examples of self-immolative spacers useful in ADCs.

In one embodiment, a spacer unit is a branched bis(hydroxymethyl) styrene (BHMS) unit as depicted below, which can be used to incorporate and release multiple drugs.

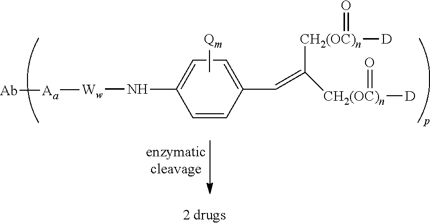

wherein Q is —C₁-C₈ alkyl, —O—(C₁-C₈alkyl), -halogen, -nitro or -cyano; m is an integer ranging from 0-4; n is 0 or 1; and p ranges ranging from 1 to about 20.

In another embodiment, linker L may be a dendritic type linker for covalent 5 attachment of more than one drug moiety through a branching, multifunctional linker moiety to an antibody (Sun et al (2002) Bioorganic & Medicinal Chemistry Letters 12:2213-5; Sun et al (2003) Bioorganic & Medicinal Chemistry 11:1761-8). Dendritic linkers can increase the molar ratio of drug to antibody, i.e. loading, which is related to the potency of the ADC. Thus, where a cysteine engineered antibody bears only one reactive cysteine thiol group, a multitude of drug moieties may be attached through a dendritic linker. 10

Exemplary linker components and combinations thereof are shown below in the context of ADCs of Formula II:

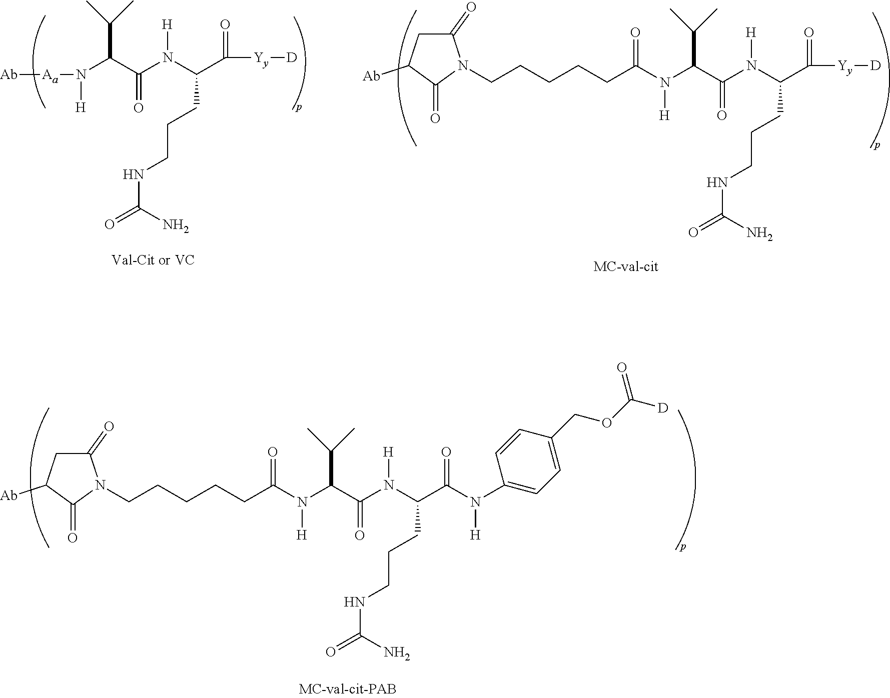

Linkers components, including stretcher, spacer, and amino acid units, may be synthesized by methods known in the art, such as those described in US 2005-0238649 A1.

Additional non-limiting examples of linkers include those described in WO 2015095953, the disclosure of which is herein incorporated by reference in its entirety.

b. Exemplary Drug Moieties

### (1) Maytansine and Maytansinoids

In some embodiments, an immunoconjugate comprises an antibody conjugated to one or more maytansinoid molecules. Maytansinoids are mitototic inhibitors which act by inhibiting tubulin polymerization. Maytansine was first isolated from the east African shrub Maytenus serrata (U.S. Pat. No. 3,896,111). Subsequently, it was discovered that certain microbes also produce maytansinoids, such as maytansinol and C-3 maytansinol esters (U.S. U.S. Pat. No. 4,151,042). Synthetic maytansinol and derivatives and analogues thereof are disclosed, for example, in U.S. Pat. Nos. 4,137,230; 4,248,870; 4,256,746; 4,260,608; 4,265,814; 4,294,757; 4,307,016; 4,308,268; 4,308,269; 4,309,428; 4,313,946; 4,315,929; 4,317,821; 4,322,348; 4,331,598; 4,361,650; 4,364,866; 4,424,219; 4,450,254; 4,362,663; and 4,371,533.

Maytansinoid drug moieties are attractive drug moieties in antibody-drug conjugates because they are: (i) relatively accessible to prepare by fermentation or chemical modification or derivatization of fermentation products, (ii) amenable to derivatization with functional groups suitable for conjugation through disulfide and non-disulfide linkers to antibodies, (iii) stable in plasma, and (iv) effective against a variety of tumor cell lines.

Maytansine compounds suitable for use as maytansinoid drug moieties are well known in the art and can be isolated from natural sources according to known methods or produced using genetic engineering and fermentation techniques (U.S. Pat. No. 6,790,952; US 2005/0170475; Yu et al (2002) PNAS 99:7968-73). Maytansinol and maytansinol analogues may also be prepared synthetically according to known methods.

Exemplary maytansinoid drug moieties include those having a modified aromatic ring, such as: C-19-dechloro (U.S. Pat. No. 4,256,746) (prepared by lithium aluminum hydride reduction of ansamytocin P2); C-20-hydroxy (or C-20-demethyl)+/−C-19-dechloro (U.S. Pat. Nos. 4,361,650 and 4,307,016) (prepared by demethylation using Streptomyces or Actinomyces or dechlorination using LAH); and C-20-demethoxy, C-20-acyloxy (—OCOR), +/−dechloro (U.S. Pat. No. 4,294,757) (prepared by acylation using acyl chlorides) and those having modifications at other positions.

Exemplary maytansinoid drug moieties also include those having modifications such as: C-9-SH (U.S. Pat. No. 4,424,219) (prepared by the reaction of maytansinol with H₂S or P₂S₅); C-14-alkoxymethyl (demethoxy/CH₂OR) (U.S. Pat. No. 4,331,598); C-14-hydroxymethyl or acyloxymethyl (CH₂OH or CH₂OAc) (U.S. Pat. No. 4,450,254) (prepared from Nocardia); C-15-hydroxy/acyloxy (U.S. Pat. No. 4,364,866) (prepared by the conversion of maytansinol by Streptomyces); C-15-methoxy (U.S. Pat. Nos. 4,313,946 and 4,315,929) (isolated from Trewia nudlflora); C-18-N-demethyl (U.S. Pat. Nos. 4,362,663 and 4,322,348) (prepared by the demethylation of maytansinol by Streptomyces); and 4,5-deoxy (U.S. Pat. No. 4,371,533) (prepared by the titanium trichloride/LAH reduction of maytansinol).

Many positions on maytansine compounds are known to be useful as the linkage position, depending upon the type of link. For example, for forming an ester linkage, the C-3 position having a hydroxyl group, the C-14 position modified with hydroxymethyl, the C-15 position modified with a hydroxyl group and the C-20 position having a hydroxyl group are all suitable (U.S. Pat. No. 5,208,020; U.S. RE39,151; U.S. Pat. Nos. 6,913,748; 7,368,565; US 2006/0167245; US 2007/0037972).

Maytansinoid drug moieties include those having the structure:

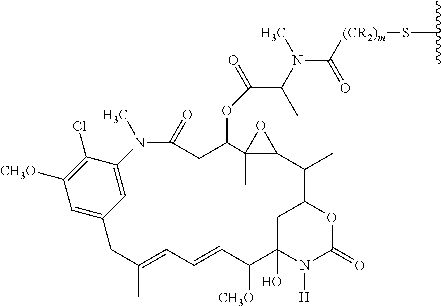

where the wavy line indicates the covalent attachment of the sulfur atom of the maytansinoid drug moiety to a linker of an ADC. R may independently be H or a C₁-C₆ alkyl. The alkylene chain attaching the amide group to the sulfur atom may be methanyl, ethanyl, or propyl, i.e., m is 1, 2, or 3 (U.S. Pat. No. 633,410; U.S. Pat. Nos. 5,208,020; 7,276,497; Chari et al (1992) Cancer Res. 52:127-31; Liu et al (1996) Proc. Natl. Acad. Sci USA 93:8618-23).

All stereoisomers of the maytansinoid drug moiety are contemplated for the compounds of the invention, i.e. any combination of R and S configurations at the chiral carbons of D. In one embodiment, the maytansinoid drug moiety will have the following stereochemistry:

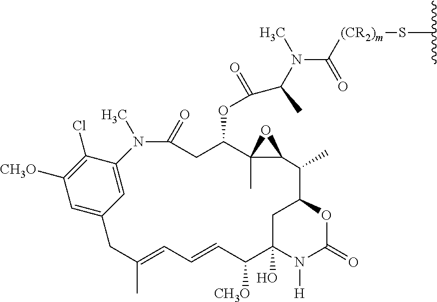

Exemplary embodiments of maytansinoid drug moieities include: DM1; DM3; and DM4, having the structures:

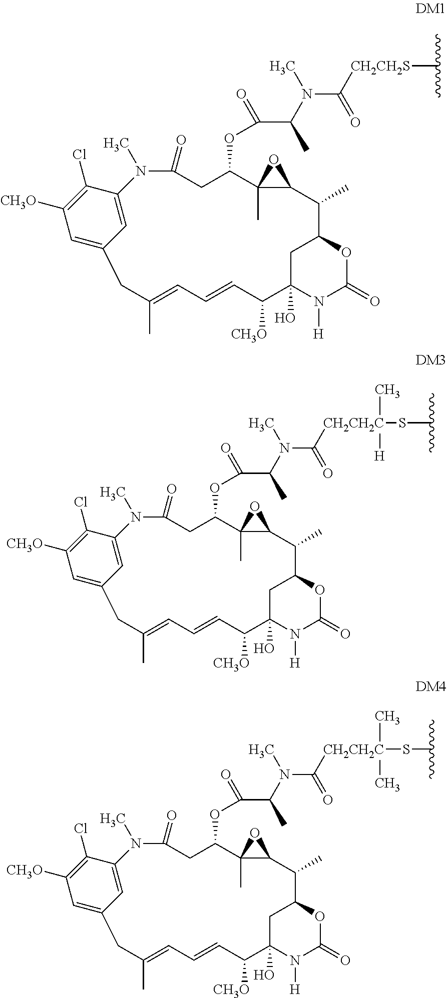

wherein the wavy line indicates the covalent attachment of the sulfur atom of the drug to a linker (L) of an antibody-drug conjugate. (WO 2005/037992; US 2005/0276812 A1).

Other exemplary maytansinoid antibody-drug conjugates have the following structures and abbreviations, (wherein Ab is antibody and p is 1 to about 8):

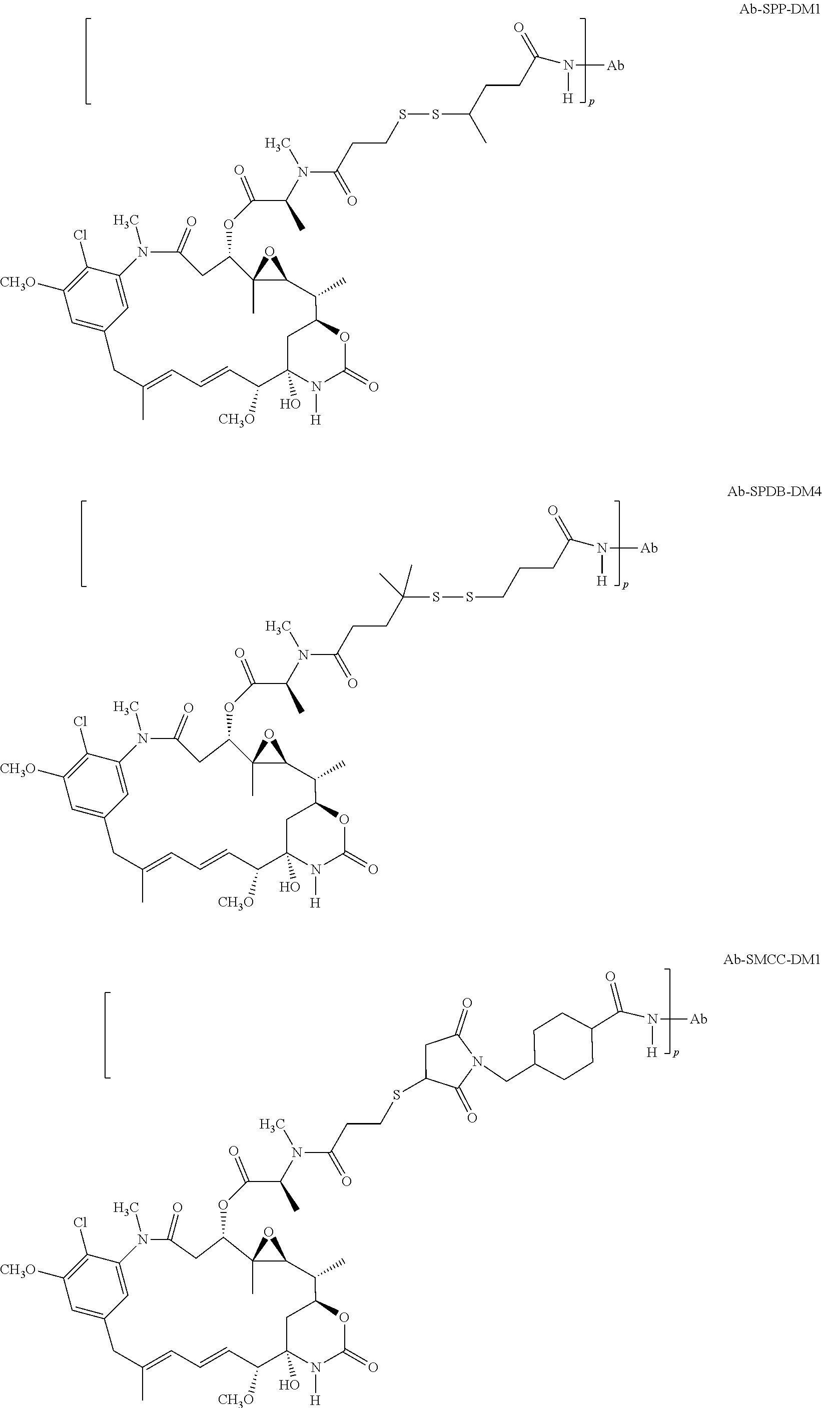

In one embodiment, the antibody-drug conjugate is formed where DM4 is linked through an SPDB linker to a thiol group of the antibody (see U.S. Pat. Nos. 6,913,748 and 7,276,497 incorporated herein by reference in their entirety).

Exemplary antibody-drug conjugates where DM1 is linked through a BMPEO linker to a thiol group of the antibody have the structure and abbreviation:

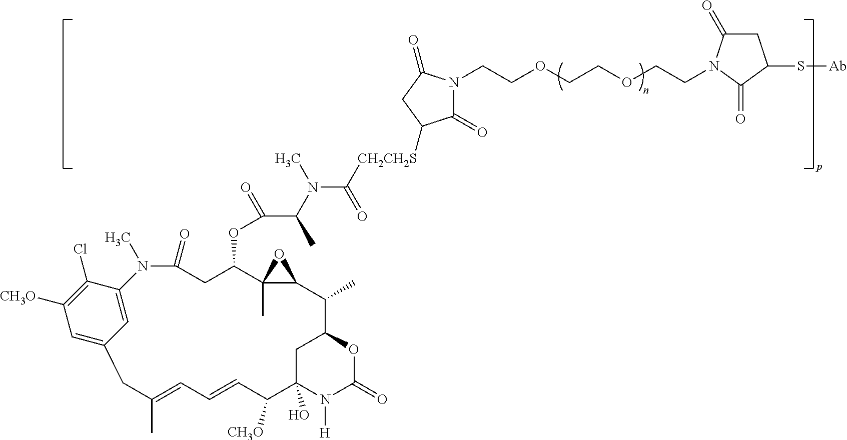

where Ab is antibody; n is 0, 1, or 2; and p is 1, 2, 3, or 4.

Immunoconjugates containing maytansinoids, methods of making the same, and their therapeutic use are disclosed, for example, in Erickson, et al (2006) Cancer Res. 66 (8): 4426-33; U.S. Pat. Nos. 5,208,020, 5,416,064, US 2005/0276812 A1, and European Patent EP 0 425 235 B1, the disclosures of which are hereby expressly incorporated by reference.

Antibody-maytansinoid conjugates are prepared by chemically linking an antibody to a maytansinoid molecule without significantly diminishing the biological activity of either the antibody or the maytansinoid molecule (see, e.g., U.S. Pat. No. 5,208,020 (the disclosure of which is hereby expressly incorporated by reference). Maytansinoids can be synthesized by known techniques or isolated from natural sources. Suitable maytansinoids are disclosed, for example, in U.S. Pat. No. 5,208,020 and in the other patents and nonpatent publications referred to hereinabove, such as maytansinol and maytansinol analogues modified in the aromatic ring or at other positions of the maytansinol molecule, such as various maytansinol esters.

There are many linking groups known in the art for making antibody-maytansinoid conjugates, including, for example, those disclosed in U.S. Pat. No. 5,208,020 or EP Patent 0 425 235 B1; Chari et al. Cancer Research 52:127-31 (1992); and US 2005/016993 A1, the disclosures of which are hereby expressly incorporated by reference. Antibody-maytansinoid conjugates comprising the linker component SMCC may be prepared as disclosed in US 2005/0276812 A1, “Antibody-drug conjugates and Methods.” The linkers comprise disulfide groups, thioether groups, acid labile groups, photolabile groups, peptidase labile groups, or esterase labile groups, as disclosed in the above-identified patents. Additional linkers are described and exemplified herein.

Conjugates of the antibody and maytansinoid may be made using a variety of bifunctional protein coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). In certain embodiments, the coupling agent is N-succinimidyl-3-(2-pyridyldithio) propionate (SPDP) (Carlsson et al., Biochem. J. 173:723-37 (1978)) or N-succinimidyl-4-(2-pyridylthio) pentanoate (SPP) to provide for a disulfide linkage.

The linker may be attached to the maytansinoid molecule at various positions, depending on the type of the link. For example, an ester linkage may be formed by reaction with a hydroxyl group using conventional coupling techniques. The reaction may occur at the C-3 position having a hydroxyl group, the C-14 position modified with hydroxymethyl, the C-15 position modified with a hydroxyl group, and the C-20 position having a hydroxyl group. In one embodiment, the linkage is formed at the C-3 position of maytansinol or a maytansinol analogue.

### (2) Auristatins and Dolastatins

In some embodiments, an immunoconjugate comprises an antibody conjugated to dolastatin or a dolastatin peptidic analog or derivative, e.g., an auristatin (U.S. Pat. Nos. 5,635,483; 5,780,588). Dolastatins and auristatins have been shown to interfere with microtubule dynamics, GTP hydrolysis, and nuclear and cellular division (Woyke et al (2001) Antimicrob. Agents and Chemother. 45 (12): 3580-4) and have anticancer (U.S. Pat. No. 5,663,149) and antifungal activity (Pettit et al (1998) Antimicrob. Agents Chemother. 42:2961-5). The dolastatin or auristatin drug moiety may be attached to the antibody through the N (amino) terminus or the C (carboxyl) terminus of the peptidic drug moiety (WO 02/088172).

Exemplary auristatin embodiments include the N-terminus linked monomethylauristatin drug moieties DE and DF (US 2005/0238649, disclosed in Senter et al, Proceedings of the American Association for Cancer Research, 45, Abstract Number 623, presented Mar. 28, 2004, the disclosure of which is expressly incorporated by reference in its entirety).

A peptidic drug moiety may be selected from Formulas D_{E} and D_{F} below:

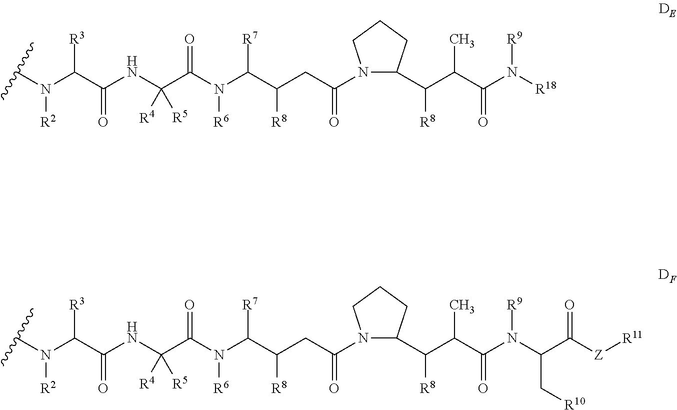

wherein the wavy line of D_{E} and D_{F} indicates the covalent attachment site to an antibody or antibody-linker component, and independently at each location:
R² is selected from H and C₁-C₈alkyl;
R³ is selected from H, C₁-C₈alkyl, C₃-C₈ carbocycle, aryl, C₁-C₈alkyl-aryl, C₁-C₈ alkyl-(C₃-C₈ carbocycle), C₃-C₈ heterocycle and C₁-C₈alkyl-(C₃-C₈ heterocycle);
R⁴ is selected from H, C₁-C₈alkyl, C₃-C₈ carbocycle, aryl, C₁-C₈alkyl-aryl, C₁-C₈ alkyl-(C₃-C₈ carbocycle), C₃-C₈ heterocycle and C₁-C₈alkyl-(C₃-C₈ heterocycle);
R⁵ is selected from H and methyl;
or R⁴ and R⁵ jointly form a carbocyclic ring and have the formula —(CR^{a}R^{b})ₙ— wherein R^{a} and R^{b} are independently selected from H, C₁-C₈alkyl and C₃-C₈ carbocycle and n is selected from 2, 3, 4, 5 and 6;
R⁶ is selected from H and C₁-C₈alkyl;
R⁷ is selected from H, C₁-C₈alkyl, C₃-C₈ carbocycle, aryl, C₁-C₈ alkyl-aryl, C₁-C₈ alkyl-(C₃-C₈ carbocycle), C₃-C₈ heterocycle and C₁-C₈alkyl-(C₃-C₈ heterocycle); each R⁸ is independently selected from H, OH, C₁-C₈alkyl, C₃-C₈ carbocycle and O—(C₁-C₈alkyl);
R⁹ is selected from H and C₁-C₈ alkyl;
R¹⁰ is selected from aryl or C₃-C₈ heterocycle;
Z is O, S, NH, or NR¹², wherein R¹² is C₁-C₈ alkyl;
R¹¹ is selected from H, C₁-C₂₀ alkyl, aryl, C₃-C₈ heterocycle, —(R¹³O)ₘ—R¹⁴, or —(R¹³O)ₘ—CH(R¹⁵)₂;
m is an integer ranging from 1-1000;
R¹³ is C₂-C₈ alkyl;
R¹⁴ is H or C₁-C₈alkyl;
each occurrence of R¹⁵ is independently H, COOH, —(CH₂)ₙ—N(R¹⁶)₂, —(CH₂)ₙ—SO₃H, or —(CH₂)ₙ—SO₃—C₁-C₈ alkyl;
each occurrence of R¹⁶ is independently H, C₁-C₈alkyl, or —(CH₂)ₙ—COOH;
R¹⁸ is selected from —C(R⁸)₂—C(R⁸)₂-aryl, —C(R⁸)₂—C(R⁸)₂—(C₃-C₈ heterocycle), and —C(R⁸)₂—C(R⁸)₂—(C₃-C₈ carbocycle); and
n is an integer ranging from 0 to 6.

In one embodiment, R³, R⁴ and R⁷ are independently isopropyl or sec-butyl and R⁵ is —H or methyl. In an exemplary embodiment, R³ and R⁴ are each isopropyl, R⁵ is —H, and R⁷ is sec-butyl.

In yet another embodiment, R² and R⁶ are each methyl, and R⁹ is —H.

In still another embodiment, each occurrence of R⁸ is —OCH₃.

In an exemplary embodiment, R³ and R⁴ are each isopropyl, R² and R⁶ are each methyl, R⁵ is —H, R⁷ is sec-butyl, each occurrence of R⁸ is —OCH₃, and R⁹ is —H.

In one embodiment, Z is —O— or —NH—.

In one embodiment, R¹⁰ is aryl.

In an exemplary embodiment, R¹⁰ is -phenyl.

In an exemplary embodiment, when Z is —O—, R¹¹ is —H, methyl or t-butyl.

In one embodiment, when Z is —NH, R¹¹ is —CH(R¹⁵)₂, wherein R¹⁵ is —(CH₂)ₙ—N(R¹⁶)₂, and R¹⁶ is —C₁-C₈ alkyl or —(CH₂)ₙ—COOH.

In another embodiment, when Z is —NH, R¹¹ is —CH(R¹⁵)₂, wherein R¹⁵ is —(CH₂)ₙ—SO₃H.

An exemplary auristatin embodiment of formula D_{E} is MMAE, wherein the wavy line indicates the covalent attachment to a linker (L) of an antibody-drug conjugate:

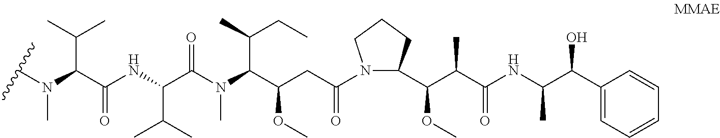 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@H](C)[C@@H](O)c1ccccc1)OC)C(C)C)C(C)C]

An exemplary auristatin embodiment of formula D_{F} is MMAF, wherein the wavy line indicates the covalent attachment to a linker (L) of an antibody-drug conjugate (see US 2005/0238649 and Doronina et al. (2006) Bioconjugate Chem. 17:114-124):

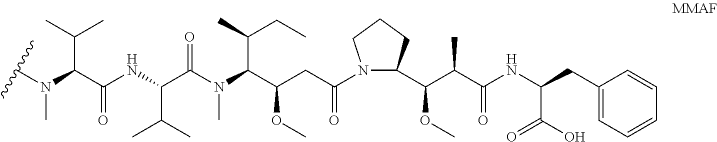 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@@H](Cc1ccccc1)C(=O)O)OC)C(C)C)C(C)C]

Other exemplary embodiments include monomethylvaline compounds having phenylalanine carboxy modifications at the C-terminus of the pentapeptide auristatin drug moiety (WO 2007/008848) and monomethylvaline compounds having phenylalanine sidechain modifications at the C-terminus of the pentapeptide auristatin drug moiety (WO 2007/008603).

Other drug moieties include the following MMAF derivatives, wherein the wavy line indicates the covalent attachment to a linker (L) of an antibody-drug conjugate:

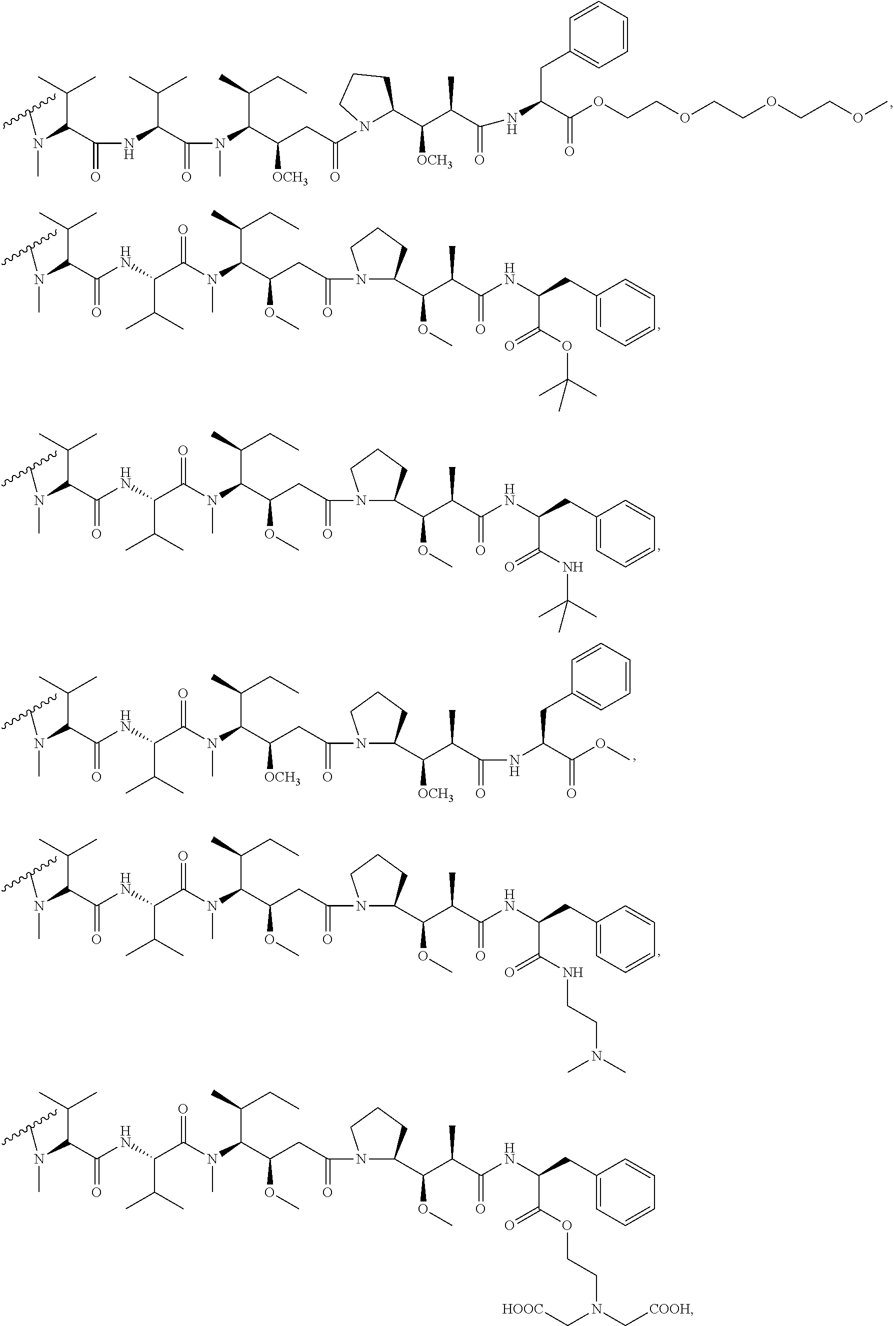

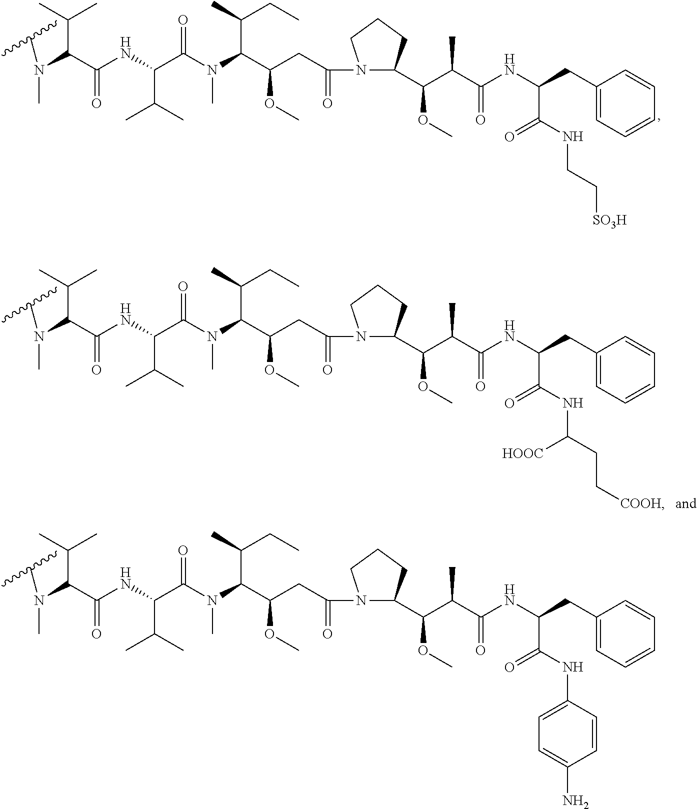

In one aspect, hydrophilic groups including but not limited to, triethylene glycol esters (TEG), as shown above, can be attached to the drug moiety at R¹¹. Without being bound by any particular theory, the hydrophilic groups assist in the internalization and non-agglomeration of the drug moiety.

Exemplary embodiments of ADCs of Formula I comprising an auristatin/dolastatin or derivative thereof are described in US 2005-0238649 and Doronina et al. (2006) Bioconjugate Chem. 17:114-24, which is expressly incorporated herein by reference. Exemplary embodiments of ADCs of Formula I comprising MMAE or MMAF and various linker components have the following structures and abbreviations (wherein “Ab” is an antibody; p is 1 to about 8, “Val-Cit” or “vc” is a valine-citrulline dipeptide; and “S” is a sulfur atom. It will be noted that in certain of the structural descriptions of sulfur linked ADC herein the antibody is represented as “Ab-S” merely to indicate the sulfur link feature and not to indicate that a particular sulfur atom bears multiple linker-drug moieties. The left parentheses of the following structures may also be placed to the left of the sulfur atom, between Ab and S, which would be an equivalent description of the ADC of the invention described throughout herein.

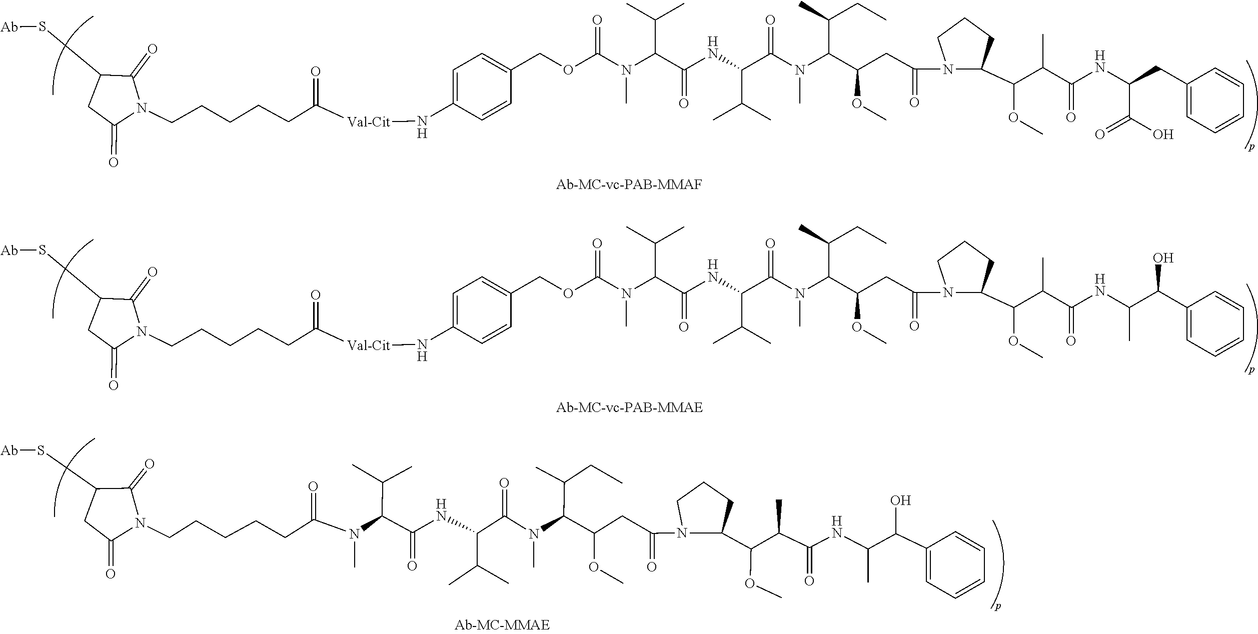

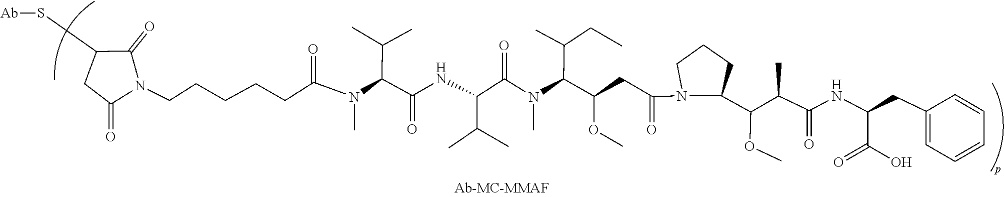

Exemplary embodiments of ADCs of Formula I comprising MMAF and various linker components further include Ab-MC-PAB-MMAF and Ab-PAB-MMAF. Interestingly, immunoconjugates comprising MMAF attached to an antibody by a linker that is not proteolytically cleavable have been shown to possess activity comparable to immunoconjugates comprising MMAF attached to an antibody by a proteolytically cleavable linker (see, Doronina et al. (2006) Bioconjugate Chem. 17:114-24. In such instances, drug release is believed to be effected by antibody degradation in the cell.

Typically, peptide-based drug moieties can be prepared by forming a peptide bond between two or more amino acids and/or peptide fragments. Such peptide bonds can be prepared, for example, according to the liquid phase synthesis method (see E. Schröder and K. Lübke, The Peptides, Vol. 1, pp 76-136, 1965, Academic Press) that is well known in the field of peptide chemistry. Auristatin/dolastatin drug moieties may be prepared according to the methods of: US 2005-0238649 A1; U.S. Pat. Nos. 5,635,483; 5,780,588; Pettit et al (1989) J. Am. Chem. Soc. 111:5463-5; Pettit et al (1998) Anti-Cancer Drug Design 13:243-77; Pettit, G. R., et al. Synthesis, 1996, 719-25; Pettit et al (1996) J. Chem. Soc. Perkin Trans. 15:859-63; and Doronina (2003) Nat. Biotechnol. 21 (7): 778-84.

In particular, auristatin/dolastatin drug moieties of formula D_{F}, such as MMAF and derivatives thereof, may be prepared using methods described in US 2005-0238649 A1 and Doronina et al. (2006) Bioconjugate Chem. 17:114-124. Auristatin/dolastatin drug moieties of formula D_{E}, such as MMAE and derivatives thereof, may be prepared using methods described in Doronina et al. (2003) Nat. Biotech. 21:778-84. Drug-linker moieties MC-MMAF, MC-MMAE, MC-vc-PAB-MMAF, and MC-vc-PAB-MMAE may be conveniently synthesized by routine methods, e.g., as described in Doronina et al. (2003) Nat. Biotech. 21:778-784, and Patent Application Publication No. US 2005/0238649 A1, and then conjugated to an antibody of interest.

### (3) Calicheamicin

In other embodiments, the immunoconjugate comprises an antibody conjugated to one or more calicheamicin molecules. The calicheamicin family of antibiotics are capable of producing double-stranded DNA breaks at sub-picomolar concentrations. For the preparation of conjugates of the calicheamicin family, see U.S. Pat. Nos. 5,712,374, 5,714,586, 5,739,116, 5,767,285, 5,770,701, 5,770,710, 5,773,001, 5,877,296 (all to American Cyanamid Company). Structural analogues of calicheamicin which may be used include, but are not limited to, γ₁^{I}, α₂^{I}, α₃^{I}, N-acetyl-γ₁^{I}, PSAG and θ¹₁ (Hinman et al., Cancer Research 53:3336-3342 (1993), Lode et al., Cancer Research 58:2925-8 (1998), and the aforementioned U.S. patents to American Cyanamid). Another anti-tumor drug to which the antibody can be conjugated is QFA, which is an antifolate. Both calicheamicin and QFA have intracellular sites of action and do not readily cross the plasma membrane. Therefore, cellular uptake of these agents through antibody-mediated internalization greatly enhances their cytotoxic effects.

### C. Other Cytotoxic Agents

Other antitumor agents that can be conjugated to an antibody include BCNU, streptozocin, vincristine and 5-fluorouracil, the family of agents known collectively as the LL-E33288 complex, described in U.S. Pat. Nos. 5,053,394, 5,770,710, as well as esperamicins (U.S. Pat. No. 5,877,296).

Enzymatically active toxins and fragments thereof which can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin and the tricothecenes. See, for example, WO 93/21232 published Oct. 28, 1993.

The present invention further contemplates an immunoconjugate formed between an antibody and a compound with nucleolytic activity (e.g., a ribonuclease or a DNA endonuclease such as a deoxyribonuclease; DNase).

In certain embodiments, an immunoconjugate may comprise a highly radioactive atom. A variety of radioactive isotopes are available for the production of radioconjugated antibodies. Examples include At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu. When the immunoconjugate is used for detection, it may comprise a radioactive atom for scintigraphic studies, for example tc^{99m} or I¹²³, or a spin label for nuclear magnetic resonance (NMR) imaging (also known as magnetic resonance imaging, mri), such as iodine-123, iodine-131, indium-111, fluorine-19, carbon-13, nitrogen-15, oxygen-17, gadolinium, manganese or iron.

The radio- or other labels may be incorporated in the immunoconjugate in known ways. For example, the peptide may be biosynthesized or may be synthesized by chemical amino acid synthesis using suitable amino acid precursors involving, for example, fluorine-19 in place of hydrogen. Labels such as tc^{99m} or I¹²³, Re¹⁸⁶, Re¹⁸⁸ and In¹¹¹ can be attached via a cysteine residue in the peptide. Yttrium-90 can be attached via a lysine residue. The IODOGEN method (Fraker et al (1978) Biochem. Biophys. Res. Commun. 80:49-57 can be used to incorporate iodine-123. “Monoclonal Antibodies in Immunoscintigraphy” (Chatal, CRC Press 1989) describes other methods in detail.

In certain embodiments, an immunoconjugate may comprise an antibody conjugated to a prodrug-activating enzyme that converts a prodrug (e.g., a peptidyl chemotherapeutic agent, see WO 81/01145) to an active drug, such as an anti-cancer drug. Such immunoconjugates are useful in antibody-dependent enzyme-mediated prodrug therapy (“ADEPT”). Enzymes that may be conjugated to an antibody include, but are not limited to, alkaline phosphatases, which are useful for converting phosphate-containing prodrugs into free drugs; arylsulfatases, which are useful for converting sulfate-containing prodrugs into free drugs; cytosine deaminase, which is useful for converting non-toxic 5-fluorocytosine into the anti-cancer drug, 5-fluorouracil; proteases, such as Serratia protease, thermolysin, subtilisin, carboxypeptidases and cathepsins (such as cathepsins B and L), which are useful for converting peptide-containing prodrugs into free drugs; D-alanylcarboxypeptidases, which are useful for converting prodrugs that contain D-amino acid substituents; carbohydrate-cleaving enzymes such as β-galactosidase and neuraminidase, which are useful for converting glycosylated prodrugs into free drugs; β-lactamase, which is useful for converting drugs derivatized with β-lactams into free drugs; and penicillin amidases, such as penicillin V amidase and penicillin G amidase, which are useful for converting drugs derivatized at their amine nitrogens with phenoxyacetyl or phenylacetyl groups, respectively, into free drugs. Enzymes may be covalently bound to antibodies by recombinant DNA techniques well known in the art (e.g., Neuberger et al., Nature 312:604-608 (1984)).

d. Drug Loading

Drug loading is represented by p, the average number of drug moieties per antibody in a molecule of Formula I. Drug loading may range from 1 to 20 drug moieties (D) per antibody. ADCs of Formula I include collections of antibodies conjugated with a range of drug moieties, from 1 to 20. The average number of drug moieties per antibody in preparations of ADC from conjugation reactions may be characterized by conventional means such as mass spectroscopy, ELISA assay, and HPLC. The quantitative distribution of ADC in terms of p may also be determined. In some instances, separation, purification, and characterization of homogeneous ADC where p is a certain value from ADC with other drug loadings may be achieved by means such as reverse phase HPLC or electrophoresis. Pharmaceutical formulations of Formula I antibody-drug conjugates may thus be a heterogeneous mixture of such conjugates with antibodies linked to 1, 2, 3, 4, or more drug moieties.

For some antibody-drug conjugates, p may be limited by the number of attachment sites on the antibody. For example, where the attachment is a cysteine thiol, as in the exemplary embodiments above, an antibody may have only one or several cysteine thiol groups, or may have only one or several sufficiently reactive thiol groups through which a linker may be attached. In certain embodiments, higher drug loading, e.g., p>5, may cause aggregation, insolubility, toxicity, or loss of cellular permeability of certain antibody-drug conjugates. In certain embodiments, the drug loading for an ADC of the invention ranges from 1 to about 8; from about 2 to about 6; or from about 3 to about 5. Indeed, it has been shown that for certain ADCs, the optimal ratio of drug moieties per antibody may be less than 8, and may be about 2 to about 5 (see US 2005-0238649 A1).

In certain embodiments, less than the theoretical maximum of drug moieties are conjugated to an antibody during a conjugation reaction. An antibody may contain, for example, lysine residues that do not react with the drug-linker intermediate or linker reagent, as discussed below. Generally, antibodies do not contain many free and reactive cysteine thiol groups which may be linked to a drug moiety; indeed most cysteine thiol residues in antibodies exist as disulfide bridges. In certain embodiments, an antibody may be reduced with a reducing agent such as dithiothreitol (DTT) or tricarbonylethylphosphine (TCEP), under partial or total reducing conditions, to generate reactive cysteine thiol groups. In certain embodiments, an antibody is subjected to denaturing conditions to reveal reactive nucleophilic groups such as lysine or cysteine.

The loading (drug/antibody ratio) of an ADC may be controlled in different ways, e.g., by: (i) limiting the molar excess of drug-linker intermediate or linker reagent relative to antibody, (ii) limiting the conjugation reaction time or temperature, and (iii) partial or limiting reductive conditions for cysteine thiol modification.

It is to be understood that where more than one nucleophilic group reacts with a drug-linker intermediate or linker reagent followed by drug moiety reagent, then the resulting product is a mixture of ADC compounds with a distribution of one or more drug moieties attached to an antibody. The average number of drugs per antibody may be calculated from the mixture by a dual ELISA antibody assay, which is specific for antibody and specific for the drug. Individual ADC molecules may be identified in the mixture by mass spectroscopy and separated by HPLC, e.g., hydrophobic interaction chromatography (see, e.g., McDonagh et al (2006) Prot. Engr. Design & Selection 19 (7): 299-307; Hamblett et al (2004) Clin. Cancer Res. 10:7063-70; Hamblett, K. J., et al. “Effect of drug loading on the pharmacology, pharmacokinetics, and toxicity of an anti-CD30 antibody-drug conjugate,” Abstract No. 624, American Association for Cancer Research, 2004 Annual Meeting, Mar. 27-31, 2004, Proceedings of the AACR, Volume 45, March 2004; Alley, S. C., et al. “Controlling the location of drug attachment in antibody-drug conjugates,” Abstract No. 627, American Association for Cancer Research, 2004 Annual Meeting, Mar. 27-31, 2004, Proceedings of the AACR, Volume 45, March 2004). In certain embodiments, a homogeneous ADC with a single loading value may be isolated from the conjugation mixture by electrophoresis or chromatography.

### Treatment with CAR Modified Immune Cells

In certain embodiments, the invention relates to compositions and methods for treating cancer including but not limited to hematologic malignancies and solid tumors. In certain embodiments, CAR modified immune cells are used. CAR-T cells can be used therapeutically for patients suffering from non-hematological tumors such as solid tumors arising from breast, CNS, and skin malignancies. In certain embodiments, CAR-NK cells can be used therapeutically for patients suffering from any one of a number of malignancies.

In certain embodiments, the present invention relates to a strategy of adoptive cell transfer of T cells or NK cells transduced to express a chimeric antigen receptor (CAR). CARs are molecules that combine antibody-based specificity for a desired antigen (e.g., tumor antigen) with, for example, a T cell receptor-activating intracellular domain to generate a chimeric protein that exhibits a specific anti-tumor cellular immune activity.

In one aspect, the present invention relates to the use of NK cells genetically modified to stably express a desired CAR. NK cells expressing a CAR are referred to herein as CAR-NK cells or CAR modified NK cells. Preferably, the cell can be genetically modified to stably express an antibody binding domain on its surface, conferring novel antigen specificity. Methods for generating CAR-NK cells are known in the art. For example, see Glienke et al., Front Pharmacol. 2015; 6:21. Services for generating CAR-NK cells are commercially avaibale. See for example Creative Biolabs Inc., 45-1 Ramsey Road, Shirley, NY 11967, USA.

In one aspect, the present invention relates to the use of T cells genetically modified to stably express a desired CAR. T cells expressing a CAR are referred to herein as CAR-T cells or CAR modified T cells. Preferably, the cell can be genetically modified to stably express an antibody binding domain on its surface, conferring novel antigen specificity that is MHC independent. In some instances, the T cell is genetically modified to stably express a CAR that combines an antigen recognition domain of a specific antibody with an intracellular domain of the CD3-zeta chain or FcγRI protein into a single chimeric protein.

In one embodiment, the CAR of the invention comprises an extracellular domain having an antigen recognition domain, a transmembrane domain, and a cytoplasmic domain. In one embodiment, the transmembrane domain that naturally is associated with one of the domains in the CAR is used. In another embodiment, the transmembrane domain can be selected or modified by amino acid substitution to avoid binding of such domains to the transmembrane domains of the same or different surface membrane proteins to minimize interactions with other members of the receptor complex. In one embodiment, the transmembrane domain is the CD8a hinge domain.

With respect to the cytoplasmic domain, the CAR of the invention can be designed to comprise the CD28 and/or 4-1BB signaling domain by itself or be combined with any other desired cytoplasmic domain(s) useful in the context of the CAR of the invention. In one embodiment, the cytoplasmic domain of the CAR can be designed to further comprise the signaling domain of CD3-zeta. For example, the cytoplasmic domain of the CAR can include but is not limited to CD3-zeta, 4-1BB and CD28 signaling modules and combinations thereof. Accordingly, the invention provides CAR T cells and methods of their use for adoptive therapy.

In one embodiment, the CAR T cells of the invention can be generated by introducing a lentiviral vector comprising a desired CAR, for example a CAR comprising anti-activin, CD8α hinge and transmembrane domain, and human 4-1BB and CD3zeta signaling domains, into the cells. The CAR T cells of the invention are able to replicate in vivo resulting in long-term persistence that can lead to sustained tumor control.

In one embodiment, the anti-activin domain comprises a heavy chain variable region comprising any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, and 69.

In one embodiment, an anti-activin domain comprises a heavy chain variable region comprising SEQ ID NO:1 and a light chain variable region comprising SEQ ID NO:2.

In one embodiment, an anti-activin domain comprises a heavy chain variable region comprising SEQ ID NO:3 and a light chain variable region comprising SEQ ID NO:4.

In one embodiment, an anti-activin domain comprises a heavy chain variable region comprising SEQ ID NO:5 and a light chain variable region comprising SEQ ID NO:6.

In one embodiment, an anti-activin domain comprises a heavy chain variable region comprising SEQ ID NO:7 and a light chain variable region comprising SEQ ID NO:8.

In one embodiment, an anti-activin domain comprises a heavy chain variable region comprising SEQ ID NO:9 and a light chain variable region comprising SEQ ID NO: 10.

In one embodiment, an anti-activin domain comprises a heavy chain variable region comprising SEQ ID NO:11 and a light chain variable region comprising SEQ ID NO: 12.

In one embodiment, an anti-activin domain comprises a heavy chain variable region comprising SEQ ID NO:13 and a light chain variable region comprising SEQ ID NO:14.

In one embodiment, an anti-activin domain comprises a heavy chain variable region comprising SEQ ID NO:15 and a light chain variable region comprising SEQ ID NO: 16.

In one embodiment, an anti-activin domain comprises a heavy chain variable region comprising SEQ ID NO:17 and a light chain variable region comprising SEQ ID NO:18.

In one embodiment, an anti-activin domain comprises a heavy chain variable region comprising SEQ ID NO: 19 and a light chain variable region comprising SEQ ID NO:20.

In one embodiment, an anti-activin domain comprises a heavy chain variable region comprising SEQ ID NO:21 and a light chain variable region comprising SEQ ID NO:22.

In one embodiment, an anti-activin domain comprises a heavy chain variable region comprising SEQ ID NO:23 and a light chain variable region comprising SEQ ID NO:24.

In one embodiment, an anti-activin domain comprises a heavy chain variable region comprising SEQ ID NO:25 and a light chain variable region comprising SEQ ID NO:26.

In one embodiment, an anti-activin domain comprises a heavy chain variable region comprising SEQ ID NO:27 and a light chain variable region comprising SEQ ID NO:28.

In one embodiment, an anti-activin domain comprises a heavy chain variable region comprising SEQ ID NO:29 and a light chain variable region comprising SEQ ID NO:30.

In one embodiment, an anti-activin domain comprises a heavy chain variable region comprising SEQ ID NO:31 and a light chain variable region comprising SEQ ID NO:32.

In one embodiment, an anti-activin domain comprises a heavy chain variable region comprising SEQ ID NO:33 and a light chain variable region comprising SEQ ID NO:34.

In one embodiment, an anti-activin domain comprises a heavy chain variable region comprising SEQ ID NO:35 and a light chain variable region comprising SEQ ID NO:36.

In one embodiment, an anti-activin domain comprises a heavy chain variable region comprising SEQ ID NO:37 and a light chain variable region comprising SEQ ID NO:38.

In one embodiment, an anti-activin domain comprises a heavy chain variable region comprising SEQ ID NO:39 and a light chain variable region comprising SEQ ID NO:40.

In one embodiment, an anti-activin domain comprises a heavy chain variable region comprising SEQ ID NO:41 and a light chain variable region comprising SEQ ID NO:42.

In one embodiment, an anti-activin domain comprises a heavy chain variable region comprising SEQ ID NO:43 and a light chain variable region comprising SEQ ID NO:44.

In one embodiment, an anti-activin domain comprises a heavy chain variable region comprising SEQ ID NO:45 and a light chain variable region comprising SEQ ID NO:46.

In one embodiment, an anti-activin domain comprises a heavy chain variable region comprising SEQ ID NO:47 and a light chain variable region comprising SEQ ID NO:48.

In one embodiment, an anti-activin domain comprises a heavy chain variable region comprising SEQ ID NO:49 and a light chain variable region comprising SEQ ID NO:50.

In one embodiment, an anti-activin domain comprises a heavy chain variable region comprising SEQ ID NO:51 and a light chain variable region comprising SEQ ID NO:52.

In one embodiment, an anti-activin domain comprises a heavy chain variable region comprising SEQ ID NO:53 and a light chain variable region comprising SEQ ID NO:54.

In one embodiment, an anti-activin domain comprises a heavy chain variable region comprising SEQ ID NO:55 and a light chain variable region comprising SEQ ID NO:56.

In one embodiment, an anti-activin domain comprises a heavy chain variable region comprising SEQ ID NO:57 and a light chain variable region comprising SEQ ID NO:58.

In one embodiment, an anti-activin domain comprises a heavy chain variable region comprising SEQ ID NO:59 and a light chain variable region comprising SEQ ID NO:60.

In one embodiment, an anti-activin domain comprises a heavy chain variable region comprising SEQ ID NO:61 and a light chain variable region comprising SEQ ID NO:62.

In one embodiment, an anti-activin domain comprises a heavy chain variable region comprising SEQ ID NO:63 and a light chain variable region comprising SEQ ID NO:64.

In one embodiment, an anti-activin domain comprises a heavy chain variable region comprising SEQ ID NO:65 and a light chain variable region comprising SEQ ID NO:66.

In one embodiment, an anti-activin domain comprises a heavy chain variable region comprising SEQ ID NO:67 and a light chain variable region comprising SEQ ID NO:68.

In one embodiment, an anti-activin domain comprises a heavy chain variable region comprising SEQ ID NO:69 and a light chain variable region comprising SEQ ID NO:70.

In one embodiment, the anti-activin domain comprises a light chain variable region comprising any one of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, and 70.

In one embodiment, the anti-activin domain comprises a light chain variable region comprising any one of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, and 70; and a heavy chain variable region comprising any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, and 69.

In one embodiment, the anti-activin domain comprises a heavy chain variable region comprising an amino acid sequence selected from the group consisting of: SEQ ID NOs: 39, 41, 43, 45, 47, 49, 63, 65, and 69.

In one embodiment, the anti-activin domain comprises a light chain variable region comprising an amino acid sequence selected from the group consisting of SEQ ID NOs: 40, 42, 44, 46, 48, 50, 64, 66, and 70.

In one embodiment, the anti-activin domain comprises a heavy chain variable region comprising an amino acid sequence selected from the group consisting of: SEQ ID

NOs: 39, 41, 43, 45, 47, 49, 63, 65, and 69, and further comprises a light chain variable region comprising an amino acid sequence selected from the group consisting of SEQ ID NOs: 40, 42, 44, 46, 48, 50, 64, 66, and 70.

In one embodiment, the anti-activin domain comprises a heavy chain variable region comprising a CDR1 sequence selected from the group consisting of SEQ ID NOs: 229-234, 241-242, and 244; a CDR2 sequence selected from the group consisting of SEQ ID NOS: 264-269, 276-277, and 279; and a CDR3 sequence selected from the group consisting of SEQ ID NOs: 299-304, 311-312, and 314.

In one embodiment, the anti-activin domain comprises a light chain variable region comprising a CDR1 sequence selected from the group consisting of SEQ ID NOs: 334-339, 346-347, and 349; a CDR2 sequence selected from the group consisting of SEQ ID NOs: 369-374, 381-382, and 384; and a CDR3 sequence selected from the group consisting of SEQ ID NOs: 404-409; 416-417; and 419.

In one embodiment, the anti-activin domain comprises a heavy chain variable region comprising a CDR1 sequence selected from the group consisting of SEQ ID NOs: 439-444, 451-452, and 454; a CDR2 sequence selected from the group consisting of SEQ ID NOs: 474-479, 486-487, and 489; and a CDR3 sequence selected from the group consisting of SEQ ID NOs: 509-514, 521-522, and 524.

In one embodiment, the anti-activin domain comprises a light chain variable region comprising a CDR1 sequence selected from the group consisting of SEQ ID NOs: 544-549, 556-557, and 559; a CDR2 sequence selected from the group consisting of SEQ ID NOs: 80-85, 92-93, and 95; and a CDR3 sequence selected from the group consisting of SEQ ID NOs: 185-190, 197-198, and 209.

In one embodiment the invention relates to administering a genetically modified T cell expressing a CAR for the treatment of a patient having cancer or at risk of having cancer using lymphocyte infusion. Preferably, autologous lymphocyte infusion is used in the treatment. Autologous PBMCs are collected from a patient in need of treatment and T cells are activated and expanded using the methods described herein and known in the art and then infused back into the patient.

The invention also includes treating a malignancy or an autoimmune disease in which chemotherapy and/or immunotherapy in a patient results in significant immunosuppression in the patient, thereby increasing the risk of the patient of developing a malignancy (e.g., CLL).

The invention includes using T cells expressing an anti-activin antibody derived CAR including both CD3-zeta and either the 4-1BB or CD28 costimulatory domain (also referred to as CARTPODO T cells). The CARTPODO T cells of the invention can undergo robust in vivo T cell expansion and can establish memory cells specific for cells displaying activin tumor epitope, which memory cells persist at high levels for an extended amount of time in blood and bone marrow.

The present invention provides chimeric antigen receptor (CAR) comprising an extracellular and intracellular domain. The extracellular domain comprises a target-specific binding element otherwise referred to as an antigen binding moiety. The intracellular domain or otherwise the cytoplasmic domain comprises, a costimulatory signaling region and a zeta chain portion. The costimulatory signaling region refers to a portion of the CAR comprising the intracellular domain of a costimulatory molecule. Costimulatory molecules are cell surface molecules other than antigens receptors or their ligands that are required for an efficient response of lymphocytes to antigen.

Between the extracellular domain and the transmembrane domain of the CAR, or between the cytoplasmic domain and the transmembrane domain of the CAR, there may be incorporated a spacer domain. As used herein, the term “spacer domain” generally means any oligo- or polypeptide that functions to link the transmembrane domain to, either the extracellular domain or, the cytoplasmic domain in the polypeptide chain. A spacer domain may comprise up to 300 amino acids, 10 to 100 amino acids and often 25 to 50 amino acids.

### Antigen Binding Moiety

In one embodiment, the CAR of the invention comprises a target-specific binding element otherwise referred to as an antigen binding moiety, or targeting arm. Antigen binding moieties used in the present invention are capable of binding the activin tumor epitope. As such, the antigen binding moiety is chosen to recognize a ligand that acts as a cell surface marker on target cells associated with a particular disease state.

A CAR of the invention is engineered to target a cell displaying the activin tumor epitope by way of engineering an appropriate antigen binding moiety that specifically binds to the activin tumor epitope.

Preferably, the antigen binding moiety portion in the CAR of the invention is scFv, or scFab wherein the nucleic acid sequence of the scFv comprises the nucleic acid sequence(s) of one or more light chain CDRs and one or more heavy chain CDRs disclosed herein for anti-activin antibodies, and wherein the nucleic acid sequence of the scFab comprises the nucleic acid sequence(s) of one or more light chain CDRs and one or more heavy chain CDRs disclosed herein for anti-activin antibodies.

Preferably, the antigen binding moiety portion in the CAR of the invention is an scFv, or scFab comprising an amino acid sequence selected from the group consisting of any one of SEQ ID NOs: 1-70.

Preferably, the antigen binding moiety portion in the CAR of the invention is an scFv, or scFab comprising an amino acid sequence selected from the group consisting of any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, and 69, more preferably an scFv or scFab comprising an amino acid sequence selected from the group consisting of SEQ ID NOs: 39, 41, 43, 45, 47, 49, 63, 65, and 69.

Preferably, the antigen binding moiety portion in the CAR of the invention is an scFv, or scFab comprising an amino acid sequence selected from the group consisting of any one of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, and 70, more preferably an scFv or scFab comprising an amino acid sequence selected from the group consisting of SEQ ID NOs: 40, 42, 44, 46, 48, 50, 64, 66, and 70.

Preferably, the antigen binding moiety portion in the CAR of the invention is an scFv, or scFab comprising an amino acid sequence selected from the group consisting of any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, and 69 and any one of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, and 70. More preferably, the antigen binding moiety portion in the CAR of the invention is an scFv, or scFab comprising an amino acid sequence selected from the group consisting of any one of SEQ ID NOs: 39, 41, 43, 45, 47, 49, 63, 65, and 69, and any one of SEQ ID NOs: 40, 42, 44, 46, 48, 50, 64, 66, and 70.

The antigen binding moiety portion in the CAR of the invention is an scFv, or scFab comprising an amino acid sequence selected from the group consisting of any CDR sequence in Table I and Table II, or in Table III and Table IV.

Preferably, the antigen binding moiety portion in the CAR of the invention is an scFv, or scFab comprising an amino acid sequence selected from the group consisting of any CDR sequence in Table I and Table II, or Table III and IV; and further comprises an amino acid sequence selected from the group consisting of any one of SEQ ID NOs: 1-70. More preferably, the antigen binding moiety portion of the CAR of the invention is an scFv, or scFab comprising a heavy chain variable region comprising a CDR1 sequence selected from the group consisting of SEQ ID NOs: 229-234, 241-242, and 244; a CDR2 sequence selected from the group consisting of SEQ ID NOs: 264-269, 276-277, and 279; and a CDR3 sequence selected from the group consisting of SEQ ID NOs: 299-304, 311-312, and 314, and a light chain variable region comprising a CDR1 sequence selected from the group consisting of SEQ ID NOs: 334-339, 346-347, and 349; a CDR2 sequence selected from the group consisting of SEQ ID NOs: 369-374, 381-382, and 384; and a CDR3 sequence selected from the group consisting of SEQ ID NOs: 404-409; 416-417; and 419; or comprising a heavy chain variable region comprising a CDR1 sequence selected from the group consisting of SEQ ID NOs: 439-444, 451-452, and 454; a CDR2 sequence selected from the group consisting of SEQ ID NOs: 474-479, 486-487, and 489; and a CDR3 sequence selected from the group consisting of SEQ ID NOs: 509-514, 521-522, and 524, and a light chain variable region comprising a CDR1 sequence selected from the group consisting of SEQ ID NOs: 544-549, 556-557, and 559; a CDR2 sequence selected from the group consisting of SEQ ID NOs: 80-85, 92-93, and 95; and a CDR3 sequence selected from the group consisting of SEQ ID NOs: 185-190, 197-198, and 209; and further comprises an amino acid sequence selected from the group consisting of any one of SEQ ID NOs: 39-50, 63-66, and 69-70.

In one embodiment, the antigen binding moiety portion in the CAR of the invention is an scFv, or scFab comprising an amino acid sequence having about 80%, 85%, 90%, or 95% identity to the SEQ ID NOs recited above.

### Transmembrane Domain

With respect to the transmembrane domain, the CAR can be designed to comprise a transmembrane domain that is fused to the extracellular domain of the CAR. In one embodiment, the transmembrane domain that naturally is associated with one of the domains in the CAR is used. In some instances, the transmembrane domain can be selected or modified by amino acid substitution to avoid binding of such domains to the transmembrane domains of the same or different surface membrane proteins to minimize interactions with other members of the receptor complex.

The transmembrane domain may be derived either from a natural or from a synthetic source. Where the source is natural, the domain may be derived from any membrane-bound or transmembrane protein. Transmembrane regions of particular use in this invention may be derived from (i.e. comprise at least the transmembrane region(s) of) the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154. Alternatively, the transmembrane domain may be synthetic, in which case it will comprise predominantly hydrophobic residues such as leucine and valine. Preferably a triplet of phenylalanine, tryptophan and valine will be found at each end of a synthetic transmembrane domain. Optionally, a short oligo- or polypeptide linker, preferably between 2 and 10 amino acids in length may form the linkage between the transmembrane domain and the cytoplasmic signaling domain of the CAR. A glycine-serine doublet provides a particularly suitable linker.

Preferably, the transmembrane domain in the CAR of the invention is the CD8 transmembrane domain. In one embodiment, the CD8 transmembrane domain comprises the nucleic acid sequence of SEQ ID NO: 16 of U.S. Pat. No. 9,102,760. In one embodiment, the CD8 transmembrane domain comprises the nucleic acid sequence that encodes the amino acid sequence of SEQ ID NO: 22 of U.S. Pat. No. 9,102,760. In another embodiment, the CD8 transmembrane domain comprises the amino acid sequence of SEQ ID NO: 22 of U.S. Pat. No. 9,102,760.

In some instances, the transmembrane domain of the CAR of the invention comprises the CD8α hinge domain. In one embodiment, the CD8 hinge domain comprises the nucleic acid sequence of SEQ ID NO: 15 of U.S. Pat. No. 9,102,760. In one embodiment, the CD8 hinge domain comprises the nucleic acid sequence that encodes the amino acid sequence of SEQ ID NO: 21 of U.S. Pat. No. 9,102,760. In another embodiment, the CD8 hinge domain comprises the amino acid sequence of SEQ ID NO: 21 of U.S. Pat. No. 9,102,760.

### Cytoplasmic Domain

The cytoplasmic domain or otherwise the intracellular signaling domain of the CAR of the invention is responsible for activation of at least one of the normal effector functions of the immune cell in which the CAR has been placed in. The term “effector function” refers to a specialized function of a cell. Effector function of a T cell, for example, may be cytolytic activity or helper activity including the secretion of cytokines. Thus, the term “intracellular signaling domain” refers to the portion of a protein which transduces the effector function signal and directs the cell to perform a specialized function. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

Preferred examples of intracellular signaling domains for use in the CAR of the invention include the cytoplasmic sequences of the T cell receptor (TCR) and co-receptors that act in concert to initiate signal transduction following antigen receptor engagement, as well as any derivative or variant of these sequences and any synthetic sequence that has the same functional capability.

It is known that signals generated through the TCR alone are insufficient for full activation of the T cell and that a secondary or co-stimulatory signal is also required. Thus, T cell activation can be said to be mediated by two distinct classes of cytoplasmic signaling sequence: those that initiate antigen-dependent primary activation through the TCR (primary cytoplasmic signaling sequences) and those that act in an antigen-independent manner to provide a secondary or co-stimulatory signal (secondary cytoplasmic signaling sequences).

Primary cytoplasmic signaling sequences regulate primary activation of the TCR complex either in a stimulatory way, or in an inhibitory way. Primary cytoplasmic signaling sequences that act in a stimulatory manner may contain signaling motifs which are known as immunoreceptor tyrosine-based activation motifs or ITAMs.

Examples of ITAM containing primary cytoplasmic signaling sequences that are of particular use in the invention include those derived from TCR zeta, FcR gamma, FcR beta, CD3 gamma, CD3 delta, CD3 epsilon, CD5, CD22, CD79a, CD79b, and CD66d. It is particularly preferred that cytoplasmic signaling molecule in the CAR of the invention comprises a cytoplasmic signaling sequence derived from CD3 zeta.

In some embodiments, the cytoplasmic domain of the CAR can be designed to comprise the CD3-zeta signaling domain by itself or combined with any other desired cytoplasmic domain(s) useful in the context of the CAR of the invention. For example, the cytoplasmic domain of the CAR can comprise a CD3 zeta chain portion and a costimulatory signaling region. The costimulatory signaling region refers to a portion of the CAR comprising the intracellular domain of a costimulatory molecule. A costimulatory molecule is a cell surface molecule other than an antigen receptor or their ligands that is required for an efficient response of lymphocytes to an antigen. Examples of such molecules include CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, and a ligand that specifically binds with CD83, and the like.

The cytoplasmic signaling sequences within the cytoplasmic signaling portion of the CAR of the invention may be linked to each other in a random or specified order. Optionally, a short oligo- or polypeptide linker, preferably between 2 and 10 amino acids in length may form the linkage. A glycine-serine doublet provides a particularly suitable linker.

In one embodiment, the cytoplasmic domain is designed to comprise the signaling domain of CD3-zeta and the signaling domain of CD28. In another embodiment, the cytoplasmic domain is designed to comprise the signaling domain of CD3-zeta and the signaling domain of 4-1BB. In yet another embodiment, the cytoplasmic domain is designed to comprise the signaling domain of CD3-zeta and the signaling domain of CD28 and 4-1BB.

In one embodiment, the cytoplasmic domain in the CAR of the invention is designed to comprise the signaling domain of 4-1BB and the signaling domain of CD3-zeta, wherein the signaling domain of 4-1BB comprises the nucleic acid sequence set forth in SEQ ID NO: 17 of U.S. Pat. No. 9,102,760 and the signaling domain of CD3-zeta comprises the nucleic acid sequence set forth in SEQ ID NO: 18 of U.S. Pat. No. 9,102,760.

In one embodiment, the cytoplasmic domain in the CAR of the invention is designed to comprise the signaling domain of 4-1BB and the signaling domain of CD3-zeta, wherein the signaling domain of 4-1BB comprises the nucleic acid sequence that encodes the amino acid sequence of SEQ ID NO: 23 of U.S. Pat. No. 9,102,760 and the signaling domain of CD3-zeta comprises the nucleic acid sequence that encodes the amino acid sequence of SEQ ID NO: 24 of U.S. Pat. No. 9,102,760.

In one embodiment, the cytoplasmic domain in the CAR of the invention is designed to comprise the signaling domain of 4-1BB and the signaling domain of CD3-zeta, wherein the signaling domain of 4-1BB comprises the amino acid sequence set forth in SEQ ID NO: 23 of U.S. Pat. No. 9,102,760 and the signaling domain of CD3-zeta comprises the amino acid sequence set forth in SEQ ID NO: 24 of U.S. Pat. No. 9,102,760.

### Vectors

The present invention encompasses a DNA construct comprising sequences of a CAR, wherein the sequence comprises the nucleic acid sequence of an antigen binding moiety operably linked to the nucleic acid sequence of an intracellular domain. An exemplary intracellular domain that can be used in the CAR of the invention includes but is not limited to the intracellular domain of CD3-zeta, CD28, 4-1BB, and the like. In some instances, the CAR can comprise any combination of CD3-zeta, CD28, 4-1BB, and the like.

In one embodiment, the CAR of the invention comprises an anti-activin antibody derived scFv, human CD8 hinge and transmembrane domain, and human 4-1BB and CD3zeta signaling domains.

The nucleic acid sequences coding for the desired molecules can be obtained using recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically, rather than cloned.

The present invention also provides vectors in which a DNA of the present invention is inserted. Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity.

In brief summary, the expression of natural or synthetic nucleic acids encoding CARs is typically achieved by operably linking a nucleic acid encoding the CAR polypeptide or portions thereof to a promoter and incorporating the construct into an expression vector. The vectors can be suitable for replication and integration eukaryotes. Typical cloning vectors contain transcription and translation terminators, initiation sequences, and promoters useful for regulation of the expression of the desired nucleic acid sequence.

In addition to the methods described above, the following methods may be used.

The expression constructs of the present invention may also be used for nucleic acid immunization and gene therapy, using standard gene delivery protocols. Methods for gene delivery are known in the art (e.g., U.S. Pat. Nos. 5,399,346, 5,580,859, 5,589,466, incorporated by reference herein in their entireties). In another embodiment, the invention provides a gene therapy vector.

The nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, for example, in Sambrook et al. (2001, Molecular Cloning: A Laboratory Manual, Cold Spring Harbor Laboratory, New York), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In one embodiment, lentivirus vectors are used.

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1α (EF-1α). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired or turning off the expression when expression is not desired. Examples of inducible promoters include, but are not limited to a metallothionine promoter, a glucocorticoid promoter, a progesterone promoter, and a tetracycline promoter.

In order to assess the expression of a CAR polypeptide or portions thereof, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479:79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001, Molecular Cloning: A Laboratory Manual, Cold Spring Harbor Laboratory, New York). One method for the introduction of a polynucleotide into a host cell is calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc. (Birmingham, AL). Stock solutions of lipids in chloroform or chloroform/methanol can be stored at about −20.degree. C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5:505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce exogenous nucleic acids into a host cell or otherwise expose a cell to the inhibitor of the present invention, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

### Sources of T Cells

Prior to expansion and genetic modification of the T cells of the invention, a source of T cells is obtained from a subject. T cells can be obtained from a number of sources, including peripheral blood mononuclear cells, bone marrow, lymph node tissue, cord blood, thymus tissue, tissue from a site of infection, ascites, pleural effusion, spleen tissue, and tumors. In certain embodiments of the present invention, any number of T cell lines available in the art, may be used. In certain embodiments of the present invention, T cells can be obtained from a unit of blood collected from a subject using any number of techniques known to the skilled artisan, such as Ficoll™ separation. In one preferred embodiment, cells from the circulating blood of an individual are obtained by apheresis. The apheresis product typically contains lymphocytes, including T cells, monocytes, granulocytes, B cells, other nucleated white blood cells, red blood cells, and platelets. In one embodiment, the cells collected by apheresis may be washed to remove the plasma fraction and to place the cells in an appropriate buffer or media for subsequent processing steps. In one embodiment of the invention, the cells are washed with phosphate buffered saline (PBS). In an alternative embodiment, the wash solution lacks calcium and may lack magnesium or may lack many if not all divalent cations. Again, surprisingly, initial activation steps in the absence of calcium lead to magnified activation. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as, for example, Ca²⁺-free, Mg²⁺-free PBS, PlasmaLyte A, or other saline solution with or without buffer. Alternatively, the undesirable components of the apheresis sample may be removed and the cells directly resuspended in culture media.

In another embodiment, T cells are isolated from peripheral blood lymphocytes by lysing the red blood cells and depleting the monocytes, for example, by centrifugation through a PERCOLL™ gradient or by counterflow centrifugal elutriation. A specific subpopulation of T cells, such as CD3⁺, CD28⁺, CD4⁺, CD8⁺, CD45RA⁺, and CD45RO⁺ T cells, can be further isolated by positive or negative selection techniques. For example, in one embodiment, T cells are isolated by incubation with anti-CD3/anti-CD28 (i.e., 3×28)-conjugated beads, such as DYNABEADS® M-450 CD3/CD28 T, for a time period sufficient for positive selection of the desired T cells. In one embodiment, the time period is about 30 minutes. In a further embodiment, the time period ranges from 30 minutes to 36 hours or longer and all integer values there between. In a further embodiment, the time period is at least 1, 2, 3, 4, 5, or 6 hours. In yet another preferred embodiment, the time period is 10 to 24 hours. In one preferred embodiment, the incubation time period is 24 hours. For isolation of T cells from patients with leukemia, use of longer incubation times, such as 24 hours, can increase cell yield. Longer incubation times may be used to isolate T cells in any situation where there are few T cells as compared to other cell types, such in isolating tumor infiltrating lymphocytes (TIL) from tumor tissue or from immune-compromised individuals. Further, use of longer incubation times can increase the efficiency of capture of CD8+ T cells. Thus, by simply shortening or lengthening the time T cells are allowed to bind to the CD3/CD28 beads and/or by increasing or decreasing the ratio of beads to T cells (as described further herein), subpopulations of T cells can be preferentially selected for or against at culture initiation or at other time points during the process. Additionally, by increasing or decreasing the ratio of anti-CD3 and/or anti-CD28 antibodies on the beads or other surface, subpopulations of T cells can be preferentially selected for or against at culture initiation or at other desired time points. The skilled artisan would recognize that multiple rounds of selection can also be used in the context of this invention. In certain embodiments, it may be desirable to perform the selection procedure and use the “unselected” cells in the activation and expansion process. “Unselected” cells can also be subjected to further rounds of selection.

Enrichment of a T cell population by negative selection can be accomplished with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD4⁺ cells by negative selection, a monoclonal antibody cocktail typically includes antibodies to CD14, CD20, CD11b, CD16, HLA-DR, and CD8. In certain embodiments, it may be desirable to enrich for or positively select for regulatory T cells which typically express CD4⁺, CD25⁺, CD62L^{hi}, GITR⁺, and FoxP3⁺. Alternatively, in certain embodiments, T regulatory cells are depleted by anti-C25 conjugated beads or other similar method of selection.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/mL is used. In one embodiment, a concentration of 1 billion cells/mL is used. In a further embodiment, greater than 100 million cells/mL is used. In a further embodiment, a concentration of cells of 10, 15, 20, 25, 30, 35, 40, 45, or 50 million cells/mL is used. In yet another embodiment, a concentration of cells from 75, 80, 85, 90, 95, or 100 million cells/mL is used. In further embodiments, concentrations of 125 or 150 million cells/mL can be used. Using high concentrations can result in increased cell yield, cell activation, and cell expansion. Further, use of high cell concentrations allows more efficient capture of cells that may weakly express target antigens of interest, such as CD28-negative T cells, or from samples where there are many tumor cells present (i.e., leukemic blood, tumor tissue, etc.). Such populations of cells may have therapeutic value and would be desirable to obtain. For example, using high concentration of cells allows more efficient selection of CD8⁺ T cells that normally have weaker CD28 expression.

In a related embodiment, it may be desirable to use lower concentrations of cells. By significantly diluting the mixture of T cells and surface (e.g., particles such as beads), interactions between the particles and cells is minimized. This selects for cells that express high amounts of desired antigens to be bound to the particles. For example, CD4⁺ T cells express higher levels of CD28 and are more efficiently captured than CD8⁺ T cells in dilute concentrations. In one embodiment, the concentration of cells used is 5×10⁶/mL. In other embodiments, the concentration used can be from about 1×10⁵/mL to 1×10⁶/mL, and any integer value in between.

In other embodiments, the cells may be incubated on a rotator for varying lengths of time at varying speeds at either 2-10° C. or at room temperature.

T cells for stimulation can also be frozen after a washing step. Wishing not to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, one method involves using PBS containing 20% DMSO and 8% human serum albumin, or culture media containing 10% Dextran 40 and 5% Dextrose, 20% Human Serum Albumin and 7.5% DMSO, or 31.25% Plasmalyte-A, 31.25% Dextrose 5%, 0.45% NaCl, 10% Dextran 40 and 5% Dextrose, 20% Human Serum Albumin, and 7.5% DMSO or other suitable cell freezing media containing for example, Hespan and PlasmaLyte A, the cells then are frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

In certain embodiments, cryopreserved cells are thawed and washed as described herein and allowed to rest for one hour at room temperature prior to activation using the methods of the present invention.

Also contemplated in the context of the invention is the collection of blood samples or apheresis product from a subject at a time period prior to when the expanded cells as described herein might be needed. As such, the source of the cells to be expanded can be collected at any time point necessary, and desired cells, such as T cells, isolated and frozen for later use in T cell therapy for any number of diseases or conditions that would benefit from T cell therapy, such as those described herein. In one embodiment a blood sample or an apheresis is taken from a generally healthy subject. In certain embodiments, a blood sample or an apheresis is taken from a generally healthy subject who is at risk of developing a disease, but who has not yet developed a disease, and the cells of interest are isolated and frozen for later use. In certain embodiments, the T cells may be expanded, frozen, and used at a later time. In certain embodiments, samples are collected from a patient shortly after diagnosis of a particular disease as described herein but prior to any treatments. In a further embodiment, the cells are isolated from a blood sample or an apheresis from a subject prior to any number of relevant treatment modalities, including but not limited to treatment with agents such as natalizumab, efalizumab, antiviral agents, chemotherapy, radiation, immunosuppressive agents, such as cyclosporin, azathioprine, methotrexate, mycophenolate, and FK506, antibodies, or other immunoablative agents such as CAMPATH, anti-CD3 antibodies, cytoxan, fludarabine, cyclosporin, FK506, rapamycin, mycophenolic acid, steroids, FR901228, and irradiation. These drugs inhibit either the calcium dependent phosphatase calcineurin (cyclosporine and FK506) or inhibit the p70S6 kinase that is important for growth factor induced signaling (rapamycin) (Liu et al., Cell 66:807-15, 1991; Henderson et al., Immun 73:316-21, 1991; Bierer et al., Curr. Opin. Immun 5:763-73, 1993). In a further embodiment, the cells are isolated for a patient and frozen for later use in conjunction with (e.g., before, simultaneously or following) bone marrow or stem cell transplantation, T cell ablative therapy using either chemotherapy agents such as, fludarabine, external-beam radiation therapy (XRT), cyclophosphamide, or antibodies such as OKT3 or CAMPATH. In another embodiment, the cells are isolated prior to and can be frozen for later use for treatment following B-cell ablative therapy such as agents that react with CD20, e.g., Rituxan.

In a further embodiment of the present invention, T cells are obtained from a patient directly following treatment. In this regard, it has been observed that following certain cancer treatments, in particular treatments with drugs that damage the immune system, shortly after treatment during the period when patients would normally be recovering from the treatment, the quality of T cells obtained may be optimal or improved for their ability to expand ex vivo. Likewise, following ex vivo manipulation using the methods described herein, these cells may be in a preferred state for enhanced engraftment and in vivo expansion. Thus, it is contemplated within the context of the present invention to collect blood cells, including T cells, dendritic cells, or other cells of the hematopoietic lineage, during this recovery phase. Further, in certain embodiments, mobilization (for example, mobilization with GM-CSF) and conditioning regimens can be used to create a condition in a subject wherein repopulation, recirculation, regeneration, and/or expansion of particular cell types is favored, especially during a defined window of time following therapy. Illustrative cell types include T cells, B cells, dendritic cells, and other cells of the immune system.

### Activation and Expansion of T Cells

Whether prior to or after genetic modification of the T cells to express a desirable CAR, the T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005.

Generally, the T cells of the invention are expanded by contact with a surface having attached thereto an agent that stimulates a CD3/TCR complex associated signal and a ligand that stimulates a co-stimulatory molecule on the surface of the T cells. In particular, T cell populations may be stimulated as described herein, such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For costimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. To stimulate proliferation of either CD4⁺ T cells or CD8⁺ T cells, an anti-CD3 antibody and an anti-CD28 antibody. Examples of an anti-CD28 antibody include 9.3, B-T3, XR-CD28 (Diaclone, Besancon, France) can be used as can other methods commonly known in the art (Berg et al., Transplant Proc. 30 (8): 3975-7, 1998; Haanen et al., J. Exp. Med. 190 (9): 13191328, 1999; Garland et al., J. Immunol Meth. 227 (1-2): 53-63, (1999)).

In certain embodiments, the primary stimulatory signal and the co-stimulatory signal for the T cell may be provided by different protocols. For example, the agents providing each signal may be in solution or coupled to a surface. When coupled to a surface, the agents may be coupled to the same surface (i.e., in “cis” formation) or to separate surfaces (i.e., in “trans” formation). Alternatively, one agent may be coupled to a surface and the other agent in solution. In one embodiment, the agent providing the co-stimulatory signal is bound to a cell surface and the agent providing the primary activation signal is in solution or coupled to a surface. In certain embodiments, both agents can be in solution. In another embodiment, the agents may be in soluble form, and then cross-linked to a surface, such as a cell expressing Fc receptors or an antibody or other binding agent which will bind to the agents. In this regard, see for example, U.S. Patent Application Publication Nos. 20040101519 and 20060034810 for artificial antigen presenting cells (aAPCs) that are contemplated for use in activating and expanding T cells in the present invention.

In one embodiment, the two agents are immobilized on beads, either on the same bead, i.e., “cis,” or to separate beads, i.e., “trans.” By way of example, the agent providing the primary activation signal is an anti-CD3 antibody or an antigen-binding fragment thereof and the agent providing the co-stimulatory signal is an anti-CD28 antibody or antigen-binding fragment thereof; and both agents are co-immobilized to the same bead in equivalent molecular amounts. In one embodiment, a 1:1 ratio of each antibody bound to the beads for CD4⁺ T cell expansion and T cell growth is used. In certain aspects of the present invention, a ratio of anti CD3:CD28 antibodies bound to the beads is used such that an increase in T cell expansion is observed as compared to the expansion observed using a ratio of 1:1. In one particular embodiment an increase of from about 1 to about 3 fold is observed as compared to the expansion observed using a ratio of 1:1. In one embodiment, the ratio of CD3:CD28 antibody bound to the beads ranges from 100:1 to 1:100 and all integer values there between. In one aspect of the present invention, more anti-CD28 antibody is bound to the particles than anti-CD3 antibody, i.e., the ratio of CD3:CD28 is less than one. In certain embodiments of the invention, the ratio of anti CD28 antibody to anti CD3 antibody bound to the beads is greater than 2:1. In one particular embodiment, a 1:100 CD3:CD28 ratio of antibody bound to beads is used. In another embodiment, a 1:75 CD3:CD28 ratio of antibody bound to beads is used. In a further embodiment, a 1:50 CD3:CD28 ratio of antibody bound to beads is used. In another embodiment, a 1:30 CD3:CD28 ratio of antibody bound to beads is used. In one preferred embodiment, a 1:10 CD3:CD28 ratio of antibody bound to beads is used. In another embodiment, a 1:3 CD3:CD28 ratio of antibody bound to the beads is used. In yet another embodiment, a 3:1 CD3:CD28 ratio of antibody bound to the beads is used.

Ratios of particles to cells from 1:500 to 500:1 and any integer values in between may be used to stimulate T cells or other target cells. As those of ordinary skill in the art can readily appreciate, the ratio of particles to cells may depend on particle size relative to the target cell. For example, small sized beads could only bind a few cells, while larger beads could bind many. In certain embodiments the ratio of cells to particles ranges from 1:100 to 100:1 and any integer values in-between and in further embodiments the ratio comprises 1:9 to 9:1 and any integer values in between, can also be used to stimulate T cells. The ratio of anti-CD3- and anti-CD28-coupled particles to T cells that result in T cell stimulation can vary as noted above, however certain preferred values include 1:100, 1:50, 1:40, 1:30, 1:20, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, and 15:1 with one preferred ratio being at least 1:1 particles per T cell. In one embodiment, a ratio of particles to cells of 1:1 or less is used. In one particular embodiment, a preferred particle:cell ratio is 1:5. In further embodiments, the ratio of particles to cells can be varied depending on the day of stimulation. For example, in one embodiment, the ratio of particles to cells is from 1:1 to 10:1 on the first day and additional particles are added to the cells every day or every other day thereafter for up to 10 days, at final ratios of from 1:1 to 1:10 (based on cell counts on the day of addition). In one particular embodiment, the ratio of particles to cells is 1:1 on the first day of stimulation and adjusted to 1:5 on the third and fifth days of stimulation. In another embodiment, particles are added on a daily or every other day basis to a final ratio of 1:1 on the first day, and 1:5 on the third and fifth days of stimulation. In another embodiment, the ratio of particles to cells is 2:1 on the first day of stimulation and adjusted to 1:10 on the third and fifth days of stimulation. In another embodiment, particles are added on a daily or every other day basis to a final ratio of 1:1 on the first day, and 1:10 on the third and fifth days of stimulation. One of skill in the art will appreciate that a variety of other ratios may be suitable for use in the present invention. In particular, ratios will vary depending on particle size and on cell size and type.

In further embodiments of the present invention, the cells, such as T cells, are combined with agent-coated beads, the beads and the cells are subsequently separated, and then the cells are cultured. In an alternative embodiment, prior to culture, the agent-coated beads and cells are not separated but are cultured together. In a further embodiment, the beads and cells are first concentrated by application of a force, such as a magnetic force, resulting in increased ligation of cell surface markers, thereby inducing cell stimulation.

By way of example, cell surface proteins may be ligated by allowing paramagnetic beads to which anti-CD3 and anti-CD28 are attached (3×28 beads) to contact the T cells. In one embodiment the cells (for example, 10⁴ to 10⁹ T cells) and beads (for example, DYNABEADS® M-450 CD3/CD28 T paramagnetic beads at a ratio of 1:1) are combined in a buffer, preferably PBS (without divalent cations such as, calcium and magnesium). Again, those of ordinary skill in the art can readily appreciate any cell concentration may be used.

For example, the target cell may be very rare in the sample and comprise only 0.01% of the sample or the entire sample (i.e., 100%) may comprise the target cell of interest. Accordingly, any cell number is within the context of the present invention. In certain embodiments, it may be desirable to significantly decrease the volume in which particles and cells are mixed together (i.e., increase the concentration of cells), to ensure maximum contact of cells and particles. For example, in one embodiment, a concentration of about 2 billion cells/mL is used. In another embodiment, greater than 100 million cells/mL is used. In a further embodiment, a concentration of cells of 10, 15, 20, 25, 30, 35, 40, 45, or 50 million cells/mL is used. In yet another embodiment, a concentration of cells from 75, 80, 85, 90, 95, or 100 million cells/mL is used. In further embodiments, concentrations of 125 or 150 million cells/mL can be used. Using high concentrations can result in increased cell yield, cell activation, and cell expansion. Further, use of high cell concentrations allows more efficient capture of cells that may weakly express target antigens of interest, such as CD28-negative T cells. Such populations of cells may have therapeutic value and would be desirable to obtain in certain embodiments. For example, using high concentration of cells allows more efficient selection of CD8⁺ T cells that normally have weaker CD28 expression.

In one embodiment of the present invention, the mixture may be cultured for several hours (about 3 hours) to about 14 days or any hourly integer value in between. In another embodiment, the mixture may be cultured for 21 days. In one embodiment of the invention the beads and the T cells are cultured together for about eight days. In another embodiment, the beads and T cells are cultured together for 2-3 days. Several cycles of stimulation may also be desired such that culture time of T cells can be 60 days or more. Conditions appropriate for T cell culture include an appropriate media (e.g., Minimal Essential Media or RPMI Media 1640 or, X-vivo 15, (Lonza)) that may contain factors necessary for proliferation and viability, including serum (e.g., fetal bovine or human serum), interleukin-2 (IL-2), insulin, IFN-γ, IL-4, IL-7, GM-CSF, IL-10, IL-12, IL-15, TGFβ, and TNF-α or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, AIM-V, DMEM, MEM, α-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO₂).

T cells that have been exposed to varied stimulation times may exhibit different characteristics. For example, typical blood or apheresed peripheral blood mononuclear cell products have a helper T cell population (T_{H}, CD4⁺) that is greater than the cytotoxic or suppressor T cell population (T_{C}, CD8⁺). Ex vivo expansion of T cells by stimulating CD3 and CD28 receptors produces a population of T cells that prior to about days 8-9 consists predominately of T_{H} cells, while after about days 8-9, the population of T cells comprises an increasingly greater population of T_{C} cells. Accordingly, depending on the purpose of treatment, infusing a subject with a T cell population comprising predominately of T_{H} cells may be advantageous. Similarly, if an antigen-specific subset of T_{C} cells has been isolated it may be beneficial to expand this subset to a greater degree.

Further, in addition to CD4 and CD8 markers, other phenotypic markers vary significantly, but in large part, reproducibly during the course of the cell expansion process. Thus, such reproducibility enables the ability to tailor an activated T cell product for specific purposes.

### Therapeutic Application

The present invention encompasses a cell (e.g., T cell) transduced with a lentiviral vector (LV). For example, the LV encodes a CAR that combines an antigen recognition domain of a specific antibody with an intracellular domain of CD3-zeta, CD28, 4-1BB, or any combinations thereof. Therefore, in some instances, the transduced T cell can elicit a CAR-mediated T-cell response.

The invention provides the use of a CAR to redirect the specificity of a primary T cell to a tumor antigen. Thus, the present invention also provides a method for stimulating a T cell-mediated immune response to a target cell population or tissue in a mammal comprising the step of administering to the mammal a T cell that expresses a CAR, wherein the CAR comprises a binding moiety that specifically interacts with a predetermined target, a zeta chain portion comprising for example the intracellular domain of human CD3zeta, and a costimulatory signaling region.

In one embodiment, the present invention includes a type of cellular therapy where T cells are genetically modified to express a CAR and the CAR T cell is infused to a recipient in need thereof. The infused cell is able to kill tumor cells in the recipient. Unlike antibody therapies, CAR T cells are able to replicate in vivo resulting in long-term persistence that can lead to sustained tumor control.

In one embodiment, the CAR T cells of the invention can undergo robust in vivo T cell expansion and can persist for an extended amount of time. In another embodiment, the CAR T cells of the invention evolve into specific memory T cells that can be reactivated to inhibit any additional tumor formation or growth.

Without wishing to be bound by any particular theory, the anti-tumor immunity response elicited by the CAR-modified T cells may be an active or a passive immune response. In addition, the CAR mediated immune response may be part of an adoptive immunotherapy approach in which CAR-modified T cells induce an immune response specific to the antigen binding moiety in the CAR.

Cancers that may be treated include tumors that are not vascularized, or not yet substantially vascularized, as well as vascularized tumors. The cancers may comprise non-solid tumors (such as hematological tumors, for example, leukemias and lymphomas) or may comprise solid tumors. Types of cancers to be treated with the CARs of the invention include, but are not limited to, carcinoma, blastoma, and sarcoma, and certain leukemia or lymphoid malignancies, benign and malignant tumors, and malignancies e.g., sarcomas, carcinomas, and melanomas. Adult tumors/cancers and pediatric tumors/cancers are also included. In certain embodiments, CAR T cells can be used therapeutically for patients suffering from non-hematological tumors such as solid tumors arising from breast, CNS, and skin malignancies.

Hematologic cancers are cancers of the blood or bone marrow. Examples of hematological (or hematogenous) cancers include leukemias, including acute leukemias (such as acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia.

Solid tumors are abnormal masses of tissue that usually do not contain cysts or liquid areas. Solid tumors can be benign or malignant. Different types of solid tumors are named for the type of cells that form them (such as sarcomas, carcinomas, and lymphomas). Examples of solid tumors, such as sarcomas and carcinomas, include fibrosarcoma, myxosarcoma, liposarcoma, chondrosarcoma, osteosarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma (such as brainstem glioma and mixed gliomas), glioblastoma (also known as glioblastoma multiforme) astrocytoma, CNS lymphoma, germinoma, medulloblastoma, Schwannoma craniopharyogioma, ependymoma, pinealoma, hemangioblastoma, acoustic neuroma, oligodendroglioma, menangioma, neuroblastoma, retinoblastoma and brain metastases).

In one aspect, CAR T cells may be used for ex vivo immunization. With respect to ex vivo immunization, at least one of the following occurs in vitro prior to administering the cell into a mammal: i) expansion of the cells, ii) introducing a nucleic acid encoding a CAR to the cells, and/or iii) cryopreservation of the cells.

Ex vivo procedures are well known in the art and are discussed more fully below. Briefly, cells are isolated from a mammal (e.g., a human) and genetically modified (i.e., transduced or transfected in vitro) with a vector expressing a CAR disclosed herein. The CAR-modified cell can be administered to a mammalian recipient to provide a therapeutic benefit. The mammalian recipient may be a human and the CAR-modified cell can be autologous with respect to the recipient. Alternatively, the cells can be allogeneic, syngeneic or xenogeneic with respect to the recipient.

The procedure for ex vivo expansion of hematopoietic stem and progenitor cells is described in U.S. Pat. No. 5,199,942, incorporated herein by reference, can be applied to the cells of the present invention. Other suitable methods are known in the art, therefore the present invention is not limited to any particular method of ex vivo expansion of the cells. Briefly, ex vivo culture and expansion of T cells comprises: (1) collecting CD34+ hematopoietic stem and progenitor cells from a mammal from peripheral blood harvest or bone marrow explants; and (2) expanding such cells ex vivo. In addition to the cellular growth factors described in U.S. Pat. No. 5,199,942, other factors such as flt3-L, IL-1, IL-3 and c-kit ligand, can be used for culturing and expansion of the cells.

In addition to using a cell-based vaccine in terms of ex vivo immunization, the present invention also provides compositions and methods for in vivo immunization to elicit an immune response directed against an antigen in a patient.

The CAR-modified T cells of the present invention may be administered either alone, or as a pharmaceutical composition in combination with diluents and/or with other components such as IL-2 or other cytokines or cell populations. Briefly, pharmaceutical compositions of the present invention may comprise a target cell population as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present invention are preferably formulated for intravenous administration.

Pharmaceutical compositions of the present invention may be administered in a manner appropriate to the disease to be treated (or prevented). The quantity and frequency of administration will be determined by such factors as the condition of the patient, and the type and severity of the patient's disease, although appropriate dosages may be determined by clinical trials.

When “an immunologically effective amount”, “an anti-tumor effective amount”, “an tumor-inhibiting effective amount”, or “therapeutic amount” is indicated, the precise amount of the compositions of the present invention to be administered can be determined by a physician with consideration of individual differences in age, weight, tumor size, extent of infection or metastasis, and condition of the patient (subject). It can generally be stated that a pharmaceutical composition comprising the T cells described herein may be administered at a dosage of 10⁴ to 10⁹ cells/kg body weight, preferably 10⁵ to 10⁶ cells/kg body weight, including all integer values within those ranges. T cell compositions may also be administered multiple times at these dosages. The cells can be administered by using infusion techniques that are commonly known in immunotherapy (see, e.g., Rosenberg et al., New Eng. J. of Med. 319:1676 (1988)). The optimal dosage and treatment regime for a particular patient can readily be determined by one skilled in the art of medicine by monitoring the patient for signs of disease and adjusting the treatment accordingly.

In certain embodiments, it may be desired to administer activated T cells to a subject and then subsequently redraw blood (or have an apheresis performed), activate T cells therefrom according to the present invention, and reinfuse the patient with these activated and expanded T cells. This process can be carried out multiple times every few weeks. In certain embodiments, T cells can be activated from blood draws of from 10 cc to 400 cc. In certain embodiments, T cells are activated from blood draws of 20 cc, 30 cc, 40 cc, 50 cc, 60 cc, 70 cc, 80 cc, 90 cc, or 100 cc. Not to be bound by theory, using this multiple blood draw/multiple reinfusion protocol may serve to select out certain populations of T cells.

The administration of the subject compositions may be carried out in any convenient manner, including by aerosol inhalation, injection, ingestion, transfusion, implantation or transplantation. The compositions described herein may be administered to a patient subcutaneously, intradermally, intratumorally, intranodally, intramedullary, intramuscularly, by intravenous (i.v.) injection, or intraperitoneally. In one embodiment, the T cell compositions of the present invention are administered to a patient by intradermal or subcutaneous injection. In another embodiment, the T cell compositions of the present invention are preferably administered by i.v. injection. The compositions of T cells may be injected directly into a tumor, lymph node, or site of infection.

In certain embodiments of the present invention, cells activated and expanded using the methods described herein, or other methods known in the art where T cells are expanded to therapeutic levels, are administered to a patient in conjunction with (e.g., before, simultaneously or following) any number of relevant treatment modalities, including but not limited to treatment with agents such as antiviral therapy, cidofovir and interleukin-2, Cytarabine (also known as ARA-C) or natalizumab treatment for MS patients or efalizumab treatment for psoriasis patients or other treatments for PML patients. In further embodiments, the T cells of the invention may be used in combination with chemotherapy, radiation, immunosuppressive agents, such as cyclosporin, azathioprine, methotrexate, mycophenolate, and FK506, antibodies, or other immunoablative agents such as CAMPATH, anti-CD3 antibodies or other antibody therapies, cytoxin, fludaribine, cyclosporin, FK506, rapamycin, mycophenolic acid, steroids, FR901228, cytokines, and irradiation. These drugs inhibit either the calcium dependent phosphatase calcineurin (cyclosporine and FK506) or inhibit the p70S6 kinase that is important for growth factor induced signaling (rapamycin) (Liu et al., Cell 66:807-15, (1991); Henderson et al., Immun 73:316-21, (1991); Bierer et al., Curr. Opin. Immun 5:763-73, (1993)). In a further embodiment, the cell compositions of the present invention are administered to a patient in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH. In another embodiment, the cell compositions of the present invention are administered following B-cell ablative therapy such as agents that react with CD20, e.g., Rituxan. For example, in one embodiment, subjects may undergo standard treatment with high dose chemotherapy followed by peripheral blood stem cell transplantation. In certain embodiments, following the transplant, subjects receive an infusion of the expanded immune cells of the present invention. In an additional embodiment, expanded cells are administered before or following surgery.

The dosage of the above treatments to be administered to a patient will vary with the precise nature of the condition being treated and the recipient of the treatment. The scaling of dosages for human administration can be performed according to art-accepted practices. The dose for CAMPATH, for example, will generally be in the range 1 to about 100 mg for an adult patient, usually administered daily for a period between 1 and 30 days. In certain embodiments, 1 to 10 mg per day is used. In other embodiments, larger doses of up to 40 mg per day may be used (for example as described in U.S. Pat. No. 6,120,766).

### H. Articles of Manufacture and Kits

Another embodiment of the invention is an article of manufacture containing materials useful for the treatment, prevention and/or diagnosis of activin-expressing cancer. The article of manufacture comprises a container and a label or package insert on or associated with the container. Suitable containers include, for example, bottles, vials, syringes, etc. The containers may be formed from a variety of materials such as glass or plastic. The container holds a composition which is effective for treating, preventing and/or diagnosing the cancer condition and may have a sterile access port (for example the container may be an intravenous solution bag or a vial having a stopper pierceable by a hypodermic injection needle). At least one active agent in the composition is an anti-activin antibody of the invention. The label or package insert indicates that the composition is used for treating cancer. The label or package insert will further comprise instructions for administering the antibody composition to the cancer patient. Additionally, the article of manufacture may further comprise a second container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

Kits are also provided that are useful for various purposes, e.g., for activin-expressing cell killing assays, for purification or immunoprecipitation of activin polypeptide from cells. For isolation and purification of activin polypeptide, the kit can contain an anti-activin antibody coupled to beads (e.g., sepharose beads). Kits can be provided which contain the antibodies for detection and quantitation of activin polypeptide in vitro, e.g., in an ELISA or a Western blot. As with the article of manufacture, the kit comprises a container and a label or package insert on or associated with the container. The container holds a composition comprising at least one anti-activin antibody of the invention. Additional containers may be included that contain, e.g., diluents and buffers, control antibodies. The label or package insert may provide a description of the composition as well as instructions for the intended in vitro or detection use.

### I. Method of Screening

Yet another embodiment of the present invention is directed to a method of determining the presence of an activin polypeptide in a sample suspected of containing the activin polypeptide, wherein the method comprises exposing the sample to an antibody that binds to the activin polypeptide and determining binding of the antibody to the activin polypeptide in the sample, wherein the presence of such binding is indicative of the presence of the activin polypeptide in the sample. Optionally, the sample may contain cells (which may be cancer cells) suspected of expressing the activin polypeptide. The antibody employed in the method may optionally be detectably labeled, attached to a solid support, or the like.

Another embodiment of the present invention is directed to a method of diagnosing the presence of a tumor in a mammal, wherein the method comprises (a) contacting a test sample comprising tissue cells obtained from the mammal with an antibody that binds to an activin polypeptide and (b) detecting the formation of a complex between the antibody and the activin polypeptide in the test sample, wherein the formation of a complex is indicative of the presence of a tumor in the mammal. Optionally, the antibody is detectably labeled, attached to a solid support, or the like, and/or the test sample of tissue cells is obtained from an individual suspected of having a cancerous tumor. Antibody detection can be achieved via different techniques as described herein, e.g., IHC and PET imaging.

### IV. Further Methods of Using Anti-Activin Antibodies

### A. Therapeutic Methods

An antibody of the invention may be used in, for example, in vitro, ex vivo, and in vivo therapeutic methods. In one aspect, the invention provides methods for inhibiting cell growth or proliferation, either in vivo or in vitro, the method comprising exposing a cell to an anti-activin antibody under conditions permissive for binding of the antibody to activin. “Inhibiting cell growth or proliferation” means decreasing a cell's growth or proliferation by at least 10%, 20%, 30%, 40%, 50%, 60%, 70%, 80%, 90%, 95%, or 100%, and includes inducing cell death. In certain embodiments, the cell is a tumor cell. In certain embodiments, the cell is a B cell. In certain embodiments, the cell is a xenograft, e.g., as exemplified herein. The antibodies may also (i) inhibit the growth or proliferation of a cell to which they bind; (ii) induce the death of a cell to which they bind; (iii) inhibit the delamination of a cell to which they bind; (iv) inhibit the metastasis of a cell to which they bind; or (v) inhibit the vascularization of a tumor comprising a cell to which they bind.

In one aspect, an antibody of the invention is used to treat or prevent a cell proliferative disorder. In certain embodiments, the cell proliferative disorder is associated with increased expression and/or activity of activin. For example, in certain embodiments, the cell proliferative disorder is associated with increased expression of activin on the surface of a cell. In certain embodiments, the cell proliferative disorder is a tumor or a cancer.

In one aspect, the invention provides methods for treating a cell proliferative disorder comprising administering to an individual an effective amount of an anti-activin antibody.

In one embodiment, an anti-activin antibody can be used in a method for binding activin in an individual suffering from a disorder associated with increased activin expression and/or activity, the method comprising administering to the individual the antibody such that activin in the individual is bound. In one embodiment, the activin is human activin, and the individual is a human individual. An anti-activin antibody can be administered to a human for therapeutic purposes. Moreover, an anti-activin antibody can be administered to a non-human mammal expressing activin with which the antibody cross-reacts (e.g., a primate, pig, rat, or mouse) for veterinary purposes or as an animal model of human disease. Regarding the latter, such animal models may be useful for evaluating the therapeutic efficacy of antibodies of the invention (e.g., testing of dosages and time courses of administration).

An antibody of the invention (and any additional therapeutic agent or adjuvant) can be administered by any suitable means, including parenteral, subcutaneous, intraperitoneal, intrapulmonary, and intranasal, and, if desired for local treatment, intralesional administration. Parenteral infusions include intramuscular, intravenous, intraarterial, intraperitoneal, or subcutaneous administration. In addition, the antibody is suitably administered by pulse infusion, particularly with declining doses of the antibody. Dosing can be by any suitable route, e.g., by injections, such as intravenous or subcutaneous injections, depending in part on whether the administration is brief or chronic.

Antibodies of the invention would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

### B. Activity Assays

Anti-activin antibodies of the invention may be characterized for their physical/chemical properties and/or biological activities by various assays known in the art.

### 1. Activity Assays

In one aspect, assays are provided for identifying anti-activin antibodies thereof having biological activity. Biological activity may include, e.g., the ability to inhibit cell growth or proliferation (e.g., “cell killing” activity), or the ability to induce cell death, including programmed cell death (apoptosis). Antibodies having such biological activity in vivo and/or in vitro are also provided.

In certain embodiments, an anti-activin antibody is tested for its ability to inhibit cell growth or proliferation in vitro. Assays for inhibition of cell growth or proliferation are well known in the art. Certain assays for cell proliferation, exemplified by the “cell killing” assays described herein, measure cell viability. One such assay is the CellTiter-Glo™ Luminescent Cell Viability Assay, which is commercially available from Promega (Madison, WI). That assay determines the number of viable cells in culture based on quantitation of ATP present, which is an indication of metabolically active cells. See Crouch et al (1993) J. Immunol. Meth. 160:81-8, U.S. Pat. No. 6,602,677. The assay may be conducted in 96- or 384-well format, making it amenable to automated high-throughput screening (HTS) (see Cree et al (1995) AntiCancer Drugs 6:398-404). The assay procedure involves adding a single reagent (CellTiter-Glo® Reagent) directly to cultured cells. This results in cell lysis and generation of a luminescent signal produced by a luciferase reaction. The luminescent signal is proportional to the amount of ATP present, which is directly proportional to the number of viable cells present in culture. Data can be recorded by luminometer or CCD camera imaging device. The luminescence output is expressed as relative light units (RLU).

Another assay for cell proliferation is the “MTT” assay, a colorimetric assay that measures the oxidation of 3-(4,5-dimethylthiazol-2-yl)-2,5-diphenyltetrazolium bromide to formazan by mitochondrial reductase. Like the CellTiter-Glo™ assay, this assay indicates the number of metabolically active cells present in a cell culture (see, e.g., Mosmann (1983) J. Immunol. Meth. 65:55-63, and Zhang et al. (2005) Cancer Res. 65:3877-82).

In one aspect, an anti-activin antibody is tested for its ability to induce cell death in vitro. Assays for induction of cell death are well known in the art. In some embodiments, such assays measure, e.g., loss of membrane integrity as indicated by uptake of propidium iodide (PI), trypan blue (see Moore et al. Cytotechnology, 17:1-11 (1995)), or 7AAD. In an exemplary PI uptake assay, cells are cultured in Dulbecco's Modified Eagle Medium (D-MEM): Ham's F-12 (50:50) supplemented with 10% heat-inactivated FBS (Hyclone) and 2 mM L-glutamine. Thus, the assay is performed in the absence of complement and immune effector cells. Cells are seeded at a density of 3×106 per dish in 100×20 mm dishes and allowed to attach overnight. The medium is removed and replaced with fresh medium alone or medium containing various concentrations of the antibody. The cells are incubated for a 3-day time period. Following treatment, monolayers are washed with PBS and detached by trypsinization. Cells are then centrifuged at 1200 rpm for 5 minutes at 4° C., the pellet resuspended in 3 mL cold Ca2+ binding buffer (10 mM Hepes, pH 7.4, 140 mM NaCl, 2.5 mM CaCl2) and aliquoted into 35 mm strainer-capped 12×75 mm tubes (1 mL per tube, 3 tubes per treatment group) for removal of cell clumps. Tubes then receive PI (10 μg/mL). Samples are analyzed using a FACSCAN™ flow cytometer and FACSCONVERT™ CellQuest software (Becton Dickinson). Antibodies which induce statistically significant levels of cell death as determined by PI uptake are thus identified.

In one aspect, an anti-activin antibody is tested for its ability to induce apoptosis (programmed cell death) in vitro. An exemplary assay for antibodies that induce apoptosis is an annexin binding assay. In an exemplary annexin binding assay, cells are cultured and seeded in dishes as discussed in the preceding paragraph. The medium is removed and replaced with fresh medium alone or medium containing 0.001 to 10 μg/mL of the antibody. Following a three-day incubation period, monolayers are washed with PBS and detached by trypsinization. Cells are then centrifuged, resuspended in Ca2+ binding buffer, and aliquoted into tubes as discussed in the preceding paragraph. Tubes then receive labeled annexin (e.g., annexin V-FITC) (1 μg/mL). Samples are analyzed using a FACSCAN™ flow cytometer and FACSCONVERT™ CellQuest software (BD Biosciences). Antibodies that induce statistically significant levels of annexin binding relative to control are thus identified. Another exemplary assay for antibodies that induce apoptosis is a histone DNA ELISA colorimetric assay for detecting internucleosomal degradation of genomic DNA. Such an assay can be performed using, e.g., the Cell Death Detection ELISA kit (Roche, Palo Alto, CA).

Cells for use in any of the above in vitro assays include cells or cell lines that naturally express activin or that have been engineered to express activin. Such cells include tumor cells that overexpress activin relative to normal cells of the same tissue origin. Such cells also include cell lines (including tumor cell lines) that express activin and cell lines that do not normally express activin but have been transfected with nucleic acid encoding activin.

In one aspect, an anti-activin antibody thereof is tested for its ability to inhibit cell growth or proliferation in vivo. In certain embodiments, an anti-activin antibody thereof is tested for its ability to inhibit tumor growth in vivo. In vivo model systems, such as xenograft models, can be used for such testing. In an exemplary xenograft system, human tumor cells are introduced into a suitably immunocompromised non-human animal, e.g., a SCID mouse. An antibody of the invention is administered to the animal. The ability of the antibody to inhibit or decrease tumor growth is measured. In certain embodiments of the above xenograft system, the human tumor cells are tumor cells from a human patient. In certain embodiments, the human tumor cells are introduced into a suitably immunocompromised non-human animal by subcutaneous injection or by transplantation into a suitable site, such as a mammary fat pad.

### 2. Binding Assays and Other Assays

In one aspect, an anti-activin antibody is tested for its antigen binding activity. For example, in certain embodiments, an anti-activin antibody is tested for its ability to bind to activin expressed on the surface of a cell. A FACS assay may be used for such testing.

In one aspect, competition assays may be used to identify a monoclonal antibody that competes with a monoclonal antibody comprising the variable domains of any one of SEQ ID NOs: 1-70 or a chimeric antibody comprising the variable domain of the monoclonal antibody comprising the sequences of Table I and Table II, or Table III and Table IV, and constant domains from IgG1 for binding to activin. In certain embodiments, such a competing antibody binds to the same epitope (e.g., a linear or a conformational epitope) that is bound by a monoclonal antibody comprising the variable domains of any one of SEQ ID NOs: 1-70 or a chimeric antibody comprising the variable domain of the monoclonal antibody comprising the sequences of Table I and Table II, or Table III and Table IV, and constant domains from IgG1. Exemplary competition assays include, but are not limited to, routine assays such as those provided in Harlow and Lane (1988) Antibodies: A Laboratory Manual ch. 14 (Cold Spring Harbor Laboratory, Cold Spring Harbor, NY). Detailed exemplary methods for mapping an epitope to which an antibody binds are provided in Morris (1996) “Epitope Mapping Protocols,” in Methods in Molecular Biology vol. 66 (Humana Press, Totowa, NJ). Two antibodies are said to bind to the same epitope if each blocks binding of the other by 50% or more.

In an exemplary competition assay, immobilized activin is incubated in a solution comprising a first labeled antibody that binds to activin (e.g., a monoclonal antibody comprising the variable domains of any one of SEQ ID NOs: 1-70 or a chimeric antibody comprising the variable domain of the monoclonal antibody comprising the sequences of Table I and Table II, or Table III and Table IV, and constant domains from IgG1) and a second unlabeled antibody that is being tested for its ability to compete with the first antibody for binding to activin. The second antibody may be present in a hybridoma supernatant. As a control, immobilized activin is incubated in a solution comprising the first labeled antibody but not the second unlabeled antibody. After incubation under conditions permissive for binding of the first antibody to activin, excess unbound antibody is removed, and the amount of label associated with immobilized activin is measured. If the amount of label associated with immobilized activin is substantially reduced in the test sample relative to the control sample, then that indicates that the second antibody is competing with the first antibody for binding to activin. In certain embodiments, immobilized activin is present on the surface of a cell or in a membrane preparation obtained from a cell expressing activin on its surface.

In one aspect, purified anti-activin antibodies can be further characterized by a series of assays including, but not limited to, N-terminal sequencing, amino acid analysis, non-denaturing size exclusion high pressure liquid chromatography (HPLC), mass spectrometry, ion exchange chromatography and papain digestion.

The following examples are offered for illustrative purposes only and are not intended to limit the scope of the present invention in any way.

All patent, patent application, and literature references cited in the present specification are hereby incorporated by reference in their entirety.

### EXAMPLES

In the Examples below, high-affinity monoclonal antibodies (mAbs) were generated that specifically bind Activin A. An initial pool of antibody candidates was narrowed to 35 antibodies in IgG1 format using primary and secondary screening to focus on functional characteristics, such as Activin A binding and inhibition. These 35 antibodies were further categorized into three groups based on their functional attributes in various assays.

As Activin A activity is involved in many diseases and disorders, rationales for using antibodies described herein in different medical applications are laid out in the Examples. In the context of cancer, Activin A may be both a marker and a driver of disease. Data was gathered which supports the role of Activin A as contributing to cancer, thus making the inhibition of one or more Activin activities a plausible medical intervention. Inhibition of Activin A in fibrotic disease is evaluated as well.

### Example 1. Antibody Discovery—Screening, Identification, and Characterization

Antibody Discovery Campaigns. High-affinity, monoclonal antibodies (mAbs) were generated that specifically bind human Activin A via six different antibody enrichment campaigns.

In order to generate a highly diverse repertoire of antibodies with varying specificities and functional profiles regarding Activin A binding and inhibition, a generation 3.0 library at Specifica was utilized (Specifica, Inc, Sante Fe, NM). This is a natural library derived from human donors but built upon more readily developable antibody scaffolds. The Gen 3.0 technology employs a phage to yeast conversion step, which allows for enrichment and/or depletion strategies to be incorporated by FACS. These strategies were leveraged to generate anti-Activin A antibody repertoires with varying antigen specificities as shown in Table 2.

TABLE 2
Description of Phage/Yeast Selection Strategies Used
in the Anti-Activin A Antibody Discovery Campaigns

| Arm | Selection Strategy - Affinity to Target |
|---|---|
| 1 | Latent Activin A |
| 2 | Mature Activin A |
| 3 | Enriched on latent Activin A, de-selected on mature Activin A |
| 4 | Enriched on Activin A, de-selected on Inhibin A |
| 5 | Enriched on Activin A pre-complexed with Controls 1 and 2 |
| 6 | Enriched on Activin A pre-complexed with follistatin |

For each selection arm an antibody discovery campaign, libraries were selected on biotinylated antigens using two rounds of panning by phage display. The enriched phage antibody repertoires were then cloned into yeast vectors and were either enriched or depleted using fluorescently labelled antigens by FACS based on the strategies listed in Table 2. Outputs from the antigen sorting were sent for next generation sequencing (NGS) and repertoire analysis. Clones from each arm were then ranked based on frequency, and the top clones from each strategy were synthesized onto a human IgG1 Fc backbone. In total, 200 mAbs were generated from all six selection arms. These antibodies were expressed in a high throughput 96-well format in Expi293 cells, generating 1 mL of supernatant for functional screening, alongside positive and negative controls.

The control antibody designated herein as Control 1 is an anti-Activin A antibody with heavy and light chain domain sequences of “A1” as set forth in U.S. Pat. No. 8,309,082 (see SEQ ID NOs: 71-72). Control 2 is an anti-Activin A antibody with heavy and light chain sequences identical to those from the clinical candidate developed by Regeneron known as garetosmab (U.S. Pat. No. 9,718,881) (see SEQ ID NOs: 73-74).

In a separate antibody discovery campaign, CBl/6 mice were immunized with Latent Activin A using a rapid immunization protocol. The presence of Activin-binding antibodies was confirmed through a serum ELISA and then spleen tissue was harvested and homogenized in Trizol reagent to extract RNA. Antibody Design Labs (San Diego, CA) amplified the VH and VL regions from the spleen RNA and subcloned into a phage display vector. Phage selections with the mouse immune library were performed at Phenomic AI using biotinylated Activin A and magnetic bead-based panning. After four rounds of selection, individual colonies were picked, screened for binding to Activin A by ELISA, and sent for Sanger sequencing. In total, 72 unique sequences were identified thorugh this campaign. Expression plasmids encoding unique clones were synthesized and expressed in 10 mL cultures of CHO cells, generating supernatant for functional screening, alongside positive and negative controls.

### Primary Functional Screening of Anti-Activin A Antibodies in IgG1 Format

A function-first approach was used to screen and characterize anti-Activin A antibodies in IgG1 format using both i) human SMAD nuclear localization and ii) human fibroblast activation functional readouts as described below.

BJ fibroblasts were grown in MEM supplemented with 10% FBS, sodium pyruvate, penicillin and streptomycin. Cells were seeded at 6000 cells per well in 96-well COC bottom imaging plates overnight. The following day, cells were washed with PBS and starved in MEM with 0.1% serum for 16 hours. Antibodies from the discovery campaigns in IgG1 format were expressed in Expi293 cells in HEK-293 medium, and medium comprising antibodies was recovered and added to the serum-starved BJ cells at 20% v/v. After the addition of antibodies, the BJ cells were stimulated with either 2.5 nM of latent Activin A (R&D Systems, Minneapolis, MN) or 2.5 nM of mature Activin A (Sino Biological, Beijing, CN) and analyzed for indicators of SMAD nuclear localization or fibroblast activation. After either 1 hour (SMAD) or 48 hours (αSMA/CD248), the samples in the plates were permeabilized with HBSS 0.1% triton for 10 minutes, blocked with HBSS 3% BSA and stained for antibodies against SMAD2/3 (Santa Cruz Biotechnology, Dallas, TX), SMAD4 (Cell Signaling Technology, Danvers, MA), alpha SMA (Abcam, Cambridge, UK), CD248 (Abcam, Cambridge, UK), Hoeschst-33342 and Phalloidin. Plates were imaged using an InCell6000 with nine images per well.

Machine learning software was used to generate quantitative data (see U.S. patent application 63/011,999). The software trained on antibodies against negative and positive controls and the probability score of Activin A blockade was calculated as an average for each antibody across duplicates. The positive control antibodies were Control 1 and Control 2. Control 3 is a negative control antibody known as palivizumab (WO 2001/055217A1) (see SEQ ID NOs: 200-201).

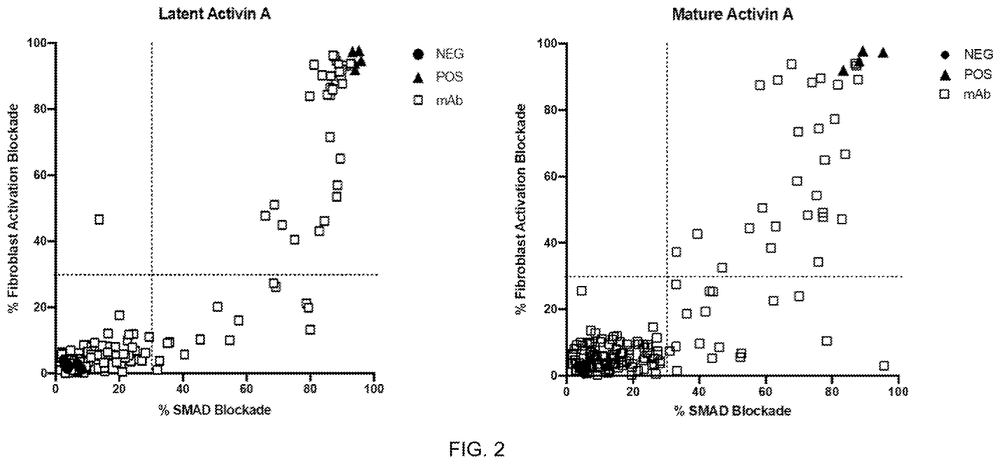

From the primary functional screens of SMAD nuclear localization and fibroblast activation, antibodies from the set of 200 were characterized having a range of inhibitory effects against both latent and mature Activin A (FIG. 2). The correlation in inhibition between the SMAD nuclear localization or fibroblast activation screens done with latent and mature Activin A showed a strong association, with R² values of 0.71 (SMAD nuclear localization screen) and 0.88 (fibroblast activation screen). Antibodies that demonstrated greater than thirty percent inhibition relative to the positive control antibodies Control 1 and Control 2 in both SMAD nuclear localization and fibroblast activation assays were carried forward for subsequent screening. We chose a more inclusive cutoff at 30% given that variation in the concentrations of the antibody in the supernatants can contribute to differences in potency in such single-dose functional assays. This resulted in 35 of the 150 (21%) of the mAbs screened being carried forward for additional screening.

### Secondary Screening of Anti-Activin a Antibodies in IgG1 Format for Antigen Binding

The 35 mAbs that were identified as having at least 30% functional activity in the single-dose SMAD nuclear localization and fibroblast activation assays were tested for binding to latent Activin A, mature Activin A, Activin B, Inhibin A, and BSA, which was used as a negative control.

Antibodies were re-expressed in 40 mL cultures of Expi293 cells and purified using protein G magnetic sepharose beads (Cytiva, Marlborough, MA) to provide larger quantities for secondary screening. Secondary screening included dose responses in both functional assays described above (SMAD nuclear localization and fibroblast activations assays) as well as in binding assays to the same antigens listed in Table 3. Plates were coated with 25 μL of 3 μg/mL antigen in 384-well Maxisorp plates overnight at 4° C. with agitation. The next day, the samples in the plates were blocked with 70 μL blocking buffer (PBS+1% BSA+0.1% Tween-20) for 2 hours at room temperature. Plates were then washed three times with 100 μL of wash buffer (PBS+0.1% Tween-20). Primary antibodies in Expi293 expression medium were diluted 1:3 in blocking buffer, 25 μL added per well, and the plates were incubated for an hour at room temperature with agitation. Plates were then washed three times and 25 μL of secondary antibody (goat anti-Human IgG, HRP conjugated; Jackson Immunoresearch) diluted 1:5000 in blocking buffer was added for 30 minutes at room temperature. The wells in the plates were washed six times with wash buffer and TMB substrate was added followed by 1 M H3PO4. The plates were read using a spectrophotometer set at OD of 450, and the results are shown in Table 3 as fold increase in signal over BSA (negative control).

Anti-Activin A antibodies Control 1 and Control 2 were included as positive controls in various experiments of these Examples for comparative purposes. The Control 3 antibody was included as a negative control in various experiments of these Examples for comparative purposes.

In this secondary screening, anti-Activin antibodies in IgG1 format were categorized as a confirmed binder to the antigen if they had greater than a 1.40-fold increase in binding for that antigen relative to BSA. Based on these analyses, antibodies were classified into four distinct bins (Table 3). Bin A represents a group of antibodies that bound to both latent and mature Activin A, but not to either Activin B or Inhibin A. Bin B represents a group of antibodies that bound to both latent and mature Activin A but were cross-reactive to Activin B (and did not bind to Inhibin A). Bin C represents a group of antibodies that bound to latent and mature Activin A, as well as Inhibin A (but were not cross-reactive to Activin B). Bin D represents an antibody that bound to latent and mature Activin A, as well as Activin B and Inhibin A. Some antibodies are members of two or more bins. Two antibodies samples tested (mAbs 431 and 330) were not conclusively allocated to any bin due to high background signal for the BSA control. Of note, both control mAbs anti-Activin A antibody Control 1 and anti-Activin A antibody Control 2 were categorized as members of Bin C, i.e. binding to all three of latent Activin A, mature Activin A and Inhibin A but not Activin B with at least 1.4-fold increase over negative control.

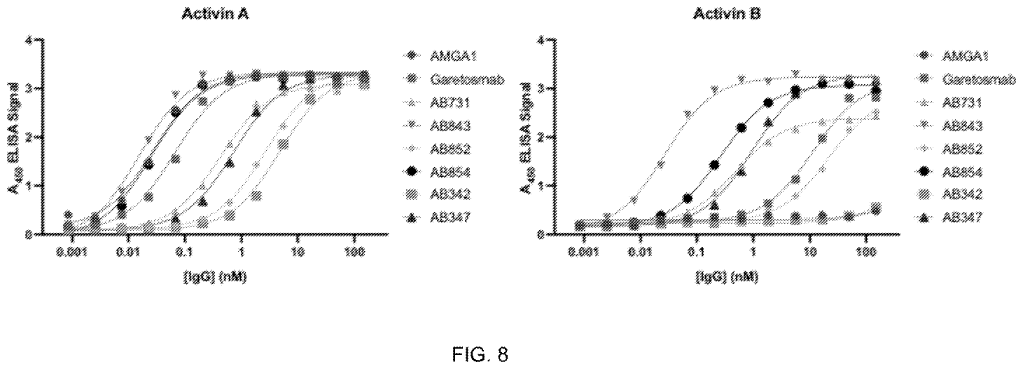

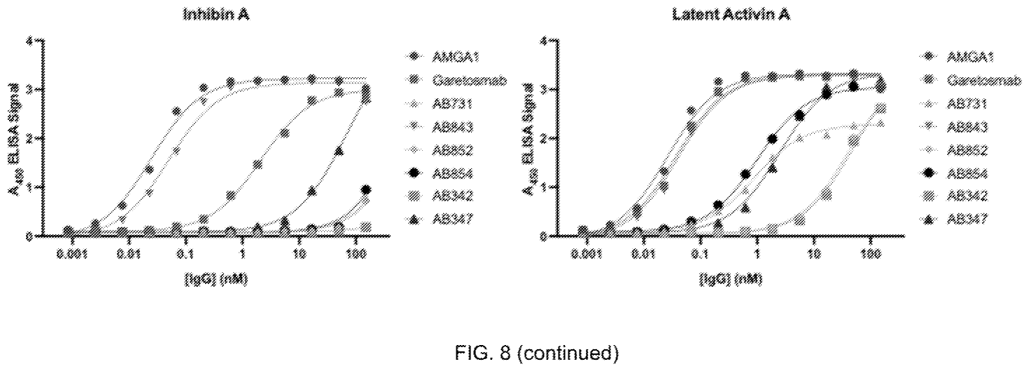

As shown in FIG. 8, select antibodies were subjected to additional binding characterization, including a dose-response assessment to determine binding potency using a similar assay with test antibodies diluted 1:3 from a starting concentration of 20 μg/mL or 133 nM. Assessment of activing binding and specificity by ELISA demonstrates specificity of select clones for mature Activin A (Activin A) or dual specificity for both mature Activin A and mature Activin B. Binding specificity was assessed by ELISA to mature Activin A, mature Activin B, Inhibin, and latent Activin A. This analysis showed that select activin antibodies suce as clones 843, 854, and 731 are potent binders of both mature Activin A and Activin B, with no detectible binding of Activin B by reference antibody Amga1 (Control 1) and garetosmab (Control 2) binding Activin B with over 100-fold weaker potency than clone 843. EC50 data corresponding to the binding curves depicted at FIG. 8, is shown in Table 4.

TABLE 3
Antigen Binding (OD 450) of Anti-Activin A IgG1
Antibodies (Fold-change over negative control)

| | | Latent | Mature | | | |
|---|---|---|---|---|---|---|
| mAb | | Activin | Activin | Activin | Inhibin | # |
| ID | Bin | A | A | B | A | mAbs |
| AB448 | A | 2.74 | 3.34 | 0.69 | 0.77 | 19 |
| AB465 | | 5.82 | 6.84 | 0.87 | 0.46 | |
| AB340 | | 1.90 | 2.33 | 1.04 | 1.11 | |
| AB342 | | 7.00 | 7.81 | 0.43 | 0.36 | |
| AB344 | | 2.35 | 3.18 | 0.75 | 1.18 | |
| AB353 | | 4.40 | 8.98 | 0.72 | 0.61 | |
| AB438 | | 2.34 | 2.83 | 0.15 | 0.23 | |
| AB366 | | 10.27 | 13.10 | 0.86 | 0.72 | |
| AB341 | | 4.09 | 6.71 | 0.98 | 0.81 | |
| AB430 | | 7.44 | 7.58 | 0.51 | 0.86 | |
| AB349 | | 4.34 | 5.14 | 0.27 | 0.22 | |
| AB449 | | 4.95 | 6.63 | 0.76 | 0.65 | |
| AB338 | | 1.44 | 1.94 | 0.68 | 0.90 | |
| AB435 | | 6.66 | 8.09 | 0.84 | 0.74 | |
| AB372 | | 5.64 | 7.16 | 0.43 | 0.43 | |
| AB439 | | 1.45 | 1.45 | 1.17 | 1.38 | |
| AB350 | | 7.33 | 10.64 | 0.74 | 0.65 | |
| AB363 | | 4.82 | 7.63 | 0.63 | 0.53 | |
| AB852 | | 6.01 | 8.73 | 0.88 | 0.94 | |
| AB352 | B | 15.22 | 16.18 | 2.03 | 0.61 | 11 |
| AB357 | | 6.59 | 6.99 | 2.44 | 0.77 | |
| AB347 | | 10.25 | 10.22 | 2.54 | 0.74 | |
| AB346 | | 13.77 | 13.99 | 2.46 | 0.58 | |
| AB433 | | 10.51 | 9.41 | 2.50 | 0.72 | |
| AB432 | | 10.40 | 8.86 | 1.72 | 0.74 | |
| AB326 | C | 5.91 | 6.01 | 0.58 | 8.53 | 4 |
| AB428 | | 5.93 | 6.34 | 0.72 | 3.09 | |
| AB426 | | 4.81 | 4.66 | 0.51 | 4.84 | |
| AB345 | | 8.12 | 9.47 | 0.76 | 3.43 | |
| AB843 | D | 24.95 | 24.74 | 24.57 | 22.90 | 1 |
| AB431 | Unknown | 1.00 | 1.04 | 0.96 | 1.13 | 2 |
| AB330 | | 0.75 | 1.39 | 0.61 | 0.97 | |
| Control | C | 18.70 | 16.27 | 0.57 | 11.67 | N/A |
| 1 | | | | | | |
| Control | | 21.57 | 20.96 | 0.71 | 15.95 | |
| 2 | | | | | | |
| Control | | 0.63 | 0.84 | 0.53 | 0.52 | |
| 3 | N/A | | | | | |

TABLE 4
EC50 Data (nM) corresponding to binding curves of FIG. 8.

| | AMGA1 | Garetosmab | AB731 | AB843 | AB852 | AB854 | AB342 | AB347 |
|---|---|---|---|---|---|---|---|---|
| Mature | 0.03 | 0.07 | 0.42 | 0.02 | 2.73 | 0.03 | 4.89 | 0.72 |
| Activin | | | | | | | | |
| A | | | | | | | | |
| Mature | 1247.28 | 11.25 | 0.49 | 0.03 | 21.28 | 0.27 | 635.33 | 0.95 |
| Activin | | | | | | | | |
| B | | | | | | | | |
| Inhibin | 0.02 | 2.08 | ND | 0.05 | ND | ND | ND | ND |
| Latent | 0.03 | 0.04 | 0.87 | 0.04 | 40.93 | 1.00 | 48.64 | 2.46 |
| Activin | | | | | | | | |
| A | | | | | | | | |

Antibodies which bind and inhibit Activin A have been described previously (WO2014121221; WO2015017576) but any cross-reactivity and functional effects of such antibodies to Inhibins and other Activin(s) are not clear. For example, do these antibodies bind Inhibin A as well? Do these antibodies cross-react with Activin B and/or Inhibin B? Do these antibodies bind both latent and active Activin A or latent Activin A, active Activin A, and Inhibin A?

Control 1, an anti-Activin-A antibody described by Santa Maria Biotherapeutics and Amgen (“anti-activin-A antibody” A1 sequences in WO2014121221) was tested for binding specificity using the assays described above. The anti-activin-A antibody “A1” named herein Control 1 bound to both Activin A and Inhibin A but not Activin B or Inhibin B in these assays.

Control 2, an anti-Activin-A antibody referred to as garetosmab and described by Regeneron (U.S. Pat. No. 9,718,881, which is incorporated by reference in its entirety), was tested for binding specificity using the assays described above. Garetosmab bound both Activin A and Inhibin A but not Activin B or Inhibin B in these assays.

TABLE 5
Illustrative Immunoglobulin Sequences-Heavy Chain Variable Region

| Name | SEQ ID | Heavy Chain Variable Region |
|---|---|---|
| AB448 | 1 | QVQLQESGPGLVKPSQTLSLTCTVSGLSLSNAKMGVNWIRQPPGKGLEWI |
| | | GYVSTTGTTDYNPSLKSRVTMSVDTSKNQFSLKVNSVTAADTAVYYCARAG |
| | | YPDVWGKGTTVTVSS |
| AB465 | 3 | QVQLQESGPGLVKPSQTLSLTCTVSGASVSNARTGVNWIRQPPGKGLEWI |
| | | GYINPSGRIDYNPSLKSRVTMSVDTSKNQFSLKVNSVTAADTAVYYCARAG |
| | | YPDVWGKGTTVTVSS |
| AB340 | 5 | EVQLVESGGGLVQPGGSLRLSCAASGFTASRNYMSWVRQAPGKGLELVA |
| | | SISGSGDYTYYPDSVKGRFTISRDNAKNSLYLQMNSLRAEDTAVYYCARSP |
| | | RGPRLLGYYYGMDVWGQGTTVTVSS |
| AB342 | 7 | EVQLVESGGGLVQPGGSLRLSCAASGFDFGEHWMSWVRQAPGKGLELVA |
| | | SIKGDASDTYYPDSVKGRFTISRDNAKNSLYLQMNSLRAEDTAVYYCVKDLS |
| | | KQWLGIDYWGQGTLVTVSS |
| AB344 | 9 | EVQLVESGGGLVQPGGSLRLSCAASGYTFSNYGMSWVRQAPGKGLELVA |
| | | SISKSGSSIYYPDSVKGRFTISRDNAKNSLYLQMNSLRAEDTAVYYCARLPR |
| | | LAWNYYYGMDVWGQGTTVTVSS |
| AB353 | 11 | QVQLQESGPGLVKPSQTLSLTCTVSGLSLSNAKMGVNWIRQPPGKGLEWI |
| | | GYVSTTGTTDYNPSLKSRVTMSVDTSKNQFSLKVNSVTAADTAVYYCARAG |
| | | YPDVWGKGTTVTVSS |
| AB438 | 13 | QVQLQESGPGLVKPSQTLSLTCTVSGVSLSNARLGVNWIRQPPGKGLEWIG |
| | | YGSVTGSTDYNPSLKSRVTMSVDTSKNQFSLKVNSVTAADTAVYYCTTSYL |
| | | VDTAMVTRWGQGTLVTVSS |
| AB366 | 15 | QVQLQESGPGLVKPSQTLSLTCTVSGYSLTDARIGVNWIRQPPGKGLEWIG |
| | | YINPSGRTDYNPSLKSRVTMSVDTSKNQFSLKVNSVTAADTAVYYCARAGY |
| | | PDWWGKGTTVTVSS |
| AB341 | 17 | EVQLVESGGGLVQPGGSLRLSCAASGYTFSNYGMSWVRQAPGKGLELVA |
| | | SISKSGSSIYYPDSVKGRFTISRDNAKNSLYLQMNSLRAEDTAVYYCARLPR |
| | | LAWNYYYGMDVWGQGTTVTVSS |
| AB430 | 19 | EVQLVESGGGLVQPGGSLRLSCAASGFTLSNYPMSWVRQAPGKGLELVAS |
| | | TTSGSSTIYYPDSVKGRFTISRDNAKNSLYLQMNSLRAEDTAVYYCARGPD |
| | | YYAPLRSYMDVWGKGTTVTVSS |
| AB349 | 21 | QVQLQESGPGLVKPSQTLSLTCTVSGVSLSNARLGVNWIRQPPGKGLEWIG |
| | | YGSVTGSTDYNPSLKSRVTMSVDTSKNQFSLKVNSVTAADTAVYYCTTSYL |
| | | VDTAMVTRWGQGTLVTVSS |
| AB449 | 23 | QVQLQESGPGLVKPSQTLSLTCTVSGLSLSNAKMGVNWIRQPPGKGLEWI |
| | | GYVSTTGTTDYNPSLKSRVTMSVDTSKNQFSLKVNSVTAADTAVYYCARAG |
| | | YPDWWGKGTTVTVSS |
| AB338 | 25 | EVQLVESGGGLVQPGGSLRLSCAASGFTASRNYMSWVRQAPGKGLELVA |
| | | SISGSGDYTYYPDSVKGRFTISRDNAKNSLYLQMNSLRAEDTAVYYCARSP |
| | | RGPRLLGYYYGMDVWGQGTTVTVSS |
| AB435 | 27 | QVQLQESGPGLVKPSQTLSLTCTVSGYSLTDARIGVNWIRQPPGKGLEWIG |
| | | YINPSGRTDYNPSLKSRVTMSVDTSKNQFSLKVNSVTAADTAVYYCARAGY |
| | | PDWWGKGTTVTVSS |
| AB372 | 29 | QVTLRESGPALVKPTQTLTLTCTVSGFSLSTSGATVNWIRQPPGKALEWIGY |
| | | ITSSGRTVYNSALKSRLTISKDTSKNQVVLTMTNMDPVDTATYYCTTTKIVGA |
| | | TLRDYWGQGTLVTVSS |
| AB439 | 31 | EVQLVQSGAEVKKPGASVKVSCKASGYTYTGYRISWVRQAPGQGLEWMG |
| | | WIDPKSGRTNYAQKLQGRGTMTTDPSTSTAYMELRSLRSDDTAVYYCARE |
| | | VKQWLVPGRGVYWFDPWGQGTLVTVSS |
| AB350 | 33 | QVQLQESGPGLVKPSQTLSLTCTVSGVSLSDARMGVNWIRQPPGKGLEWI |
| | | GYVSTTGTTDYNPSLKSRVTMSVDTSKNQFSLKVNSVTAADTAVYYCARAG |
| | | YPDVWGKGTLVTVSS |
| AB363 | 35 | QVTLRESGPALVKPTQTLTLTCTVSGFSLSTSGATVNWIRQPPGKALEWIGY |
| | | ITSSGRTVYNSALKSRLTISKDTSKNQVVLTMTNMDPVDTATYYCTTTKIVGA |
| | | TLRDYWGQGTLVTVSS |
| AB367 | 37 | QVTLRESGPALVKPTQTLTLTCTVSGFTLSNPRMGVNWIRQPPGKGLEWIG |
| | | YISTSGRTDYNPSLKSRVTMSVDTSKNQFSLKVNSVTAADTAVYYCARAGY |
| | | PDVWGKGTTVTVSS |
| AB352 | 39 | QVQLQESGPGLVKPSQTLSLTCTVSGFPLSTSRMGVNWIRQPPGKGLEWI |
| | | GYINPSGATDYNPSLKSRVTMSVDTSKNQFSLKVNSVTAADTAVYYCARAG |
| | | YPDWWGKGTTVTVSS |
| AB357 | 41 | EVQLVQSGAEVKKPGASVKVSCKASGYTFPSHYISWVRQAPGQGLEWMG |
| | | GMNPITGHTIYAQKLQGRGTMTTDPSTSTAYMELRSLRSDDTAVYYCARDM |
| | | RYDILPGRSYYYGMDVWGQGTTVTVSS |
| AB347 | 43 | QVQLQESGPGLVKPSQTLSLTCTVSGFPLSTSRMGVNWIRQPPGKGLEWI |
| | | GYINPSGATDYNPSLKSRVTMSVDTSKNQFSLKVNSVTAADTAVYYCARAG |
| | | YPDVWGKGTTVTVSS |
| AB346 | 45 | QVQLQESGPGLVKPSQTLSLTCTVSGFPLSTSRMGVNWIRQPPGKGLEWI |
| | | GYINPSGATDYNPSLKSRVTMSVDTSKNQFSLKVNSVTAADTAVYYCARAG |
| | | YPDVWGKGTTVTVSS |
| AB433 | 47 | QVQLQESGPGLVKPSQTLSLTCTVSGFPLSTSRMGVNWIRQPPGKGLEWI |
| | | GYINPSGATDYNPSLKSRVTMSVDTSKNQFSLKVNSVTAADTAVYYCARAG |
| | | YPDVWGKGTTVTVSS |
| AB432 | 49 | QVQLQESGPGLVKPSQTLSLTCTVSGVSLSNVRMGVNWIRQPPGKGLEWI |
| | | GYVNPSGSTDYNPSLKSRVTMSVDTSKNQFSLKVNSVTAADTAVYYCARA |
| | | GYPDVWGKGTLVTVSS |
| AB326 | 51 | QVQLVQSGAEVKKPGASVKVSCKVSGYTFTSSAISWVRQAPGKGLEWMG |
| | | GIYPGSGHTIYAQKFQGRVTMTEDTSTDTAYMELSSLKSEDTAVYYCAREG |
| | | DIFVMDVWGLGTAVTVSS |
| AB428 | 53 | EVQLVESGGGLVQPGGSLRLSCAASGFTSSNHWMSWVRQAPGKGLELVA |
| | | SIGPKGLGTYYPDSVKGRFTISRDNAKNSLYLQMNSLRAEDTAVYYCASIRD |
| | | YGDKKGGSWFDPWGQGTLVTVSS |
| AB426 | 55 | EVQLVESGGGLVQPGGSLRLSCAASGFTFSDHHMSWVRQAPGKGLELVA |
| | | SISPSGDFTYYPDSVKGRFTISRDNAKNSLYLQMNSLRAEDTAVYYCARDV |
| | | SPVYYWGQGALVTVSS |
| AB345 | 57 | EVQLVESGGGLVQPGGSLRLSCAASGFTSSGYWMSWFRQAPGKGLELVA |
| | | SIGTAGVTYYPDSVKGRFTISRDNAKNSLYLQMNSLRAEDTAVYYCARGPH |
| | | GPQQRLRFDPWGQGTLVTVSS |
| AB431 | 59 | EVQLVESGGGLVQPGGSLRLSCAASGFTSSNHWMSWVRQAPGKGLELVA |
| | | SIGPKGLGTYYPDSVKGRFTISRDNAKNSLYLQMNSLRAEDTAVYYCARDV |
| | | RGWRALYYYYGMDVWGQGTTVTVSS |
| AB330 | 61 | QVQLVQSGAEVKKPGASVKVSCKVSGYPSPATISWVRQAPGKGLEWMGW |
| | | MNPKSGYTNYAQKFQGRVTMTEDTSTDTAYMELSSLKSEDTAVYYCATLH |
| | | GSGLAFDIWGQGTMVTVSS |
| AB731 | 63 | QVQLQQPGTELVKPGASVKLSCKASGYTFTSYWMHWVKQRPGQGLEWIG |
| | | NINPSNGGTNYNEKFKSKATLTVDKSSSTAYMQLSSLTSEDSAVYYCSITTV |
| | | VAYWYFDVWGTGTTVTVSS |
| AB843 | 65 | EVQLVQSGAEVKKPGASVKVSCKASGDTSSHSHIHWVRQAPGQGLEWMG |
| | | WINPRSGATNYAQKLQGRGTMTTDPSTSTAYMELRSLRSDDTAVYYCARD |
| | | RGGRTNGVGAFDIWGQGTMVTVSS |
| AB852 | 67 | QVTLRESGPALVKPTQTLTLTCTVSGFSLSDTRWGVNWIRQPPGKALEWIG |
| | | YINDRGTTDYNSALKSRLTISKDTSKNQVVLTMTNMDPVDTATYYCAHTLHT |
| | | REFVGMDVWGQGTTVTVSS |
| AB854 | 69 | EVQLVQSGAEVKKPGASVKVSCKASGYTFPSHYISWVRQAPGQGLEWMG |
| | | GMNPITGHTIYAQKLQGRGTMTTDPSTSTAYMELRSLRSDDTAVYYCARDM |
| | | RYDILPGRSYYYGMDVWGQGTTVTVSS |

TABLE 6
Illustrative Immunoglobulin Sequences-Light Chain Variable Region

| Name | SEQ ID | Light Chain Variable Region |
|---|---|---|
| AB448 | 2 | DIQMTQSPSSVSASVGDRVTITCRASQNIGAALAWYQQKPGKAPKLLIYGAS |
| | | TREYGVPSRFSGSGSGTDFTLTISSLQPEDFANYYCQQYTAYPLTFGGGTK |
| | | VEIK |
| AB465 | 4 | EIVMTQSPATLSLSPGERATLSCRASQTVSSHLAWYQQKPGQAPRLLIYDV |
| | | SHRASGIPARFSGSGSGTDFTLTISSLEPEDFAVYYCQQYFTLPTFGGGTKV |
| | | EIK |
| AB340 | 6 | DIVMTQSPLSLPVTPGEPASISCRSSRSLVHGSGHNYLHWYLQKPGQSPQL |
| | | LIYGASTRVAGVPDRFSGSGSGTDFTLKISRVEAEDVGVYYCMQGTQAPRT |
| | | FGGGTKVEIK |
| AB342 | 8 | DIVMTQSPLSLPVTPGEPASISCRSSQSLLHSNEKNYLHWYLQKPGQSPQL |
| | | LIYDASKRASGVPDRFSGSGSGTDFTLKISRVEAEDVGVYYCVQDLHTPFTF |
| | | GGGTKVEIK |
| AB344 | 10 | DIVMTQSPLSLPVTPGEPASISCRSSRSLVHGSGDNYLHWYLQKPGQSPQL |
| | | LIYMASNRAPGVPDRFSGSGSGTDFTLKISRVEAEDVGVYYCVQTLQTPST |
| | | FGGGTKVEIK |
| AB353 | 12 | EIVMTQSPATLSLSPGERATLSCRASQNVYTNLAWYQQKPGQAPRLLIYGA |
| | | SARASGIPARFSGSGSGTDFTLTISSLEPEDFAVYYCLQYGSSPPLFGGGTK |
| | | VEIK |
| AB438 | 14 | EIVMTQSPATLSLSPGERATLSCRASQSLGTNLAWYQQKPGQAPRLLIYGA |
| | | SSRPSGIPARFSGSGSGTDFTLTISSLEPEDFAVYYCQQYDTHPLSFGGGTK |
| | | VEIK |
| AB366 | 16 | DIQMTQSPSSVSASVGDRVTITCRASQTISRSLAWYQQKPGKAPKLLIYDAS |
| | | SLAGGVPSRFSGSGSGTDFTLTISSLQPEDFANYYCQQYSSYPSLFGGGTK |
| | | VEIK |
| AB341 | 18 | DIVMTQSPLSLPVTPGEPASISCRSSQSLLHSNEKNYLHWYLQKPGQSPQL |
| | | LIYDASKRASGVPDRFSGSGSGTDFTLKISRVEAEDVGVYYCVQDLHTPFTF |
| | | GGGTKVEIK |
| AB430 | 20 | DIVMTQSPLSLPVTPGEPASISCRSSQSLQQSEGRTYLHWYLQKPGQSPQL |
| | | LIYDASKRASGVPDRFSGSGSGTDFTLKISRVEAEDVGVYYCMQVLRTPITF |
| | | GGGTKVEIK |
| AB349 | 22 | EIVMTQSPATLSLSPGERATLSCRASQRIGSDLAWYQQKPGQAPRLLIYSAS |
| | | SRAPGIPARFSGSGSGTDFTLTISSLEPEDFAVYYCSTMAIPFGGGTKVEIK |
| AB449 | 24 | DIQMTQSPSSVSASVGDRVTITCRASQGISRDLAWYQQKPGKAPKLLIYAAS |
| | | TLVSGVPSRFSGSGSGTDFTLTISSLQPEDFANYYCQQYVSAPPYTFGGGT |
| | | KVEIK |
| AB338 | 26 | DIVMTQSPLSLPVTPGEPASISCRSSQSLLHSNEKNYLHWYLQKPGQSPQL |
| | | LIYDASKRASGVPDRFSGSGSGTDFTLKISRVEAEDVGVYYCVQDLHTPFTF |
| | | GGGTKVEIK |
| AB435 | 28 | EIVMTQSPATLSLSPGERATLSCRASQSIGTKLAWYQQKPGQAPRLLIYDAS |
| | | SRVPGIPARFSGSGSGTDFTLTISSLEPEDFAVYYCQQYGSSPPVYSFGGG |
| | | TKVEIK |
| AB372 | 30 | DIVMTQSPDSLAVSLGERATINCKSSQSVLDTSTNRNYLAWYQQKPGQPPK |
| | | LLIYGASIRAGGVPDRFSGSGSGTDFTLTISSLQAEDVAVYYCQQSYSHPPT |
| | | FGGGTKVEIK |
| AB439 | 32 | ESALTQPASVSGSPGQSITISCTGTNIGLRSVSWYQQHPGKAPKLMIYDTNI |
| | | RSSGVSNRFSGSKSGNTASLTISGLQAEDEADYYCSSYTSSSTDVFGGGTK |
| | | LTVL |
| AB350 | 34 | EIVMTQSPATLSLSPGERATLSCRASESVRSNLAWYQQKPGQAPRLLIYGA |
| | | SNRASGIPARFSGSGSGTDFTLTISSLEPEDFAVYYCQQYHTWPPWAFGG |
| | | GTKVEIK |
| AB363 | 36 | DIQMTQSPSSVSASVGDRVTITCRASQGISNDLAWYQQKPGKAPKLLIYDVS |
| | | NRASGVPSRFSGSGSGTDFTLTISSLQPEDFANYYCQQYDNPPSFGGGTK |
| | | VEIK |
| AB367 | 38 | DIQMTQSPSSVSASVGDRVTITCRASQGIGIDLAWYQQKPGKAPKLLIYDAS |
| | | KLQTGVPSRFSGSGSGTDFTLTISSLQPEDFANYYCRQYATFPQTFGGGTK |
| | | VEIK |
| AB352 | 40 | EIVMTQSPATLSLSPGERATLSCRASQSVSTHLAWYQQKPGQAPRLLIYDA |
| | | TNRATGIPARFSGSGSGTDFTLTISSLEPEDFAVYYCLQYTNYPWTFGGGTK |
| | | VEIK |
| AB357 | 42 | ESALTQPASVSGSPGQSITISCTGTDIRGMSVSWYQQHPGKAPKLMIYEVN |
| | | QRPSGVSNRFSGSKSGNTASLTISGLQAEDEADYYCAAWDASVAGYVFGG |
| | | GTKLTVL |
| AB347 | 44 | EIVMTQSPATLSLSPGERATLSCRASQNIGHSLAWYQQKPGQAPRLLIYGAS |
| | | IRAPGIPARFSGSGSGTDFTLTISSLEPEDFAVYYCQQYITWPSTFGGGTKV |
| | | EIK |
| AB346 | 46 | EIVMTQSPATLSLSPGERATLSCRASRSVSSDLAWYQQKPGQAPRLLIYDV |
| | | STRATGIPARFSGSGSGTDFTLTISSLEPEDFAVYYCQQYGSSLPPTFGGGT |
| | | KVEIK |
| AB433 | 48 | EIVMTQSPATLSLSPGERATLSCRASQSVRTSLAWYQQKPGQAPRLLIYDT |
| | | SIRAAGIPARFSGSGSGTDFTLTISSLEPEDFAVYYCQQYGNWHFGGGTKV |
| | | EIK |
| AB432 | 50 | EIVMTQSPATLSLSPGERATLSCRASQSISSSQLAWYQQKPGQAPRLLIYGA |
| | | STRATGIPARFSGSGSGTDFTLTISSLEPEDFAVYYCQQYNDRPRTFGGGT |
| | | KVEIK |
| AB326 | 52 | DIQMTQSPSSVSASVGDRVTITCRASMDIRNDLAWYQQKPGKAPKLLIYDAS |
| | | KLETGVPSRFSGSGSGTDFTLTISSLQPEDFANYYCQQAKSFPPTFGGGTK |
| | | VEIK |
| AB428 | 54 | DIVMTQSPLSLPVTPGEPASISCRSSQSLLHSSGYNFLHWYLQKPGQSPQL |
| | | LIYMATNRASGVPDRFSGSGSGTDFTLKISRVEAEDVGVYYCVQDLHTLPQ |
| | | FGGGTKVEIK |
| AB426 | 56 | DIVMTQSPLSLPVTPGEPASISCRSSQSLLHWYLQKPGQSPQLLIYMGSQR |
| | | AAGVPDRFSGSGSGTDFTLKISRVEAEDVGVYYCQSGVTFGGGTKVEIK |
| AB345 | 58 | DIVMTQSPLSLPVTPGEPASISCRSSQSLLSSYGYHNLHWYLQKPGQSPQL |
| | | LIYMGSIRAPGVPDRFSGSGSGTDFTLKISRVEAEDVGVYYCMQGTQAPRT |
| | | FGGGTKVEIK |
| AB431 | 60 | DIVMTQSPLSLPVTPGEPASISCRSSQSLLHVTGYNYLHWYLQKPGQSPQL |
| | | LIYGASSRAAGVPDRFSGSGSGTDFTLKISRVEAEDVGVYYCMQSTQWPHT |
| | | FGGGTKVEIK |
| AB330 | 62 | DIQMTQSPSSVSASVGDRVTITCRASQDIKSDLAWYQQKPGKAPKLLIYRAS |
| | | ARESGVPSRFSGSGSGTDFTLTISSLQPEDFANYYCQQYRSFPWSFGGGT |
| | | KVEIK |
| AB731 | 64 | ENVLTQSPAIMSASPGEKVTISCSASSSVSYMYWYQQKPGSSPKPWIFRTY |
| | | NLASGVPARFSGSGSGTSYSLTISNMEAEDAATYYCQQRSSYPWTFGGGT |
| | | KLEIK |
| AB843 | 66 | ESALTQPASVSGSPGQSITISCTGTSGDIGRYNYVSWYQQHPGKAPKLMIY |
| | | EDIERPSGVSNRFSGSKSGNTASLTISGLQAEDEADYYCQVWDATLDLVFG |
| | | GGTKLTVL |
| AB852 | 68 | DIVMTQSPDSLAVSLGERATINCKSSQSVLDTSTNRNYLAWYQQKPGQPPK |
| | | LLIYGASIRAGGVPDRFSGSGSGTDFTLTISSLQAEDVAVYYCQQYETWPW |
| | | TFGGGTKVEIK |
| AB854 | 70 | ESALTQPASVSGSPGQSITISCTGTTGSIASNYVSWYQQHPGKAPKLMIYDT |
| | | TNRPPGVSNRFSGSKSGNTASLTISGLQAEDEADYYCQSTERGRSDVIFGG |
| | | GTKLTVL |

### Example 2. Activin Inhibition in Cancer

In the context of cancer, inhibiting Activin A and B will prevent immune suppression and fibroblast activation.

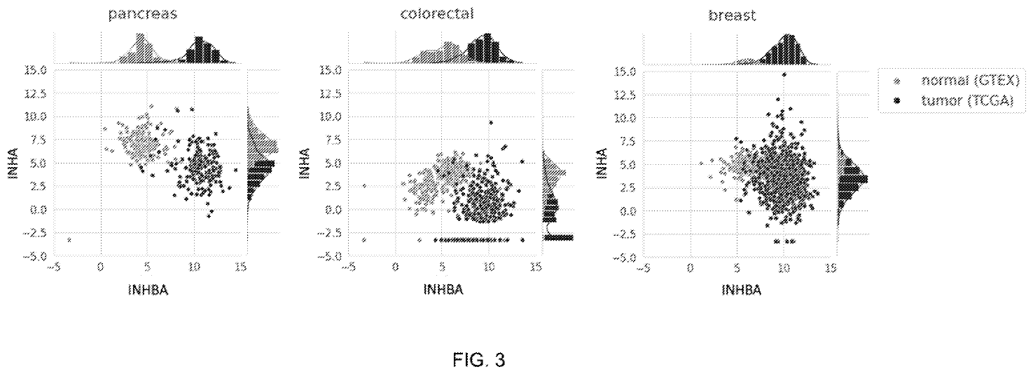

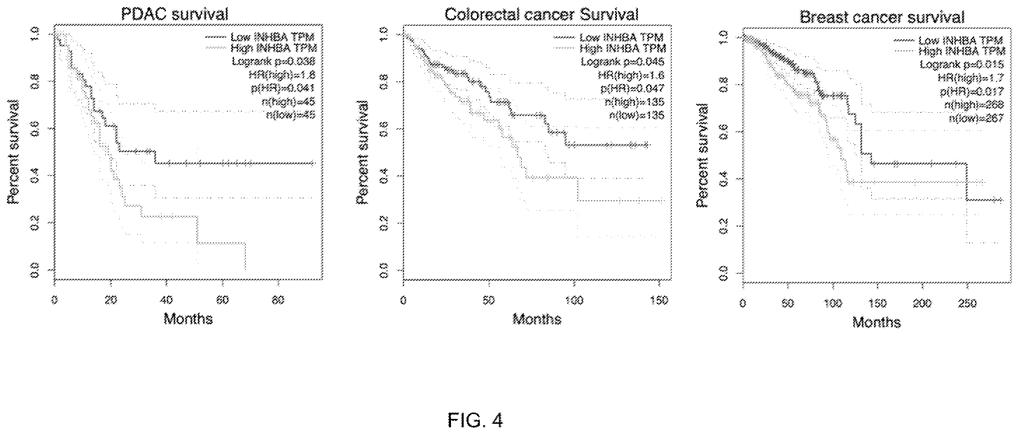

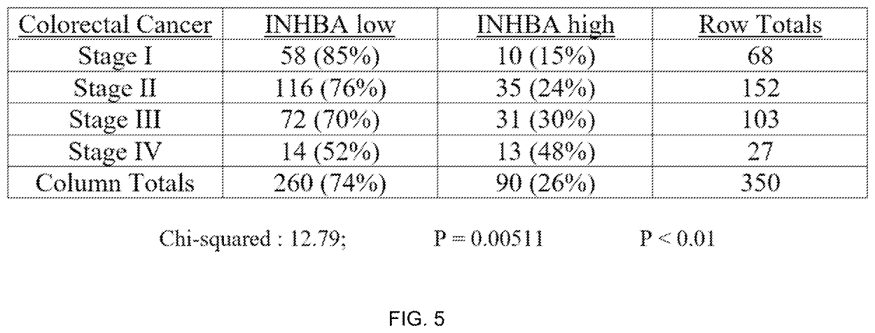

Data was gathered which supports the idea that Activin A and B are both upregulated in and contributing to cancer. In this Example, Activin A expression is shown to be upregulated in several solid tumor indications, including pancreas, colorectal and breast cancer (FIG. 3). Activin A is shown to be a prognostic indicator of survival in these indications (FIG. 4). Activin A expression is shown to correlate with stage of cancer disease progression in colorectal cancer (FIG. 5). Activin A is shown to be expressed differentially from TGF-β under experimental conditions where fibroblasts are co-cultured with cancer cells versus monocultures of the same cells (FIG. 4).

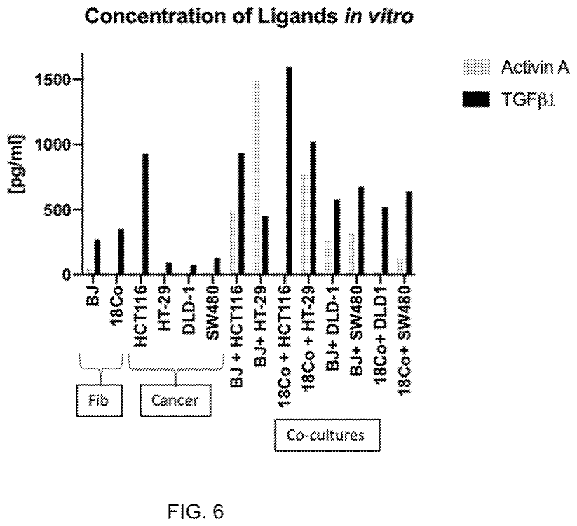

The expression of Activin A and TGF-beta was analyzed in various tissues and cell-types. FIG. 3 shows Activin A was highly expressed in multiple solid tumor indications (breast, colon, and pancreatic) compared to normal tissue (x-axis). In contrast, INHA gene expression varied across these solid tumor indications. Analysis was done using data obtained from University of California Santa Cruz's GTEx database. FIG. 6 shows INHBA expression in fibroblasts, tumor cells and co-cultures of fibroblast/tumor cells. Cancer and fibroblast cell lines were grown in MEM and seeded at either 6000 cells/well (monocultures) or 12,000 cells/well (co-cultures, 6000 cells each). After 48 hours in culture, supernatants were collected and analyzed by ELISA to quantitate either Activin A (using a human Activin A Quantikine kit, catalog no. DACOOB, R&D Systems, Minneapolis, MN) or TGFβ1. FIG. 6 shows Activin A was not significantly expressed in individual fibroblast or tumor cell monocultures, unlike TGF-β, which was expressed in individual fibroblast and tumor cell monocultures as well as in all the co-cultures of fibroblast/tumor cells tested. Thus, Activin A is upregulated in fibroblast: tumor co-cultures, and, unlike TGF-β1, Activin A is not significantly expressed in individual fibroblast or tumor cell monocultures.

The expression of Activin was analyzed for correlations with neoplastic disease and disease progression. FIG. 4-A shows expression of Activin A is prognostic in multiple solid tumor indications (breast, colon, and pancreatic cancers). Analysis was done using data from The Cancer Genome Atlas (TCGA). For three solid tumor types (breast, colon, and pancreatic cancers) that express high levels of Activin A, hazard ratios were calculated between the patients expressing greater or less than the median level of Activin A; resulting in a 50:50 split. FIG. 4-B shows survival curves plotted highlighting differences between the two groups (solid line), alongside confidence intervals at P=0.05, indicating where significant divergence is seen between the two groups. For all three tumor types, statistically significant variation between survival times in the two groups were observed.

FIG. 5 shows Activin A expression levels in colorectal cancer correlated with the stages of disease. Data was taken from The Cancer Genome Atlas and plotted using the online tool GEPIA.

### Activin A Activity in Transformed Fibroblasts in Tissue Culture

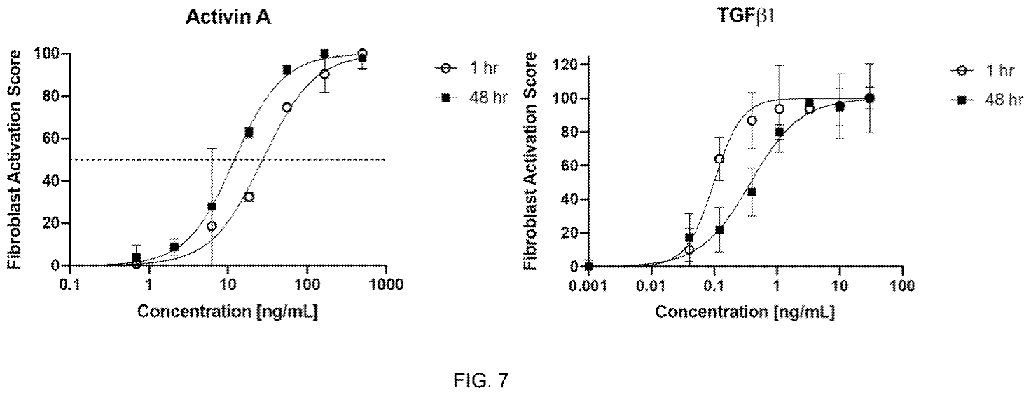

The effects of Activin A on fibroblasts was studied in vitro. FIG. 7 shows Activin A treatment stimulated SMAD2/3 and SMAD4 nuclear localization and markers of BJ fibroblast activation (CD248 and αSMA) comparable to levels stimulated by TGF-β ligand treatment. Fibroblast activation scores were determined by upregulation of αSMA and downregulation of CD248 using machine learning software (see U.S. patent application 63/011,999). In this assay, the half-maximal effective concentration EC50 for Activin A was measured to be 27.5 ng/ml (1.1 nM) at 1 hour and 12.1 ng/mL (484 pM) at 48 hours.

### Example 3. In Vivo Model Data

The INHBA gene is expressed in various murine syngeneic tumor models, such as the breast cancer models 4T1 and EMT6, and the colon cancer models CT26 and MC38. Murine tumor models are treated with antibodies of the invention and evaluated for reductions or elimination of tumor growth. Adminsitration of one or more of the 35 mAbs described in Example 1 results in reductions in tumor growth in one or more murine syngeneic tumor models.

### Example 4. Colorectal Cancer Tumor Explant Data

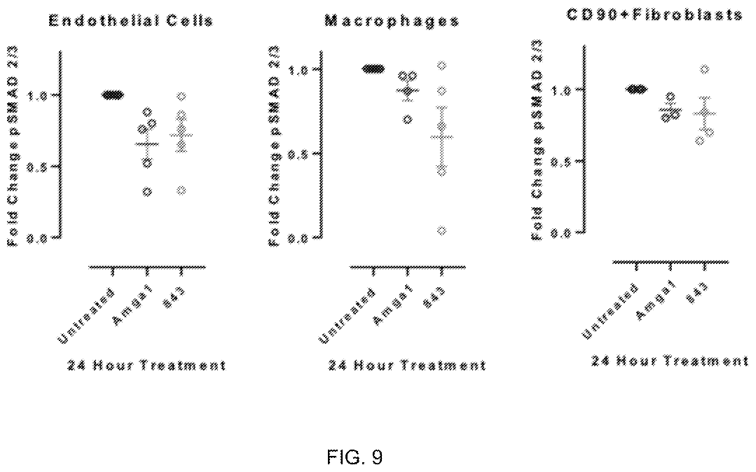

Bioinformatic analysis demonstrated significant enrichment of Activin in colorectal cancer, increasing as disease progresses. This led us to test effects of activin antibodies to inhibit downstream SMAD signaling in tissue explant studies. In this system, tumors were obtained from colorectal cancer patients at time of surgical resection. Fresh viable tumors were sectioned to approximately 100 uM thick sections and grown at a liquid air interface in standard tissue culture dishes. Antibodies were added and allowed to incubate for 24 hours. At 24 hours, tissues were processed to single cell suspensions and assessed for SMAD2/3 phosphorylation on endothelial cells, macrophages, and CD90+ fibroblasts by flow cytometry. As shown in FIG. 9, administration of activin antibodies, as demonstrated with both Amga1 (Control 1) and clone 843 result in decreased SMAD phosphorylation. This protocol was adapted from published protocol in Knoblich et al., PLOS Biology 2018. Treatment of colorectal cancer tissue slices ex vivo by anti-Activin antibodies demonstrates reduction in pSMAD2/3 in endothelial cells, macrophages, and fibroblasts.

### Example 5: TCGA Analysis of Activin a and/or Activin B in Oncology Indications

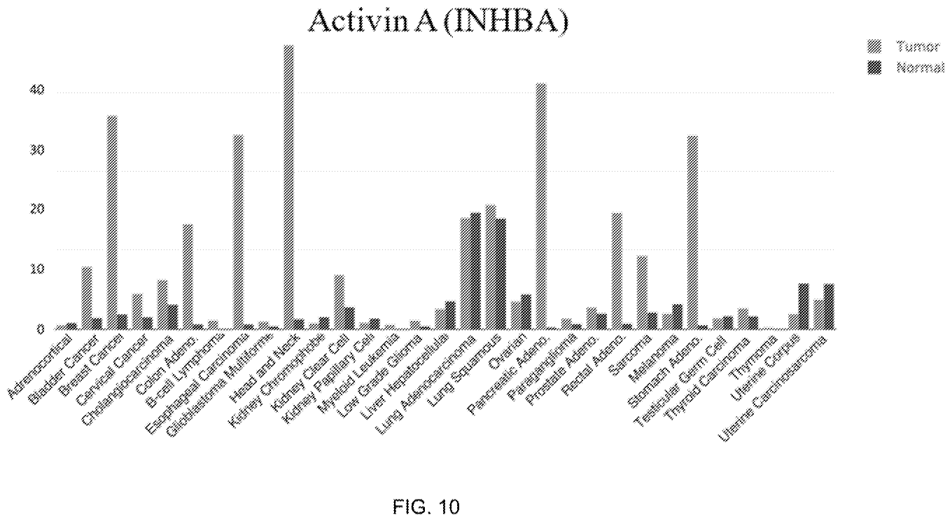

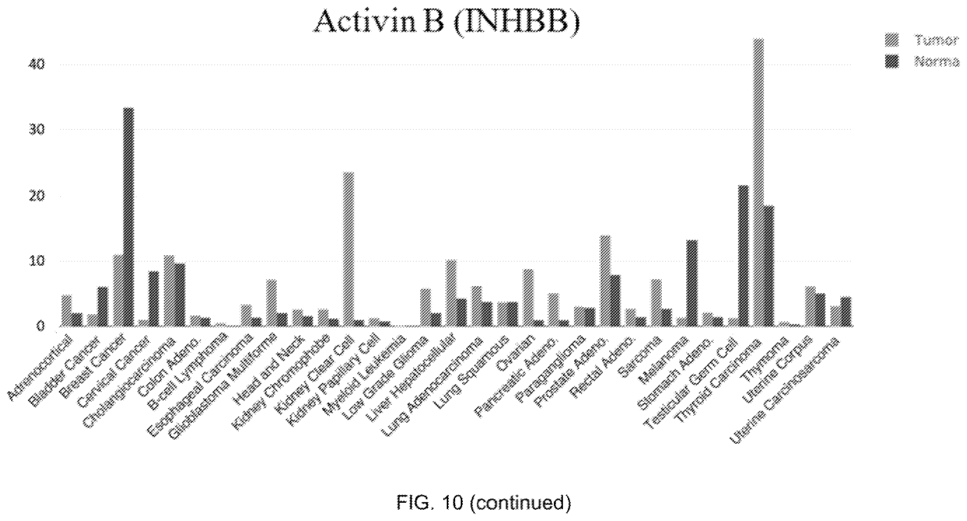

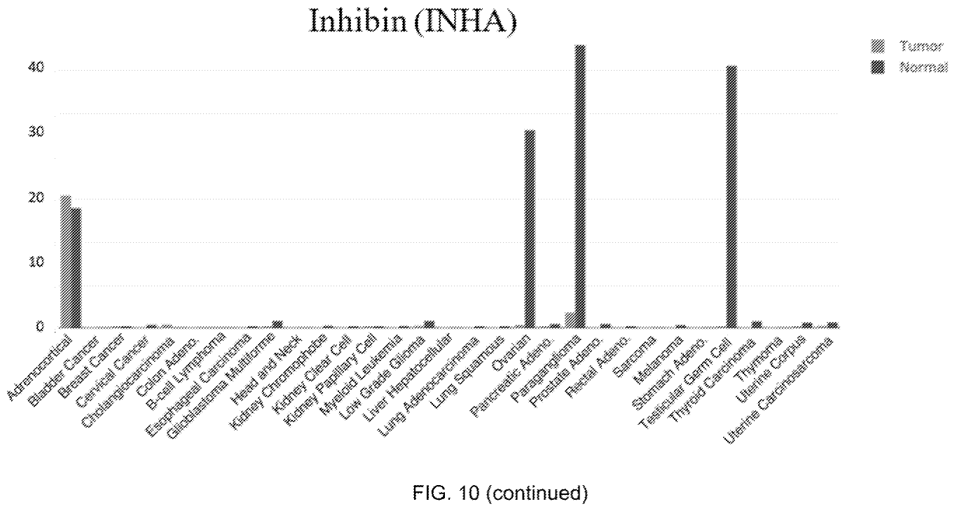

The Cancer Genome Atlas (TCGA) is a cancer genomics program that includes sequences and molecular characterization of over 20000 cases of primary cancer samples and matched normal samples spanning over 30 cancer types. TCGA analysis was performed with regard to Actvin A (INHBA), Activin B (INHBB) and Inhibin (INHA) for a variety of cancers. The data is depicted in FIG. 10. The data illustrates increased expression of Activin A and/or Activin B in oncology indications, demonstrating importance of antibodies that bind both Activin A and Activin B isoforms. Activin B expression is higher overall, and inhibin expression is not increased in tumors relative to normal tissue and has low/non-detectable expression.

TABLE 7
Additional Biological Sequences

| SEQ | | |
|---|---|---|
| ID | | |
| NO. | Name | Sequence |
| 71 | Control | QVQLVQSGAEVKKPGASVKVSCKASGYTFTSYGLSWVRQAPGQG |
| | 1 Ab | LEWMGWIIPYNGNTNSAQKLQGRVTMTTDTSTSTAYMELRSLRSD |
| | (heavy | DTAVYFCARDRDYGVNYDAFDIWGQGTMVTVSS |
| | chain) | |
| 72 | Control | SYEVTQAPSVSVSPGQTASITCSGDKLGDKYACWYQQKPGQSPVL |
| | 1 Ab | VIYQDSKRPSGIPERFSGSNSGNTATLTISGTQAMDEADYYCQAWD |
| | (light | SSTAVFGGGTKLTVL |
| | chain) | |
| 73 | Control | QVQLQESGPGLVKPSETLSLTCTVSGGSFSSHFWSWIRQPPGKGLE |
| | 2 Ab | WIGYILYTGGTSFNPSLKSRVSMSVGTSKNQFSLKLSSVTAADTAV |
| | (heavy | YYCARARSGITFTGIIVPGSFDIWGQGTMVTVSSASTKGPSVFPLAP |
| | chain) | CSRSTSESTAALGCLVKDYFPEPVTVSWNSGALTSGVHTFPAVLQS |
| | | SGLYSLSSVVTVPSSSLGTKTYTCNVDHKPSNTKVDKRVESKYGPP |
| | | CPPCPAPEFLGGPSVFLFPPKPKDTLMISRTPEVTCVVVDVSQEDPE |
| | | VQFNWYVDGVEVHNAKTKPREEQFNSTYRVVSVLTVLHQDWLNG |
| | | KEYKCKVSNKGLPSSIEKTISKAKGQPREPQVYTLPPSQEEMTKNQ |
| | | VSLTCLVKGFYPSDIAVEWESNGQPENNYKTTPPVLDSDGSFFLYS |
| | | RLTVDKSRWQEGNVFSCSVMHEALHNHYTQKSLSLSLGK |
| 74 | Control | EIVLTQSPGTLSLSPGERATLSCRASQSVSSSYLAWYQQKPGQAPRL |
| | 2 Ab | LIYGASSRATGIPDRFSGSGSGTDFTLTISRLEPEDFAVYYCQQYGSS |
| | (light | PWTFGQGTKVEIKRTVAAPSVFIFPPSDEQLKSGTASVVCLLNNFYP |
| | chain) | REAKVQWKVDNALQSGNSQESVTEQDSKDSTYSLSSTLTLSKADY |
| | | EKHKVYACEVTHQGLSSPVTKSFNRGEC |
| 75 | Human | ASTKGPSVFPLAPSSKSTSGGTAALGCLVKDYFPEPVTVSWNSGAL |
| | IgG | TSGVHTFPAVLQSSGLYSLSSVVTVPSSSLGTQTYICNVNHKPSNTK |
| | heavy | VDKKVEPKSCDKTHTCPPCPAPELLGGPSVFLFPPKPKDTLMISRTP |
| | chain | EVTCVVVDVSHEDPEVKFNWYVDGVEVHNAKTKPREEQYNSTYR |
| | component | VVSVLTVLHQDWLNGKEYKCKVSNKALPAPIEKTISKAKGQPREP |
| | | QVYTLPPSRDELTKNQVSLTCLVKGFYPSDIAVEWESNGQPENNYK |
| | | TTPPVLDSDGSFFLYSKLTVDKSRWQQGNVFSCSVMHEALHNHYT |
| | | QKSLSLSPGK |
| 76 | Human | RTVAAPSVFIFPPSDEQLKSGTASVVCLLNNFYPREAKVQWKVDNA |
| | IgG light | LQSGNSQESVTEQDSKDSTYSLSSTLTLSKADYEKHKVYACEVTHQ |
| | chain | GLSSPVTKSFNRGEC |
| | component | |
| 77 | Human | ASTKGPSVFPLAPSSKSTSGGTAALGCLVKDYFPEPVTVSWNSGAL |
| | IgG | TSGVHTFPAVLQSSGLYSLSSVVTVPSSSLGTQTYICNVNHKPSNTK |
| | heavy | VDKKVEPKSCDKTHTCPPCPAPELLGGPSVFLFPPKPKDTLMISRTP |
| | chain | EVTCVVVDVSHEDPEVKFNWYVDGVEVHNAKTKPREEQYNSTYR |
| | component | VVSVLTVLHQDWLNGKEYKCKVSNKALPAPIEKTISKAKGQPREP |
| | | QVYTLPPSRDELTKNQVSLTCLVKGFYPSDIAVEWESNGQPENNYK |
| | | TTPPVLDSDGSFFLYSKLTVDKSRWQQGNVFSCSVMHEALHNHYT |
| | | QKSLSLSPGK |
| 100 | AB448 | CAAGTCCAGCTGCAAGAATCAGGCCCCGGACTCGTTAAGCCCAG |
| | heavy | TCAGACCTTGTCCCTTACCTGCACTGTCTCAGGTCTCAGCCTCAG |
| | chain | CAATGCGAAAATGGGAGTGAATTGGATCCGGCAACCACCAGGA |
| | poly- | AAGGGTCTTGAGTGGATTGGGTATGTTTCAACTACTGGAACTAC |
| | nucleotide | CGATTACAATCCATCTCTCAAGTCCCGGGTGACAATGAGTGTAG |
| | | ATACTTCTAAGAACCAATTTAGCTTGAAAGTGAATTCCGTAACG |
| | | GCTGCGGACACGGCAGTCTATTACTGCGCTCGAGCCGGCTACCC |
| | | TGACGTTTGGGGCAAAGGCACAACAGTCACTGTGTCCTCT |
| 101 | AB465 | CAAGTCCAACTCCAAGAATCCGGGCCCGGCCTTGTCAAACCCAG |
| | heavy | TCAAACACTCTCTCTCACATGCACAGTAAGCGGGGCCTCAGTAA |
| | chain | GCAATGCACGAACTGGAGTGAACTGGATCCGGCAACCCCCAGG |
| | poly- | GAAAGGACTCGAATGGATTGGATATATTAACCCCAGTGGGAGA |
| | nucleotide | ATCGATTATAATCCATCCCTCAAAAGCAGGGTCACCATGTCCGT |
| | | GGACACATCCAAGAATCAATTCAGCTTGAAGGTGAACTCCGTAA |
| | | CAGCGGCTGACACTGCCGTGTATTACTGTGCACGGGCAGGATAC |
| | | CCTGATGTTTGGGGCAAGGGAACCACTGTCACAGTGAGTAGT |
| 102 | AB340 | GAAGTTCAGCTCGTGGAAAGCGGTGGCGGATTGGTGCAACCTGG |
| | heavy | CGGATCCCTTAGACTGAGTTGCGCCGCTTCTGGGTTTACTGCGA |
| | chain | GCAGAAACTATATGTCATGGGTACGACAGGCCCCGGGTAAAGG |
| | poly- | ACTGGAGCTGGTGGCATCTATCTCAGGGTCAGGTGACTACACCT |
| | nucleotide | ACTACCCTGATAGCGTTAAAGGACGGTTCACAATCAGCAGGGAT |
| | | AATGCTAAGAACTCCTTGTATCTCCAGATGAACAGCCTCAGGGC |
| | | CGAGGATACAGCAGTGTACTATTGCGCGCGTAGTCCCAGAGGGC |
| | | CGCGTCTGCTGGGCTATTACTATGGGATGGACGTGTGGGGACAA |
| | | GGAACTACAGTGACTGTCTCATCA |
| 103 | AB342 | GAAGTTCAATTGGTAGAATCTGGGGGGGGGTTGGTTCAGCCTGG |
| | heavy | CGGCTCACTGAGGCTTAGCTGTGCAGCTTCCGGGTTCGATTTTGG |
| | chain | AGAACATTGGATGTCCTGGGTAAGACAAGCACCAGGAAAAGGG |
| | poly- | CTTGAACTGGTGGCCTCTATAAAGGGAGATGCATCCGACACCTA |
| | nucleotide | CTACCCCGATTCCGTCAAAGGCCGATTTACCATCTCACGGGATA |
| | | ACGCTAAGAATTCTCTTTACTTGCAAATGAATTCTCTCCGCGCTG |
| | | AAGACACCGCAGTCTATTATTGTGTAAAGGACTTGTCAAAGCAG |
| | | TGGCTGGGCATTGACTACTGGGGCCAAGGGACCCTTGTGACCGT |
| | | TAGTAGT |
| 104 | AB344 | GAAGTTCAACTGGTAGAATCTGGTGGCGGTCTTGTGCAACCCGG |
| | heavy | CGGCAGTCTGCGACTCTCCTGTGCAGCGAGCGGGTATACATTCT |
| | chain | CTAACTACGGGATGTCATGGGTTCGTCAAGCGCCAGGGAAAGGC |
| | poly- | CTCGAACTCGTCGCTAGTATATCCAAGTCTGGTTCTTCCATCTAC |
| | nucleotide | TATCCAGACTCCGTGAAGGGAAGGTTTACCATCAGCAGAGATAA |
| | | TGCGAAGAACTCACTTTACCTGCAGATGAACTCCTTGCGCGCGG |
| | | AAGACACAGCCGTCTACTATTGCGCCCGACTTCCCAGGTTGGCT |
| | | TGGAATTATTATTACGGGATGGATGTTTGGGGACAAGGCACTAC |
| | | TGTCACTGTTTCCAGC |
| 105 | AB353 | CAAGTTCAACTTCAGGAATCTGGCCCTGGCTTGGTCAAACCGTC |
| | heavy | CCAAACTCTCAGTCTCACTTGCACTGTTTCAGGACTTTCTCTTAG |
| | chain | TAATGCGAAGATGGGCGTGAATTGGATAAGGCAACCACCCGGT |
| | poly- | AAAGGTCTCGAATGGATTGGCTATGTTTCAACCACTGGCACTAC |
| | nucleotide | GGATTATAATCCAAGTCTGAAATCTCGAGTAACAATGAGCGTTG |
| | | ATACCAGCAAGAATCAGTTCAGCCTGAAGGTGAACAGTGTTACA |
| | | GCCGCTGACACAGCTGTGTACTATTGTGCTAGGGCTGGATACCC |
| | | TGATGTGTGGGGTAAGGGGACAACTGTCACGGTAAGCTCA |
| 106 | AB438 | CAAGTCCAACTCCAAGAGAGCGGCCCCGGGCTCGTCAAACCTAG |
| | heavy | CCAAACCCTGTCACTGACATGCACAGTCTCTGGAGTGTCTCTGTC |
| | chain | CAACGCACGCTTGGGCGTTAACTGGATACGCCAGCCACCCGGAA |
| | poly- | AAGGTCTCGAATGGATTGGTTATGGGAGCGTGACAGGCTCTACC |
| | nucleotide | GATTATAACCCAAGTCTTAAATCCAGGGTCACGATGTCTGTTGA |
| | | CACAAGCAAGAATCAATTTAGCTTGAAGGTCAACTCCGTCACTG |
| | | CTGCAGACACTGCAGTATATTACTGTACAACCAGCTATCTGGTG |
| | | GACACGGCTATGGTAACCAGGTGGGGTCAGGGTACACTCGTCAC |
| | | AGTGTCCTCT |
| 107 | AB366 | CAAGTTCAGCTCCAAGAATCCGGTCCAGGCCTGGTGAAGCCCTC |
| | heavy | TCAAACATTGAGCCTCACCTGCACCGTAAGCGGCTACAGTTTGA |
| | chain | CAGATGCTAGGATAGGTGTTAACTGGATCCGTCAACCGCCGGGC |
| | poly- | AAAGGTTTGGAGTGGATTGGCTATATAAACCCAAGCGGGCGCAC |
| | nucleotide | AGATTACAATCCCTCACTCAAGTCCCGTGTGACTATGTCAGTCG |
| | | ATACTAGCAAGAATCAATTCAGCCTTAAGGTGAATTCAGTAACC |
| | | GCAGCCGATACAGCCGTCTATTACTGTGCTAGAGCCGGGTATCC |
| | | TGACGTCTGGGGCAAAGGGACAACAGTAACAGTTAGTAGT |
| 108 | AB341 | GAAGTGCAACTTGTAGAATCTGGTGGCGGGCTCGTCCAACCTGG |
| | heavy | CGGATCTCTGCGTTTGTCCTGTGCCGCAAGCGGCTATACCTTTAG |
| | chain | CAATTATGGTATGAGTTGGGTCAGGCAAGCCCCGGGCAAGGGTT |
| | poly- | TGGAATTGGTCGCGAGCATATCAAAGTCTGGGAGTAGCATTTAC |
| | nucleotide | TATCCCGACAGTGTAAAGGGGCGCTTTACCATAAGCCGCGATAA |
| | | CGCTAAGAATTCCCTCTATCTCCAAATGAATTCCCTTCGTGCAGA |
| | | AGATACAGCAGTTTACTATTGCGCGAGACTTCCACGTCTTGCAT |
| | | GGAACTACTATTATGGAATGGACGTTTGGGGCCAAGGTACTACC |
| | | GTCACAGTAAGTTCA |
| 109 | AB430 | GAAGTGCAACTCGTGGAATCAGGTGGCGGATTGGTACAACCCG |
| | heavy | GCGGCAGCTTGCGTCTCTCTTGTGCCGCCTCTGGTTTCACTCTGA |
| | chain | GCAACTACCCAATGAGTTGGGTAAGACAAGCACCTGGAAAGGG |
| | poly- | CCTGGAGCTTGTAGCGAGTACTACAAGCGGTAGCTCCACCATCT |
| | nucleotide | ACTACCCGGACTCAGTCAAAGGGCGATTCACGATTAGCCGAGAC |
| | | AACGCTAAGAACTCTTTGTATCTTCAAATGAACTCTCTGCGTGCT |
| | | GAAGACACCGCGGTCTATTACTGCGCTCGCGGTCCAGACTATTA |
| | | TGCACCGCTTAGGTCCTACATGGATGTCTGGGGCAAGGGAACCA |
| | | CCGTGACTGTATCTTCT |
| 110 | AB349 | CAAGTGCAACTCCAAGAAAGTGGGCCTGGACTCGTGAAGCCCA |
| | heavy | GCCAAACTCTGAGTCTTACCTGCACCGTCAGCGGAGTCAGTCTC |
| | chain | TCCAACGCTCGTCTTGGTGTAAATTGGATCCGGCAGCCACCAGG |
| | poly- | GAAAGGACTGGAGTGGATCGGTTATGGATCCGTCACGGGAAGC |
| | nucleotide | ACCGATTACAATCCCTCTCTCAAAAGTAGGGTGACTATGAGCGT |
| | | CGATACTTCTAAGAATCAATTCAGCCTCAAAGTTAATTCTGTTAC |
| | | CGCAGCTGACACTGCGGTGTATTATTGCACGACTAGCTATTTGG |
| | | TGGACACAGCTATGGTCACGCGGTGGGGCCAAGGAACACTGGTT |
| | | ACTGTCTCTTCT |
| 111 | AB449 | CAAGTTCAACTGCAAGAAAGCGGCCCAGGCCTCGTGAAACCAA |
| | heavy | GCCAAACACTCAGCCTGACATGCACTGTATCTGGCCTCAGCCTT |
| | chain | AGTAACGCCAAAATGGGCGTGAACTGGATTCGGCAACCACCGG |
| | poly- | GCAAAGGGCTTGAATGGATTGGCTACGTGTCCACAACCGGTACG |
| | nucleotide | ACGGATTACAATCCATCCCTGAAATCCCGTGTCACCATGAGCGT |
| | | TGACACGTCCAAGAACCAATTCAGCTTGAAAGTAAATAGCGTAA |
| | | CAGCCGCAGACACAGCTGTCTATTATTGTGCGCGGGCAGGGTAT |
| | | CCAGATGTCTGGGGCAAGGGCACTACAGTCACTGTTAGCAGC |
| 112 | AB338 | GAAGTGCAATTGGTTGAAAGTGGTGGTGGATTGGTCCAACCTGG |
| | heavy | TGGCTCCCTGAGACTCAGCTGCGCTGCATCCGGTTTCACAGCTTC |
| | chain | TCGAAATTACATGAGCTGGGTACGACAAGCACCAGGGAAAGGA |
| | poly- | CTTGAACTGGTTGCCAGTATCAGTGGCAGTGGGGACTATACATA |
| | nucleotide | CTATCCCGATTCTGTCAAAGGCAGGTTCACCATTAGCCGCGACA |
| | | ACGCTAAGAATTCACTTTATTTGCAGATGAATTCACTTAGGGCT |
| | | GAGGACACTGCTGTCTATTACTGTGCCAGATCACCCAGGGGCCC |
| | | GCGGTTGCTGGGGTATTATTATGGAATGGACGTATGGGGCCAGG |
| | | GAACAACCGTCACTGTTTCTTCT |
| 113 | AB435 | CAGGTGCAACTCCAAGAATCAGGCCCGGGGCTCGTTAAACCCTC |
| | heavy | ACAGACCCTCAGCCTGACATGTACCGTCAGTGGTTATTCTCTGA |
| | chain | CGGACGCCAGAATCGGTGTCAACTGGATAAGGCAACCACCAGG |
| | poly- | GAAAGGCTTGGAATGGATCGGTTATATTAACCCTAGTGGGCGCA |
| | nucleotide | CTGACTATAACCCCAGCCTTAAAAGCCGGGTGACTATGTCAGTA |
| | | GATACTTCTAAGAATCAATTCAGTCTGAAAGTCAACTCCGTGAC |
| | | GGCAGCGGATACCGCCGTCTATTATTGCGCCAGGGCTGGTTATC |
| | | CTGACGTGTGGGGCAAGGGCACCACTGTTACGGTTTCTTCT |
| 114 | AB372 | CAAGTTACCCTGCGAGAAAGTGGTCCCGCTCTTGTCAAGCCCAC |
| | heavy | CCAAACCTTGACCCTCACTTGTACCGTAAGTGGCTTTAGCTTGTC |
| | chain | CACATCCGGAGCAACAGTCAATTGGATCCGACAACCCCCGGGG |
| | poly- | AAGGCCCTCGAATGGATTGGTTATATAACATCCTCAGGACGCAC |
| | nucleotide | TGTTTATAATTCCGCTCTGAAGAGTCGCTTGACCATAAGCAAAG |
| | | ACACCTCAAAGAATCAGGTGGTGCTGACCATGACTAATATGGAC |
| | | CCCGTCGATACTGCCACCTATTATTGTACCACCACCAAAATTGTG |
| | | GGCGCTACCCTGAGAGATTATTGGGGTCAAGGAACACTCGTAAC |
| | | GGTTTCCAGT |
| 115 | AB439 | GAAGTTCAACTGGTGCAGTCAGGAGCCGAAGTAAAGAAACCAG |
| | heavy | GCGCATCTGTGAAAGTTTCATGTAAGGCTTCCGGCTACACTTAC |
| | chain | ACAGGGTATCGAATCAGCTGGGTCCGGCAGGCTCCCGGGCAAG |
| | poly- | GTCTGGAATGGATGGGGTGGATAGATCCGAAGAGTGGGCGGAC |
| | nucleotide | TAACTACGCACAGAAACTTCAAGGTCGGGGCACAATGACTACCG |
| | | ATCCATCTACAAGTACTGCCTATATGGAACTTCGCTCCTTGAGAT |
| | | CTGATGATACCGCGGTCTACTATTGTGCCCGTGAAGTAAAACAG |
| | | TGGCTTGTCCCAGGACGTGGAGTGTACTGGTTTGACCCATGGGG |
| | | ACAAGGTACACTTGTAACTGTCAGCTCC |
| 116 | AB350 | CAGGTGCAACTGCAAGAGTCAGGCCCCGGCCTCGTTAAACCTTC |
| | heavy | TCAAACACTCAGCCTCACCTGCACGGTTAGCGGAGTTTCCCTGT |
| | chain | CCGATGCGCGGATGGGCGTCAACTGGATTAGGCAACCACCCGGC |
| | poly- | AAGGGTCTTGAGTGGATCGGATATGTCAGCACCACAGGAACCAC |
| | nucleotide | CGACTACAACCCCTCATTGAAGTCACGGGTGACCATGTCTGTGG |
| | | ACACCTCTAAGAACCAATTTTCCCTCAAAGTGAACTCCGTGACC |
| | | GCTGCGGACACTGCAGTTTATTATTGTGCCAGGGCCGGGTACCC |
| | | GGACGTGTGGGGTAAGGGTACTTTGGTGACAGTGAGTAGT |
| 117 | AB363 | CAAGTCACCTTGAGAGAATCCGGACCCGCGCTCGTAAAACCCAC |
| | heavy | TCAAACCCTGACATTGACTTGTACTGTCTCAGGTTTCAGTCTCTC |
| | chain | AACTAGCGGAGCGACAGTCAACTGGATACGTCAACCACCAGGA |
| | poly- | AAGGCCCTGGAGTGGATTGGATACATTACATCTAGTGGCAGGAC |
| | nucleotide | CGTGTATAATTCTGCCCTCAAATCCCGCCTTACAATCTCCAAAGA |
| | | CACATCCAAGAATCAAGTAGTCCTGACCATGACTAATATGGACC |
| | | CTGTGGACACGGCCACCTACTATTGCACAACAACCAAAATTGTT |
| | | GGCGCGACCCTGCGTGATTATTGGGGCCAAGGCACTCTGGTGAC |
| | | CGTTAGCAGC |
| 118 | AB367 | CAGGTGACCTTGAGGGAGTCCGGGCCAGCACTTGTTAAACCAAC |
| | heavy | GCAAACCCTTACTCTCACGTGCACTGTATCTGGGTTCACCCTGTC |
| | chain | AAATCCTAGAATGGGCGTTAACTGGATACGCCAACCGCCAGGTA |
| | poly- | AGGGACTGGAGTGGATCGGGTACATAAGCACCAGCGGACGCAC |
| | nucleotide | CGACTATAACCCTAGTCTCAAAAGTCGCGTCACGATGTCAGTGG |
| | | ACACCTCAAAGAATCAATTCAGCCTGAAGGTCAATTCAGTTACT |
| | | GCTGCAGATACGGCTGTTTATTACTGTGCCAGAGCTGGCTACCC |
| | | AGACGTATGGGGAAAAGGAACGACAGTGACAGTCAGTAGC |
| 119 | AB352 | CAAGTGCAGCTGCAAGAAAGTGGACCAGGACTGGTGAAACCGA |
| | heavy | GCCAAACATTGTCCCTCACCTGTACCGTCTCTGGGTTTCCCTTGA |
| | chain | GTACCAGCCGCATGGGCGTGAACTGGATTCGGCAGCCACCTGGA |
| | poly- | AAGGGCCTGGAATGGATTGGTTACATCAATCCTTCCGGGGCCAC |
| | nucleotide | TGATTATAACCCTTCCTTGAAAAGCAGGGTTACTATGTCTGTGG |
| | | ACACCAGCAAGAACCAATTTAGTCTTAAGGTGAACTCCGTGACC |
| | | GCTGCCGATACTGCGGTTTATTACTGCGCACGTGCGGGGTATCC |
| | | AGATGTATGGGGCAAAGGAACTACTGTGACTGTATCTTCT |
| 120 | AB357 | GAAGTCCAATTGGTGCAAAGCGGCGCGGAAGTGAAGAAACCTG |
| | heavy | GAGCCTCAGTTAAAGTCAGTTGTAAAGCTTCTGGGTACACCTTC |
| | chain | CCGTCTCACTACATATCCTGGGTCCGGCAGGCCCCAGGGCAAGG |
| | poly- | TTTGGAATGGATGGGCGGGATGAACCCAATTACAGGGCACACTA |
| | nucleotide | TTTATGCCCAAAAGCTGCAAGGAAGGGGCACAATGACTACCGA |
| | | CCCATCCACATCCACTGCATATATGGAATTGCGTAGTCTCCGGA |
| | | GCGATGATACCGCCGTGTACTATTGTGCAAGAGATATGCGCTAC |
| | | GATATCCTGCCAGGGCGGTCATACTATTATGGTATGGATGTTTG |
| | | GGGCCAAGGCACTACCGTCACGGTTTCATCA |
| 121 | AB347 | CAAGTCCAATTGCAAGAGTCTGGCCCGGGGCTGGTAAAACCTTC |
| | heavy | TCAAACGCTTAGCCTGACTTGCACAGTGTCCGGGTTTCCCTTGTC |
| | chain | AACTAGCCGTATGGGCGTTAATTGGATTCGACAACCACCAGGTA |
| | poly- | AAGGACTTGAGTGGATAGGCTATATCAACCCCTCTGGCGCGACC |
| | nucleotide | GATTACAACCCTAGTCTCAAATCCCGGGTCACCATGAGTGTGGA |
| | | TACCTCAAAGAATCAGTTCTCCCTCAAAGTAAACTCCGTGACGG |
| | | CTGCAGACACCGCAGTGTATTACTGTGCACGGGCTGGTTACCCC |
| | | GATGTGTGGGGCAAAGGAACAACAGTTACTGTCTCTTCT |
| 122 | AB346 | CAAGTCCAACTGCAAGAGTCAGGCCCTGGTCTCGTTAAGCCATC |
| | heavy | CCAAACGCTGAGTCTTACCTGTACGGTATCCGGTTTCCCCCTCTC |
| | chain | CACTAGTCGAATGGGCGTAAACTGGATTCGGCAGCCACCAGGCA |
| | poly- | AAGGACTGGAATGGATAGGGTATATAAATCCGAGTGGCGCCAC |
| | nucleotide | CGATTATAACCCTAGCCTGAAATCACGTGTGACTATGAGTGTGG |
| | | ACACCAGCAAGAATCAATTTAGCTTGAAAGTCAATAGTGTGACT |
| | | GCAGCAGACACAGCAGTCTACTACTGTGCCCGCGCCGGCTATCC |
| | | TGACGTCTGGGGTAAAGGTACAACGGTGACGGTGTCATCA |
| 123 | AB433 | CAAGTCCAATTGCAAGAAAGCGGCCCAGGTCTGGTCAAGCCCA |
| | heavy | GTCAGACACTCTCACTGACATGTACCGTTTCAGGCTTTCCCCTTT |
| | chain | CAACTTCTCGTATGGGCGTCAACTGGATTAGGCAGCCACCAGGA |
| | poly- | AAAGGCCTGGAGTGGATAGGATATATCAATCCATCTGGGGCGAC |
| | nucleotide | TGATTATAATCCCAGCTTGAAGAGCCGCGTTACAATGAGTGTCG |
| | | ATACCAGCAAGAATCAATTCTCCCTGAAAGTTAACAGTGTTACA |
| | | GCCGCAGATACAGCGGTTTACTACTGCGCCCGCGCCGGATACCC |
| | | CGACGTGTGGGGAAAAGGTACCACCGTCACAGTCAGCTCC |
| 124 | AB432 | CAAGTGCAACTGCAAGAATCAGGTCCCGGCCTCGTTAAGCCCAG |
| | heavy | CCAAACCCTCAGTCTTACCTGTACCGTGAGTGGCGTGAGTTTGTC |
| | chain | TAATGTACGCATGGGCGTCAATTGGATCCGACAACCACCCGGGA |
| | poly- | AGGGACTTGAGTGGATTGGGTATGTTAATCCTTCCGGGTCAACT |
| | nucleotide | GATTATAATCCATCACTGAAATCACGGGTCACCATGTCTGTTGA |
| | | TACTTCCAAGAACCAATTTTCATTGAAAGTTAACTCCGTGACGG |
| | | CAGCAGACACAGCAGTTTATTACTGTGCCCGCGCAGGATATCCT |
| | | GATGTGTGGGGTAAGGGCACTCTCGTAACGGTTTCCTCT |
| 125 | AB326 | CAAGTCCAACTTGTGCAAAGCGGCGCTGAAGTTAAGAAACCTGG |
| | heavy | AGCTAGTGTCAAGGTCAGCTGCAAAGTGAGCGGTTATACCTTTA |
| | chain | CAAGTAGCGCAATCAGTTGGGTACGGCAAGCTCCAGGCAAAGG |
| | poly- | CCTGGAATGGATGGGCGGGATATACCCAGGAAGCGGACACACT |
| | nucleotide | ATTTACGCTCAAAAGTTTCAAGGCCGCGTGACGATGACCGAGGA |
| | | TACATCCACGGATACGGCTTATATGGAGTTGTCTTCACTGAAGT |
| | | CTGAAGACACTGCTGTGTACTATTGTGCTCGGGAAGGCGACATC |
| | | TTTGTGATGGATGTTTGGGGTCTGGGTACAGCCGTCACGGTCAG |
| | | TTCT |
| 126 | AB428 | GAAGTACAGCTTGTTGAAAGTGGCGGTGGGCTCGTCCAACCCGG |
| | heavy | TGGATCCCTCAGACTCAGCTGTGCAGCGAGCGGCTTCACTTCAT |
| | chain | CAAATCATTGGATGAGTTGGGTGAGGCAAGCCCCAGGAAAGGG |
| | poly- | CTTGGAACTTGTGGCCAGTATTGGCCCTAAAGGATTGGGTACTT |
| | nucleotide | ACTACCCCGATTCTGTCAAGGGCCGCTTTACAATCAGCAGGGAC |
| | | AATGCCAAGAACTCTTTGTATCTGCAAATGAATTCTCTTCGCGCT |
| | | GAAGACACCGCCGTTTACTATTGTGCATCTATTAGAGACTATGG |
| | | AGACAAGAAGGGCGGCAGCTGGTTTGACCCCTGGGGTCAGGGA |
| | | ACACTTGTTACCGTAAGTAGT |
| 127 | AB426 | GAAGTTCAACTGGTGGAATCTGGTGGTGGTCTGGTGCAACCTGG |
| | heavy | TGGTTCATTGCGTCTCTCTTGCGCAGCCTCAGGTTTCACATTTTC |
| | chain | TGATCATCATATGAGCTGGGTGCGACAGGCGCCCGGTAAAGGCC |
| | poly- | TCGAGCTTGTCGCTTCAATCAGCCCTAGTGGTGATTTCACCTACT |
| | nucleotide | ACCCAGATAGCGTCAAAGGACGGTTTACCATCTCCCGGGATAAT |
| | | GCAAAGAACAGTCTGTATTTGCAAATGAATAGCTTGCGTGCAGA |
| | | GGATACAGCTGTCTATTATTGCGCTAGGGATGTGAGCCCCGTGT |
| | | ACTACTGGGGCCAAGGCGCTCTGGTAACCGTAAGCTCC |
| 128 | AB345 | GAAGTACAGCTTGTGGAGAGTGGAGGAGGTTTGGTGCAACCTG |
| | heavy | GTGGGAGCCTGCGACTCTCCTGTGCTGCATCCGGATTCACTAGC |
| | chain | TCCGGATATTGGATGTCATGGTTTAGACAGGCTCCAGGGAAGGG |
| | poly- | GCTTGAGCTCGTGGCGAGTATCGGGACTGCCGGAGTTACCTATT |
| | nucleotide | ATCCCGATTCAGTGAAAGGACGGTTCACCATTAGCCGCGACAAT |
| | | GCCAAGAATTCCCTGTACCTTCAAATGAACTCCTTGAGAGCGGA |
| | | AGATACAGCAGTTTATTACTGCGCGAGAGGCCCTCATGGTCCCC |
| | | AACAAAGATTGCGCTTCGATCCATGGGGACAAGGCACTCTGGTA |
| | | ACTGTTAGTAGT |
| 129 | AB431 | GAAGTGCAGCTCGTGGAGAGTGGAGGAGGTCTTGTACAACCCG |
| | heavy | GTGGATCCCTGCGACTCTCCTGCGCTGCCAGCGGATTCACAAGT |
| | chain | AGTAACCATTGGATGTCATGGGTGAGGCAGGCACCCGGTAAAG |
| | poly- | GCCTGGAGTTGGTCGCTTCAATAGGACCCAAGGGCCTTGGTACC |
| | nucleotide | TATTATCCAGACAGTGTGAAGGGTCGGTTCACTATTAGTCGCGA |
| | | TAACGCTAAGAATTCACTGTATCTTCAGATGAATTCTTTGCGTGC |
| | | CGAAGATACTGCTGTCTATTACTGTGCTAGAGACGTTCGAGGTT |
| | | GGAGAGCACTGTATTATTATTACGGTATGGACGTGTGGGGTCAA |
| | | GGGACCACCGTGACCGTCTCTTCT |
| 130 | AB330 | CAGGTGCAACTTGTTCAGAGCGGAGCCGAAGTAAAGAAACCAG |
| | heavy | GGGCCTCTGTTAAAGTGTCTTGCAAAGTTTCAGGGTACCCAAGC |
| | chain | CCTGCTACCATATCTTGGGTACGGCAAGCGCCCGGCAAAGGACT |
| | poly- | CGAATGGATGGGATGGATGAATCCCAAAAGTGGATATACGAAT |
| | nucleotide | TACGCACAAAAGTTTCAAGGCAGAGTCACCATGACGGAAGATA |
| | | CCTCAACTGATACCGCTTATATGGAACTCTCATCACTTAAATCTG |
| | | AAGATACCGCTGTCTATTACTGCGCAACACTCCACGGTAGTGGT |
| | | CTCGCTTTTGATATTTGGGGACAGGGGACTATGGTCACGGTCAG |
| | | CAGC |
| 131 | AB731 | CAAGTGCAACTACAACAACCAGGTACTGAACTGGTGAAACCCG |
| | heavy | GCGCTTCCGTGAAGCTGTCCTGCAAGGCCTCTGGCTACACCTTC |
| | chain | ACCTCCTACTGGATGCACTGGGTCAAGCAGAGACCTGGCCAGGG |
| | poly- | CCTGGAATGGATCGGCAACATCAATCCTTCCAACGGCGGAACCA |
| | nucleotide | ACTACAACGAGAAGTTCAAGTCCAAGGCTACCCTGACCGTGGAC |
| | | AAGTCTAGCTCCACCGCCTACATGCAGCTGTCTTCTCTGACCTCT |
| | | GAGGACTCTGCCGTGTACTACTGTTCCATCACCACAGTGGTGGC |
| | | CTATTGGTACTTTGATGTGTGGGGCACAGGAACCACCGTGACCG |
| | | TGTCCAGC |
| 132 | AB843 | GAAGTGCAACTAGTGCAAAGTGGTGCAGAAGTCAAGAAGCCTG |
| | heavy | GAGCTTCCGTGAAAGTGTCCTGCAAGGCCTCCGGCGACACATCC |
| | chain | TCTCATAGCCACATCCACTGGGTGCGGCAGGCTCCTGGCCAAGG |
| | poly- | CCTGGAGTGGATGGGCTGGATCAACCCCCGCTCTGGCGCTACCA |
| | nucleotide | ACTACGCTCAGAAGCTGCAGGGCAGAGGAACCATGACCACCGA |
| | | TCCTTCTACCTCTACAGCCTACATGGAACTGCGGTCCCTGAGATC |
| | | CGACGACACCGCCGTGTACTACTGTGCCAGAGATAGAGGCGGTC |
| | | GGACCAACGGCGTGGGCGCCTTCGACATCTGGGGCCAGGGCAC |
| | | CATGGTGACCGTGTCTAGC |
| 133 | AB852 | CAAGTGACTCTACGAGAAAGTGGTCCAGCACTGGTGAAGCCCAC |
| | heavy | CCAAACACTCACACTGACCTGTACCGTGTCCGGCTTCAGCCTGT |
| | chain | CTGATACCAGATGGGGCGTGAATTGGATCCGGCAGCCTCCTGGC |
| | poly- | AAGGCCCTGGAATGGATCGGCTACATCAACGACCGGGGAACCA |
| | nucleotide | CCGACTACAACAGCGCTCTGAAGTCCAGACTGACCATCTCCAAA |
| | | GACACCTCCAAGAACCAGGTGGTGCTGACCATGACCAACATGG |
| | | ATCCTGTGGACACCGCTACCTACTACTGCGCCCACACCCTGCAC |
| | | ACAAGAGAGTTCGTGGGCATGGACGTGTGGGGCCAGGGCACCA |
| | | CAGTGACCGTCTCTTCT |
| 134 | AB854 | GAAGTGCAACTAGTGCAAAGTGGTGCAGAAGTCAAGAAGCCTG |
| | heavy | GCGCTTCCGTGAAAGTGAGCTGCAAGGCCTCCGGCTACACCTTC |
| | chain | CCCTCTCACTACATCTCCTGGGTGCGGCAGGCTCCAGGCCAGGG |
| | poly- | CCTGGAGTGGATGGGCGGCATGAACCCTATCACCGGCCACACCA |
| | nucleotide | TCTACGCCCAGAAGCTGCAGGGCAGAGGCACCATGACCACAGA |
| | | TCCTTCTACCTCCACCGCCTACATGGAACTGCGGTCTCTGAGATC |
| | | CGACGACACCGCTGTGTACTATTGTGCCAGAGATATGAGATACG |
| | | ACATCCTGCCTGGACGGTCCTACTACTACGGCATGGACGTGTGG |
| | | GGCCAAGGAACAACCGTGACCGTGTCTAGC |
| 135 | AB448 | GATATTCAAATGACTCAATCTCCGTCTAGCGTGTCAGCTTCTGTC |
| | light | GGCGATCGCGTGACCATAACGTGTCGTGCAAGTCAAAATATTGG |
| | chain | GGCTGCCCTCGCTTGGTATCAACAAAAGCCCGGAAAGGCACCAA |
| | poly- | AGTTGCTGATTTATGGAGCATCTACCCGCGAATATGGCGTGCCT |
| | nucleotide | TCCCGGTTTTCCGGTTCCGGGAGTGGTACTGACTTTACACTGACC |
| | | ATCTCTTCACTGCAACCCGAAGACTTCGCAAACTATTATTGCCA |
| | | ACAATATACTGCATACCCCCTCACATTCGGTGGCGGAACCAAGG |
| | | TAGAGATAAAG |
| 136 | AB465 | GAGATCGTGATGACACAAAGCCCAGCTACCCTTTCACTTAGCCC |
| | light | CGGTGAAAGGGCCACTCTTTCCTGTCGCGCAAGCCAGACAGTGT |
| | chain | CATCTCATCTGGCGTGGTATCAGCAAAAGCCTGGACAAGCACCC |
| | poly- | CGGCTTCTTATCTATGACGTGTCACATCGGGCATCAGGAATTCCC |
| | nucleotide | GCCAGGTTTTCAGGGTCAGGCAGCGGAACCGATTTCACCCTCAC |
| | | CATCAGTTCACTCGAGCCAGAGGATTTCGCGGTGTACTATTGTC |
| | | AACAATATTTTACGTTGCCCACCTTTGGAGGAGGAACTAAGGTC |
| | | GAGATAAAA |
| 137 | AB340 | GACATCGTCATGACCCAAAGCCCTCTCAGTCTCCCTGTCACACCT |
| | light | GGTGAACCCGCCAGTATCTCATGCCGTTCCAGCAGATCTCTGGT |
| | chain | TCATGGTTCAGGCCATAATTACCTGCATTGGTACCTGCAAAAGC |
| | poly- | CCGGCCAATCCCCACAGTTGTTGATATATGGAGCATCTACCCGG |
| | nucleotide | GTCGCAGGCGTCCCCGACAGGTTCTCTGGCTCTGGATCCGGTAC |
| | | TGATTTCACCTTGAAGATTAGCAGAGTCGAAGCCGAAGATGTGG |
| | | GAGTATACTATTGTATGCAGGGCACGCAAGCTCCCAGGACCTTC |
| | | GGTGGCGGTACCAAGGTGGAAATTAAA |
| 138 | AB342 | GATATTGTAATGACGCAAAGTCCCCTGAGTCTGCCCGTCACACC |
| | light | CGGTGAGCCCGCAAGTATTTCCTGCAGGAGCAGTCAAAGTCTGC |
| | chain | TGCATTCCAATGAAAAGAACTATCTGCATTGGTACCTTCAGAAA |
| | poly- | CCAGGCCAAAGCCCTCAGTTGTTGATATATGATGCGTCAAAGAG |
| | nucleotide | GGCCAGTGGCGTCCCCGACCGGTTCAGCGGGTCCGGAAGCGGC |
| | | ACTGATTTTACACTCAAGATTAGCCGGGTCGAAGCTGAAGATGT |
| | | AGGAGTCTATTATTGCGTGCAAGACTTGCACACACCTTTCACATT |
| | | TGGAGGAGGTACCAAGGTTGAAATCAAG |
| 139 | AB344 | GATATAGTAATGACACAAAGTCCTCTTAGCCTGCCGGTCACTCC |
| | light | TGGTGAGCCTGCTTCTATAAGCTGTAGATCAAGTAGGTCTCTGG |
| | chain | TGCACGGGTCAGGCGACAACTACTTGCATTGGTACCTGCAAAAG |
| | poly- | CCTGGGCAGAGCCCTCAACTGCTCATCTATATGGCAAGTAACCG |
| | nucleotide | GGCTCCTGGCGTTCCGGATCGATTTTCTGGAAGCGGATCCGGAA |
| | | CAGACTTCACTCTCAAAATCTCTAGAGTCGAGGCCGAAGATGTG |
| | | GGCGTGTATTACTGCGTCCAAACCTTGCAGACCCCATCCACTTTT |
| | | GGCGGCGGAACCAAGGTCGAAATCAAA |
| 140 | AB353 | GAGATCGTGATGACCCAGTCCCCTGCAACATTGAGCCTCAGTCC |
| | light | GGGTGAAAGGGCGACCCTGTCTTGTCGCGCTTCCCAAAATGTCT |
| | chain | ATACCAACCTCGCCTGGTACCAACAAAAGCCTGGCCAGGCTCCA |
| | poly- | CGCCTGTTGATCTATGGTGCTTCCGCTCGGGCTAGCGGTATACCT |
| | nucleotide | GCCCGCTTCAGTGGCAGTGGCTCTGGAACCGACTTTACCCTTAC |
| | | CATCTCTTCTTTGGAACCAGAGGATTTCGCTGTGTACTACTGCCT |
| | | GCAGTACGGCAGTAGCCCACCCCTCTTCGGAGGAGGAACGAAA |
| | | GTGGAAATTAAG |
| 141 | AB438 | GAAATAGTAATGACTCAAAGCCCAGCTACTCTTTCATTGAGTCC |
| | light | CGGGGAACGGGCCACTCTGTCTTGCCGGGCCTCTCAATCATTGG |
| | chain | GAACCAATCTTGCATGGTACCAGCAAAAGCCCGGTCAAGCCCCT |
| | poly- | CGGTTGTTGATTTATGGAGCTTCAAGTCGGCCTAGCGGCATACC |
| | nucleotide | TGCCAGATTTTCTGGTTCCGGGTCCGGCACTGATTTTACTCTGAC |
| | | AATATCTTCCCTTGAACCAGAGGACTTTGCAGTATACTATTGCCA |
| | | ACAATATGATACACATCCGCTCAGCTTTGGTGGTGGCACTAAAG |
| | | TCGAAATCAAA |
| 142 | AB366 | GATATACAGATGACGCAATCCCCCAGCAGCGTGAGCGCTTCAGT |
| | light | CGGCGATCGGGTAACCATTACATGCAGAGCCAGTCAAACTATAA |
| | chain | GTCGGTCTTTGGCTTGGTACCAGCAGAAACCCGGAAAAGCCCCA |
| | poly- | AAACTGCTCATCTACGACGCCAGCAGTCTTGCTGGCGGAGTGCC |
| | nucleotide | ATCTCGGTTTTCCGGTTCTGGGTCTGGAACAGATTTCACACTGAC |
| | | TATCTCAAGCTTGCAACCCGAAGATTTTGCTAATTATTATTGCCA |
| | | ACAATATAGTTCATATCCAAGCCTCTTTGGCGGCGGCACAAAAG |
| | | TAGAGATTAAG |
| 143 | AB341 | GATATCGTAATGACACAATCCCCGCTGTCTCTCCCGGTGACGCC |
| | light | AGGCGAGCCTGCGTCTATCTCCTGTAGGAGTTCACAAAGTCTCC |
| | chain | TGCACTCTAACGAGAAGAACTACCTGCATTGGTACTTGCAAAAG |
| | poly- | CCGGGACAGAGCCCTCAGCTCCTCATCTACGATGCATCAAAACG |
| | nucleotide | AGCCTCAGGCGTCCCTGATCGGTTCTCCGGAAGTGGATCTGGGA |
| | | CGGACTTTACTTTGAAGATTAGCCGCGTAGAAGCCGAGGATGTG |
| | | GGCGTCTATTATTGCGTACAAGATCTCCACACACCGTTTACCTTC |
| | | GGTGGTGGGACCAAGGTGGAGATCAAA |
| 144 | AB430 | GATATCGTCATGACTCAATCACCTCTCTCCTTGCCCGTGACTCCC |
| | light | GGAGAACCGGCCAGCATTAGTTGCCGGTCCAGTCAAAGTTTGCA |
| | chain | ACAAAGCGAAGGCCGGACCTACCTGCACTGGTACCTGCAAAAG |
| | poly- | CCCGGGCAGAGTCCGCAGCTGCTGATTTACGACGCCAGTAAGCG |
| | nucleotide | CGCATCCGGTGTTCCGGATAGATTTTCCGGTAGTGGAAGCGGAA |
| | | CAGATTTTACTTTGAAGATTAGCCGCGTTGAGGCTGAGGACGTT |
| | | GGCGTTTACTATTGCATGCAAGTCCTTAGAACACCGATTACTTTC |
| | | GGAGGAGGCACGAAGGTGGAAATCAAA |
| 145 | AB349 | GAAATTGTCATGACACAATCCCCAGCTACCCTTAGCCTTTCCCCT |
| | light | GGAGAGAGGGCAACACTTTCTTGTCGTGCCAGCCAACGAATTGG |
| | chain | ATCCGACTTGGCATGGTACCAACAGAAGCCGGGTCAAGCACCAC |
| | poly- | GTCTCCTCATCTATTCTGCTAGCTCCCGAGCGCCGGGGATTCCTG |
| | nucleotide | CCCGGTTTAGCGGCAGTGGGAGCGGAACTGACTTTACCCTTACC |
| | | ATCTCCTCACTTGAGCCAGAGGACTTTGCTGTTTATTACTGCTCC |
| | | ACGATGGCAATTCCCTTTGGAGGAGGAACCAAGGTTGAAATAA |
| | | AG |
| 146 | AB449 | GATATCCAAATGACACAAAGCCCATCATCTGTCTCAGCCAGTGT |
| | light | TGGAGACCGAGTTACAATTACCTGCAGGGCTAGCCAAGGCATCT |
| | chain | CCAGAGACCTGGCTTGGTATCAACAAAAGCCCGGGAAAGCGCC |
| | poly- | AAAGTTGCTGATATATGCAGCAAGCACACTTGTTTCCGGCGTTC |
| | nucleotide | CCAGTCGCTTCAGCGGATCCGGCAGTGGCACAGATTTTACGCTT |
| | | ACAATAAGCAGTTTGCAGCCTGAAGATTTTGCAAATTACTACTG |
| | | TCAACAGTATGTATCAGCTCCGCCATATACGTTTGGCGGTGGTA |
| | | CCAAAGTCGAGATCAAA |
| 147 | AB338 | GATATCGTTATGACGCAAAGCCCACTGAGCCTGCCGGTTACCCC |
| | light | CGGGGAGCCGGCTAGTATCAGCTGCCGGAGCAGCCAATCACTGT |
| | chain | TGCACTCAAACGAAAAGAATTACTTGCACTGGTATCTGCAGAAA |
| | poly- | CCCGGCCAATCACCTCAACTGCTCATTTACGACGCCAGTAAACG |
| | nucleotide | CGCATCCGGTGTTCCAGATCGCTTTTCCGGATCTGGTTCAGGCAC |
| | | TGATTTTACCTTGAAAATTTCCCGCGTAGAGGCTGAGGATGTTG |
| | | GGGTATACTACTGCGTACAAGATCTCCACACACCCTTCACATTC |
| | | GGCGGCGGAACAAAAGTAGAAATTAAG |
| 148 | AB435 | GAAATCGTCATGACACAGTCTCCAGCTACGTTGAGTCTCAGCCC |
| | light | TGGTGAACGGGCCACATTGAGTTGTAGGGCATCCCAAAGTATAG |
| | chain | GAACAAAGCTTGCGTGGTACCAACAAAAGCCAGGGCAAGCTCC |
| | poly- | CCGACTCCTCATTTATGATGCGAGCTCTCGCGTGCCTGGAATCCC |
| | nucleotide | TGCAAGGTTTTCAGGGTCCGGATCTGGGACAGATTTTACACTGA |
| | | CTATTTCCTCTCTCGAGCCAGAAGACTTTGCCGTCTACTACTGTC |
| | | AACAATATGGGAGTAGCCCACCCGTCTACTCCTTTGGGGGGGGA |
| | | ACTAAAGTAGAAATCAAG |
| 149 | AB372 | GACATAGTCATGACTCAAAGCCCCGACAGCCTCGCAGTTTCTCT |
| | light | CGGGGAACGCGCAACTATCAACTGCAAATCATCTCAGAGTGTGC |
| | chain | TGGATACTTCAACCAACAGAAATTACTTGGCATGGTATCAGCAA |
| | poly- | AAGCCCGGCCAACCACCCAAACTCCTCATTTATGGGGCTTCAAT |
| | nucleotide | CAGAGCGGGCGGCGTACCCGATCGGTTTTCCGGGAGCGGAAGT |
| | | GGAACAGACTTCACACTGACTATTAGCAGTTTGCAAGCAGAAGA |
| | | CGTGGCGGTATACTACTGCCAACAAAGCTACTCACATCCGCCAA |
| | | CTTTTGGTGGTGGTACTAAAGTGGAAATCAAG |
| 150 | AB439 | GAGTCTGCATTGACACAACCCGCATCAGTTAGTGGAAGCCCCGG |
| | light | CCAGTCTATTACAATTAGTTGTACAGGTACAAATATTGGGCTCC |
| | chain | GGAGTGTGAGCTGGTATCAACAACATCCAGGTAAAGCCCCGAA |
| | poly- | GCTGATGATCTATGATACGAATATTAGGTCTAGCGGCGTGTCTA |
| | nucleotide | ATAGGTTTTCCGGGTCCAAATCCGGGAATACCGCTTCTCTTACCA |
| | | TTAGCGGGCTGCAGGCCGAAGATGAAGCAGACTATTACTGCTCT |
| | | TCCTACACATCATCCAGTACTGACGTCTTCGGTGGCGGTACCAA |
| | | ACTGACAGTCCTT |
| 151 | AB350 | GAAATCGTTATGACTCAGAGCCCCGCAACACTCAGTTTGAGCCC |
| | light | CGGTGAGCGCGCCACGTTGAGCTGTCGTGCTAGCGAATCCGTTC |
| | chain | GCTCAAACCTCGCGTGGTACCAGCAAAAGCCAGGTCAGGCCCCC |
| | poly- | AGGCTCCTGATCTACGGCGCTAGCAACCGAGCGTCCGGTATTCC |
| | nucleotide | GGCAAGGTTTTCAGGGTCTGGGAGCGGGACCGACTTCACTCTCA |
| | | CAATATCATCACTGGAACCCGAGGATTTTGCGGTGTATTATTGC |
| | | CAGCAATACCACACTTGGCCGCCATGGGCCTTTGGTGGTGGTAC |
| | | GAAGGTCGAGATAAAG |
| 152 | AB363 | GATATCCAAATGACACAATCCCCTAGCTCAGTCTCCGCATCTGT |
| | light | GGGAGATAGGGTCACTATTACATGCCGTGCTTCTCAAGGTATTT |
| | chain | CCAACGACTTGGCTTGGTATCAACAAAAGCCTGGAAAGGCACCA |
| | poly- | AAGCTGCTGATCTATGATGTCTCCAATCGGGCTTCCGGCGTCCCT |
| | nucleotide | AGCCGGTTCTCTGGGAGCGGATCTGGCACCGACTTCACACTTAC |
| | | TATTAGCTCTCTTCAACCCGAGGATTTTGCCAATTATTACTGTCA |
| | | ACAATACGATAATCCCCCATCCTTTGGCGGCGGCACAAAGGTGG |
| | | AAATCAAA |
| 153 | AB367 | GACATTCAAATGACACAATCACCTAGTAGTGTTTCTGCAAGCGT |
| | light | GGGAGATAGGGTCACGATTACGTGCAGAGCCAGTCAAGGAATT |
| | chain | GGAATTGATCTCGCTTGGTATCAACAAAAGCCCGGCAAGGCTCC |
| | poly- | CAAGTTGCTCATTTACGATGCTAGTAAACTGCAGACGGGTGTCC |
| | nucleotide | CGTCACGGTTCTCCGGGTCTGGATCCGGTACAGATTTTACGCTCA |
| | | CAATTAGCTCTCTTCAACCCGAAGATTTCGCGAACTACTACTGTC |
| | | GTCAGTATGCTACCTTTCCTCAAACATTCGGGGGGGGCACCAAG |
| | | GTAGAAATTAAA |
| 154 | AB352 | GAAATTGTGATGACACAATCTCCTGCCACGCTTTCCCTCAGCCCC |
| | light | GGGGAAAGGGCTACGTTGTCTTGTCGCGCTTCCCAAAGTGTCAG |
| | chain | CACCCACCTGGCATGGTATCAACAAAAGCCCGGACAAGCACCG |
| | poly- | AGGCTTCTGATCTACGATGCCACCAATCGGGCAACAGGCATTCC |
| | nucleotide | TGCCCGGTTCAGTGGGTCCGGGTCCGGGACTGATTTTACCCTCA |
| | | CCATCTCTAGCCTCGAGCCGGAAGACTTTGCTGTCTACTATTGTC |
| | | TGCAATATACGAATTACCCCTGGACATTTGGTGGCGGTACCAAG |
| | | GTCGAGATTAAA |
| 155 | AB357 | GAATCCGCCCTGACCCAACCAGCAAGTGTTTCCGGTTCCCCAGG |
| | light | TCAATCAATCACTATCTCCTGTACTGGGACCGACATCCGCGGTA |
| | chain | TGTCAGTCTCCTGGTATCAGCAGCACCCCGGGAAGGCCCCCAAA |
| | poly- | CTTATGATCTATGAGGTCAACCAAAGGCCGAGTGGAGTATCCAA |
| | nucleotide | TCGGTTTAGCGGCTCTAAAAGCGGGAATACAGCTTCTCTCACCA |
| | | TCTCCGGCCTTCAAGCTGAAGACGAAGCTGATTATTACTGCGCT |
| | | GCCTGGGACGCAAGCGTTGCAGGATACGTCTTTGGAGGAGGGA |
| | | CGAAACTGACGGTTCTG |
| 156 | AB347 | GAAATCGTGATGACCCAGTCACCCGCCACATTGTCCTTGAGCCC |
| | light | CGGGGAACGGGCCACCCTCAGCTGTCGAGCCAGCCAGAACATT |
| | chain | GGCCACAGTTTGGCTTGGTATCAACAGAAACCAGGGCAGGCTCC |
| | poly- | TAGACTGCTTATATACGGGGCTAGTATAAGGGCTCCCGGTATCC |
| | nucleotide | CGGCGCGGTTCTCAGGCTCCGGCAGTGGAACGGACTTCACTCTG |
| | | ACAATATCCAGTTTGGAGCCGGAAGACTTTGCAGTTTATTATTG |
| | | CCAACAATATATCACCTGGCCCAGTACTTTTGGCGGTGGAACAA |
| | | AAGTTGAAATAAAA |
| 157 | AB346 | GAGATTGTCATGACCCAATCCCCTGCTACTCTGTCTCTGTCTCCC |
| | light | GGGGAACGCGCTACACTTAGTTGCAGGGCATCCCGGTCTGTATC |
| | chain | AAGTGATCTGGCGTGGTACCAGCAAAAGCCCGGGCAAGCCCCC |
| | poly- | AGGCTCTTGATTTACGACGTCTCCACTCGGGCCACGGGAATACC |
| | nucleotide | TGCTAGATTCTCTGGGTCAGGAAGCGGAACCGATTTTACCCTCA |
| | | CTATTAGTAGCTTGGAGCCTGAGGATTTCGCCGTCTATTATTGTC |
| | | AGCAATACGGCAGTAGTCTCCCGCCCACCTTCGGTGGCGGAACC |
| | | AAGGTTGAAATCAAG |
| 158 | AB433 | GAAATCGTCATGACCCAAAGCCCTGCAACCCTCTCATTGTCACC |
| | light | CGGAGAGCGCGCAACGCTGAGCTGTCGGGCCTCTCAAAGCGTG |
| | chain | AGAACCAGTCTCGCATGGTACCAACAAAAGCCAGGCCAAGCTC |
| | poly- | CACGGTTGCTTATCTATGATACAAGCATTAGGGCCGCAGGCATA |
| | nucleotide | CCGGCACGGTTCAGTGGCTCTGGTAGTGGTACCGACTTTACCCT |
| | | CACGATTTCAAGCCTGGAACCCGAAGACTTTGCGGTCTACTATT |
| | | GTCAACAATACGGGAATTGGCACTTTGGCGGCGGAACGAAAGT |
| | | GGAAATTAAG |
| 159 | AB432 | GAAATTGTCATGACCCAAAGTCCGGCGACGCTTTCCCTCTCTCCC |
| | light | GGCGAAAGGGCAACCCTGAGCTGTCGCGCTAGTCAATCAATATC |
| | chain | CTCTTCACAGCTCGCGTGGTACCAACAGAAACCCGGCCAAGCAC |
| | poly- | CTAGACTCCTGATATATGGAGCTTCTACGAGAGCCACCGGGATT |
| | nucleotide | CCAGCTAGGTTTTCTGGTTCAGGGTCCGGGACGGATTTTACACTC |
| | | ACAATCAGCTCCCTCGAGCCAGAGGATTTTGCGGTTTACTATTG |
| | | CCAACAATATAATGACAGACCCAGGACATTCGGCGGCGGAACT |
| | | AAGGTTGAGATTAAA |
| 160 | AB326 | GATATACAAATGACGCAAAGTCCCTCAAGTGTAAGCGCGTCTGT |
| | light | CGGAGACCGGGTGACTATAACATGCAGAGCTTCTATGGATATAA |
| | chain | GGAATGATCTCGCGTGGTACCAACAGAAGCCAGGCAAGGCGCC |
| | poly- | TAAACTGTTGATATATGATGCGTCAAAATTGGAGACAGGAGTGC |
| | nucleotide | CCAGCAGGTTCTCAGGTTCCGGAAGTGGGACCGATTTTACACTC |
| | | ACCATTTCATCTCTGCAACCAGAGGACTTCGCCAATTATTATTGT |
| | | CAACAAGCTAAGTCCTTCCCGCCCACCTTTGGCGGTGGAACCAA |
| | | GGTGGAAATAAAA |
| 161 | AB428 | GATATTGTGATGACGCAGTCACCTCTCAGCCTGCCTGTGACACC |
| | light | CGGGGAGCCAGCCTCTATTAGCTGCCGGAGCTCACAGAGTCTCT |
| | chain | TGCATTCCAGCGGGTACAACTTTCTCCACTGGTATCTGCAAAAG |
| | poly- | CCGGGTCAAAGTCCACAGTTGCTTATTTACATGGCGACAAACCG |
| | nucleotide | TGCGTCAGGAGTGCCGGATAGATTCAGCGGCAGTGGCTCCGGGA |
| | | CAGATTTCACCCTTAAGATTTCCCGCGTTGAGGCAGAAGACGTC |
| | | GGCGTTTATTATTGTGTTCAGGATCTTCATACATTGCCACAATTT |
| | | GGTGGTGGGACAAAGGTAGAAATAAAG |
| 162 | AB426 | GATATCGTGATGACACAAAGCCCCCTTAGTCTTCCGGTCACTCC |
| | light | CGGGGAACCTGCCAGTATTAGTTGCAGAAGCTCCCAATCACTGC |
| | chain | TGCATTGGTATCTCCAGAAACCAGGACAATCTCCCCAATTGCTC |
| | poly- | ATCTATATGGGATCCCAAAGGGCCGCGGGCGTTCCTGATCGTTT |
| | nucleotide | CTCAGGCAGCGGGTCAGGGACCGACTTCACGCTCAAGATTAGCC |
| | | GCGTGGAAGCAGAAGATGTGGGAGTCTATTATTGCCAATCAGGC |
| | | GTCACTTTCGGCGGTGGTACGAAGGTTGAGATCAAG |
| 163 | AB345 | GATATTGTTATGACACAAAGTCCCCTCTCCCTTCCTGTTACTCCG |
| | light | GGAGAGCCGGCAAGCATAAGCTGTAGATCTTCACAGTCACTTCT |
| | chain | TAGCTCATACGGATACCATAATCTGCATTGGTACCTTCAGAAAC |
| | poly- | CTGGACAAAGCCCACAACTTTTGATCTACATGGGATCAATCCGA |
| | nucleotide | GCCCCCGGAGTTCCTGACCGCTTTTCTGGCTCCGGCAGCGGGAC |
| | | AGATTTTACCCTGAAAATTTCAAGGGTAGAAGCTGAAGACGTCG |
| | | GTGTATATTATTGTATGCAGGGAACACAAGCTCCTAGAACGTTT |
| | | GGAGGAGGCACAAAGGTAGAAATAAAA |
| 164 | AB431 | GATATAGTCATGACGCAAAGTCCTCTCAGCCTGCCAGTCACTCC |
| | light | CGGTGAACCGGCGAGTATTTCCTGTCGGTCCAGCCAATCCCTTCT |
| | chain | GCACGTTACGGGTTATAATTACCTGCATTGGTATCTCCAGAAAC |
| | poly- | CCGGCCAATCCCCTCAACTTTTGATCTATGGGGCAAGTAGTCGC |
| | nucleotide | GCTGCCGGCGTTCCCGACAGGTTCTCAGGTAGCGGTAGCGGAAC |
| | | GGACTTCACACTGAAAATTTCCCGTGTTGAAGCCGAGGATGTAG |
| | | GCGTATATTATTGTATGCAGAGTACACAATGGCCACACACCTTC |
| | | GGTGGTGGGACTAAAGTGGAAATCAAA |
| 165 | AB330 | GATATTCAAATGACGCAATCCCCATCCTCTGTTTCAGCTTCCGTC |
| | light | GGGGATCGGGTCACCATAACTTGTCGCGCCTCTCAAGATATCAA |
| | chain | ATCTGATTTGGCCTGGTACCAACAGAAACCGGGGAAAGCACCCA |
| | poly- | AGCTCCTCATTTATAGGGCATCCGCACGGGAAAGCGGGGTACCA |
| | nucleotide | TCTCGCTTTAGTGGATCCGGCTCTGGTACGGACTTCACTCTGACG |
| | | ATTAGCAGCCTGCAACCCGAGGACTTTGCAAATTACTACTGCCA |
| | | ACAATATCGCAGTTTTCCATGGAGTTTTGGTGGTGGGACAAAGG |
| | | TCGAGATCAAG |
| 166 | AB731 | GAAAATGTGCTAACTCAAAGTCCAGCAATCATGTCCGCCTCTCC |
| | light | AGGCGAGAAAGTGACCATCAGCTGTTCTGCTTCTTCCTCCGTGTC |
| | chain | CTACATGTACTGGTACCAGCAGAAGCCTGGCTCCTCTCCTAAGC |
| | poly- | CCTGGATCTTCAGAACCTACAACCTGGCCTCCGGCGTGCCTGCT |
| | nucleotide | AGATTTAGCGGATCTGGCTCTGGCACCTCTTACTCCCTGACCATC |
| | | TCCAACATGGAAGCCGAGGACGCCGCTACATACTACTGCCAGCA |
| | | ACGGTCCAGCTATCCTTGGACCTTCGGCGGCGGAACCAAGCTGG |
| | | AGATCAAG |
| 167 | AB843 | GAAAGTGCACTAACTCAACCAGCAAGTGTGAGCGGATCTCCTGG |
| | light | CCAGTCCATCACCATCTCCTGCACCGGCACCAGCGGCGACATCG |
| | chain | GCAGATACAACTATGTCTCTTGGTACCAGCAGCACCCCGGCAAG |
| | poly- | GCCCCTAAACTGATGATCTACGAAGATATCGAGAGACCTTCTGG |
| | nucleotide | CGTGTCCAACCGGTTCTCCGGCTCCAAGTCCGGCAACACCGCTT |
| | | CTCTCACAATCTCTGGCCTGCAGGCCGAGGACGAGGCCGACTAC |
| | | TACTGTCAAGTGTGGGATGCTACCCTGGACCTGGTGTTCGGCGG |
| | | CGGAACCAAGCTGACCGTGCTG |
| 168 | AB852 | GATATCGTGATGACTCAAAGTCCAGATAGTCTCGCTGTCTCTCTG |
| | light | GGCGAGAGAGCCACAATCAACTGCAAGTCCTCTCAGTCCGTGCT |
| | chain | GGACACCTCCACCAACCGGAACTACCTGGCCTGGTATCAGCAGA |
| | poly- | AACCAGGCCAGCCTCCTAAGCTGCTGATCTACGGCGCTTCCATC |
| | nucleotide | AGAGCTGGCGGCGTGCCCGATCGGTTTAGCGGCTCTGGATCTGG |
| | | CACCGACTTCACCCTGACCATCTCCAGCCTGCAGGCCGAGGACG |
| | | TGGCCGTGTACTACTGTCAACAGTACGAGACATGGCCTTGGACC |
| | | TTCGGCGGAGGCACCAAGGTGGAAATCAAG |
| 169 | AB854 | GAAAGTGCACTAACTCAACCAGCAAGTGTGAGCGGCTCTCCAGG |
| | light | CCAGTCCATCACCATCTCCTGCACCGGCACCACCGGATCTATCG |
| | chain | CCTCCAACTACGTGTCTTGGTATCAGCAGCACCCCGGCAAAGCT |
| | poly- | CCTAAGCTGATGATCTACGACACAACCAACCGGCCTCCTGGCGT |
| | nucleotide | GTCCAATCGGTTCTCCGGCTCCAAGTCCGGCAACACCGCTAGCC |
| | | TGACAATCTCTGGCCTGCAGGCCGAGGACGAGGCCGACTACTAC |
| | | TGTCAATCTACCGAAAGAGGCAGAAGCGATGTCATCTTCGGCGG |
| | | CGGAACCAAGCTGACCGTGCTG |
| 200 | Control | QVTLRESGPALVKPTQTLTLTCTFSGFSLSTSGMSVGWIRQPPGKAL |
| | 3 Ab | EWLADIWWDDKKDYNPSLKSRLTISKDTSKNQVVLKVTNMDPAD |
| | (heavy | TATYYCARSMITNWYFDVWGAGTTVTVSSASTKGPSVFPLAPSSKS |
| | chain) | TSGGTAALGCLVKDYFPEPVTVSWNSGALTSGVHTFPAVLQSSGLY |
| | | SLSSVVTVPSSSLGTQTYICNVNHKPSNTKVDKRVEPKSCDKTHTCP |
| | | PCPAPELLGGPSVFLFPPKPKDTLMISRTPEVTCVVVDVSHEDPEVK |
| | | FNWYVDGVEVHNAKTKPREEQYNSTYRVVSVLTVLHQDWLNGKE |
| | | YKCKVSNKALPAPIEKTISKAKGQPREPQVYTLPPSREEMTKNQVS |
| | | LTCLVKGFYPSDIAVEWESNGQPENNYKTTPPVLDSDGSFFLYSKL |
| | | TVDKSRWQQGNVFSCSVMHEALHNHYTQKSLSLSPGK |
| 201 | Control | DIQMTQSPSTLSASVGDRVTITCKCQLSVGYMHWYQQKPGKAPKL |
| | 3 Ab | LIYDTSKLASGVPSRFSGSGSGTEFTLTISSLQPDDFATYYCFQGSGY |
| | (light | PFTFGGGTKLEIKRTVAAPSVFIFPPSDEQLKSGTASVVCLLNNFYPR |
| | chain) | EAKVQWKVDNALQSGNSQESVTEQDSKDSTYSLSSTLTLSKADYE |
| | | KHKVYACEVTHQGLSSPVTKSFNRGEC |
| 202 | Human | MPLLWLRGFLLASCWIIVRSSPTPGSEGHSAAPDCPSCALAALPKDV |
| | inhibin | PNSQPEMVEAVKKHILNMLHLKKRPDVTQPVPKAALLNAIRKLHV |
| | beta-A | GKVGENGYVEIEDDIGRRAEMNELMEQTSEIITFAESGTARKTLHFE |
| | Pre- | ISKEGSDLSVVERAEVWLFLKVPKANRTRTKVTIRLFQQQKHPQGS |
| | protein | LDTGEEAEEVGLKGERSELLLSEKVVDARKSTWHVFPVSSSIQRLL |
| | [GenBank | DQGKSSLDVRIACEQCQESGASLVLLGKKKKKEEEGEGKKKGGGE |
| | NP_002183] | GGAGADEEKEQSHRPFLMLQARQSEDHPHRRRRRGLECDGKVNIC |
| | | CKKQFFVSFKDIGWNDWIIAPSGYHANYCEGECPSHIAGTSGSSLSF |
| | | HSTVINHYRMRGHSPFANLKSCCVPTKLRPMSMLYYDDGQNIIKK |
| | | DIQNMIVEECGCS |
| 203 | Human | MDGLPGRALGAACLLLLAAGWLGPEAWGSPTPPPTPAAPPPPPPPG |
| | inhibin | SPGGSQDTCTSCGGFRRPEELGRVDGDFLEAVKRHILSRLQMRGRP |
| | beta-B | NITHAVPKAAMVTALRKLHAGKVREDGRVEIPHLDGHASPGADGQ |
| | Pre- | ERVSEIISFAETDGLASSRVRLYFFISNEGNQNLFVVQASLWLYLKL |
| | protein | LPYVLEKGSRRKVRVKVYFQEQGHGDRWNMVEKRVDLKRSGWH |
| | [GenBank | TFPLTEAIQALFERGERRLNLDVQCDSCQELAVVPVFVDPGEESHRP |
| | AAA59451.1] | FVVVQARLGDSRHRIRKRGLECDGRTNLCCRQQFFIDFRLIGWND |
| | | WIIAPTGYYGNYCEGSCPAYLAGVPGSASSFHTAVVNQYRMRGLN |
| | | PGTVNSCCIPTKLSTMSMLYFDDEYNIVKRDVPNMIVEECGCA |
| 204 | Human | MTSSLLLAFLLLAPTTVATPRAGGQCPACGGPTLELESQRELLLDLA |
| | inhibin | KRSILDKLHLTQRPTLNRPVSRAALRTALQHLHGVPQGALLEDNRE |
| | beta-C | QECEIISFAETGLSTINQTRLDFHFSSDRTAGDREVQQASLMFFVQLP |
| | Pre- | SNTTWTLKVRVLVLGPHNTNLTLATQYLLEVDASGWHQLPLGPEA |
| | protein | QAACSQGHLTLELVLEGQVAQSSVILGGAAHRPFVAARVRVGGKH |
| | [GenBank | QIHRRGIDCQGGSRMCCRQEFFVDFREIGWHDWIIQPEGYAMNFCI |
| | EAW97011.1] | GQCPLHIAGMPGIAASFHTAVLNLLKANTAAGTTGGGSCCVPTARR |
| | | PLSLLYYDRDSNIVKTDIPDMVVEACGCS |
| 205 | INHBA | GLECDGKVNICCKKQFFVSFKDIGWNDWIIAPSGYHANYCEGECPS |
| | mature | HIAGTSGSSLSFHSTVINHYRMRGHSPFANLKSCCVPTKLRPMSML |
| | | YYDDGQNIIKKDIQNMIVEECGCS |
| 206 | INHBA | SPTPGSEGHSAAPDCPSCALAALPKDVPNSQPEMVEAVKKHILNML |
| | latent | HLKKRPDVTQPVPKAALLNAIRKLHVGKVGENGYVEIEDDIGRRAE |
| | | MNELMEQTSEIITFAESGTARKTLHFEISKEGSDLSVVERAEVWLFL |
| | | KVPKANRTRTKVTIRLFQQQKHPQGSLDTGEEAEEVGLKGERSELL |
| | | LSEKVVDARKSTWHVFPVSSSIQRLLDQGKSSLDVRIACEQCQESG |
| | | ASLVLLGKKKKKEEEGEGKKKGGGEGGAGADEEKEQSHRPFLML |
| | | QARQSEDHPHRRRRRGLECDGKVNICCKKQFFVSFKDIGWNDWIIA |
| | | PSGYHANYCEGECPSHIAGTSGSSLSFHSTVINHYRMRGHSPFANLK |
| | | SCCVPTKLRPMSMLYYDDGQNIIKKDIQNMIVEECGCS |
| 207 | INHBB | GLECDGRTNLCCRQQFFIDFRLIGWNDWIIAPTGYYGNYCEGSCPA |
| | mature | YLAGVPGSASSFHTAVVNQYRMRGLNPGTVNSCCIPTKLSTMSML |
| | | YFDDEYNIVKRDVPNMIVEECGCA |
| 208 | INHA | STPLMSWPWSPSALRLLQRPPEEPAAHANCHRVALNISFQELGWER |
| | mature | WIVYPPSFIFHYCHGGCGLHIPPNLSLPVPGAPPTPAQPYSLLPGAQP |
| | | CCAALPGTMRPLHVRTTSDGGYSFKYETVPNLLTQHCACI |

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually indicated to be incorporated by reference for all purposes.

What is claimed is:

1. An anti-activin antibody which binds to Activin A.

2. The anti-activin antibody according to claim 1, which binds Activin B.

3. The anti-activin antibody according to claim 1 or claim 2, which binds latent Activin A.

4. The anti-activin antibody according to any one of claims 1-3, which binds active Activin A.

5. The anti-activin antibody according to claim 1, comprising a heavy chain variable region comprising an amino acid sequence selected from the group consisting of: SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, and 69.

6. The anti-activin antibody according to claim 1, comprising a heavy chain variable region comprising an amino acid sequence selected from Table I or Table III.

7. The anti-activin antibody claim 1, comprising a light chain variable region comprising an amino acid sequence selected from the group consisting of: SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, and 70.

8. The anti-activin antibody according to any one of claim 1, comprising a light chain variable region comprising an amino acid sequence selected from Table II or Table IV.

9. The anti-activin antibody according to any one of claims 2-4, comprising a heavy chain variable region comprising an amino acid sequence selected from the group consisting of: SEQ ID NOs: 39, 41, 43, 45, 47, 49, 63, 65, and 69.

10. The anti-activin antibody according to any one of claims 2-4, comprising a light chain variable region comprising an amino acid sequence selected from the group consisting of SEQ ID NOs: 40, 42, 44, 46, 48, 50, 64, 66, and 70.

11. The anti-activin antibody according to any one of claims 2-4, comprising a heavy chain variable region comprising a CDR1 sequence selected from the group consisting of SEQ ID NOs: 229-234, 241-242, and 244; a CDR2 sequence selected from the group consisting of SEQ ID NOs: 264-269, 276-277, and 279; and a CDR3 sequence selected from the group consisting of SEQ ID NOs: 299-304, 311-312, and 314.

12. The anti-activin antibody according to any one of claims 2-4, comprising a light chain variable region comprising a CDR1 sequence selected from the group consisting of SEQ ID NOs: 334-339, 346-347, and 349; a CDR2 sequence selected from the group consisting of SEQ ID NOs: 369-374, 381-382, and 384; and a CDR3 sequence selected from the group consisting of SEQ ID NOs: 404-409; 416-417; and 419.

13. The anti-activin antibody according to any one of claims 2-4, comprising a heavy chain variable region comprising a CDR1 sequence selected from the group consisting of SEQ ID NOs: 439-444, 451-452, and 454; a CDR2 sequence selected from the group consisting of SEQ ID NOs: 474-479, 486-487, and 489; and a CDR3 sequence selected from the group consisting of SEQ ID NOs: 509-514, 521-522, and 524.

14. The anti-activin antibody according to any one of claims 2-4, comprising a light chain variable region comprising a CDR1 sequence selected from the group consisting of SEQ ID NOs: 544-549, 556-557, and 559; a CDR2 sequence selected from the group consisting of SEQ ID NOs: 589-594, 601-602, and 604; and a CDR3 sequence selected from the group consisting of SEQ ID NOs: 624-629, 636-637, and 639.

15. The anti-activin antibody according to any one of claims 2-4, comprising a heavy chain variable region comprising SEQ ID NO: 39 and a light chain variable region comprising SEQ ID NO: 40.

16. The anti-activin antibody according to any one of claims 2-4, comprising a heavy chain variable region comprising SEQ ID NO: 41 and a light chain variable region comprising SEQ ID NO: 42.

17. The anti-activin antibody according to any one of claims 2-4, comprising a heavy chain variable region comprising SEQ ID NO: 43 and a light chain variable region comprising SEQ ID NO: 44.

18. The anti-activin antibody according to any one of claims 2-4, comprising a heavy chain variable region comprising SEQ ID NO: 45 and a light chain variable region comprising SEQ ID NO: 46.

19. The anti-activin antibody according to any one of claims 2-4, comprising a heavy chain variable region comprising SEQ ID NO: 47 and a light chain variable region comprising SEQ ID NO: 48.

20. The anti-activin antibody according to any one of claims 2-4, comprising a heavy chain variable region comprising SEQ ID NO: 49 and a light chain variable region comprising SEQ ID NO: 50.

21. The anti-activin antibody according to any one of claims 2-4, comprising a heavy chain variable region comprising SEQ ID NO: 63 and a light chain variable region comprising SEQ ID NO: 64.

22. The anti-activin antibody according to any one of claims 2-4, comprising a heavy chain variable region comprising SEQ ID NO: 65 and a light chain variable region comprising SEQ ID NO: 66.

23. The anti-activin antibody according to any one of claims 2-4, comprising a heavy chain variable region comprising SEQ ID NO:69 and a light chain variable region comprising SEQ ID NO:70.

24. The anti-activin antibody according to claim 1, comprising a heavy chain variable region/light chain variable region sequence pair selected from the group consisting of SEQ ID NOs: 1/2, 3/4, 5/6, 7/8, 9/10, 11/12, 13/14, 15/16, 17/18, 19/20, 21/22, 23/24, 25/26, 27/28, 29/30, 31/32, 33/34, 35/36, 37/38, 39/40, 41/42, 43/44, 45/46, 47/48, 49/50, 51/52, 53/54, 55/56, 57/58, 59/60, 61/62, 63/64, 65/66, 67/68, and 69/70.

25. The anti-activin antibody according to any one of claims 1-4, wherein the anti-activin antibody is a chimeric, humanized, or human antibody.

26. The anti-activin antibody according to any one of claims 1-24, wherein the anti-activin antibody is a monoclonal antibody.

27. The anti-activin antibody according to any one of claims 1-24, wherein the anti-activin antibody is an antibody fragment.

28. The anti-activin antibody according to any one of claims 1-24, wherein the anti-activin antibody comprises a single-chain antibody.

29. The antibody according to any one of claims 1-6, 9, 11, or 13, wherein the anti-activin antibody is heavy-chain only antibody (single domain antibody).

30. A modified immune cell, comprising a chimeric antigen receptor (CAR), wherein said CAR comprises the anti-activin antibody according to claim 28.

31. The modified immune cell according to claim 30, wherein said modified immune cell is a modified T cell.

32. The modified immune cell according to claim 30, wherein said modified immune cell is a modified NK cell.

33. The modified immune cell according to claim 30, wherein said modified immune cell is a modified macrophage.

34. An antibody-drug conjugate (ADC), comprising the antibody according to any one of claims 1-24.

35. A method of inhibiting the growth of a cell that displays an activin tumor epitope shared between Activin A and Activin B, comprising contacting said cell with the anti-activin antibody according to claim 1, a modified immune cell comprising a CAR, wherein said CAR comprises the anti-activin antibody according to claim 1, or an ADC comprising the antibody according to claim 1.

36. A method of treating a subject having cancer, comprising administering to said subject the anti-activin antibody according to any one of claims 1-24, the modified immune cell according to claim 30, or the ADC according to claim 34.

37. The method according to claim 36, wherein said subject is a human.

38. The method according to claim 36, wherein said cancer is selected from the group consisting of cholangiocarcinoma, colon adenocarcinoma, B-cell lymphoma, esophageal carcinoma, glioblastoma, head and neck cancer, kidney clear cell cancer, low grade glioma, pancreatic adenocarcinoma, paraganglioma, prostate adenocarcinoma, rectal adenocarcinoma, sarcoma, stomach adenocarcinoma, and thyroid carcinoma.

39. The method according to claim 36, wherein said cancer is selected from the group consisting of adrenocortical cancer, cholangiocarcinoma, colon adenocarcinoma, B-cell lymphoma, esophageal carcinoma, glioblastoma, head and neck cancer, kidney chromophobe, kidney clear cell cancer, kidney papillary cell cancer, low grade glioma, liver hepatocellular cancer, lung adenocarcinoma, ovarian cancer, pancreatic adenocarcinoma, paraganglioma, rectal adenocarcinoma, sarcoma, stomach adenocarcinoma, thyroid carcinoma, and uterine corpus cancer.

40. A pharmaceutical composition comprising an antibody according to any one of claims 1-24, and a pharmaceutically acceptable carrier.

41. A pharmaceutical composition comprising the modified immune cell according to claim 30, and a pharmaceutically acceptable carrier.

42. Use of the antibody according to any one of claims 1-24 in the preparation of a medicament for the treatment of cancer.

43. Use of the modified immune cell according to claim 30 in the preparation of a medicament for the treatment of cancer.

44. Use of the ADC according to claim 34 in the preparation of a medicament for the treatment of cancer.


SEQUENCE LISTING (576 sequences)

SEQ ID NO: 1 - protein, 116 aa, Artificial Sequence
  Description of Artificial Sequence Synthetic polypeptide
QVQLQESGPGLVKPSQTLSLTCTVSGLSLSNAKMGVNWIRQPPGKGLEWIGYVSTTGTTDYNPSLKSRVTMSVDTSKNQFSLKVNSVTAADTAVYYCARAGYPDVWGKGTTVTVSS

SEQ ID NO: 2 - protein, 107 aa, Artificial Sequence
  Description of Artificial Sequence Synthetic polypeptide
DIQMTQSPSSVSASVGDRVTITCRASQNIGAALAWYQQKPGKAPKLLIYGASTREYGVPSRFSGSGSGTDFTLTISSLQPEDFANYYCQQYTAYPLTFGGGTKVEIK

SEQ ID NO: 3 - protein, 116 aa, Artificial Sequence
  Description of Artificial Sequence Synthetic polypeptide
QVQLQESGPGLVKPSQTLSLTCTVSGASVSNARTGVNWIRQPPGKGLEWIGYINPSGRIDYNPSLKSRVTMSVDTSKNQFSLKVNSVTAADTAVYYCARAGYPDVWGKGTTVTVSS

SEQ ID NO: 4 - protein, 106 aa, Artificial Sequence
  Description of Artificial Sequence Synthetic polypeptide
EIVMTQSPATLSLSPGERATLSCRASQTVSSHLAWYQQKPGQAPRLLIYDVSHRASGIPARFSGSGSGTDFTLTISSLEPEDFAVYYCQQYFTLPTFGGGTKVEIK

SEQ ID NO: 5 - protein, 125 aa, Artificial Sequence
  Description of Artificial Sequence Synthetic polypeptide
EVQLVESGGGLVQPGGSLRLSCAASGFTASRNYMSWVRQAPGKGLELVASISGSGDYTYYPDSVKGRFTISRDNAKNSLYLQMNSLRAEDTAVYYCARSPRGPRLLGYYYGMDVWGQGTTVTVSS

SEQ ID NO: 6 - protein, 112 aa, Artificial Sequence
  Description of Artificial Sequence Synthetic polypeptide
DIVMTQSPLSLPVTPGEPASISCRSSRSLVHGSGHNYLHWYLQKPGQSPQLLIYGASTRVAGVPDRFSGSGSGTDFTLKISRVEAEDVGVYYCMQGTQAPRTFGGGTKVEIK

SEQ ID NO: 7 - protein, 120 aa, Artificial Sequence
  Description of Artificial Sequence Synthetic polypeptide
EVQLVESGGGLVQPGGSLRLSCAASGFDFGEHWMSWVRQAPGKGLELVASIKGDASDTYYPDSVKGRFTISRDNAKNSLYLQMNSLRAEDTAVYYCVKDLSKQWLGIDYWGQGTLVTVSS

SEQ ID NO: 8 - protein, 112 aa, Artificial Sequence
  Description of Artificial Sequence Synthetic polypeptide
DIVMTQSPLSLPVTPGEPASISCRSSQSLLHSNEKNYLHWYLQKPGQSPQLLIYDASKRASGVPDRFSGSGSGTDFTLKISRVEAEDVGVYYCVQDLHTPFTFGGGTKVEIK

SEQ ID NO: 9 - protein, 123 aa, Artificial Sequence
  Description of Artificial Sequence Synthetic polypeptide
EVQLVESGGGLVQPGGSLRLSCAASGYTFSNYGMSWVRQAPGKGLELVASISKSGSSIYYPDSVKGRFTISRDNAKNSLYLQMNSLRAEDTAVYYCARLPRLAWNYYYGMDVWGQGTTVTVSS

SEQ ID NO: 10 - protein, 112 aa, Artificial Sequence
  Description of Artificial Sequence Synthetic polypeptide
DIVMTQSPLSLPVTPGEPASISCRSSRSLVHGSGDNYLHWYLQKPGQSPQLLIYMASNRAPGVPDRFSGSGSGTDFTLKISRVEAEDVGVYYCVQTLQTPSTFGGGTKVEIK

SEQ ID NO: 11 - protein, 116 aa, Artificial Sequence; identical to SEQ ID NO: 1
  Description of Artificial Sequence Synthetic polypeptide

SEQ ID NO: 12 - protein, 107 aa, Artificial Sequence
  Description of Artificial Sequence Synthetic polypeptide
EIVMTQSPATLSLSPGERATLSCRASQNVYTNLAWYQQKPGQAPRLLIYGASARASGIPARFSGSGSGTDFTLTISSLEPEDFAVYYCLQYGSSPPLFGGGTKVEIK

SEQ ID NO: 13 - protein, 121 aa, Artificial Sequence
  Description of Artificial Sequence Synthetic polypeptide
QVQLQESGPGLVKPSQTLSLTCTVSGVSLSNARLGVNWIRQPPGKGLEWIGYGSVTGSTDYNPSLKSRVTMSVDTSKNQFSLKVNSVTAADTAVYYCTTSYLVDTAMVTRWGQGTLVTVSS

SEQ ID NO: 14 - protein, 107 aa, Artificial Sequence
  Description of Artificial Sequence Synthetic polypeptide
EIVMTQSPATLSLSPGERATLSCRASQSLGTNLAWYQQKPGQAPRLLIYGASSRPSGIPARFSGSGSGTDFTLTISSLEPEDFAVYYCQQYDTHPLSFGGGTKVEIK

SEQ ID NO: 15 - protein, 116 aa, Artificial Sequence
  Description of Artificial Sequence Synthetic polypeptide
QVQLQESGPGLVKPSQTLSLTCTVSGYSLTDARIGVNWIRQPPGKGLEWIGYINPSGRTDYNPSLKSRVTMSVDTSKNQFSLKVNSVTAADTAVYYCARAGYPDVWGKGTTVTVSS

SEQ ID NO: 16 - protein, 107 aa, Artificial Sequence
  Description of Artificial Sequence Synthetic polypeptide
DIQMTQSPSSVSASVGDRVTITCRASQTISRSLAWYQQKPGKAPKLLIYDASSLAGGVPSRFSGSGSGTDFTLTISSLQPEDFANYYCQQYSSYPSLFGGGTKVEIK

SEQ ID NO: 17 - protein, 123 aa, Artificial Sequence; identical to SEQ ID NO: 9
  Description of Artificial Sequence Synthetic polypeptide

SEQ ID NO: 18 - protein, 112 aa, Artificial Sequence; identical to SEQ ID NO: 8
  Description of Artificial Sequence Synthetic polypeptide

SEQ ID NO: 19 - protein, 123 aa, Artificial Sequence
  Description of Artificial Sequence Synthetic polypeptide
EVQLVESGGGLVQPGGSLRLSCAASGFTLSNYPMSWVRQAPGKGLELVASTTSGSSTIYYPDSVKGRFTISRDNAKNSLYLQMNSLRAEDTAVYYCARGPDYYAPLRSYMDVWGKGTTVTVSS

SEQ ID NO: 20 - protein, 112 aa, Artificial Sequence
  Description of Artificial Sequence Synthetic polypeptide
DIVMTQSPLSLPVTPGEPASISCRSSQSLQQSEGRTYLHWYLQKPGQSPQLLIYDASKRASGVPDRFSGSGSGTDFTLKISRVEAEDVGVYYCMQVLRTPITFGGGTKVEIK

SEQ ID NO: 21 - protein, 121 aa, Artificial Sequence; identical to SEQ ID NO: 13
  Description of Artificial Sequence Synthetic polypeptide

SEQ ID NO: 22 - protein, 104 aa, Artificial Sequence
  Description of Artificial Sequence Synthetic polypeptide
EIVMTQSPATLSLSPGERATLSCRASQRIGSDLAWYQQKPGQAPRLLIYSASSRAPGIPARFSGSGSGTDFTLTISSLEPEDFAVYYCSTMAIPFGGGTKVEIK

SEQ ID NO: 23 - protein, 116 aa, Artificial Sequence; identical to SEQ ID NO: 1
  Description of Artificial Sequence Synthetic polypeptide

SEQ ID NO: 24 - protein, 108 aa, Artificial Sequence
  Description of Artificial Sequence Synthetic polypeptide
DIQMTQSPSSVSASVGDRVTITCRASQGISRDLAWYQQKPGKAPKLLIYAASTLVSGVPSRFSGSGSGTDFTLTISSLQPEDFANYYCQQYVSAPPYTFGGGTKVEIK

SEQ ID NO: 25 - protein, 125 aa, Artificial Sequence; identical to SEQ ID NO: 5
  Description of Artificial Sequence Synthetic polypeptide

SEQ ID NO: 26 - protein, 112 aa, Artificial Sequence; identical to SEQ ID NO: 8
  Description of Artificial Sequence Synthetic polypeptide

SEQ ID NO: 27 - protein, 116 aa, Artificial Sequence; identical to SEQ ID NO: 15
  Description of Artificial Sequence Synthetic polypeptide

SEQ ID NO: 28 - protein, 109 aa, Artificial Sequence
  Description of Artificial Sequence Synthetic polypeptide
EIVMTQSPATLSLSPGERATLSCRASQSIGTKLAWYQQKPGQAPRLLIYDASSRVPGIPARFSGSGSGTDFTLTISSLEPEDFAVYYCQQYGSSPPVYSFGGGTKVEIK

SEQ ID NO: 29 - protein, 121 aa, Artificial Sequence
  Description of Artificial Sequence Synthetic polypeptide
QVTLRESGPALVKPTQTLTLTCTVSGFSLSTSGATVNWIRQPPGKALEWIGYITSSGRTVYNSALKSRLTISKDTSKNQVVLTMTNMDPVDTATYYCTTTKIVGATLRDYWGQGTLVTVSS

SEQ ID NO: 30 - protein, 113 aa, Artificial Sequence
  Description of Artificial Sequence Synthetic polypeptide
DIVMTQSPDSLAVSLGERATINCKSSQSVLDTSTNRNYLAWYQQKPGQPPKLLIYGASIRAGGVPDRFSGSGSGTDFTLTISSLQAEDVAVYYCQQSYSHPPTFGGGTKVEIK

SEQ ID NO: 31 - protein, 126 aa, Artificial Sequence
  Description of Artificial Sequence Synthetic polypeptide
EVQLVQSGAEVKKPGASVKVSCKASGYTYTGYRISWVRQAPGQGLEWMGWIDPKSGRTNYAQKLQGRGTMTTDPSTSTAYMELRSLRSDDTAVYYCAREVKQWLVPGRGVYWFDPWGQGTLVTVSS

SEQ ID NO: 32 - protein, 107 aa, Artificial Sequence
  Description of Artificial Sequence Synthetic polypeptide
ESALTQPASVSGSPGQSITISCTGTNIGLRSVSWYQQHPGKAPKLMIYDTNIRSSGVSNRFSGSKSGNTASLTISGLQAEDEADYYCSSYTSSSTDVFGGGTKLTVL

SEQ ID NO: 33 - protein, 116 aa, Artificial Sequence
  Description of Artificial Sequence Synthetic polypeptide
QVQLQESGPGLVKPSQTLSLTCTVSGVSLSDARMGVNWIRQPPGKGLEWIGYVSTTGTTDYNPSLKSRVTMSVDTSKNQFSLKVNSVTAADTAVYYCARAGYPDVWGKGTLVTVSS

SEQ ID NO: 34 - protein, 108 aa, Artificial Sequence
  Description of Artificial Sequence Synthetic polypeptide
EIVMTQSPATLSLSPGERATLSCRASESVRSNLAWYQQKPGQAPRLLIYGASNRASGIPARFSGSGSGTDFTLTISSLEPEDFAVYYCQQYHTWPPWAFGGGTKVEIK

SEQ ID NO: 35 - protein, 121 aa, Artificial Sequence; identical to SEQ ID NO: 29
  Description of Artificial Sequence Synthetic polypeptide

SEQ ID NO: 36 - protein, 106 aa, Artificial Sequence
  Description of Artificial Sequence Synthetic polypeptide
DIQMTQSPSSVSASVGDRVTITCRASQGISNDLAWYQQKPGKAPKLLIYDVSNRASGVPSRFSGSGSGTDFTLTISSLQPEDFANYYCQQYDNPPSFGGGTKVEIK

SEQ ID NO: 37 - protein, 116 aa, Artificial Sequence
  Description of Artificial Sequence Synthetic polypeptide
QVTLRESGPALVKPTQTLTLTCTVSGFTLSNPRMGVNWIRQPPGKGLEWIGYISTSGRTDYNPSLKSRVTMSVDTSKNQFSLKVNSVTAADTAVYYCARAGYPDVWGKGTTVTVSS

SEQ ID NO: 38 - protein, 107 aa, Artificial Sequence
  Description of Artificial Sequence Synthetic polypeptide
DIQMTQSPSSVSASVGDRVTITCRASQGIGIDLAWYQQKPGKAPKLLIYDASKLQTGVPSRFSGSGSGTDFTLTISSLQPEDFANYYCRQYATFPQTFGGGTKVEIK

SEQ ID NO: 39 - protein, 116 aa, Artificial Sequence
  Description of Artificial Sequence Synthetic polypeptide
QVQLQESGPGLVKPSQTLSLTCTVSGFPLSTSRMGVNWIRQPPGKGLEWIGYINPSGATDYNPSLKSRVTMSVDTSKNQFSLKVNSVTAADTAVYYCARAGYPDVWGKGTTVTVSS

SEQ ID NO: 40 - protein, 107 aa, Artificial Sequence
  Description of Artificial Sequence Synthetic polypeptide
EIVMTQSPATLSLSPGERATLSCRASQSVSTHLAWYQQKPGQAPRLLIYDATNRATGIPARFSGSGSGTDFTLTISSLEPEDFAVYYCLQYTNYPWTFGGGTKVEIK

SEQ ID NO: 41 - protein, 127 aa, Artificial Sequence
  Description of Artificial Sequence Synthetic polypeptide
EVQLVQSGAEVKKPGASVKVSCKASGYTFPSHYISWVRQAPGQGLEWMGGMNPITGHTIYAQKLQGRGTMTTDPSTSTAYMELRSLRSDDTAVYYCARDMRYDILPGRSYYYGMDVWGQGTTVTVSS

SEQ ID NO: 42 - protein, 108 aa, Artificial Sequence
  Description of Artificial Sequence Synthetic polypeptide
ESALTQPASVSGSPGQSITISCTGTDIRGMSVSWYQQHPGKAPKLMIYEVNQRPSGVSNRFSGSKSGNTASLTISGLQAEDEADYYCAAWDASVAGYVFGGGTKLTVL

SEQ ID NO: 43 - protein, 116 aa, Artificial Sequence; identical to SEQ ID NO: 39
  Description of Artificial Sequence Synthetic polypeptide

SEQ ID NO: 44 - protein, 107 aa, Artificial Sequence
  Description of Artificial Sequence Synthetic polypeptide
EIVMTQSPATLSLSPGERATLSCRASQNIGHSLAWYQQKPGQAPRLLIYGASIRAPGIPARFSGSGSGTDFTLTISSLEPEDFAVYYCQQYITWPSTFGGGTKVEIK

SEQ ID NO: 45 - protein, 116 aa, Artificial Sequence; identical to SEQ ID NO: 39
  Description of Artificial Sequence Synthetic polypeptide

SEQ ID NO: 46 - protein, 108 aa, Artificial Sequence
  Description of Artificial Sequence Synthetic polypeptide
EIVMTQSPATLSLSPGERATLSCRASRSVSSDLAWYQQKPGQAPRLLIYDVSTRATGIPARFSGSGSGTDFTLTISSLEPEDFAVYYCQQYGSSLPPTFGGGTKVEIK

SEQ ID NO: 47 - protein, 116 aa, Artificial Sequence; identical to SEQ ID NO: 39
  Description of Artificial Sequence Synthetic polypeptide

SEQ ID NO: 48 - protein, 105 aa, Artificial Sequence
  Description of Artificial Sequence Synthetic polypeptide
EIVMTQSPATLSLSPGERATLSCRASQSVRTSLAWYQQKPGQAPRLLIYDTSIRAAGIPARFSGSGSGTDFTLTISSLEPEDFAVYYCQQYGNWHFGGGTKVEIK

SEQ ID NO: 49 - protein, 116 aa, Artificial Sequence
  Description of Artificial Sequence Synthetic polypeptide
QVQLQESGPGLVKPSQTLSLTCTVSGVSLSNVRMGVNWIRQPPGKGLEWIGYVNPSGSTDYNPSLKSRVTMSVDTSKNQFSLKVNSVTAADTAVYYCARAGYPDVWGKGTLVTVSS

SEQ ID NO: 50 - protein, 108 aa, Artificial Sequence
  Description of Artificial Sequence Synthetic polypeptide
EIVMTQSPATLSLSPGERATLSCRASQSISSSQLAWYQQKPGQAPRLLIYGASTRATGIPARFSGSGSGTDFTLTISSLEPEDFAVYYCQQYNDRPRTFGGGTKVEIK

SEQ ID NO: 51 - protein, 118 aa, Artificial Sequence
  Description of Artificial Sequence Synthetic polypeptide
QVQLVQSGAEVKKPGASVKVSCKVSGYTFTSSAISWVRQAPGKGLEWMGGIYPGSGHTIYAQKFQGRVTMTEDTSTDTAYMELSSLKSEDTAVYYCAREGDIFVMDVWGLGTAVTVSS

SEQ ID NO: 52 - protein, 107 aa, Artificial Sequence
  Description of Artificial Sequence Synthetic polypeptide
DIQMTQSPSSVSASVGDRVTITCRASMDIRNDLAWYQQKPGKAPKLLIYDASKLETGVPSRFSGSGSGTDFTLTISSLQPEDFANYYCQQAKSFPPTFGGGTKVEIK

SEQ ID NO: 53 - protein, 124 aa, Artificial Sequence
  Description of Artificial Sequence Synthetic polypeptide
EVQLVESGGGLVQPGGSLRLSCAASGFTSSNHWMSWVRQAPGKGLELVASIGPKGLGTYYPDSVKGRFTISRDNAKNSLYLQMNSLRAEDTAVYYCASIRDYGDKKGGSWFDPWGQGTLVTVSS

SEQ ID NO: 54 - protein, 112 aa, Artificial Sequence
  Description of Artificial Sequence Synthetic polypeptide
DIVMTQSPLSLPVTPGEPASISCRSSQSLLHSSGYNFLHWYLQKPGQSPQLLIYMATNRASGVPDRFSGSGSGTDFTLKISRVEAEDVGVYYCVQDLHTLPQFGGGTKVEIK

SEQ ID NO: 55 - protein, 116 aa, Artificial Sequence
  Description of Artificial Sequence Synthetic polypeptide
EVQLVESGGGLVQPGGSLRLSCAASGFTFSDHHMSWVRQAPGKGLELVASISPSGDFTYYPDSVKGRFTISRDNAKNSLYLQMNSLRAEDTAVYYCARDVSPVYYWGQGALVTVSS

SEQ ID NO: 56 - protein, 100 aa, Artificial Sequence
  Description of Artificial Sequence Synthetic polypeptide
DIVMTQSPLSLPVTPGEPASISCRSSQSLLHWYLQKPGQSPQLLIYMGSQRAAGVPDRFSGSGSGTDFTLKISRVEAEDVGVYYCQSGVTFGGGTKVEIK

SEQ ID NO: 57 - protein, 121 aa, Artificial Sequence
  Description of Artificial Sequence Synthetic polypeptide
EVQLVESGGGLVQPGGSLRLSCAASGFTSSGYWMSWFRQAPGKGLELVASIGTAGVTYYPDSVKGRFTISRDNAKNSLYLQMNSLRAEDTAVYYCARGPHGPQQRLRFDPWGQGTLVTVSS

SEQ ID NO: 58 - protein, 112 aa, Artificial Sequence
  Description of Artificial Sequence Synthetic polypeptide
DIVMTQSPLSLPVTPGEPASISCRSSQSLLSSYGYHNLHWYLQKPGQSPQLLIYMGSIRAPGVPDRFSGSGSGTDFTLKISRVEAEDVGVYYCMQGTQAPRTFGGGTKVEIK

SEQ ID NO: 59 - protein, 125 aa, Artificial Sequence
  Description of Artificial Sequence Synthetic polypeptide
EVQLVESGGGLVQPGGSLRLSCAASGFTSSNHWMSWVRQAPGKGLELVASIGPKGLGTYYPDSVKGRFTISRDNAKNSLYLQMNSLRAEDTAVYYCARDVRGWRALYYYYGMDVWGQGTTVTVSS

SEQ ID NO: 60 - protein, 112 aa, Artificial Sequence
  Description of Artificial Sequence Synthetic polypeptide
DIVMTQSPLSLPVTPGEPASISCRSSQSLLHVTGYNYLHWYLQKPGQSPQLLIYGASSRAAGVPDRFSGSGSGTDFTLKISRVEAEDVGVYYCMQSTQWPHTFGGGTKVEIK

SEQ ID NO: 61 - protein, 118 aa, Artificial Sequence
  Description of Artificial Sequence Synthetic polypeptide
QVQLVQSGAEVKKPGASVKVSCKVSGYPSPATISWVRQAPGKGLEWMGWMNPKSGYTNYAQKFQGRVTMTEDTSTDTAYMELSSLKSEDTAVYYCATLHGSGLAFDIWGQGTMVTVSS

SEQ ID NO: 62 - protein, 107 aa, Artificial Sequence
  Description of Artificial Sequence Synthetic polypeptide
DIQMTQSPSSVSASVGDRVTITCRASQDIKSDLAWYQQKPGKAPKLLIYRASARESGVPSRFSGSGSGTDFTLTISSLQPEDFANYYCQQYRSFPWSFGGGTKVEIK

SEQ ID NO: 63 - protein, 120 aa, Artificial Sequence
  Description of Artificial Sequence Synthetic polypeptide
QVQLQQPGTELVKPGASVKLSCKASGYTFTSYWMHWVKQRPGQGLEWIGNINPSNGGTNYNEKFKSKATLTVDKSSSTAYMQLSSLTSEDSAVYYCSITTVVAYWYFDVWGTGTTVTVSS

SEQ ID NO: 64 - protein, 106 aa, Artificial Sequence
  Description of Artificial Sequence Synthetic polypeptide
ENVLTQSPAIMSASPGEKVTISCSASSSVSYMYWYQQKPGSSPKPWIFRTYNLASGVPARFSGSGSGTSYSLTISNMEAEDAATYYCQQRSSYPWTFGGGTKLEIK

SEQ ID NO: 65 - protein, 123 aa, Artificial Sequence
  Description of Artificial Sequence Synthetic polypeptide
EVQLVQSGAEVKKPGASVKVSCKASGDTSSHSHIHWVRQAPGQGLEWMGWINPRSGATNYAQKLQGRGTMTTDPSTSTAYMELRSLRSDDTAVYYCARDRGGRTNGVGAFDIWGQGTMVTVSS

SEQ ID NO: 66 - protein, 110 aa, Artificial Sequence
  Description of Artificial Sequence Synthetic polypeptide
ESALTQPASVSGSPGQSITISCTGTSGDIGRYNYVSWYQQHPGKAPKLMIYEDIERPSGVSNRFSGSKSGNTASLTISGLQAEDEADYYCQVWDATLDLVFGGGTKLTVL

SEQ ID NO: 67 - protein, 122 aa, Artificial Sequence
  Description of Artificial Sequence Synthetic polypeptide
QVTLRESGPALVKPTQTLTLTCTVSGFSLSDTRWGVNWIRQPPGKALEWIGYINDRGTTDYNSALKSRLTISKDTSKNQVVLTMTNMDPVDTATYYCAHTLHTREFVGMDVWGQGTTVTVSS

SEQ ID NO: 68 - protein, 113 aa, Artificial Sequence
  Description of Artificial Sequence Synthetic polypeptide
DIVMTQSPDSLAVSLGERATINCKSSQSVLDTSTNRNYLAWYQQKPGQPPKLLIYGASIRAGGVPDRFSGSGSGTDFTLTISSLQAEDVAVYYCQQYETWPWTFGGGTKVEIK

SEQ ID NO: 69 - protein, 127 aa, Artificial Sequence; identical to SEQ ID NO: 41
  Description of Artificial Sequence Synthetic polypeptide

SEQ ID NO: 70 - protein, 110 aa, Artificial Sequence
  Description of Artificial Sequence Synthetic polypeptide
ESALTQPASVSGSPGQSITISCTGTTGSIASNYVSWYQQHPGKAPKLMIYDTTNRPPGVSNRFSGSKSGNTASLTISGLQAEDEADYYCQSTERGRSDVIFGGGTKLTVL

SEQ ID NO: 71 - protein, 122 aa, Artificial Sequence
  Description of Artificial Sequence Synthetic polypeptide
QVQLVQSGAEVKKPGASVKVSCKASGYTFTSYGLSWVRQAPGQGLEWMGWIIPYNGNTNSAQKLQGRVTMTTDTSTSTAYMELRSLRSDDTAVYFCARDRDYGVNYDAFDIWGQGTMVTVSS

SEQ ID NO: 72 - protein, 106 aa, Artificial Sequence
  Description of Artificial Sequence Synthetic polypeptide
SYEVTQAPSVSVSPGQTASITCSGDKLGDKYACWYQQKPGQSPVLVIYQDSKRPSGIPERFSGSNSGNTATLTISGTQAMDEADYYCQAWDSSTAVFGGGTKLTVL

SEQ ID NO: 73 - protein, 453 aa, Artificial Sequence
  Description of Artificial Sequence Synthetic polypeptide
QVQLQESGPGLVKPSETLSLTCTVSGGSFSSHFWSWIRQPPGKGLEWIGYILYTGGTSFNPSLKSRVSMSVGTSKNQFSLKLSSVTAADTAVYYCARARSGITFTGIIVPGSFDIWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 74 - protein, 215 aa, Artificial Sequence
  Description of Artificial Sequence Synthetic polypeptide
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 75 - protein, 330 aa, Artificial Sequence [sequence omitted]

SEQ ID NO: 76 - protein, 107 aa, Artificial Sequence [sequence omitted]

SEQ ID NO: 77 - protein, 330 aa, Artificial Sequence [sequence omitted]

SEQ ID NO: 78 - protein, 7 aa, Artificial Sequence
  Description of Artificial Sequence Synthetic peptide
DVSNRAS

SEQ ID NO: 79 - protein, 7 aa, Artificial Sequence
  Description of Artificial Sequence Synthetic peptide
DASKLQT

SEQ ID NO: 80 - protein, 7 aa, Artificial Sequence
  Description of Artificial Sequence Synthetic peptide
DATNRAT

SEQ ID NO: 81 - protein, 7 aa, Artificial Sequence
  Description of Artificial Sequence Synthetic peptide
EVNQRPS

SEQ ID NO: 82 - protein, 7 aa, Artificial Sequence
  Description of Artificial Sequence Synthetic peptide
GASIRAP

SEQ ID NO: 83 - protein, 7 aa, Artificial Sequence
  Description of Artificial Sequence Synthetic peptide
DVSTRAT

SEQ ID NO: 84 - protein, 7 aa, Artificial Sequence
  Description of Artificial Sequence Synthetic peptide
DTSIRAA

SEQ ID NO: 85 - protein, 7 aa, Artificial Sequence
  Description of Artificial Sequence Synthetic peptide
GASTRAT

SEQ ID NO: 86 - protein, 7 aa, Artificial Sequence
  Description of Artificial Sequence Synthetic peptide
DASKLET

SEQ ID NO: 87 - protein, 7 aa, Artificial Sequence
  Description of Artificial Sequence Synthetic peptide
MATNRAS

SEQ ID NO: 88 - protein, 7 aa, Artificial Sequence
  Description of Artificial Sequence Synthetic peptide
MGSQRAA

SEQ ID NO: 89 - protein, 7 aa, Artificial Sequence
  Description of Artificial Sequence Synthetic peptide
MGSIRAP

SEQ ID NO: 90 - protein, 7 aa, Artificial Sequence
  Description of Artificial Sequence Synthetic peptide
GASSRAA

SEQ ID NO: 91 - protein, 7 aa, Artificial Sequence
  Description of Artificial Sequence Synthetic peptide
RASARES

SEQ ID NO: 92 - protein, 7 aa, Artificial Sequence
  Description of Artificial Sequence Synthetic peptide
RTYNLAS

SEQ ID NO: 93 - protein, 7 aa, Artificial Sequence
  Description of Artificial Sequence Synthetic peptide
EDIERPS

SEQ ID NO: 94 - protein, 7 aa, Artificial Sequence
  Description of Artificial Sequence Synthetic peptide
GASIRAG

SEQ ID NO: 95 - protein, 7 aa, Artificial Sequence
  Description of Artificial Sequence Synthetic peptide
DTTNRPP

SEQ ID NO: 96 - protein, 9 aa, Artificial Sequence
  Description of Artificial Sequence Synthetic peptide
QQYTAYPLT

SEQ ID NO: 97 - protein, 8 aa, Artificial Sequence
  Description of Artificial Sequence Synthetic peptide
QQYFTLPT

SEQ ID NO: 98 - protein, 9 aa, Artificial Sequence
  Description of Artificial Sequence Synthetic peptide
MQGTQAPRT

SEQ ID NO: 99 - protein, 9 aa, Artificial Sequence
  Description of Artificial Sequence Synthetic peptide
VQDLHTPFT

SEQ ID NO: 100 - DNA, 348 bp, Artificial Sequence
  Description of Artificial Sequence Synthetic polynucleotide
caagtccagctgcaagaatcaggccccggactcgttaagcccagtcagaccttgtcccttacctgcactgtctcaggtctcagcctcagcaatgcgaaaatgggagtgaattggatccggcaaccaccaggaaagggtcttgagtggattgggtatgtttcaactactggaactaccgattacaatccatctctcaagtcccgggtgacaatgagtgtagatacttctaagaaccaatttagcttgaaagtgaattccgtaacggctgcggacacggcagtctattactgcgctcgagccggctaccctgacgtttggggcaaaggcacaacagtcactgtgtcctct

SEQ ID NO: 101 - DNA, 348 bp, Artificial Sequence
  Description of Artificial Sequence Synthetic polynucleotide
caagtccaactccaagaatccgggcccggccttgtcaaacccagtcaaacactctctctcacatgcacagtaagcggggcctcagtaagcaatgcacgaactggagtgaactggatccggcaacccccagggaaaggactcgaatggattggatatattaaccccagtgggagaatcgattataatccatccctcaaaagcagggtcaccatgtccgtggacacatccaagaatcaattcagcttgaaggtgaactccgtaacagcggctgacactgccgtgtattactgtgcacgggcaggataccctgatgtttggggcaagggaaccactgtcacagtgagtagt

SEQ ID NO: 102 - DNA, 375 bp, Artificial Sequence
  Description of Artificial Sequence Synthetic polynucleotide
gaagttcagctcgtggaaagcggtggcggattggtgcaacctggcggatcccttagactgagttgcgccgcttctgggtttactgcgagcagaaactatatgtcatgggtacgacaggccccgggtaaaggactggagctggtggcatctatctcagggtcaggtgactacacctactaccctgatagcgttaaaggacggttcacaatcagcagggataatgctaagaactccttgtatctccagatgaacagcctcagggccgaggatacagcagtgtactattgcgcgcgtagtcccagagggccgcgtctgctgggctattactatgggatggacgtgtggggacaaggaactacagtgactgtctcatca

SEQ ID NO: 103 - DNA, 360 bp, Artificial Sequence
  Description of Artificial Sequence Synthetic polynucleotide
gaagttcaattggtagaatctgggggggggttggttcagcctggcggctcactgaggcttagctgtgcagcttccgggttcgattttggagaacattggatgtcctgggtaagacaagcaccaggaaaagggcttgaactggtggcctctataaagggagatgcatccgacacctactaccccgattccgtcaaaggccgatttaccatctcacgggataacgctaagaattctctttacttgcaaatgaattctctccgcgctgaagacaccgcagtctattattgtgtaaaggacttgtcaaagcagtggctgggcattgactactggggccaagggacccttgtgaccgttagtagt

SEQ ID NO: 104 - DNA, 369 bp, Artificial Sequence
  Description of Artificial Sequence Synthetic polynucleotide
gaagttcaactggtagaatctggtggcggtcttgtgcaacccggcggcagtctgcgactctcctgtgcagcgagcgggtatacattctctaactacgggatgtcatgggttcgtcaagcgccagggaaaggcctcgaactcgtcgctagtatatccaagtctggttcttccatctactatccagactccgtgaagggaaggtttaccatcagcagagataatgcgaagaactcactttacctgcagatgaactccttgcgcgcggaagacacagccgtctactattgcgcccgacttcccaggttggcttggaattattattacgggatggatgtttggggacaaggcactactgtcactgtttccagc

SEQ ID NO: 105 - DNA, 348 bp, Artificial Sequence
  Description of Artificial Sequence Synthetic polynucleotide
caagttcaacttcaggaatctggccctggcttggtcaaaccgtcccaaactctcagtctcacttgcactgtttcaggactttctcttagtaatgcgaagatgggcgtgaattggataaggcaaccacccggtaaaggtctcgaatggattggctatgtttcaaccactggcactacggattataatccaagtctgaaatctcgagtaacaatgagcgttgataccagcaagaatcagttcagcctgaaggtgaacagtgttacagccgctgacacagctgtgtactattgtgctagggctggataccctgatgtgtggggtaaggggacaactgtcacggtaagctca

SEQ ID NO: 106 - DNA, 363 bp, Artificial Sequence
  Description of Artificial Sequence Synthetic polynucleotide
caagtccaactccaagagagcggccccgggctcgtcaaacctagccaaaccctgtcactgacatgcacagtctctggagtgtctctgtccaacgcacgcttgggcgttaactggatacgccagccacccggaaaaggtctcgaatggattggttatgggagcgtgacaggctctaccgattataacccaagtcttaaatccagggtcacgatgtctgttgacacaagcaagaatcaatttagcttgaaggtcaactccgtcactgctgcagacactgcagtatattactgtacaaccagctatctggtggacacggctatggtaaccaggtggggtcagggtacactcgtcacagtgtcctct

SEQ ID NO: 107 - DNA, 348 bp, Artificial Sequence
  Description of Artificial Sequence Synthetic polynucleotide
caagttcagctccaagaatccggtccaggcctggtgaagccctctcaaacattgagcctcacctgcaccgtaagcggctacagtttgacagatgctaggataggtgttaactggatccgtcaaccgccgggcaaaggtttggagtggattggctatataaacccaagcgggcgcacagattacaatccctcactcaagtcccgtgtgactatgtcagtcgatactagcaagaatcaattcagccttaaggtgaattcagtaaccgcagccgatacagccgtctattactgtgctagagccgggtatcctgacgtctggggcaaagggacaacagtaacagttagtagt

SEQ ID NO: 108 - DNA, 369 bp, Artificial Sequence
  Description of Artificial Sequence Synthetic polynucleotide
gaagtgcaacttgtagaatctggtggcgggctcgtccaacctggcggatctctgcgtttgtcctgtgccgcaagcggctatacctttagcaattatggtatgagttgggtcaggcaagccccgggcaagggtttggaattggtcgcgagcatatcaaagtctgggagtagcatttactatcccgacagtgtaaaggggcgctttaccataagccgcgataacgctaagaattccctctatctccaaatgaattcccttcgtgcagaagatacagcagtttactattgcgcgagacttccacgtcttgcatggaactactattatggaatggacgtttggggccaaggtactaccgtcacagtaagttca

SEQ ID NO: 109 - DNA, 369 bp, Artificial Sequence
  Description of Artificial Sequence Synthetic polynucleotide
gaagtgcaactcgtggaatcaggtggcggattggtacaacccggcggcagcttgcgtctctcttgtgccgcctctggtttcactctgagcaactacccaatgagttgggtaagacaagcacctggaaagggcctggagcttgtagcgagtactacaagcggtagctccaccatctactacccggactcagtcaaagggcgattcacgattagccgagacaacgctaagaactctttgtatcttcaaatgaactctctgcgtgctgaagacaccgcggtctattactgcgctcgcggtccagactattatgcaccgcttaggtcctacatggatgtctggggcaagggaaccaccgtgactgtatcttct

SEQ ID NO: 110 - DNA, 363 bp, Artificial Sequence
  Description of Artificial Sequence Synthetic polynucleotide
caagtgcaactccaagaaagtgggcctggactcgtgaagcccagccaaactctgagtcttacctgcaccgtcagcggagtcagtctctccaacgctcgtcttggtgtaaattggatccggcagccaccagggaaaggactggagtggatcggttatggatccgtcacgggaagcaccgattacaatccctctctcaaaagtagggtgactatgagcgtcgatacttctaagaatcaattcagcctcaaagttaattctgttaccgcagctgacactgcggtgtattattgcacgactagctatttggtggacacagctatggtcacgcggtggggccaaggaacactggttactgtctcttct

SEQ ID NO: 111 - DNA, 348 bp, Artificial Sequence
  Description of Artificial Sequence Synthetic polynucleotide
caagttcaactgcaagaaagcggcccaggcctcgtgaaaccaagccaaacactcagcctgacatgcactgtatctggcctcagccttagtaacgccaaaatgggcgtgaactggattcggcaaccaccgggcaaagggcttgaatggattggctacgtgtccacaaccggtacgacggattacaatccatccctgaaatcccgtgtcaccatgagcgttgacacgtccaagaaccaattcagcttgaaagtaaatagcgtaacagccgcagacacagctgtctattattgtgcgcgggcagggtatccagatgtctggggcaagggcactacagtcactgttagcagc

SEQ ID NO: 112 - DNA, 375 bp, Artificial Sequence
  Description of Artificial Sequence Synthetic polynucleotide
gaagtgcaattggttgaaagtggtggtggattggtccaacctggtggctccctgagactcagctgcgctgcatccggtttcacagcttctcgaaattacatgagctgggtacgacaagcaccagggaaaggacttgaactggttgccagtatcagtggcagtggggactatacatactatcccgattctgtcaaaggcaggttcaccattagccgcgacaacgctaagaattcactttatttgcagatgaattcacttagggctgaggacactgctgtctattactgtgccagatcacccaggggcccgcggttgctggggtattattatggaatggacgtatggggccagggaacaaccgtcactgtttcttct

SEQ ID NO: 113 - DNA, 348 bp, Artificial Sequence
  Description of Artificial Sequence Synthetic polynucleotide
caggtgcaactccaagaatcaggcccggggctcgttaaaccctcacagaccctcagcctgacatgtaccgtcagtggttattctctgacggacgccagaatcggtgtcaactggataaggcaaccaccagggaaaggcttggaatggatcggttatattaaccctagtgggcgcactgactataaccccagccttaaaagccgggtgactatgtcagtagatacttctaagaatcaattcagtctgaaagtcaactccgtgacggcagcggataccgccgtctattattgcgccagggctggttatcctgacgtgtggggcaagggcaccactgttacggtttcttct

SEQ ID NO: 114 - DNA, 363 bp, Artificial Sequence
  Description of Artificial Sequence Synthetic polynucleotide
caagttaccctgcgagaaagtggtcccgctcttgtcaagcccacccaaaccttgaccctcacttgtaccgtaagtggctttagcttgtccacatccggagcaacagtcaattggatccgacaacccccggggaaggccctcgaatggattggttatataacatcctcaggacgcactgtttataattccgctctgaagagtcgcttgaccataagcaaagacacctcaaagaatcaggtggtgctgaccatgactaatatggaccccgtcgatactgccacctattattgtaccaccaccaaaattgtgggcgctaccctgagagattattggggtcaaggaacactcgtaacggtttccagt

SEQ ID NO: 115 - DNA, 378 bp, Artificial Sequence
  Description of Artificial Sequence Synthetic polynucleotide
gaagttcaactggtgcagtcaggagccgaagtaaagaaaccaggcgcatctgtgaaagtttcatgtaaggcttccggctacacttacacagggtatcgaatcagctgggtccggcaggctcccgggcaaggtctggaatggatggggtggatagatccgaagagtgggcggactaactacgcacagaaacttcaaggtcggggcacaatgactaccgatccatctacaagtactgcctatatggaacttcgctccttgagatctgatgataccgcggtctactattgtgcccgtgaagtaaaacagtggcttgtcccaggacgtggagtgtactggtttgacccatggggacaaggtacacttgtaactgtcagctcc

SEQ ID NO: 116 - DNA, 348 bp, Artificial Sequence
  Description of Artificial Sequence Synthetic polynucleotide
caggtgcaactgcaagagtcaggccccggcctcgttaaaccttctcaaacactcagcctcacctgcacggttagcggagtttccctgtccgatgcgcggatgggcgtcaactggattaggcaaccacccggcaagggtcttgagtggatcggatatgtcagcaccacaggaaccaccgactacaacccctcattgaagtcacgggtgaccatgtctgtggacacctctaagaaccaattttccctcaaagtgaactccgtgaccgctgcggacactgcagtttattattgtgccagggccgggtacccggacgtgtggggtaagggtactttggtgacagtgagtagt

SEQ ID NO: 117 - DNA, 363 bp, Artificial Sequence
  Description of Artificial Sequence Synthetic polynucleotide
caagtcaccttgagagaatccggacccgcgctcgtaaaacccactcaaaccctgacattgacttgtactgtctcaggtttcagtctctcaactagcggagcgacagtcaactggatacgtcaaccaccaggaaaggccctggagtggattggatacattacatctagtggcaggaccgtgtataattctgccctcaaatcccgccttacaatctccaaagacacatccaagaatcaagtagtcctgaccatgactaatatggaccctgtggacacggccacctactattgcacaacaaccaaaattgttggcgcgaccctgcgtgattattggggccaaggcactctggtgaccgttagcagc

SEQ ID NO: 118 - DNA, 348 bp, Artificial Sequence
  Description of Artificial Sequence Synthetic polynucleotide
caggtgaccttgagggagtccgggccagcacttgttaaaccaacgcaaacccttactctcacgtgcactgtatctgggttcaccctgtcaaatcctagaatgggcgttaactggatacgccaaccgccaggtaagggactggagtggatcgggtacataagcaccagcggacgcaccgactataaccctagtctcaaaagtcgcgtcacgatgtcagtggacacctcaaagaatcaattcagcctgaaggtcaattcagttactgctgcagatacggctgtttattactgtgccagagctggctacccagacgtatggggaaaaggaacgacagtgacagtcagtagc

SEQ ID NO: 119 - DNA, 348 bp, Artificial Sequence
  Description of Artificial Sequence Synthetic polynucleotide
caagtgcagctgcaagaaagtggaccaggactggtgaaaccgagccaaacattgtccctcacctgtaccgtctctgggtttcccttgagtaccagccgcatgggcgtgaactggattcggcagccacctggaaagggcctggaatggattggttacatcaatccttccggggccactgattataacccttccttgaaaagcagggttactatgtctgtggacaccagcaagaaccaatttagtcttaaggtgaactccgtgaccgctgccgatactgcggtttattactgcgcacgtgcggggtatccagatgtatggggcaaaggaactactgtgactgtatcttct

SEQ ID NO: 120 - DNA, 381 bp, Artificial Sequence
  Description of Artificial Sequence Synthetic polynucleotide
gaagtccaattggtgcaaagcggcgcggaagtgaagaaacctggagcctcagttaaagtcagttgtaaagcttctgggtacaccttcccgtctcactacatatcctgggtccggcaggccccagggcaaggtttggaatggatgggcgggatgaacccaattacagggcacactatttatgcccaaaagctgcaaggaaggggcacaatgactaccgacccatccacatccactgcatatatggaattgcgtagtctccggagcgatgataccgccgtgtactattgtgcaagagatatgcgctacgatatcctgccagggcggtcatactattatggtatggatgtttggggccaaggcactaccgtcacggtttcatca

SEQ ID NO: 121 - DNA, 348 bp, Artificial Sequence
  Description of Artificial Sequence Synthetic polynucleotide
caagtccaattgcaagagtctggcccggggctggtaaaaccttctcaaacgcttagcctgacttgcacagtgtccgggtttcccttgtcaactagccgtatgggcgttaattggattcgacaaccaccaggtaaaggacttgagtggataggctatatcaacccctctggcgcgaccgattacaaccctagtctcaaatcccgggtcaccatgagtgtggatacctcaaagaatcagttctccctcaaagtaaactccgtgacggctgcagacaccgcagtgtattactgtgcacgggctggttaccccgatgtgtggggcaaaggaacaacagttactgtctcttct

SEQ ID NO: 122 - DNA, 348 bp, Artificial Sequence
  Description of Artificial Sequence Synthetic polynucleotide
caagtccaactgcaagagtcaggccctggtctcgttaagccatcccaaacgctgagtcttacctgtacggtatccggtttccccctctccactagtcgaatgggcgtaaactggattcggcagccaccaggcaaaggactggaatggatagggtatataaatccgagtggcgccaccgattataaccctagcctgaaatcacgtgtgactatgagtgtggacaccagcaagaatcaatttagcttgaaagtcaatagtgtgactgcagcagacacagcagtctactactgtgcccgcgccggctatcctgacgtctggggtaaaggtacaacggtgacggtgtcatca

SEQ ID NO: 123 - DNA, 348 bp, Artificial Sequence
  Description of Artificial Sequence Synthetic polynucleotide
caagtccaattgcaagaaagcggcccaggtctggtcaagcccagtcagacactctcactgacatgtaccgtttcaggctttcccctttcaacttctcgtatgggcgtcaactggattaggcagccaccaggaaaaggcctggagtggataggatatatcaatccatctggggcgactgattataatcccagcttgaagagccgcgttacaatgagtgtcgataccagcaagaatcaattctccctgaaagttaacagtgttacagccgcagatacagcggtttactactgcgcccgcgccggataccccgacgtgtggggaaaaggtaccaccgtcacagtcagctcc

SEQ ID NO: 124 - DNA, 348 bp, Artificial Sequence
  Description of Artificial Sequence Synthetic polynucleotide
caagtgcaactgcaagaatcaggtcccggcctcgttaagcccagccaaaccctcagtcttacctgtaccgtgagtggcgtgagtttgtctaatgtacgcatgggcgtcaattggatccgacaaccacccgggaagggacttgagtggattgggtatgttaatccttccgggtcaactgattataatccatcactgaaatcacgggtcaccatgtctgttgatacttccaagaaccaattttcattgaaagttaactccgtgacggcagcagacacagcagtttattactgtgcccgcgcaggatatcctgatgtgtggggtaagggcactctcgtaacggtttcctct

SEQ ID NO: 125 - DNA, 354 bp, Artificial Sequence
  Description of Artificial Sequence Synthetic polynucleotide
caagtccaacttgtgcaaagcggcgctgaagttaagaaacctggagctagtgtcaaggtcagctgcaaagtgagcggttatacctttacaagtagcgcaatcagttgggtacggcaagctccaggcaaaggcctggaatggatgggcgggatatacccaggaagcggacacactatttacgctcaaaagtttcaaggccgcgtgacgatgaccgaggatacatccacggatacggcttatatggagttgtcttcactgaagtctgaagacactgctgtgtactattgtgctcgggaaggcgacatctttgtgatggatgtttggggtctgggtacagccgtcacggtcagttct

SEQ ID NO: 126 - DNA, 372 bp, Artificial Sequence
  Description of Artificial Sequence Synthetic polynucleotide
gaagtacagcttgttgaaagtggcggtgggctcgtccaacccggtggatccctcagactcagctgtgcagcgagcggcttcacttcatcaaatcattggatgagttgggtgaggcaagccccaggaaagggcttggaacttgtggccagtattggccctaaaggattgggtacttactaccccgattctgtcaagggccgctttacaatcagcagggacaatgccaagaactctttgtatctgcaaatgaattctcttcgcgctgaagacaccgccgtttactattgtgcatctattagagactatggagacaagaagggcggcagctggtttgacccctggggtcagggaacacttgttaccgtaagtagt

SEQ ID NO: 127 - DNA, 348 bp, Artificial Sequence
  Description of Artificial Sequence Synthetic polynucleotide
gaagttcaactggtggaatctggtggtggtctggtgcaacctggtggttcattgcgtctctcttgcgcagcctcaggtttcacattttctgatcatcatatgagctgggtgcgacaggcgcccggtaaaggcctcgagcttgtcgcttcaatcagccctagtggtgatttcacctactacccagatagcgtcaaaggacggtttaccatctcccgggataatgcaaagaacagtctgtatttgcaaatgaatagcttgcgtgcagaggatacagctgtctattattgcgctagggatgtgagccccgtgtactactggggccaaggcgctctggtaaccgtaagctcc

SEQ ID NO: 128 - DNA, 363 bp, Artificial Sequence
  Description of Artificial Sequence Synthetic polynucleotide
gaagtacagcttgtggagagtggaggaggtttggtgcaacctggtgggagcctgcgactctcctgtgctgcatccggattcactagctccggatattggatgtcatggtttagacaggctccagggaaggggcttgagctcgtggcgagtatcgggactgccggagttacctattatcccgattcagtgaaaggacggttcaccattagccgcgacaatgccaagaattccctgtaccttcaaatgaactccttgagagcggaagatacagcagtttattactgcgcgagaggccctcatggtccccaacaaagattgcgcttcgatccatggggacaaggcactctggtaactgttagtagt

SEQ ID NO: 129 - DNA, 375 bp, Artificial Sequence
  Description of Artificial Sequence Synthetic polynucleotide
gaagtgcagctcgtggagagtggaggaggtcttgtacaacccggtggatccctgcgactctcctgcgctgccagcggattcacaagtagtaaccattggatgtcatgggtgaggcaggcacccggtaaaggcctggagttggtcgcttcaataggacccaagggccttggtacctattatccagacagtgtgaagggtcggttcactattagtcgcgataacgctaagaattcactgtatcttcagatgaattctttgcgtgccgaagatactgctgtctattactgtgctagagacgttcgaggttggagagcactgtattattattacggtatggacgtgtggggtcaagggaccaccgtgaccgtctcttct

SEQ ID NO: 130 - DNA, 354 bp, Artificial Sequence
  Description of Artificial Sequence Synthetic polynucleotide
caggtgcaacttgttcagagcggagccgaagtaaagaaaccaggggcctctgttaaagtgtcttgcaaagtttcagggtacccaagccctgctaccatatcttgggtacggcaagcgcccggcaaaggactcgaatggatgggatggatgaatcccaaaagtggatatacgaattacgcacaaaagtttcaaggcagagtcaccatgacggaagatacctcaactgataccgcttatatggaactctcatcacttaaatctgaagataccgctgtctattactgcgcaacactccacggtagtggtctcgcttttgatatttggggacaggggactatggtcacggtcagcagc

SEQ ID NO: 131 - DNA, 360 bp, Artificial Sequence
  Description of Artificial Sequence Synthetic polynucleotide
caagtgcaactacaacaaccaggtactgaactggtgaaacccggcgcttccgtgaagctgtcctgcaaggcctctggctacaccttcacctcctactggatgcactgggtcaagcagagacctggccagggcctggaatggatcggcaacatcaatccttccaacggcggaaccaactacaacgagaagttcaagtccaaggctaccctgaccgtggacaagtctagctccaccgcctacatgcagctgtcttctctgacctctgaggactctgccgtgtactactgttccatcaccacagtggtggcctattggtactttgatgtgtggggcacaggaaccaccgtgaccgtgtccagc

SEQ ID NO: 132 - DNA, 369 bp, Artificial Sequence
  Description of Artificial Sequence Synthetic polynucleotide
gaagtgcaactagtgcaaagtggtgcagaagtcaagaagcctggagcttccgtgaaagtgtcctgcaaggcctccggcgacacatcctctcatagccacatccactgggtgcggcaggctcctggccaaggcctggagtggatgggctggatcaacccccgctctggcgctaccaactacgctcagaagctgcagggcagaggaaccatgaccaccgatccttctacctctacagcctacatggaactgcggtccctgagatccgacgacaccgccgtgtactactgtgccagagatagaggcggtcggaccaacggcgtgggcgccttcgacatctggggccagggcaccatggtgaccgtgtctagc

SEQ ID NO: 133 - DNA, 366 bp, Artificial Sequence
  Description of Artificial Sequence Synthetic polynucleotide
caagtgactctacgagaaagtggtccagcactggtgaagcccacccaaacactcacactgacctgtaccgtgtccggcttcagcctgtctgataccagatggggcgtgaattggatccggcagcctcctggcaaggccctggaatggatcggctacatcaacgaccggggaaccaccgactacaacagcgctctgaagtccagactgaccatctccaaagacacctccaagaaccaggtggtgctgaccatgaccaacatggatcctgtggacaccgctacctactactgcgcccacaccctgcacacaagagagttcgtgggcatggacgtgtggggccagggcaccacagtgaccgtctcttct

SEQ ID NO: 134 - DNA, 381 bp, Artificial Sequence
  Description of Artificial Sequence Synthetic polynucleotide
gaagtgcaactagtgcaaagtggtgcagaagtcaagaagcctggcgcttccgtgaaagtgagctgcaaggcctccggctacaccttcccctctcactacatctcctgggtgcggcaggctccaggccagggcctggagtggatgggcggcatgaaccctatcaccggccacaccatctacgcccagaagctgcagggcagaggcaccatgaccacagatccttctacctccaccgcctacatggaactgcggtctctgagatccgacgacaccgctgtgtactattgtgccagagatatgagatacgacatcctgcctggacggtcctactactacggcatggacgtgtggggccaaggaacaaccgtgaccgtgtctagc

SEQ ID NO: 135 - DNA, 321 bp, Artificial Sequence
  Description of Artificial Sequence Synthetic polynucleotide
gatattcaaatgactcaatctccgtctagcgtgtcagcttctgtcggcgatcgcgtgaccataacgtgtcgtgcaagtcaaaatattggggctgccctcgcttggtatcaacaaaagcccggaaaggcaccaaagttgctgatttatggagcatctacccgcgaatatggcgtgccttcccggttttccggttccgggagtggtactgactttacactgaccatctcttcactgcaacccgaagacttcgcaaactattattgccaacaatatactgcataccccctcacattcggtggcggaaccaaggtagagataaag

SEQ ID NO: 136 - DNA, 318 bp, Artificial Sequence
  Description of Artificial Sequence Synthetic polynucleotide
gagatcgtgatgacacaaagcccagctaccctttcacttagccccggtgaaagggccactctttcctgtcgcgcaagccagacagtgtcatctcatctggcgtggtatcagcaaaagcctggacaagcaccccggcttcttatctatgacgtgtcacatcgggcatcaggaattcccgccaggttttcagggtcaggcagcggaaccgatttcaccctcaccatcagttcactcgagccagaggatttcgcggtgtactattgtcaacaatattttacgttgcccacctttggaggaggaactaaggtcgagataaaa

SEQ ID NO: 137 - DNA, 336 bp, Artificial Sequence
  Description of Artificial Sequence Synthetic polynucleotide
gacatcgtcatgacccaaagccctctcagtctccctgtcacacctggtgaacccgccagtatctcatgccgttccagcagatctctggttcatggttcaggccataattacctgcattggtacctgcaaaagcccggccaatccccacagttgttgatatatggagcatctacccgggtcgcaggcgtccccgacaggttctctggctctggatccggtactgatttcaccttgaagattagcagagtcgaagccgaagatgtgggagtatactattgtatgcagggcacgcaagctcccaggaccttcggtggcggtaccaaggtggaaattaaa

SEQ ID NO: 138 - DNA, 336 bp, Artificial Sequence
  Description of Artificial Sequence Synthetic polynucleotide
gatattgtaatgacgcaaagtcccctgagtctgcccgtcacacccggtgagcccgcaagtatttcctgcaggagcagtcaaagtctgctgcattccaatgaaaagaactatctgcattggtaccttcagaaaccaggccaaagccctcagttgttgatatatgatgcgtcaaagagggccagtggcgtccccgaccggttcagcgggtccggaagcggcactgattttacactcaagattagccgggtcgaagctgaagatgtaggagtctattattgcgtgcaagacttgcacacacctttcacatttggaggaggtaccaaggttgaaatcaag

SEQ ID NO: 139 - DNA, 336 bp, Artificial Sequence
  Description of Artificial Sequence Synthetic polynucleotide
gatatagtaatgacacaaagtcctcttagcctgccggtcactcctggtgagcctgcttctataagctgtagatcaagtaggtctctggtgcacgggtcaggcgacaactacttgcattggtacctgcaaaagcctgggcagagccctcaactgctcatctatatggcaagtaaccgggctcctggcgttccggatcgattttctggaagcggatccggaacagacttcactctcaaaatctctagagtcgaggccgaagatgtgggcgtgtattactgcgtccaaaccttgcagaccccatccacttttggcggcggaaccaaggtcgaaatcaaa

SEQ ID NO: 140 - DNA, 321 bp, Artificial Sequence
  Description of Artificial Sequence Synthetic polynucleotide
gagatcgtgatgacccagtcccctgcaacattgagcctcagtccgggtgaaagggcgaccctgtcttgtcgcgcttcccaaaatgtctataccaacctcgcctggtaccaacaaaagcctggccaggctccacgcctgttgatctatggtgcttccgctcgggctagcggtatacctgcccgcttcagtggcagtggctctggaaccgactttacccttaccatctcttctttggaaccagaggatttcgctgtgtactactgcctgcagtacggcagtagcccacccctcttcggaggaggaacgaaagtggaaattaag

SEQ ID NO: 141 - DNA, 321 bp, Artificial Sequence
  Description of Artificial Sequence Synthetic polynucleotide
gaaatagtaatgactcaaagcccagctactctttcattgagtcccggggaacgggccactctgtcttgccgggcctctcaatcattgggaaccaatcttgcatggtaccagcaaaagcccggtcaagcccctcggttgttgatttatggagcttcaagtcggcctagcggcatacctgccagattttctggttccgggtccggcactgattttactctgacaatatcttcccttgaaccagaggactttgcagtatactattgccaacaatatgatacacatccgctcagctttggtggtggcactaaagtcgaaatcaaa

SEQ ID NO: 142 - DNA, 321 bp, Artificial Sequence
  Description of Artificial Sequence Synthetic polynucleotide
gatatacagatgacgcaatcccccagcagcgtgagcgcttcagtcggcgatcgggtaaccattacatgcagagccagtcaaactataagtcggtctttggcttggtaccagcagaaacccggaaaagccccaaaactgctcatctacgacgccagcagtcttgctggcggagtgccatctcggttttccggttctgggtctggaacagatttcacactgactatctcaagcttgcaacccgaagattttgctaattattattgccaacaatatagttcatatccaagcctctttggcggcggcacaaaagtagagattaag

SEQ ID NO: 143 - DNA, 336 bp, Artificial Sequence
  Description of Artificial Sequence Synthetic polynucleotide
gatatcgtaatgacacaatccccgctgtctctcccggtgacgccaggcgagcctgcgtctatctcctgtaggagttcacaaagtctcctgcactctaacgagaagaactacctgcattggtacttgcaaaagccgggacagagccctcagctcctcatctacgatgcatcaaaacgagcctcaggcgtccctgatcggttctccggaagtggatctgggacggactttactttgaagattagccgcgtagaagccgaggatgtgggcgtctattattgcgtacaagatctccacacaccgtttaccttcggtggtgggaccaaggtggagatcaaa

SEQ ID NO: 144 - DNA, 336 bp, Artificial Sequence
  Description of Artificial Sequence Synthetic polynucleotide
gatatcgtcatgactcaatcacctctctccttgcccgtgactcccggagaaccggccagcattagttgccggtccagtcaaagtttgcaacaaagcgaaggccggacctacctgcactggtacctgcaaaagcccgggcagagtccgcagctgctgatttacgacgccagtaagcgcgcatccggtgttccggatagattttccggtagtggaagcggaacagattttactttgaagattagccgcgttgaggctgaggacgttggcgtttactattgcatgcaagtccttagaacaccgattactttcggaggaggcacgaaggtggaaatcaaa

SEQ ID NO: 145 - DNA, 312 bp, Artificial Sequence
  Description of Artificial Sequence Synthetic polynucleotide
gaaattgtcatgacacaatccccagctacccttagcctttcccctggagagagggcaacactttcttgtcgtgccagccaacgaattggatccgacttggcatggtaccaacagaagccgggtcaagcaccacgtctcctcatctattctgctagctcccgagcgccggggattcctgcccggtttagcggcagtgggagcggaactgactttacccttaccatctcctcacttgagccagaggactttgctgtttattactgctccacgatggcaattccctttggaggaggaaccaaggttgaaataaag

SEQ ID NO: 146 - DNA, 324 bp, Artificial Sequence
  Description of Artificial Sequence Synthetic polynucleotide
gatatccaaatgacacaaagcccatcatctgtctcagccagtgttggagaccgagttacaattacctgcagggctagccaaggcatctccagagacctggcttggtatcaacaaaagcccgggaaagcgccaaagttgctgatatatgcagcaagcacacttgtttccggcgttcccagtcgcttcagcggatccggcagtggcacagattttacgcttacaataagcagtttgcagcctgaagattttgcaaattactactgtcaacagtatgtatcagctccgccatatacgtttggcggtggtaccaaagtcgagatcaaa

SEQ ID NO: 147 - DNA, 336 bp, Artificial Sequence
  Description of Artificial Sequence Synthetic polynucleotide
gatatcgttatgacgcaaagcccactgagcctgccggttacccccggggagccggctagtatcagctgccggagcagccaatcactgttgcactcaaacgaaaagaattacttgcactggtatctgcagaaacccggccaatcacctcaactgctcatttacgacgccagtaaacgcgcatccggtgttccagatcgcttttccggatctggttcaggcactgattttaccttgaaaatttcccgcgtagaggctgaggatgttggggtatactactgcgtacaagatctccacacacccttcacattcggcggcggaacaaaagtagaaattaag

SEQ ID NO: 148 - DNA, 327 bp, Artificial Sequence
  Description of Artificial Sequence Synthetic polynucleotide
gaaatcgtcatgacacagtctccagctacgttgagtctcagccctggtgaacgggccacattgagttgtagggcatcccaaagtataggaacaaagcttgcgtggtaccaacaaaagccagggcaagctccccgactcctcatttatgatgcgagctctcgcgtgcctggaatccctgcaaggttttcagggtccggatctgggacagattttacactgactatttcctctctcgagccagaagactttgccgtctactactgtcaacaatatgggagtagcccacccgtctactcctttggggggggaactaaagtagaaatcaag

SEQ ID NO: 149 - DNA, 339 bp, Artificial Sequence
  Description of Artificial Sequence Synthetic polynucleotide
gacatagtcatgactcaaagccccgacagcctcgcagtttctctcggggaacgcgcaactatcaactgcaaatcatctcagagtgtgctggatacttcaaccaacagaaattacttggcatggtatcagcaaaagcccggccaaccacccaaactcctcatttatggggcttcaatcagagcgggcggcgtacccgatcggttttccgggagcggaagtggaacagacttcacactgactattagcagtttgcaagcagaagacgtggcggtatactactgccaacaaagctactcacatccgccaacttttggtggtggtactaaagtggaaatcaag

SEQ ID NO: 150 - DNA, 321 bp, Artificial Sequence
  Description of Artificial Sequence Synthetic polynucleotide
gagtctgcattgacacaacccgcatcagttagtggaagccccggccagtctattacaattagttgtacaggtacaaatattgggctccggagtgtgagctggtatcaacaacatccaggtaaagccccgaagctgatgatctatgatacgaatattaggtctagcggcgtgtctaataggttttccgggtccaaatccgggaataccgcttctcttaccattagcgggctgcaggccgaagatgaagcagactattactgctcttcctacacatcatccagtactgacgtcttcggtggcggtaccaaactgacagtcctt

SEQ ID NO: 151 - DNA, 324 bp, Artificial Sequence
  Description of Artificial Sequence Synthetic polynucleotide
gaaatcgttatgactcagagccccgcaacactcagtttgagccccggtgagcgcgccacgttgagctgtcgtgctagcgaatccgttcgctcaaacctcgcgtggtaccagcaaaagccaggtcaggcccccaggctcctgatctacggcgctagcaaccgagcgtccggtattccggcaaggttttcagggtctgggagcgggaccgacttcactctcacaatatcatcactggaacccgaggattttgcggtgtattattgccagcaataccacacttggccgccatgggcctttggtggtggtacgaaggtcgagataaag

SEQ ID NO: 152 - DNA, 318 bp, Artificial Sequence
  Description of Artificial Sequence Synthetic polynucleotide
gatatccaaatgacacaatcccctagctcagtctccgcatctgtgggagatagggtcactattacatgccgtgcttctcaaggtatttccaacgacttggcttggtatcaacaaaagcctggaaaggcaccaaagctgctgatctatgatgtctccaatcgggcttccggcgtccctagccggttctctgggagcggatctggcaccgacttcacacttactattagctctcttcaacccgaggattttgccaattattactgtcaacaatacgataatcccccatcctttggcggcggcacaaaggtggaaatcaaa

SEQ ID NO: 153 - DNA, 321 bp, Artificial Sequence
  Description of Artificial Sequence Synthetic polynucleotide
gacattcaaatgacacaatcacctagtagtgtttctgcaagcgtgggagatagggtcacgattacgtgcagagccagtcaaggaattggaattgatctcgcttggtatcaacaaaagcccggcaaggctcccaagttgctcatttacgatgctagtaaactgcagacgggtgtcccgtcacggttctccgggtctggatccggtacagattttacgctcacaattagctctcttcaacccgaagatttcgcgaactactactgtcgtcagtatgctacctttcctcaaacattcggggggggcaccaaggtagaaattaaa

SEQ ID NO: 154 - DNA, 321 bp, Artificial Sequence
  Description of Artificial Sequence Synthetic polynucleotide
gaaattgtgatgacacaatctcctgccacgctttccctcagccccggggaaagggctacgttgtcttgtcgcgcttcccaaagtgtcagcacccacctggcatggtatcaacaaaagcccggacaagcaccgaggcttctgatctacgatgccaccaatcgggcaacaggcattcctgcccggttcagtgggtccgggtccgggactgattttaccctcaccatctctagcctcgagccggaagactttgctgtctactattgtctgcaatatacgaattacccctggacatttggtggcggtaccaaggtcgagattaaa

SEQ ID NO: 155 - DNA, 324 bp, Artificial Sequence
  Description of Artificial Sequence Synthetic polynucleotide
gaatccgccctgacccaaccagcaagtgtttccggttccccaggtcaatcaatcactatctcctgtactgggaccgacatccgcggtatgtcagtctcctggtatcagcagcaccccgggaaggcccccaaacttatgatctatgaggtcaaccaaaggccgagtggagtatccaatcggtttagcggctctaaaagcgggaatacagcttctctcaccatctccggccttcaagctgaagacgaagctgattattactgcgctgcctgggacgcaagcgttgcaggatacgtctttggaggagggacgaaactgacggttctg

SEQ ID NO: 156 - DNA, 321 bp, Artificial Sequence
  Description of Artificial Sequence Synthetic polynucleotide
gaaatcgtgatgacccagtcacccgccacattgtccttgagccccggggaacgggccaccctcagctgtcgagccagccagaacattggccacagtttggcttggtatcaacagaaaccagggcaggctcctagactgcttatatacggggctagtataagggctcccggtatcccggcgcggttctcaggctccggcagtggaacggacttcactctgacaatatccagtttggagccggaagactttgcagtttattattgccaacaatatatcacctggcccagtacttttggcggtggaacaaaagttgaaataaaa

SEQ ID NO: 157 - DNA, 324 bp, Artificial Sequence
  Description of Artificial Sequence Synthetic polynucleotide
gagattgtcatgacccaatcccctgctactctgtctctgtctcccggggaacgcgctacacttagttgcagggcatcccggtctgtatcaagtgatctggcgtggtaccagcaaaagcccgggcaagcccccaggctcttgatttacgacgtctccactcgggccacgggaatacctgctagattctctgggtcaggaagcggaaccgattttaccctcactattagtagcttggagcctgaggatttcgccgtctattattgtcagcaatacggcagtagtctcccgcccaccttcggtggcggaaccaaggttgaaatcaag

SEQ ID NO: 158 - DNA, 315 bp, Artificial Sequence
  Description of Artificial Sequence Synthetic polynucleotide
gaaatcgtcatgacccaaagccctgcaaccctctcattgtcacccggagagcgcgcaacgctgagctgtcgggcctctcaaagcgtgagaaccagtctcgcatggtaccaacaaaagccaggccaagctccacggttgcttatctatgatacaagcattagggccgcaggcataccggcacggttcagtggctctggtagtggtaccgactttaccctcacgatttcaagcctggaacccgaagactttgcggtctactattgtcaacaatacgggaattggcactttggcggcggaacgaaagtggaaattaag

SEQ ID NO: 159 - DNA, 324 bp, Artificial Sequence
  Description of Artificial Sequence Synthetic polynucleotide
gaaattgtcatgacccaaagtccggcgacgctttccctctctcccggcgaaagggcaaccctgagctgtcgcgctagtcaatcaatatcctcttcacagctcgcgtggtaccaacagaaacccggccaagcacctagactcctgatatatggagcttctacgagagccaccgggattccagctaggttttctggttcagggtccgggacggattttacactcacaatcagctccctcgagccagaggattttgcggtttactattgccaacaatataatgacagacccaggacattcggcggcggaactaaggttgagattaaa

SEQ ID NO: 160 - DNA, 321 bp, Artificial Sequence
  Description of Artificial Sequence Synthetic polynucleotide
gatatacaaatgacgcaaagtccctcaagtgtaagcgcgtctgtcggagaccgggtgactataacatgcagagcttctatggatataaggaatgatctcgcgtggtaccaacagaagccaggcaaggcgcctaaactgttgatatatgatgcgtcaaaattggagacaggagtgcccagcaggttctcaggttccggaagtgggaccgattttacactcaccatttcatctctgcaaccagaggacttcgccaattattattgtcaacaagctaagtccttcccgcccacctttggcggtggaaccaaggtggaaataaaa

SEQ ID NO: 161 - DNA, 336 bp, Artificial Sequence
  Description of Artificial Sequence Synthetic polynucleotide
gatattgtgatgacgcagtcacctctcagcctgcctgtgacacccggggagccagcctctattagctgccggagctcacagagtctcttgcattccagcgggtacaactttctccactggtatctgcaaaagccgggtcaaagtccacagttgcttatttacatggcgacaaaccgtgcgtcaggagtgccggatagattcagcggcagtggctccgggacagatttcacccttaagatttcccgcgttgaggcagaagacgtcggcgtttattattgtgttcaggatcttcatacattgccacaatttggtggtgggacaaaggtagaaataaag

SEQ ID NO: 162 - DNA, 300 bp, Artificial Sequence
  Description of Artificial Sequence Synthetic polynucleotide
gatatcgtgatgacacaaagcccccttagtcttccggtcactcccggggaacctgccagtattagttgcagaagctcccaatcactgctgcattggtatctccagaaaccaggacaatctccccaattgctcatctatatgggatcccaaagggccgcgggcgttcctgatcgtttctcaggcagcgggtcagggaccgacttcacgctcaagattagccgcgtggaagcagaagatgtgggagtctattattgccaatcaggcgtcactttcggcggtggtacgaaggttgagatcaag

SEQ ID NO: 163 - DNA, 336 bp, Artificial Sequence
  Description of Artificial Sequence Synthetic polynucleotide
gatattgttatgacacaaagtcccctctcccttcctgttactccgggagagccggcaagcataagctgtagatcttcacagtcacttcttagctcatacggataccataatctgcattggtaccttcagaaacctggacaaagcccacaacttttgatctacatgggatcaatccgagcccccggagttcctgaccgcttttctggctccggcagcgggacagattttaccctgaaaatttcaagggtagaagctgaagacgtcggtgtatattattgtatgcagggaacacaagctcctagaacgtttggaggaggcacaaaggtagaaataaaa

SEQ ID NO: 164 - DNA, 336 bp, Artificial Sequence
  Description of Artificial Sequence Synthetic polynucleotide
gatatagtcatgacgcaaagtcctctcagcctgccagtcactcccggtgaaccggcgagtatttcctgtcggtccagccaatcccttctgcacgttacgggttataattacctgcattggtatctccagaaacccggccaatcccctcaacttttgatctatggggcaagtagtcgcgctgccggcgttcccgacaggttctcaggtagcggtagcggaacggacttcacactgaaaatttcccgtgttgaagccgaggatgtaggcgtatattattgtatgcagagtacacaatggccacacaccttcggtggtgggactaaagtggaaatcaaa

SEQ ID NO: 165 - DNA, 321 bp, Artificial Sequence
  Description of Artificial Sequence Synthetic polynucleotide
gatattcaaatgacgcaatccccatcctctgtttcagcttccgtcggggatcgggtcaccataacttgtcgcgcctctcaagatatcaaatctgatttggcctggtaccaacagaaaccggggaaagcacccaagctcctcatttatagggcatccgcacgggaaagcggggtaccatctcgctttagtggatccggctctggtacggacttcactctgacgattagcagcctgcaacccgaggactttgcaaattactactgccaacaatatcgcagttttccatggagttttggtggtgggacaaaggtcgagatcaag

SEQ ID NO: 166 - DNA, 318 bp, Artificial Sequence
  Description of Artificial Sequence Synthetic polynucleotide
gaaaatgtgctaactcaaagtccagcaatcatgtccgcctctccaggcgagaaagtgaccatcagctgttctgcttcttcctccgtgtcctacatgtactggtaccagcagaagcctggctcctctcctaagccctggatcttcagaacctacaacctggcctccggcgtgcctgctagatttagcggatctggctctggcacctcttactccctgaccatctccaacatggaagccgaggacgccgctacatactactgccagcaacggtccagctatccttggaccttcggcggcggaaccaagctggagatcaag

SEQ ID NO: 167 - DNA, 330 bp, Artificial Sequence
  Description of Artificial Sequence Synthetic polynucleotide
gaaagtgcactaactcaaccagcaagtgtgagcggatctcctggccagtccatcaccatctcctgcaccggcaccagcggcgacatcggcagatacaactatgtctcttggtaccagcagcaccccggcaaggcccctaaactgatgatctacgaagatatcgagagaccttctggcgtgtccaaccggttctccggctccaagtccggcaacaccgcttctctcacaatctctggcctgcaggccgaggacgaggccgactactactgtcaagtgtgggatgctaccctggacctggtgttcggcggcggaaccaagctgaccgtgctg

SEQ ID NO: 168 - DNA, 339 bp, Artificial Sequence
  Description of Artificial Sequence Synthetic polynucleotide
gatatcgtgatgactcaaagtccagatagtctcgctgtctctctgggcgagagagccacaatcaactgcaagtcctctcagtccgtgctggacacctccaccaaccggaactacctggcctggtatcagcagaaaccaggccagcctcctaagctgctgatctacggcgcttccatcagagctggcggcgtgcccgatcggtttagcggctctggatctggcaccgacttcaccctgaccatctccagcctgcaggccgaggacgtggccgtgtactactgtcaacagtacgagacatggccttggaccttcggcggaggcaccaaggtggaaatcaag

SEQ ID NO: 169 - DNA, 330 bp, Artificial Sequence
  Description of Artificial Sequence Synthetic polynucleotide
gaaagtgcactaactcaaccagcaagtgtgagcggctctccaggccagtccatcaccatctcctgcaccggcaccaccggatctatcgcctccaactacgtgtcttggtatcagcagcaccccggcaaagctcctaagctgatgatctacgacacaaccaaccggcctcctggcgtgtccaatcggttctccggctccaagtccggcaacaccgctagcctgacaatctctggcctgcaggccgaggacgaggccgactactactgtcaatctaccgaaagaggcagaagcgatgtcatcttcggcggcggaaccaagctgaccgtgctg

SEQ ID NO: 170 - protein, 9 aa, Artificial Sequence
  Description of Artificial Sequence Synthetic peptide
VQTLQTPST

SEQ ID NO: 171 - protein, 9 aa, Artificial Sequence
  Description of Artificial Sequence Synthetic peptide
LQYGSSPPL

SEQ ID NO: 172 - protein, 9 aa, Artificial Sequence
  Description of Artificial Sequence Synthetic peptide
QQYDTHPLS

SEQ ID NO: 173 - protein, 9 aa, Artificial Sequence
  Description of Artificial Sequence Synthetic peptide
QQYSSYPSL

SEQ ID NO: 174 - protein, 9 aa, Artificial Sequence
  Description of Artificial Sequence Synthetic peptide
VQDLHTPFT

SEQ ID NO: 175 - protein, 9 aa, Artificial Sequence
  Description of Artificial Sequence Synthetic peptide
MQVLRTPIT

SEQ ID NO: 176 - protein, 6 aa, Artificial Sequence
  Description of Artificial Sequence Synthetic peptide
STMAIP

SEQ ID NO: 177 - protein, 10 aa, Artificial Sequence
  Description of Artificial Sequence Synthetic peptide
QQYVSAPPYT

SEQ ID NO: 178 - protein, 9 aa, Artificial Sequence
  Description of Artificial Sequence Synthetic peptide
VQDLHTPFT

SEQ ID NO: 179 - protein, 11 aa, Artificial Sequence
  Description of Artificial Sequence Synthetic peptide
QQYGSSPPVYS

SEQ ID NO: 180 - protein, 9 aa, Artificial Sequence
  Description of Artificial Sequence Synthetic peptide
QQSYSHPPT

SEQ ID NO: 181 - protein, 10 aa, Artificial Sequence
  Description of Artificial Sequence Synthetic peptide
SSYTSSSTDV

SEQ ID NO: 182 - protein, 10 aa, Artificial Sequence
  Description of Artificial Sequence Synthetic peptide
QQYHTWPPWA

SEQ ID NO: 183 - protein, 8 aa, Artificial Sequence
  Description of Artificial Sequence Synthetic peptide
QQYDNPPS

SEQ ID NO: 184 - protein, 9 aa, Artificial Sequence
  Description of Artificial Sequence Synthetic peptide
RQYATFPQT

SEQ ID NO: 185 - protein, 9 aa, Artificial Sequence
  Description of Artificial Sequence Synthetic peptide
LQYTNYPWT

SEQ ID NO: 186 - protein, 11 aa, Artificial Sequence
  Description of Artificial Sequence Synthetic peptide
AAWDASVAGYV

SEQ ID NO: 187 - protein, 9 aa, Artificial Sequence
  Description of Artificial Sequence Synthetic peptide
QQYITWPST

SEQ ID NO: 188 - protein, 10 aa, Artificial Sequence
  Description of Artificial Sequence Synthetic peptide
QQYGSSLPPT

SEQ ID NO: 189 - protein, 7 aa, Artificial Sequence
  Description of Artificial Sequence Synthetic peptide
QQYGNWH

SEQ ID NO: 190 - protein, 9 aa, Artificial Sequence
  Description of Artificial Sequence Synthetic peptide
QQYNDRPRT

SEQ ID NO: 191 - protein, 9 aa, Artificial Sequence
  Description of Artificial Sequence Synthetic peptide
QQAKSFPPT

SEQ ID NO: 192 - protein, 9 aa, Artificial Sequence
  Description of Artificial Sequence Synthetic peptide
VQDLHTLPQ

SEQ ID NO: 193 - protein, 5 aa, Artificial Sequence
  Description of Artificial Sequence Synthetic peptide
QSGVT

SEQ ID NO: 194 - protein, 9 aa, Artificial Sequence
  Description of Artificial Sequence Synthetic peptide
MQGTQAPRT

SEQ ID NO: 195 - protein, 9 aa, Artificial Sequence
  Description of Artificial Sequence Synthetic peptide
MQSTQWPHT

SEQ ID NO: 196 - protein, 9 aa, Artificial Sequence
  Description of Artificial Sequence Synthetic peptide
QQYRSFPWS

SEQ ID NO: 197 - protein, 9 aa, Artificial Sequence
  Description of Artificial Sequence Synthetic peptide
QQRSSYPWT

SEQ ID NO: 198 - protein, 10 aa, Artificial Sequence
  Description of Artificial Sequence Synthetic peptide
QVWDATLDLV

SEQ ID NO: 199 - protein, 9 aa, Artificial Sequence
  Description of Artificial Sequence Synthetic peptide
QQYETWPWT

SEQ ID NO: 200 - protein, 450 aa, Artificial Sequence
  Description of Artificial Sequence Synthetic polypeptide
QVTLRESGPALVKPTQTLTLTCTFSGFSLSTSGMSVGWIRQPPGKALEWLADIWWDDKKDYNPSLKSRLTISKDTSKNQVVLKVTNMDPADTATYYCARSMITNWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 201 - protein, 213 aa, Artificial Sequence
  Description of Artificial Sequence Synthetic polypeptide
DIQMTQSPSTLSASVGDRVTITCKCQLSVGYMHWYQQKPGKAPKLLIYDTSKLASGVPSRFSGSGSGTEFTLTISSLQPDDFATYYCFQGSGYPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 202 - protein, 426 aa, Homo sapiens
MPLLWLRGFLLASCWIIVRSSPTPGSEGHSAAPDCPSCALAALPKDVPNSQPEMVEAVKKHILNMLHLKKRPDVTQPVPKAALLNAIRKLHVGKVGENGYVEIEDDIGRRAEMNELMEQTSEIITFAESGTARKTLHFEISKEGSDLSVVERAEVWLFLKVPKANRTRTKVTIRLFQQQKHPQGSLDTGEEAEEVGLKGERSELLLSEKVVDARKSTWHVFPVSSSIQRLLDQGKSSLDVRIACEQCQESGASLVLLGKKKKKEEEGEGKKKGGGEGGAGADEEKEQSHRPFLMLQARQSEDHPHRRRRRGLECDGKVNICCKKQFFVSFKDIGWNDWIIAPSGYHANYCEGECPSHIAGTSGSSLSFHSTVINHYRMRGHSPFANLKSCCVPTKLRPMSMLYYDDGQNIIKKDIQNMIVEECGCS

SEQ ID NO: 203 - protein, 407 aa, Homo sapiens
MDGLPGRALGAACLLLLAAGWLGPEAWGSPTPPPTPAAPPPPPPPGSPGGSQDTCTSCGGFRRPEELGRVDGDFLEAVKRHILSRLQMRGRPNITHAVPKAAMVTALRKLHAGKVREDGRVEIPHLDGHASPGADGQERVSEIISFAETDGLASSRVRLYFFISNEGNQNLFVVQASLWLYLKLLPYVLEKGSRRKVRVKVYFQEQGHGDRWNMVEKRVDLKRSGWHTFPLTEAIQALFERGERRLNLDVQCDSCQELAVVPVFVDPGEESHRPFVVVQARLGDSRHRIRKRGLECDGRTNLCCRQQFFIDFRLIGWNDWIIAPTGYYGNYCEGSCPAYLAGVPGSASSFHTAVVNQYRMRGLNPGTVNSCCIPTKLSTMSMLYFDDEYNIVKRDVPNMIVEECGCA

SEQ ID NO: 204 - protein, 352 aa, Homo sapiens
MTSSLLLAFLLLAPTTVATPRAGGQCPACGGPTLELESQRELLLDLAKRSILDKLHLTQRPTLNRPVSRAALRTALQHLHGVPQGALLEDNREQECEIISFAETGLSTINQTRLDFHFSSDRTAGDREVQQASLMFFVQLPSNTTWTLKVRVLVLGPHNTNLTLATQYLLEVDASGWHQLPLGPEAQAACSQGHLTLELVLEGQVAQSSVILGGAAHRPFVAARVRVGGKHQIHRRGIDCQGGSRMCCRQEFFVDFREIGWHDWIIQPEGYAMNFCIGQCPLHIAGMPGIAASFHTAVLNLLKANTAAGTTGGGSCCVPTARRPLSLLYYDRDSNIVKTDIPDMVVEACGCS

SEQ ID NO: 205 - protein, 116 aa, Homo sapiens [sequence omitted]

SEQ ID NO: 206 - protein, 406 aa, Homo sapiens
SPTPGSEGHSAAPDCPSCALAALPKDVPNSQPEMVEAVKKHILNMLHLKKRPDVTQPVPKAALLNAIRKLHVGKVGENGYVEIEDDIGRRAEMNELMEQTSEIITFAESGTARKTLHFEISKEGSDLSVVERAEVWLFLKVPKANRTRTKVTIRLFQQQKHPQGSLDTGEEAEEVGLKGERSELLLSEKVVDARKSTWHVFPVSSSIQRLLDQGKSSLDVRIACEQCQESGASLVLLGKKKKKEEEGEGKKKGGGEGGAGADEEKEQSHRPFLMLQARQSEDHPHRRRRRGLECDGKVNICCKKQFFVSFKDIGWNDWIIAPSGYHANYCEGECPSHIAGTSGSSLSFHSTVINHYRMRGHSPFANLKSCCVPTKLRPMSMLYYDDGQNIIKKDIQNMIVEECGCS

SEQ ID NO: 207 - protein, 115 aa, Homo sapiens
GLECDGRTNLCCRQQFFIDFRLIGWNDWIIAPTGYYGNYCEGSCPAYLAGVPGSASSFHTAVVNQYRMRGLNPGTVNSCCIPTKLSTMSMLYFDDEYNIVKRDVPNMIVEECGCA

SEQ ID NO: 208 - protein, 134 aa, Homo sapiens
STPLMSWPWSPSALRLLQRPPEEPAAHANCHRVALNISFQELGWERWIVYPPSFIFHYCHGGCGLHIPPNLSLPVPGAPPTPAQPYSLLPGAQPCCAALPGTMRPLHVRTTSDGGYSFKYETVPNLLTQHCACI

SEQ ID NO: 209 - protein, 11 aa, Artificial Sequence
  Description of Artificial Sequence Synthetic peptide
QSTERGRSDVI

SEQ ID NO: 210 - protein, 10 aa, Artificial Sequence
  Description of Artificial Sequence Synthetic peptide
GLSLSNAKMG

SEQ ID NO: 211 - protein, 10 aa, Artificial Sequence
  Description of Artificial Sequence Synthetic peptide
GASVSNARTG

SEQ ID NO: 212 - protein, 8 aa, Artificial Sequence
  Description of Artificial Sequence Synthetic peptide
GFTASRNY

SEQ ID NO: 213 - protein, 8 aa, Artificial Sequence
  Description of Artificial Sequence Synthetic peptide
GFDFGEHW

SEQ ID NO: 214 - protein, 8 aa, Artificial Sequence
  Description of Artificial Sequence Synthetic peptide
GYTFSNYG

SEQ ID NO: 215 - protein, 10 aa, Artificial Sequence
  Description of Artificial Sequence Synthetic peptide
GLSLSNAKMG

SEQ ID NO: 216 - protein, 10 aa, Artificial Sequence
  Description of Artificial Sequence Synthetic peptide
GVSLSNARLG

SEQ ID NO: 217 - protein, 10 aa, Artificial Sequence
  Description of Artificial Sequence Synthetic peptide
GYSLTDARIG

SEQ ID NO: 218 - protein, 8 aa, Artificial Sequence
  Description of Artificial Sequence Synthetic peptide
GYTFSNYG

SEQ ID NO: 219 - protein, 8 aa, Artificial Sequence
  Description of Artificial Sequence Synthetic peptide
GFTLSNYP

SEQ ID NO: 220 - protein, 10 aa, Artificial Sequence
  Description of Artificial Sequence Synthetic peptide
GVSLSNARLG

SEQ ID NO: 221 - protein, 10 aa, Artificial Sequence
  Description of Artificial Sequence Synthetic peptide
GLSLSNAKMG

SEQ ID NO: 222 - protein, 8 aa, Artificial Sequence
  Description of Artificial Sequence Synthetic peptide
GFTASRNY

SEQ ID NO: 223 - protein, 10 aa, Artificial Sequence
  Description of Artificial Sequence Synthetic peptide
GYSLTDARIG

SEQ ID NO: 224 - protein, 10 aa, Artificial Sequence
  Description of Artificial Sequence Synthetic peptide
GFSLSTSGAT

SEQ ID NO: 225 - protein, 8 aa, Artificial Sequence
  Description of Artificial Sequence Synthetic peptide
GYTYTGYR

SEQ ID NO: 226 - protein, 10 aa, Artificial Sequence
  Description of Artificial Sequence Synthetic peptide
GVSLSDARMG

SEQ ID NO: 227 - protein, 10 aa, Artificial Sequence
  Description of Artificial Sequence Synthetic peptide
GFSLSTSGAT

SEQ ID NO: 228 - protein, 10 aa, Artificial Sequence
  Description of Artificial Sequence Synthetic peptide
GFTLSNPRMG

SEQ ID NO: 229 - protein, 10 aa, Artificial Sequence
  Description of Artificial Sequence Synthetic peptide
GFPLSTSRMG

SEQ ID NO: 230 - protein, 8 aa, Artificial Sequence
  Description of Artificial Sequence Synthetic peptide
GYTFPSHY

SEQ ID NO: 231 - protein, 10 aa, Artificial Sequence
  Description of Artificial Sequence Synthetic peptide
GFPLSTSRMG

SEQ ID NO: 232 - protein, 10 aa, Artificial Sequence
  Description of Artificial Sequence Synthetic peptide
GFPLSTSRMG

SEQ ID NO: 233 - protein, 10 aa, Artificial Sequence
  Description of Artificial Sequence Synthetic peptide
GFPLSTSRMG

SEQ ID NO: 234 - protein, 10 aa, Artificial Sequence
  Description of Artificial Sequence Synthetic peptide
GVSLSNVRMG

SEQ ID NO: 235 - protein, 8 aa, Artificial Sequence
  Description of Artificial Sequence Synthetic peptide
GYTFTSSA

SEQ ID NO: 236 - protein, 8 aa, Artificial Sequence
  Description of Artificial Sequence Synthetic peptide
GFTSSNHW

SEQ ID NO: 237 - protein, 8 aa, Artificial Sequence
  Description of Artificial Sequence Synthetic peptide
GFTFSDHH

SEQ ID NO: 238 - protein, 8 aa, Artificial Sequence
  Description of Artificial Sequence Synthetic peptide
GFTSSGYW

SEQ ID NO: 239 - protein, 8 aa, Artificial Sequence
  Description of Artificial Sequence Synthetic peptide
GFTSSNHW

SEQ ID NO: 240 - protein, 7 aa, Artificial Sequence
  Description of Artificial Sequence Synthetic peptide
GYPSPAT

SEQ ID NO: 241 - protein, 8 aa, Artificial Sequence
  Description of Artificial Sequence Synthetic peptide
GYTFTSYW

SEQ ID NO: 242 - protein, 8 aa, Artificial Sequence
  Description of Artificial Sequence Synthetic peptide
GDTSSHSH

SEQ ID NO: 243 - protein, 10 aa, Artificial Sequence
  Description of Artificial Sequence Synthetic peptide
GFSLSDTRWG

SEQ ID NO: 244 - protein, 8 aa, Artificial Sequence
  Description of Artificial Sequence Synthetic peptide
GYTFPSHY

SEQ ID NO: 245 - protein, 7 aa, Artificial Sequence
  Description of Artificial Sequence Synthetic peptide
VSTTGTT

SEQ ID NO: 246 - protein, 7 aa, Artificial Sequence
  Description of Artificial Sequence Synthetic peptide
INPSGRI

SEQ ID NO: 247 - protein, 8 aa, Artificial Sequence
  Description of Artificial Sequence Synthetic peptide
ISGSGDYT

SEQ ID NO: 248 - protein, 8 aa, Artificial Sequence
  Description of Artificial Sequence Synthetic peptide
IKGDASDT

SEQ ID NO: 249 - protein, 8 aa, Artificial Sequence
  Description of Artificial Sequence Synthetic peptide
ISKSGSSI

SEQ ID NO: 250 - protein, 7 aa, Artificial Sequence
  Description of Artificial Sequence Synthetic peptide
VSTTGTT

SEQ ID NO: 251 - protein, 7 aa, Artificial Sequence
  Description of Artificial Sequence Synthetic peptide
GSVTGST

SEQ ID NO: 252 - protein, 7 aa, Artificial Sequence
  Description of Artificial Sequence Synthetic peptide
INPSGRT

SEQ ID NO: 253 - protein, 8 aa, Artificial Sequence
  Description of Artificial Sequence Synthetic peptide
ISKSGSSI

SEQ ID NO: 254 - protein, 8 aa, Artificial Sequence
  Description of Artificial Sequence Synthetic peptide
TTSGSSTI

SEQ ID NO: 255 - protein, 7 aa, Artificial Sequence
  Description of Artificial Sequence Synthetic peptide
GSVTGST

SEQ ID NO: 256 - protein, 7 aa, Artificial Sequence
  Description of Artificial Sequence Synthetic peptide
VSTTGTT

SEQ ID NO: 257 - protein, 8 aa, Artificial Sequence
  Description of Artificial Sequence Synthetic peptide
ISGSGDYT

SEQ ID NO: 258 - protein, 7 aa, Artificial Sequence
  Description of Artificial Sequence Synthetic peptide
INPSGRT

SEQ ID NO: 259 - protein, 6 aa, Artificial Sequence
  Description of Artificial Sequence Synthetic peptide
ITSSGR

SEQ ID NO: 260 - protein, 8 aa, Artificial Sequence
  Description of Artificial Sequence Synthetic peptide
IDPKSGRT

SEQ ID NO: 261 - protein, 7 aa, Artificial Sequence
  Description of Artificial Sequence Synthetic peptide
VSTTGTT

SEQ ID NO: 262 - protein, 6 aa, Artificial Sequence
  Description of Artificial Sequence Synthetic peptide
ITSSGR

SEQ ID NO: 263 - protein, 7 aa, Artificial Sequence
  Description of Artificial Sequence Synthetic peptide
ISTSGRT

SEQ ID NO: 264 - protein, 7 aa, Artificial Sequence
  Description of Artificial Sequence Synthetic peptide
INPSGAT

SEQ ID NO: 265 - protein, 8 aa, Artificial Sequence
  Description of Artificial Sequence Synthetic peptide
MNPITGHT

SEQ ID NO: 266 - protein, 7 aa, Artificial Sequence
  Description of Artificial Sequence Synthetic peptide
INPSGAT

SEQ ID NO: 267 - protein, 7 aa, Artificial Sequence
  Description of Artificial Sequence Synthetic peptide
INPSGAT

SEQ ID NO: 268 - protein, 7 aa, Artificial Sequence
  Description of Artificial Sequence Synthetic peptide
INPSGAT

SEQ ID NO: 269 - protein, 7 aa, Artificial Sequence
  Description of Artificial Sequence Synthetic peptide
VNPSGST

SEQ ID NO: 270 - protein, 8 aa, Artificial Sequence
  Description of Artificial Sequence Synthetic peptide
IYPGSGHT

SEQ ID NO: 271 - protein, 8 aa, Artificial Sequence
  Description of Artificial Sequence Synthetic peptide
IGPKGLGT

SEQ ID NO: 272 - protein, 8 aa, Artificial Sequence
  Description of Artificial Sequence Synthetic peptide
ISPSGDFT

SEQ ID NO: 273 - protein, 7 aa, Artificial Sequence
  Description of Artificial Sequence Synthetic peptide
IGTAGVT

SEQ ID NO: 274 - protein, 8 aa, Artificial Sequence
  Description of Artificial Sequence Synthetic peptide
IGPKGLGT

SEQ ID NO: 275 - protein, 8 aa, Artificial Sequence
  Description of Artificial Sequence Synthetic peptide
MNPKSGYT

SEQ ID NO: 276 - protein, 8 aa, Artificial Sequence
  Description of Artificial Sequence Synthetic peptide
INPSNGGT

SEQ ID NO: 277 - protein, 8 aa, Artificial Sequence
  Description of Artificial Sequence Synthetic peptide
INPRSGAT

SEQ ID NO: 278 - protein, 7 aa, Artificial Sequence
  Description of Artificial Sequence Synthetic peptide
INDRGTT

SEQ ID NO: 279 - protein, 8 aa, Artificial Sequence
  Description of Artificial Sequence Synthetic peptide
MNPITGHT

SEQ ID NO: 280 - protein, 8 aa, Artificial Sequence
  Description of Artificial Sequence Synthetic peptide
ARAGYPDV

SEQ ID NO: 281 - protein, 8 aa, Artificial Sequence
  Description of Artificial Sequence Synthetic peptide
ARAGYPDV

SEQ ID NO: 282 - protein, 18 aa, Artificial Sequence
  Description of Artificial Sequence Synthetic peptide
ARSPRGPRLLGYYYGMDV

SEQ ID NO: 283 - protein, 13 aa, Artificial Sequence
  Description of Artificial Sequence Synthetic peptide
VKDLSKQWLGIDY

SEQ ID NO: 284 - protein, 16 aa, Artificial Sequence
  Description of Artificial Sequence Synthetic peptide
ARLPRLAWNYYYGMDV

SEQ ID NO: 285 - protein, 8 aa, Artificial Sequence
  Description of Artificial Sequence Synthetic peptide
ARAGYPDV

SEQ ID NO: 286 - protein, 13 aa, Artificial Sequence
  Description of Artificial Sequence Synthetic peptide
TTSYLVDTAMVTR

SEQ ID NO: 287 - protein, 8 aa, Artificial Sequence
  Description of Artificial Sequence Synthetic peptide
ARAGYPDV

SEQ ID NO: 288 - protein, 16 aa, Artificial Sequence
  Description of Artificial Sequence Synthetic peptide
ARLPRLAWNYYYGMDV

SEQ ID NO: 289 - protein, 16 aa, Artificial Sequence
  Description of Artificial Sequence Synthetic peptide
ARGPDYYAPLRSYMDV

SEQ ID NO: 290 - protein, 13 aa, Artificial Sequence
  Description of Artificial Sequence Synthetic peptide
TTSYLVDTAMVTR

SEQ ID NO: 291 - protein, 8 aa, Artificial Sequence
  Description of Artificial Sequence Synthetic peptide
ARAGYPDV

SEQ ID NO: 292 - protein, 18 aa, Artificial Sequence
  Description of Artificial Sequence Synthetic peptide
ARSPRGPRLLGYYYGMDV

SEQ ID NO: 293 - protein, 8 aa, Artificial Sequence
  Description of Artificial Sequence Synthetic peptide
ARAGYPDV

SEQ ID NO: 294 - protein, 13 aa, Artificial Sequence
  Description of Artificial Sequence Synthetic peptide
TTTKIVGATLRDY

SEQ ID NO: 295 - protein, 19 aa, Artificial Sequence
  Description of Artificial Sequence Synthetic peptide
AREVKQWLVPGRGVYWFDP

SEQ ID NO: 296 - protein, 8 aa, Artificial Sequence
  Description of Artificial Sequence Synthetic peptide
ARAGYPDV

SEQ ID NO: 297 - protein, 13 aa, Artificial Sequence
  Description of Artificial Sequence Synthetic peptide
TTTKIVGATLRDY

SEQ ID NO: 298 - protein, 8 aa, Artificial Sequence
  Description of Artificial Sequence Synthetic peptide
ARAGYPDV

SEQ ID NO: 299 - protein, 8 aa, Artificial Sequence
  Description of Artificial Sequence Synthetic peptide
ARAGYPDV

SEQ ID NO: 300 - protein, 20 aa, Artificial Sequence
  Description of Artificial Sequence Synthetic peptide
ARDMRYDILPGRSYYYGMDV

SEQ ID NO: 301 - protein, 8 aa, Artificial Sequence
  Description of Artificial Sequence Synthetic peptide
ARAGYPDV

SEQ ID NO: 302 - protein, 8 aa, Artificial Sequence
  Description of Artificial Sequence Synthetic peptide
ARAGYPDV

SEQ ID NO: 303 - protein, 8 aa, Artificial Sequence
  Description of Artificial Sequence Synthetic peptide
ARAGYPDV

SEQ ID NO: 304 - protein, 8 aa, Artificial Sequence
  Description of Artificial Sequence Synthetic peptide
ARAGYPDV

SEQ ID NO: 305 - protein, 11 aa, Artificial Sequence
  Description of Artificial Sequence Synthetic peptide
AREGDIFVMDV

SEQ ID NO: 306 - protein, 17 aa, Artificial Sequence
  Description of Artificial Sequence Synthetic peptide
ASIRDYGDKKGGSWFDP

SEQ ID NO: 307 - protein, 9 aa, Artificial Sequence
  Description of Artificial Sequence Synthetic peptide
ARDVSPVYY

SEQ ID NO: 308 - protein, 15 aa, Artificial Sequence
  Description of Artificial Sequence Synthetic peptide
ARGPHGPQQRLRFDP

SEQ ID NO: 309 - protein, 18 aa, Artificial Sequence
  Description of Artificial Sequence Synthetic peptide
ARDVRGWRALYYYYGMDV

SEQ ID NO: 310 - protein, 12 aa, Artificial Sequence
  Description of Artificial Sequence Synthetic peptide
ATLHGSGLAFDI

SEQ ID NO: 311 - protein, 13 aa, Artificial Sequence
  Description of Artificial Sequence Synthetic peptide
SITTVVAYWYFDV

SEQ ID NO: 312 - protein, 16 aa, Artificial Sequence
  Description of Artificial Sequence Synthetic peptide
ARDRGGRTNGVGAFDI

SEQ ID NO: 313 - protein, 14 aa, Artificial Sequence
  Description of Artificial Sequence Synthetic peptide
AHTLHTREFVGMDV

SEQ ID NO: 314 - protein, 20 aa, Artificial Sequence
  Description of Artificial Sequence Synthetic peptide
ARDMRYDILPGRSYYYGMDV

SEQ ID NO: 315 - protein, 6 aa, Artificial Sequence
  Description of Artificial Sequence Synthetic peptide
QNIGAA

SEQ ID NO: 316 - protein, 6 aa, Artificial Sequence
  Description of Artificial Sequence Synthetic peptide
QTVSSH

SEQ ID NO: 317 - protein, 11 aa, Artificial Sequence
  Description of Artificial Sequence Synthetic peptide
RSLVHGSGHNY

SEQ ID NO: 318 - protein, 11 aa, Artificial Sequence
  Description of Artificial Sequence Synthetic peptide
QSLLHSNEKNY

SEQ ID NO: 319 - protein, 11 aa, Artificial Sequence
  Description of Artificial Sequence Synthetic peptide
RSLVHGSGDNY

SEQ ID NO: 320 - protein, 6 aa, Artificial Sequence
  Description of Artificial Sequence Synthetic peptide
QNVYTN

SEQ ID NO: 321 - protein, 6 aa, Artificial Sequence
  Description of Artificial Sequence Synthetic peptide
QSLGTN

SEQ ID NO: 322 - protein, 6 aa, Artificial Sequence
  Description of Artificial Sequence Synthetic peptide
QTISRS

SEQ ID NO: 323 - protein, 11 aa, Artificial Sequence
  Description of Artificial Sequence Synthetic peptide
QSLLHSNEKNY

SEQ ID NO: 324 - protein, 11 aa, Artificial Sequence
  Description of Artificial Sequence Synthetic peptide
QSLQQSEGRTY

SEQ ID NO: 325 - protein, 6 aa, Artificial Sequence
  Description of Artificial Sequence Synthetic peptide
QRIGSD

SEQ ID NO: 326 - protein, 6 aa, Artificial Sequence
  Description of Artificial Sequence Synthetic peptide
QGISRD

SEQ ID NO: 327 - protein, 11 aa, Artificial Sequence
  Description of Artificial Sequence Synthetic peptide
QSLLHSNEKNY

SEQ ID NO: 328 - protein, 6 aa, Artificial Sequence
  Description of Artificial Sequence Synthetic peptide
QSIGTK

SEQ ID NO: 329 - protein, 12 aa, Artificial Sequence
  Description of Artificial Sequence Synthetic peptide
QSVLDTSTNRNY

SEQ ID NO: 330 - protein, 6 aa, Artificial Sequence
  Description of Artificial Sequence Synthetic peptide
NIGLRS

SEQ ID NO: 331 - protein, 6 aa, Artificial Sequence
  Description of Artificial Sequence Synthetic peptide
ESVRSN

SEQ ID NO: 332 - protein, 6 aa, Artificial Sequence
  Description of Artificial Sequence Synthetic peptide
QGISND

SEQ ID NO: 333 - protein, 6 aa, Artificial Sequence
  Description of Artificial Sequence Synthetic peptide
QGIGID

SEQ ID NO: 334 - protein, 6 aa, Artificial Sequence
  Description of Artificial Sequence Synthetic peptide
QSVSTH

SEQ ID NO: 335 - protein, 6 aa, Artificial Sequence
  Description of Artificial Sequence Synthetic peptide
DIRGMS

SEQ ID NO: 336 - protein, 6 aa, Artificial Sequence
  Description of Artificial Sequence Synthetic peptide
QNIGHS

SEQ ID NO: 337 - protein, 6 aa, Artificial Sequence
  Description of Artificial Sequence Synthetic peptide
RSVSSD

SEQ ID NO: 338 - protein, 6 aa, Artificial Sequence
  Description of Artificial Sequence Synthetic peptide
QSVRTS

SEQ ID NO: 339 - protein, 7 aa, Artificial Sequence
  Description of Artificial Sequence Synthetic peptide
QSISSSQ

SEQ ID NO: 340 - protein, 6 aa, Artificial Sequence
  Description of Artificial Sequence Synthetic peptide
MDIRND

SEQ ID NO: 341 - protein, 11 aa, Artificial Sequence
  Description of Artificial Sequence Synthetic peptide
QSLLHSSGYNF

SEQ ID NO: 342 - protein, 3 aa, Artificial Sequence
  Description of Artificial Sequence Synthetic peptide
QSL

SEQ ID NO: 343 - protein, 11 aa, Artificial Sequence
  Description of Artificial Sequence Synthetic peptide
QSLLSSYGYHN

SEQ ID NO: 344 - protein, 11 aa, Artificial Sequence
  Description of Artificial Sequence Synthetic peptide
QSLLHVTGYNY

SEQ ID NO: 345 - protein, 6 aa, Artificial Sequence
  Description of Artificial Sequence Synthetic peptide
QDIKSD

SEQ ID NO: 346 - protein, 5 aa, Artificial Sequence
  Description of Artificial Sequence Synthetic peptide
SSVSY

SEQ ID NO: 347 - protein, 9 aa, Artificial Sequence
  Description of Artificial Sequence Synthetic peptide
SGDIGRYNY

SEQ ID NO: 348 - protein, 12 aa, Artificial Sequence
  Description of Artificial Sequence Synthetic peptide
QSVLDTSTNRNY

SEQ ID NO: 349 - protein, 8 aa, Artificial Sequence
  Description of Artificial Sequence Synthetic peptide
TGSIASNY

SEQ ID NO: 350 - protein, 3 aa, Artificial Sequence
  Description of Artificial Sequence Synthetic peptide
GAS

SEQ ID NO: 351 - protein, 3 aa, Artificial Sequence
  Description of Artificial Sequence Synthetic peptide
DVS

SEQ ID NO: 352 - protein, 3 aa, Artificial Sequence
  Description of Artificial Sequence Synthetic peptide
GAS

SEQ ID NO: 353 - protein, 3 aa, Artificial Sequence
  Description of Artificial Sequence Synthetic peptide
DAS

SEQ ID NO: 354 - protein, 3 aa, Artificial Sequence
  Description of Artificial Sequence Synthetic peptide
MAS

SEQ ID NO: 355 - protein, 3 aa, Artificial Sequence
  Description of Artificial Sequence Synthetic peptide
GAS

SEQ ID NO: 356 - protein, 3 aa, Artificial Sequence
  Description of Artificial Sequence Synthetic peptide
GAS

SEQ ID NO: 357 - protein, 3 aa, Artificial Sequence
  Description of Artificial Sequence Synthetic peptide
DAS

SEQ ID NO: 358 - protein, 3 aa, Artificial Sequence
  Description of Artificial Sequence Synthetic peptide
DAS

SEQ ID NO: 359 - protein, 3 aa, Artificial Sequence
  Description of Artificial Sequence Synthetic peptide
DAS

SEQ ID NO: 360 - protein, 3 aa, Artificial Sequence
  Description of Artificial Sequence Synthetic peptide
SAS

SEQ ID NO: 361 - protein, 3 aa, Artificial Sequence
  Description of Artificial Sequence Synthetic peptide
AAS

SEQ ID NO: 362 - protein, 3 aa, Artificial Sequence
  Description of Artificial Sequence Synthetic peptide
DAS

SEQ ID NO: 363 - protein, 3 aa, Artificial Sequence
  Description of Artificial Sequence Synthetic peptide
DAS

SEQ ID NO: 364 - protein, 3 aa, Artificial Sequence
  Description of Artificial Sequence Synthetic peptide
GAS

SEQ ID NO: 365 - protein, 3 aa, Artificial Sequence
  Description of Artificial Sequence Synthetic peptide
DTN

SEQ ID NO: 366 - protein, 3 aa, Artificial Sequence
  Description of Artificial Sequence Synthetic peptide
GAS

SEQ ID NO: 367 - protein, 3 aa, Artificial Sequence
  Description of Artificial Sequence Synthetic peptide
DVS

SEQ ID NO: 368 - protein, 3 aa, Artificial Sequence
  Description of Artificial Sequence Synthetic peptide
DAS

SEQ ID NO: 369 - protein, 3 aa, Artificial Sequence
  Description of Artificial Sequence Synthetic peptide
DAT

SEQ ID NO: 370 - protein, 3 aa, Artificial Sequence
  Description of Artificial Sequence Synthetic peptide
EVN

SEQ ID NO: 371 - protein, 3 aa, Artificial Sequence
  Description of Artificial Sequence Synthetic peptide
GAS

SEQ ID NO: 372 - protein, 3 aa, Artificial Sequence
  Description of Artificial Sequence Synthetic peptide
DVS

SEQ ID NO: 373 - protein, 3 aa, Artificial Sequence
  Description of Artificial Sequence Synthetic peptide
DTS

SEQ ID NO: 374 - protein, 3 aa, Artificial Sequence
  Description of Artificial Sequence Synthetic peptide
GAS

SEQ ID NO: 375 - protein, 3 aa, Artificial Sequence
  Description of Artificial Sequence Synthetic peptide
DAS

SEQ ID NO: 376 - protein, 3 aa, Artificial Sequence
  Description of Artificial Sequence Synthetic peptide
MAT

SEQ ID NO: 377 - protein, 3 aa, Artificial Sequence
  Description of Artificial Sequence Synthetic peptide
MGS

SEQ ID NO: 378 - protein, 3 aa, Artificial Sequence
  Description of Artificial Sequence Synthetic peptide
MGS

SEQ ID NO: 379 - protein, 3 aa, Artificial Sequence
  Description of Artificial Sequence Synthetic peptide
GAS

SEQ ID NO: 380 - protein, 3 aa, Artificial Sequence
  Description of Artificial Sequence Synthetic peptide
RAS

SEQ ID NO: 381 - protein, 3 aa, Artificial Sequence
  Description of Artificial Sequence Synthetic peptide
RTY

SEQ ID NO: 382 - protein, 3 aa, Artificial Sequence
  Description of Artificial Sequence Synthetic peptide
EDI

SEQ ID NO: 383 - protein, 3 aa, Artificial Sequence
  Description of Artificial Sequence Synthetic peptide
GAS

SEQ ID NO: 384 - protein, 3 aa, Artificial Sequence
  Description of Artificial Sequence Synthetic peptide
DTT

SEQ ID NO: 385 - protein, 9 aa, Artificial Sequence
  Description of Artificial Sequence Synthetic peptide
QQYTAYPLT

SEQ ID NO: 386 - protein, 8 aa, Artificial Sequence
  Description of Artificial Sequence Synthetic peptide
QQYFTLPT

SEQ ID NO: 387 - protein, 9 aa, Artificial Sequence
  Description of Artificial Sequence Synthetic peptide
MQGTQAPRT

SEQ ID NO: 388 - protein, 9 aa, Artificial Sequence
  Description of Artificial Sequence Synthetic peptide
VQDLHTPFT

SEQ ID NO: 389 - protein, 9 aa, Artificial Sequence
  Description of Artificial Sequence Synthetic peptide
VQTLQTPST

SEQ ID NO: 390 - protein, 9 aa, Artificial Sequence
  Description of Artificial Sequence Synthetic peptide
LQYGSSPPL

SEQ ID NO: 391 - protein, 9 aa, Artificial Sequence
  Description of Artificial Sequence Synthetic peptide
QQYDTHPLS

SEQ ID NO: 392 - protein, 9 aa, Artificial Sequence
  Description of Artificial Sequence Synthetic peptide
QQYSSYPSL

SEQ ID NO: 393 - protein, 9 aa, Artificial Sequence
  Description of Artificial Sequence Synthetic peptide
VQDLHTPFT

SEQ ID NO: 394 - protein, 9 aa, Artificial Sequence
  Description of Artificial Sequence Synthetic peptide
MQVLRTPIT

SEQ ID NO: 395 - protein, 6 aa, Artificial Sequence
  Description of Artificial Sequence Synthetic peptide
STMAIP

SEQ ID NO: 396 - protein, 10 aa, Artificial Sequence
  Description of Artificial Sequence Synthetic peptide
QQYVSAPPYT

SEQ ID NO: 397 - protein, 9 aa, Artificial Sequence
  Description of Artificial Sequence Synthetic peptide
VQDLHTPFT

SEQ ID NO: 398 - protein, 11 aa, Artificial Sequence
  Description of Artificial Sequence Synthetic peptide
QQYGSSPPVYS

SEQ ID NO: 399 - protein, 9 aa, Artificial Sequence
  Description of Artificial Sequence Synthetic peptide
QQSYSHPPT

SEQ ID NO: 400 - protein, 10 aa, Artificial Sequence
  Description of Artificial Sequence Synthetic peptide
SSYTSSSTDV

SEQ ID NO: 401 - protein, 10 aa, Artificial Sequence
  Description of Artificial Sequence Synthetic peptide
QQYHTWPPWA

SEQ ID NO: 402 - protein, 8 aa, Artificial Sequence
  Description of Artificial Sequence Synthetic peptide
QQYDNPPS

SEQ ID NO: 403 - protein, 9 aa, Artificial Sequence
  Description of Artificial Sequence Synthetic peptide
RQYATFPQT

SEQ ID NO: 404 - protein, 9 aa, Artificial Sequence
  Description of Artificial Sequence Synthetic peptide
LQYTNYPWT

SEQ ID NO: 405 - protein, 11 aa, Artificial Sequence
  Description of Artificial Sequence Synthetic peptide
AAWDASVAGYV

SEQ ID NO: 406 - protein, 9 aa, Artificial Sequence
  Description of Artificial Sequence Synthetic peptide
QQYITWPST

SEQ ID NO: 407 - protein, 10 aa, Artificial Sequence
  Description of Artificial Sequence Synthetic peptide
QQYGSSLPPT

SEQ ID NO: 408 - protein, 7 aa, Artificial Sequence
  Description of Artificial Sequence Synthetic peptide
QQYGNWH

SEQ ID NO: 409 - protein, 9 aa, Artificial Sequence
  Description of Artificial Sequence Synthetic peptide
QQYNDRPRT

SEQ ID NO: 410 - protein, 9 aa, Artificial Sequence
  Description of Artificial Sequence Synthetic peptide
QQAKSFPPT

SEQ ID NO: 411 - protein, 9 aa, Artificial Sequence
  Description of Artificial Sequence Synthetic peptide
VQDLHTLPQ

SEQ ID NO: 412 - protein, 5 aa, Artificial Sequence
  Description of Artificial Sequence Synthetic peptide
QSGVT

SEQ ID NO: 413 - protein, 9 aa, Artificial Sequence
  Description of Artificial Sequence Synthetic peptide
MQGTQAPRT

SEQ ID NO: 414 - protein, 9 aa, Artificial Sequence
  Description of Artificial Sequence Synthetic peptide
MQSTQWPHT

SEQ ID NO: 415 - protein, 9 aa, Artificial Sequence
  Description of Artificial Sequence Synthetic peptide
QQYRSFPWS

SEQ ID NO: 416 - protein, 9 aa, Artificial Sequence
  Description of Artificial Sequence Synthetic peptide
QQRSSYPWT

SEQ ID NO: 417 - protein, 10 aa, Artificial Sequence
  Description of Artificial Sequence Synthetic peptide
QVWDATLDLV

SEQ ID NO: 418 - protein, 9 aa, Artificial Sequence
  Description of Artificial Sequence Synthetic peptide
QQYETWPWT

SEQ ID NO: 419 - protein, 11 aa, Artificial Sequence
  Description of Artificial Sequence Synthetic peptide
QSTERGRSDVI

SEQ ID NO: 420 - protein, 7 aa, Artificial Sequence
  Description of Artificial Sequence Synthetic peptide
NAKMGVN

SEQ ID NO: 421 - protein, 7 aa, Artificial Sequence
  Description of Artificial Sequence Synthetic peptide
NARTGVN

SEQ ID NO: 422 - protein, 5 aa, Artificial Sequence
  Description of Artificial Sequence Synthetic peptide
RNYMS

SEQ ID NO: 423 - protein, 5 aa, Artificial Sequence
  Description of Artificial Sequence Synthetic peptide
EHWMS

SEQ ID NO: 424 - protein, 5 aa, Artificial Sequence
  Description of Artificial Sequence Synthetic peptide
NYGMS

SEQ ID NO: 425 - protein, 7 aa, Artificial Sequence
  Description of Artificial Sequence Synthetic peptide
NAKMGVN

SEQ ID NO: 426 - protein, 7 aa, Artificial Sequence
  Description of Artificial Sequence Synthetic peptide
NARLGVN

SEQ ID NO: 427 - protein, 7 aa, Artificial Sequence
  Description of Artificial Sequence Synthetic peptide
DARIGVN

SEQ ID NO: 428 - protein, 5 aa, Artificial Sequence
  Description of Artificial Sequence Synthetic peptide
NYGMS

SEQ ID NO: 429 - protein, 5 aa, Artificial Sequence
  Description of Artificial Sequence Synthetic peptide
NYPMS

SEQ ID NO: 430 - protein, 7 aa, Artificial Sequence
  Description of Artificial Sequence Synthetic peptide
NARLGVN

SEQ ID NO: 431 - protein, 7 aa, Artificial Sequence
  Description of Artificial Sequence Synthetic peptide
NAKMGVN

SEQ ID NO: 432 - protein, 5 aa, Artificial Sequence
  Description of Artificial Sequence Synthetic peptide
RNYMS

SEQ ID NO: 433 - protein, 7 aa, Artificial Sequence
  Description of Artificial Sequence Synthetic peptide
DARIGVN

SEQ ID NO: 434 - protein, 7 aa, Artificial Sequence
  Description of Artificial Sequence Synthetic peptide
TSGATVN

SEQ ID NO: 435 - protein, 5 aa, Artificial Sequence
  Description of Artificial Sequence Synthetic peptide
GYRIS

SEQ ID NO: 436 - protein, 7 aa, Artificial Sequence
  Description of Artificial Sequence Synthetic peptide
DARMGVN

SEQ ID NO: 437 - protein, 7 aa, Artificial Sequence
  Description of Artificial Sequence Synthetic peptide
TSGATVN

SEQ ID NO: 438 - protein, 7 aa, Artificial Sequence
  Description of Artificial Sequence Synthetic peptide
NPRMGVN

SEQ ID NO: 439 - protein, 7 aa, Artificial Sequence
  Description of Artificial Sequence Synthetic peptide
TSRMGVN

SEQ ID NO: 440 - protein, 5 aa, Artificial Sequence
  Description of Artificial Sequence Synthetic peptide
SHYIS

SEQ ID NO: 441 - protein, 7 aa, Artificial Sequence
  Description of Artificial Sequence Synthetic peptide
TSRMGVN

SEQ ID NO: 442 - protein, 7 aa, Artificial Sequence
  Description of Artificial Sequence Synthetic peptide
TSRMGVN

SEQ ID NO: 443 - protein, 7 aa, Artificial Sequence
  Description of Artificial Sequence Synthetic peptide
TSRMGVN

SEQ ID NO: 444 - protein, 7 aa, Artificial Sequence
  Description of Artificial Sequence Synthetic peptide
NVRMGVN

SEQ ID NO: 445 - protein, 5 aa, Artificial Sequence
  Description of Artificial Sequence Synthetic peptide
SSAIS

SEQ ID NO: 446 - protein, 5 aa, Artificial Sequence
  Description of Artificial Sequence Synthetic peptide
NHWMS

SEQ ID NO: 447 - protein, 5 aa, Artificial Sequence
  Description of Artificial Sequence Synthetic peptide
DHHMS

SEQ ID NO: 448 - protein, 5 aa, Artificial Sequence
  Description of Artificial Sequence Synthetic peptide
GYWMS

SEQ ID NO: 449 - protein, 5 aa, Artificial Sequence
  Description of Artificial Sequence Synthetic peptide
NHWMS

SEQ ID NO: 450 - protein, 4 aa, Artificial Sequence
  Description of Artificial Sequence Synthetic peptide
ATIS

SEQ ID NO: 451 - protein, 5 aa, Artificial Sequence
  Description of Artificial Sequence Synthetic peptide
SYWMH

SEQ ID NO: 452 - protein, 5 aa, Artificial Sequence
  Description of Artificial Sequence Synthetic peptide
HSHIH

SEQ ID NO: 453 - protein, 7 aa, Artificial Sequence
  Description of Artificial Sequence Synthetic peptide
DTRWGVN

SEQ ID NO: 454 - protein, 5 aa, Artificial Sequence
  Description of Artificial Sequence Synthetic peptide
SHYIS

SEQ ID NO: 455 - protein, 16 aa, Artificial Sequence
  Description of Artificial Sequence Synthetic peptide
YVSTTGTTDYNPSLKS

SEQ ID NO: 456 - protein, 16 aa, Artificial Sequence
  Description of Artificial Sequence Synthetic peptide
YINPSGRIDYNPSLKS

SEQ ID NO: 457 - protein, 17 aa, Artificial Sequence
  Description of Artificial Sequence Synthetic peptide
SISGSGDYTYYPDSVKG

SEQ ID NO: 458 - protein, 17 aa, Artificial Sequence
  Description of Artificial Sequence Synthetic peptide
SIKGDASDTYYPDSVKG

SEQ ID NO: 459 - protein, 17 aa, Artificial Sequence
  Description of Artificial Sequence Synthetic peptide
SISKSGSSIYYPDSVKG

SEQ ID NO: 460 - protein, 16 aa, Artificial Sequence
  Description of Artificial Sequence Synthetic peptide
YVSTTGTTDYNPSLKS

SEQ ID NO: 461 - protein, 16 aa, Artificial Sequence
  Description of Artificial Sequence Synthetic peptide
YGSVTGSTDYNPSLKS

SEQ ID NO: 462 - protein, 16 aa, Artificial Sequence
  Description of Artificial Sequence Synthetic peptide
YINPSGRTDYNPSLKS

SEQ ID NO: 463 - protein, 17 aa, Artificial Sequence
  Description of Artificial Sequence Synthetic peptide
SISKSGSSIYYPDSVKG

SEQ ID NO: 464 - protein, 17 aa, Artificial Sequence
  Description of Artificial Sequence Synthetic peptide
STTSGSSTIYYPDSVKG

SEQ ID NO: 465 - protein, 16 aa, Artificial Sequence
  Description of Artificial Sequence Synthetic peptide
YGSVTGSTDYNPSLKS

SEQ ID NO: 466 - protein, 16 aa, Artificial Sequence
  Description of Artificial Sequence Synthetic peptide
YVSTTGTTDYNPSLKS

SEQ ID NO: 467 - protein, 17 aa, Artificial Sequence
  Description of Artificial Sequence Synthetic peptide
SISGSGDYTYYPDSVKG

SEQ ID NO: 468 - protein, 16 aa, Artificial Sequence
  Description of Artificial Sequence Synthetic peptide
YINPSGRTDYNPSLKS

SEQ ID NO: 469 - protein, 16 aa, Artificial Sequence
  Description of Artificial Sequence Synthetic peptide
YITSSGRTVYNSALKS

SEQ ID NO: 470 - protein, 17 aa, Artificial Sequence
  Description of Artificial Sequence Synthetic peptide
WIDPKSGRTNYAQKLQG

SEQ ID NO: 471 - protein, 16 aa, Artificial Sequence
  Description of Artificial Sequence Synthetic peptide
YVSTTGTTDYNPSLKS

SEQ ID NO: 472 - protein, 16 aa, Artificial Sequence
  Description of Artificial Sequence Synthetic peptide
YITSSGRTVYNSALKS

SEQ ID NO: 473 - protein, 16 aa, Artificial Sequence
  Description of Artificial Sequence Synthetic peptide
YISTSGRTDYNPSLKS

SEQ ID NO: 474 - protein, 16 aa, Artificial Sequence
  Description of Artificial Sequence Synthetic peptide
YINPSGATDYNPSLKS

SEQ ID NO: 475 - protein, 17 aa, Artificial Sequence
  Description of Artificial Sequence Synthetic peptide
GMNPITGHTIYAQKLQG

SEQ ID NO: 476 - protein, 16 aa, Artificial Sequence
  Description of Artificial Sequence Synthetic peptide
YINPSGATDYNPSLKS

SEQ ID NO: 477 - protein, 16 aa, Artificial Sequence
  Description of Artificial Sequence Synthetic peptide
YINPSGATDYNPSLKS

SEQ ID NO: 478 - protein, 16 aa, Artificial Sequence
  Description of Artificial Sequence Synthetic peptide
YINPSGATDYNPSLKS

SEQ ID NO: 479 - protein, 16 aa, Artificial Sequence
  Description of Artificial Sequence Synthetic peptide
YVNPSGSTDYNPSLKS

SEQ ID NO: 480 - protein, 17 aa, Artificial Sequence
  Description of Artificial Sequence Synthetic peptide
GIYPGSGHTIYAQKFQG

SEQ ID NO: 481 - protein, 17 aa, Artificial Sequence
  Description of Artificial Sequence Synthetic peptide
SIGPKGLGTYYPDSVKG

SEQ ID NO: 482 - protein, 17 aa, Artificial Sequence
  Description of Artificial Sequence Synthetic peptide
SISPSGDFTYYPDSVKG

SEQ ID NO: 483 - protein, 16 aa, Artificial Sequence
  Description of Artificial Sequence Synthetic peptide
SIGTAGVTYYPDSVKG

SEQ ID NO: 484 - protein, 17 aa, Artificial Sequence
  Description of Artificial Sequence Synthetic peptide
SIGPKGLGTYYPDSVKG

SEQ ID NO: 485 - protein, 17 aa, Artificial Sequence
  Description of Artificial Sequence Synthetic peptide
WMNPKSGYTNYAQKFQG

SEQ ID NO: 486 - protein, 17 aa, Artificial Sequence
  Description of Artificial Sequence Synthetic peptide
NINPSNGGTNYNEKFKS

SEQ ID NO: 487 - protein, 17 aa, Artificial Sequence
  Description of Artificial Sequence Synthetic peptide
WINPRSGATNYAQKLQG

SEQ ID NO: 488 - protein, 16 aa, Artificial Sequence
  Description of Artificial Sequence Synthetic peptide
YINDRGTTDYNSALKS

SEQ ID NO: 489 - protein, 17 aa, Artificial Sequence
  Description of Artificial Sequence Synthetic peptide
GMNPITGHTIYAQKLQG

SEQ ID NO: 490 - protein, 6 aa, Artificial Sequence
  Description of Artificial Sequence Synthetic peptide
AGYPDV

SEQ ID NO: 491 - protein, 6 aa, Artificial Sequence
  Description of Artificial Sequence Synthetic peptide
AGYPDV

SEQ ID NO: 492 - protein, 16 aa, Artificial Sequence
  Description of Artificial Sequence Synthetic peptide
SPRGPRLLGYYYGMDV

SEQ ID NO: 493 - protein, 11 aa, Artificial Sequence
  Description of Artificial Sequence Synthetic peptide
DLSKQWLGIDY

SEQ ID NO: 494 - protein, 14 aa, Artificial Sequence
  Description of Artificial Sequence Synthetic peptide
LPRLAWNYYYGMDV

SEQ ID NO: 495 - protein, 6 aa, Artificial Sequence
  Description of Artificial Sequence Synthetic peptide
AGYPDV

SEQ ID NO: 496 - protein, 11 aa, Artificial Sequence
  Description of Artificial Sequence Synthetic peptide
SYLVDTAMVTR

SEQ ID NO: 497 - protein, 6 aa, Artificial Sequence
  Description of Artificial Sequence Synthetic peptide
AGYPDV

SEQ ID NO: 498 - protein, 14 aa, Artificial Sequence
  Description of Artificial Sequence Synthetic peptide
LPRLAWNYYYGMDV

SEQ ID NO: 499 - protein, 14 aa, Artificial Sequence
  Description of Artificial Sequence Synthetic peptide
GPDYYAPLRSYMDV

SEQ ID NO: 500 - protein, 11 aa, Artificial Sequence
  Description of Artificial Sequence Synthetic peptide
SYLVDTAMVTR

SEQ ID NO: 501 - protein, 6 aa, Artificial Sequence
  Description of Artificial Sequence Synthetic peptide
AGYPDV

SEQ ID NO: 502 - protein, 16 aa, Artificial Sequence
  Description of Artificial Sequence Synthetic peptide
SPRGPRLLGYYYGMDV

SEQ ID NO: 503 - protein, 6 aa, Artificial Sequence
  Description of Artificial Sequence Synthetic peptide
AGYPDV

SEQ ID NO: 504 - protein, 11 aa, Artificial Sequence
  Description of Artificial Sequence Synthetic peptide
TKIVGATLRDY

SEQ ID NO: 505 - protein, 17 aa, Artificial Sequence
  Description of Artificial Sequence Synthetic peptide
EVKQWLVPGRGVYWFDP

SEQ ID NO: 506 - protein, 6 aa, Artificial Sequence
  Description of Artificial Sequence Synthetic peptide
AGYPDV

SEQ ID NO: 507 - protein, 11 aa, Artificial Sequence
  Description of Artificial Sequence Synthetic peptide
TKIVGATLRDY

SEQ ID NO: 508 - protein, 6 aa, Artificial Sequence
  Description of Artificial Sequence Synthetic peptide
AGYPDV

SEQ ID NO: 509 - protein, 6 aa, Artificial Sequence
  Description of Artificial Sequence Synthetic peptide
AGYPDV

SEQ ID NO: 510 - protein, 18 aa, Artificial Sequence
  Description of Artificial Sequence Synthetic peptide
DMRYDILPGRSYYYGMDV

SEQ ID NO: 511 - protein, 6 aa, Artificial Sequence
  Description of Artificial Sequence Synthetic peptide
AGYPDV

SEQ ID NO: 512 - protein, 6 aa, Artificial Sequence
  Description of Artificial Sequence Synthetic peptide
AGYPDV

SEQ ID NO: 513 - protein, 6 aa, Artificial Sequence
  Description of Artificial Sequence Synthetic peptide
AGYPDV

SEQ ID NO: 514 - protein, 6 aa, Artificial Sequence
  Description of Artificial Sequence Synthetic peptide
AGYPDV

SEQ ID NO: 515 - protein, 9 aa, Artificial Sequence
  Description of Artificial Sequence Synthetic peptide
EGDIFVMDV

SEQ ID NO: 516 - protein, 15 aa, Artificial Sequence
  Description of Artificial Sequence Synthetic peptide
IRDYGDKKGGSWFDP

SEQ ID NO: 517 - protein, 7 aa, Artificial Sequence
  Description of Artificial Sequence Synthetic peptide
DVSPVYY

SEQ ID NO: 518 - protein, 13 aa, Artificial Sequence
  Description of Artificial Sequence Synthetic peptide
GPHGPQQRLRFDP

SEQ ID NO: 519 - protein, 16 aa, Artificial Sequence
  Description of Artificial Sequence Synthetic peptide
DVRGWRALYYYYGMDV

SEQ ID NO: 520 - protein, 10 aa, Artificial Sequence
  Description of Artificial Sequence Synthetic peptide
LHGSGLAFDI

SEQ ID NO: 521 - protein, 11 aa, Artificial Sequence
  Description of Artificial Sequence Synthetic peptide
TTVVAYWYFDV

SEQ ID NO: 522 - protein, 14 aa, Artificial Sequence
  Description of Artificial Sequence Synthetic peptide
DRGGRTNGVGAFDI

SEQ ID NO: 523 - protein, 12 aa, Artificial Sequence
  Description of Artificial Sequence Synthetic peptide
TLHTREFVGMDV

SEQ ID NO: 524 - protein, 18 aa, Artificial Sequence
  Description of Artificial Sequence Synthetic peptide
DMRYDILPGRSYYYGMDV

SEQ ID NO: 525 - protein, 11 aa, Artificial Sequence
  Description of Artificial Sequence Synthetic peptide
RASQNIGAALA

SEQ ID NO: 526 - protein, 11 aa, Artificial Sequence
  Description of Artificial Sequence Synthetic peptide
RASQTVSSHLA

SEQ ID NO: 527 - protein, 16 aa, Artificial Sequence
  Description of Artificial Sequence Synthetic peptide
RSSRSLVHGSGHNYLH

SEQ ID NO: 528 - protein, 16 aa, Artificial Sequence
  Description of Artificial Sequence Synthetic peptide
RSSQSLLHSNEKNYLH

SEQ ID NO: 529 - protein, 16 aa, Artificial Sequence
  Description of Artificial Sequence Synthetic peptide
RSSRSLVHGSGDNYLH

SEQ ID NO: 530 - protein, 11 aa, Artificial Sequence
  Description of Artificial Sequence Synthetic peptide
RASQNVYTNLA

SEQ ID NO: 531 - protein, 11 aa, Artificial Sequence
  Description of Artificial Sequence Synthetic peptide
RASQSLGTNLA

SEQ ID NO: 532 - protein, 11 aa, Artificial Sequence
  Description of Artificial Sequence Synthetic peptide
RASQTISRSLA

SEQ ID NO: 533 - protein, 16 aa, Artificial Sequence
  Description of Artificial Sequence Synthetic peptide
RSSQSLLHSNEKNYLH

SEQ ID NO: 534 - protein, 16 aa, Artificial Sequence
  Description of Artificial Sequence Synthetic peptide
RSSQSLQQSEGRTYLH

SEQ ID NO: 535 - protein, 11 aa, Artificial Sequence
  Description of Artificial Sequence Synthetic peptide
RASQRIGSDLA

SEQ ID NO: 536 - protein, 11 aa, Artificial Sequence
  Description of Artificial Sequence Synthetic peptide
RASQGISRDLA

SEQ ID NO: 537 - protein, 16 aa, Artificial Sequence
  Description of Artificial Sequence Synthetic peptide
RSSQSLLHSNEKNYLH

SEQ ID NO: 538 - protein, 11 aa, Artificial Sequence
  Description of Artificial Sequence Synthetic peptide
RASQSIGTKLA

SEQ ID NO: 539 - protein, 17 aa, Artificial Sequence
  Description of Artificial Sequence Synthetic peptide
KSSQSVLDTSTNRNYLA

SEQ ID NO: 540 - protein, 11 aa, Artificial Sequence
  Description of Artificial Sequence Synthetic peptide
TGTNIGLRSVS

SEQ ID NO: 541 - protein, 11 aa, Artificial Sequence
  Description of Artificial Sequence Synthetic peptide
RASESVRSNLA

SEQ ID NO: 542 - protein, 11 aa, Artificial Sequence
  Description of Artificial Sequence Synthetic peptide
RASQGISNDLA

SEQ ID NO: 543 - protein, 11 aa, Artificial Sequence
  Description of Artificial Sequence Synthetic peptide
RASQGIGIDLA

SEQ ID NO: 544 - protein, 11 aa, Artificial Sequence
  Description of Artificial Sequence Synthetic peptide
RASQSVSTHLA

SEQ ID NO: 545 - protein, 11 aa, Artificial Sequence
  Description of Artificial Sequence Synthetic peptide
TGTDIRGMSVS

SEQ ID NO: 546 - protein, 11 aa, Artificial Sequence
  Description of Artificial Sequence Synthetic peptide
RASQNIGHSLA

SEQ ID NO: 547 - protein, 11 aa, Artificial Sequence
  Description of Artificial Sequence Synthetic peptide
RASRSVSSDLA

SEQ ID NO: 548 - protein, 11 aa, Artificial Sequence
  Description of Artificial Sequence Synthetic peptide
RASQSVRTSLA

SEQ ID NO: 549 - protein, 12 aa, Artificial Sequence
  Description of Artificial Sequence Synthetic peptide
RASQSISSSQLA

SEQ ID NO: 550 - protein, 11 aa, Artificial Sequence
  Description of Artificial Sequence Synthetic peptide
RASMDIRNDLA

SEQ ID NO: 551 - protein, 16 aa, Artificial Sequence
  Description of Artificial Sequence Synthetic peptide
RSSQSLLHSSGYNFLH

SEQ ID NO: 552 - protein, 8 aa, Artificial Sequence
  Description of Artificial Sequence Synthetic peptide
RSSQSLLH

SEQ ID NO: 553 - protein, 16 aa, Artificial Sequence
  Description of Artificial Sequence Synthetic peptide
RSSQSLLSSYGYHNLH

SEQ ID NO: 554 - protein, 16 aa, Artificial Sequence
  Description of Artificial Sequence Synthetic peptide
RSSQSLLHVTGYNYLH

SEQ ID NO: 555 - protein, 11 aa, Artificial Sequence
  Description of Artificial Sequence Synthetic peptide
RASQDIKSDLA

SEQ ID NO: 556 - protein, 10 aa, Artificial Sequence
  Description of Artificial Sequence Synthetic peptide
SASSSVSYMY

SEQ ID NO: 557 - protein, 14 aa, Artificial Sequence
  Description of Artificial Sequence Synthetic peptide
TGTSGDIGRYNYVS

SEQ ID NO: 558 - protein, 17 aa, Artificial Sequence
  Description of Artificial Sequence Synthetic peptide
KSSQSVLDTSTNRNYLA

SEQ ID NO: 559 - protein, 13 aa, Artificial Sequence
  Description of Artificial Sequence Synthetic peptide
TGTTGSIASNYVS

SEQ ID NO: 560 - protein, 7 aa, Artificial Sequence
  Description of Artificial Sequence Synthetic peptide
GASTREY

SEQ ID NO: 561 - protein, 7 aa, Artificial Sequence
  Description of Artificial Sequence Synthetic peptide
DVSHRAS

SEQ ID NO: 562 - protein, 7 aa, Artificial Sequence
  Description of Artificial Sequence Synthetic peptide
GASTRVA

SEQ ID NO: 563 - protein, 7 aa, Artificial Sequence
  Description of Artificial Sequence Synthetic peptide
DASKRAS

SEQ ID NO: 564 - protein, 7 aa, Artificial Sequence
  Description of Artificial Sequence Synthetic peptide
MASNRAP

SEQ ID NO: 565 - protein, 7 aa, Artificial Sequence
  Description of Artificial Sequence Synthetic peptide
GASARAS

SEQ ID NO: 566 - protein, 7 aa, Artificial Sequence
  Description of Artificial Sequence Synthetic peptide
GASSRPS

SEQ ID NO: 567 - protein, 7 aa, Artificial Sequence
  Description of Artificial Sequence Synthetic peptide
DASSLAG

SEQ ID NO: 568 - protein, 7 aa, Artificial Sequence
  Description of Artificial Sequence Synthetic peptide
DASKRAS

SEQ ID NO: 569 - protein, 7 aa, Artificial Sequence
  Description of Artificial Sequence Synthetic peptide
DASKRAS

SEQ ID NO: 570 - protein, 7 aa, Artificial Sequence
  Description of Artificial Sequence Synthetic peptide
SASSRAP

SEQ ID NO: 571 - protein, 7 aa, Artificial Sequence
  Description of Artificial Sequence Synthetic peptide
AASTLVS

SEQ ID NO: 572 - protein, 7 aa, Artificial Sequence
  Description of Artificial Sequence Synthetic peptide
DASKRAS

SEQ ID NO: 573 - protein, 7 aa, Artificial Sequence
  Description of Artificial Sequence Synthetic peptide
DASSRVP

SEQ ID NO: 574 - protein, 7 aa, Artificial Sequence
  Description of Artificial Sequence Synthetic peptide
GASIRAG

SEQ ID NO: 575 - protein, 7 aa, Artificial Sequence
  Description of Artificial Sequence Synthetic peptide
DTNIRSS

SEQ ID NO: 576 - protein, 7 aa, Artificial Sequence
  Description of Artificial Sequence Synthetic peptide
GASNRAS